(19) United States
(12) Patent Application Publication
(10) Pub. No.: US 2026/0250362 A1
(43) Pub. Date: Aug. 27, 2026

(54) ANTIBODIES AGAINST ORTHOHANTAVIRUSES

(71) Applicants: Clas AHLM, Umeå (SE); Mattias FORSELL, Umeå (SE); MAPP BIOPHARMACEUTICAL, INC., San Diego, CA (US); ALBERT EINSTEIN COLLEGE OF MEDICINE, Bronx, NY (US); ADIMAB, LLC, Lebanon, NH (US); INSTITUT PASTEUR, Paris (FR); UNIVERSITY OF NEW MEXICO, Albuquerque, NM (US); THE GOVERNMENT OF THE UNITED STATES, AS REPRESENTED BY THE SECRETARY OF THE ARMY, Frederick, MD (US)

(72) Inventors: Clas AHLM, Umeå (SE); Mattias FORSELL, Umeå (SE); Laura M. WALKER, Lebanon, NH (US); Anna Z. WEC, Lebanon, NH (US); Pablo GUARDADO-CALVO, Paris (FR); Felix A. REY, Paris (FR); Eva MITTLER, Bronx, NY (US); Kartik CHANDRAN, Bronx, NY (US); Steven B. BRADFUTE, Albuquerque, NM (US); Dafna M. ABELSON, San Diego, CA (US); Andrew S. Herbert, Frederick, MD (US); John DYE, Frederick, MD (US)

(21) Appl. No.: 19/165,540

(22) PCT Filed: Mar. 14, 2024
(86) PCT No.: PCT/US2024/019994

§ 371 Date: Sep. 15, 2025

Related U.S. Application Data
(60) Provisional application No. 63/490,215, filed on Mar. 14, 2023.

(51) Int. Cl. C07K 16/10; A61K 39/00; A61P 31/14

(52) U.S. Cl. CPC C07K 16/10; A61P 31/14; A61K 2039/505; C07K 2317/10

(57) ABSTRACT

Provided herein are hantivirus antibodies. These hantivirus antibodies bind to the Gn and/or Gc subunits of a hantavirus glycoprotein and have broad neutralizing activity against an epitope of different hantavirus species. Such antibodies are used in methods of inducing an immune response and methods of inhibiting hantavirus infection. Additionally provided are methods of treating an infectious disease using such antibodies.

### CROSS-REFERENCE TO RELATED APPLICATIONS

This application claims the benefit of U.S. Provisional Patent Application No. 63/490,215 filed Mar. 14, 2023. The contents this provisional application are incorporated by reference in their entirety.

### STATEMENT REGARDING FEDERALLY SPONSORED RESEARCH

This invention was made with Government support under grant U19AI142777, awarded by the National Institutes of Health. The government has certain rights to this invention.

### SEQUENCE LISTING INCORPORATED BY REFERENCE

This application contains an ST.26 compliant Sequence Listing, which is submitted concurrently in .xml format via Patent Center and is hereby incorporated by reference in its entirety. The .xml copy, created on Mar. 12, 2024, is named 1298078031WO00.xml and is 99,000 bytes in size.

### BACKGROUND

Rodent-borne orthohantaviruses (hantaviruses) are a large, globally distributed group of enveloped viruses with segmented negative-strand RNA genomes. Hantavirus infections are associated with over 50,000 annual diagnosed cases worldwide, primarily caused by rodent-to-human transmission and direct human-to-human contact. The ‘New World’ hantaviruses Andes virus (ANDV) and Sin Nombre virus (SNV) are the primary etiologic agents of hantavirus cardiopulmonary syndrome (HCPS) in South and North America, respectively. Case-fatality rates for both viruses are the highest among hantaviruses, reaching ~40%, with cardiogenic shock and respiratory failure being the primary causes of death. The ‘Old World’ hantaviruses Puumala virus (PUUV) and related agents, including Dobrava-Belgrade and Hantaan viruses, are endemic in different regions of Europe and Asia or worldwide (Seoul virus) and cause hemorrhagic fever with renal syndrome (HFRS) with case-fatality rates of up to ~15%. A third of PUUV-infected individuals require hospitalization, and 5% require intensive-care stays due to shock, hemorrhage, and cardiopulmonary complications. Unexpectedly large PUUV outbreaks in Sweden, Finland, and Germany between 2007-2021 have had significant socioeconomic impacts in endemic regions where the incidence may exceed 50-300 cases per 100,000 residents annually. An SNV outbreak among overnight visitors in California's Yosemite National Park in 2012 and a singular ANDV super-spreader outbreak in Argentina in 2018-19 highlights the public health risks posed by hantaviruses. Compounding these concerns is the unknown potential for the emergence of novel hantaviruses into human populations from rodent or non-rodent reservoirs; the likelihood of such zoonotic events with unpredictable disease and epidemic potential is expected to grow in the coming decades in concert with habitat loss and climate change. The lack of FDA-approved or emergency use-authorized vaccines and therapeutics represents a critical gap in preparedness for a public health emergency caused by a large hantavirus outbreak and further highlights the need to pre-position broad anti-hantavirus countermeasures for human use.

### SUMMARY

The present technology comprises antibodies that bind to epitopes of Orthohantavirus (hantavirus). In some embodiments, the antibodies are broad neutralizing antibodies capable of binding to one or more epitopes located in an N-terminal (Gn) subunit and/or a C-terminal (Gc) subunit of spike proteins (Gn/Gc spikes) of at least two hantavirus species. The broad neutralizing antibodies of the present technology may target a hantavirus in a variety of ways, for example, it may reduce infection by blocking viral entry into a cell. In some embodiments, these antibodies block Gn/Gc dissociation prior to viral membrane fusion, Gc target membrane insertion, and/or post-fusion homotorizerization. The blocking does not interfere with the attachment of a virion to a target cell. The antibodies may target both Old Word (e.g., Puumala virus (PUUV)) and New World (e.g., Andes virus (ANDV) and Sin Nombre virus (SNV)) clades. In some embodiments, the antibodies protect against divergent hantaviruses and for clinical development in treating, preventing, or otherwise ameliorating hantavirus infections such as cardiopulmonary syndrome (HCPS) and hemorrhagic fever with renal syndrome (HFRS).

In some embodiments, the present technology comprises an isolated antibody that binds to a spike protein of a hantavirus.

In some embodiments, the isolated antibody comprises a heavy chain having a variable heavy (VH) region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 1-5, 68, or 69.

In some embodiments, the heavy chain comprises one or more complementarity determining regions (CDRs) (HCDRs) comprising or consisting of an amino acid sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NOS: 6-20 or 72-77.

In some embodiments, the isolated antibody comprises a light chain having a variable light (VL) region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 21-25, 70, or 81.

In some embodiments, the light chain comprises one or more CDRs (LCDRs) comprising or consisting of an amino acid sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NOS: 35-40 or 78-80.

In some embodiments, the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 6, 11, and 16.

In some embodiments, the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 7, 12, and 17.

In some embodiments, the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 8, 13, and 18.

In some embodiments, the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 9, 14, and 19.

In some embodiments, the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 10, 15, and 20.

In some embodiments, the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 72, 73, and 74.

In some embodiments, the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 75, 76, and 77.

In some embodiments, the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 26, 31, and 36.

In some embodiments, the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 82, 31, and 36.

In some embodiments, the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 27, 32, and 37.

In some embodiments, the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 28, 33, and 38.

In some embodiments, the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 29, 34, and 39.

In some embodiments, the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 30, 35, and 40.

In some embodiments, the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 78, 79, and 80.

In some embodiments, the isolated antibody binds to an epitope located in an N-terminal (Gn) subunit (SEQ ID NO: 42) and/or a C-terminal (Gc) subunit (SEQ ID NO: 43) of the spike protein.

In some embodiments, the antibody binds to an epitope located in the capping loop of Gn (SEQ ID NO: 41).

In some embodiments, the antibody binds to a quaternary epitope encompassing both the Gn subunit and the Gc subunit.

In some embodiments, the epitope comprises one or more of E95, Q98 or S101 in the Gn subunit.

In some embodiments, the epitope comprises one or more of D682, S830, 1844, P852, D959, or S966 in the Gc subunit.

In some embodiments, the antibody does not bind to Gn in the absence of Gc.

In some embodiments, the epitope comprises one or both of E88 and S101 of the Gn subunit.

In some embodiments, the isolated antibody binds to the spike protein of a hantavirus or an epitope of the hantavirus in an environment with an acidic pH.

In some embodiments, the isolated antibody neutralizes one or more hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV).

In some embodiments, the isolated antibody comprises:
a HCDR comprising:
   an HCDR1 comprising a sequence of any one of SEQ ID NOS: 6-10, 72, 75 or a variant HCDR1 in which 1, 2, 3, 4, or 5 amino acids are substituted relative to the sequence;
   an HCDR2 comprising a sequence of any one of SEQ ID NOS: 11-15, 73, 76 or a variant HCDR2 in which 1, 2, 3, 4, or 5 amino acids are substituted relative to the sequence; and
   an HCDR3 comprising a sequence of any one of SEQ ID NOS: 16-20, 74, 77, or a variant HCDR3 in which 1, 2, 3, 4, or 5 amino acids are substituted relative to the sequence; and
a LCDR comprising:
   an LCDR1 comprising a sequence of any one of SEQ ID NOS: 82, 26-30, 78, or a variant LCDR1 in which 1, 2, 3, 4, or 5 amino acids are substituted relative to the sequence;
   an LCDR2 comprising a sequence of any one of SEQ ID NOS: 31-35, 79 or a variant LCDR2 in which 1, 2, 3, 4, or 5 amino acids are substituted relative to the sequence; and
   an LCDR3 comprising a sequence of any one of SEQ ID NOS: 36-40, 80 or a variant LCDR3 in which 1, 2, 3, 4, or 5 amino acids are substituted relative to the sequence.

In some embodiments, the isolated antibody comprises:
a HCDR comprising:
   an HCDR1 comprising a sequence of SEQ ID NOS: 72 or a variant HCDR1 in which 1, 2, 3, 4, or 5 amino acids are substituted relative to the sequence;
   an HCDR2 comprising a sequence of SEQ ID NOS: 73 or a variant HCDR2 in which 1, 2, 3, 4, or 5 amino acids are substituted relative to the sequence; and
   an HCDR3 comprising a sequence of SEQ ID NOS: 74, or a variant HCDR3 in which 1, 2, 3, 4, or 5 amino acids are substituted relative to the sequence; and
a LCDR comprising:
   an LCDR1 comprising a sequence of SEQ ID NOS: 78, or a variant LCDR1 in which 1, 2, 3, 4, or5 amino acids are substituted relative to the sequence;
   an LCDR2 comprising a sequence of SEQ ID NOS: 79, or a variant LCDR2 in which 1, 2, 3, 4, or 5 amino acids are substituted relative to the sequence; and
   an LCDR3 comprising a sequence of SEQ ID NOS: 80, or a variant LCDR3 in which 1, 2, 3, 4, or 5 amino acids are substituted relative to the sequence.

In some embodiments, the isolated antibody comprises:
a heavy chain complementarity-determining region (HCDR) comprising:
   an HCDR1 comprising a sequence of SEQ ID NOS: 75 or a variant HCDR1 in which 1, 2, 3, 4, or 5 amino acids are substituted relative to the sequence;
   an HCDR2 comprising a sequence of SEQ ID NOS: 76 or a variant HCDR2 in which 1, 2, 3, 4, or 5 amino acids are substituted relative to the sequence; and
   an HCDR3 comprising a sequence of SEQ ID NOS: 77, or a variant HCDR3 in which 1, 2, 3, 4, or 5 amino acids are substituted relative to the sequence; and
a light chain complementarity-determining region (LCDR) comprising:
   an LCDR1 comprising a sequence of SEQ ID NOS: 78, or a variant LCDR1 in which 1, 2, 3, 4, or 5 amino acids are substituted relative to the sequence;
   an LCDR2 comprising a sequence of SEQ ID NOS: 79, or a variant LCDR2 in which 1, 2, 3, 4, or 5 amino acids are substituted relative to the sequence; and
   an LCDR3 comprising a sequence of SEQ ID NOS: 80, or a variant LCDR3 in which 1, 2, 3, 4, or 5 amino acids are substituted relative to the sequence.

In some embodiments, the V_{H} comprises an amino acid sequence having at least 95% identity to a V_{H} of any one of SEQ ID NOS: 1-5, and the V_{L} comprises an amino sequence having at 95% identity to a V_{L} of any one of SEQ ID NOS: 21-25; wherein the V_{H} preferably comprises an amino acid sequence having at least 95% identity to a V_{H} of SEQ ID NO: 1, and the V_{L} preferably comprises an amino sequence having at 95% identity to a V_{L} of SEQ ID NO: 21.

In some embodiments, the V_{H} comprises an amino acid sequence having at least 95% identity to a V_{H} of SEQ ID NOS: 68 or 69 and the V_{L} comprises an amino sequence having at 95% identity to a V_{L} of SEQ ID NOS: 70.

In some embodiments, the antibody comprises:
a V_{H} region comprising amino acid sequence SEQ ID NO: 1 and a V_{L} region comprising amino acid sequence SEQ ID NO: 21;
a V_{H} region comprising amino acid sequence SEQ ID NO: 2 and a V_{L} region comprising amino acid sequence SEQ ID NO: 22;
a V_{H} region comprising amino acid sequence SEQ ID NO: 3 and a V_{L} region comprising amino acid sequence SEQ ID NO: 23;
a V_{H} region comprising amino acid sequence SEQ ID NO: 4 and a V_{L} region comprising amino acid sequence SEQ ID NO: 24;
a V_{H} region comprising amino acid sequence SEQ ID NO: 5 and a V_{L} region comprising amino acid sequence SEQ ID NO: 25;
a V_{H} region comprising amino acid sequence SEQ ID NO: 68 and a V_{L} region comprising amino acid sequence SEQ ID NO: 70; or
a V_{H} region comprising amino acid sequence SEQ ID NO: 69 and a V_{L} region comprising amino acid sequence SEQ ID NO: 70.

In some embodiments, the antibody comprises:
a heavy chain variable (V_{H}) region and a light chain variable (V_{L}) region, wherein:
(a) the V_{H} region has at least 70% identity to SEQ ID NO: 1; and comprises a CDR1 of SEQ ID NO:6, or the CDR1 of SEQ ID NO: 6 in which 1, 2, 3, 4, or 5 amino acids are substituted; a CDR2 of SEQ ID NO: 11, or the CDR2 of SEQ ID NO: 11 in which 1, 2, 3, 4, or 5 amino acids are substituted; a CDR3 of SEQ ID NO: 16 or the CDR3 of SEQ ID NO: 16 in which 1, 2, 3, 4, or 5 are substituted; and
(b) the V_{L} region has at least 70% identity to SEQ ID NO: 21, and comprises a CDR1 of SEQ ID NO: 82 or 26 or the CDR1 of SEQ ID NO: 82 or 26 in which 1, 2, 3, 4, or 5 amino acids are substituted; a CDR2 of SEQ ID NO: 31, or the CDR2 of SEQ ID NO: 31 in which 1, 2, 3, 4, or 5 amino acids are substituted; a CDR3 of SEQ ID NO: 36 or the CDR3 of SEQ ID NO: 36 in which 1, 2, 3, 4, or 5 are substituted.

In some embodiments, the antibody comprises:
a heavy chain variable (V_{H}) region and a light chain variable (V_{L}) region, wherein:
(a) the V_{H} region has at least 70% identity to SEQ ID NO: 1; and comprises a CDR1 of SEQ ID NO:6, or the CDR1 of SEQ ID NO: 6 in which 1, 2, 3, 4, or 5 amino acids are substituted; a CDR2 of SEQ ID NO: 11, or the CDR2 of SEQ ID NO: 11 in which 1, 2, 3, 4, or 5 amino acids are substituted; a CDR3 of SEQ ID NO: 16 or the CDR3 of SEQ ID NO: 16 in which 1, 2, 3, 4, or 5 are substituted; and
(b) the V_{L} region has at least 70% identity to SEQ ID NO: 21, and comprises a CDR1 of SEQ ID NO: 82 or 26 or the CDR1 of SEQ ID NO: 82 or 26 in which 1, 2, 3, 4, or 5 amino acids are substituted; a CDR2 of SEQ ID NO: 31, or the CDR2 of SEQ ID NO: 31 in which 1, 2, 3, 4, or 5 amino acids are substituted; a CDR3 of SEQ ID NO: 36 or the CDR3 of SEQ ID NO: 36 in which 1, 2, 3, 4, or 5 are substituted.

In some embodiments, the antibody comprises a heavy chain variable (V_{H}) region and a light chain variable (V_{L}) region, wherein:
(a) the V_{H} region has at least 70% identity to SEQ ID NO: 68; and comprises a CDR1 of SEQ ID NO:72, or the CDR1 of SEQ ID NO: 72 in which 1, 2, 3, 4, or 5 amino acids are substituted; a CDR2 of SEQ ID NO: 73, or the CDR2 of SEQ ID NO: 73 in which 1, 2, 3, 4, or 5 amino acids are substituted; a CDR3 of SEQ ID NO: 74 or the CDR3 of SEQ ID NO: 74 in which 1, 2, 3, 4, or 5 are substituted; and
(b) the V_{L} region has at least 70% identity to SEQ ID NO: 70, and comprises a CDR1 of SEQ ID NO: 78 or the CDR1 of SEQ ID NO: 78 in which 1, 2, 3, 4, or 5 amino acids are substituted; a CDR2 of SEQ ID NO: 79, or the CDR2 of SEQ ID NO: 79 in which 1, 2, 3, 4, or 5 amino acids are substituted; a CDR3 of SEQ ID NO: 80 or the CDR3 of SEQ ID NO: 80 in which 1, 2, 3, 4, or 5 are substituted.

In some embodiments, the antibody comprises:
a heavy chain variable (V_{H}) region and a light chain variable (V_{L}) region, wherein:
(a) the V_{H} region has at least 70% identity to SEQ ID NO: 69; and comprises a CDR1 of SEQ ID NO:75, or the CDR1 of SEQ ID NO: 75 in which 1, 2, 3, 4, or 5 amino acids are substituted; a CDR2 of SEQ ID NO: 76, or the CDR2 of SEQ ID NO: 76 in which 1, 2, 3, 4, or 5 amino acids are substituted; a CDR3 of SEQ ID NO: 77 or the CDR3 of SEQ ID NO: 77 in which 1, 2, 3, 4, or 5 are substituted; and
(b) the V_{L} region has at least 70% identity to SEQ ID NO: 70, and comprises a CDR1 of SEQ ID NO: 78 or the CDR1 of SEQ ID NO: 78 in which 1, 2, 3, 4, or 5 amino acids are substituted; a CDR2 of SEQ ID NO: 79, or the CDR2 of SEQ ID NO: 79 in which 1, 2, 3, 4, or 5 amino acids are substituted; a CDR3 of SEQ ID NO: 80 or the CDR3 of SEQ ID NO: 80 in which 1, 2, 3, 4, or 5 are substituted.

In some embodiments, the antibody comprises an HCDR1, HCDR2, HCDR3, LCDR1, LCDR2, and LCDR3 of an antibody designated as ADI-42898, ADI-42877, ADI-42098, ADI-42885, ADI-37236, ADI-65533, ADI-65534 or a variant thereof in which at least one, two, three, four, five, or all six of the CDRs contain 1 or 2 amino acid substitutions compared to the corresponding CDR.

In some embodiments, the antibody comprises a heavy chain variable region and a light chain variable region of an antibody designated as ADI-42898, ADI-42877, ADI-42098, ADI-42885, ADI-37236, ADI-65533, ADI-65534 or a variant thereof, wherein the variant comprises a heavy chain variable region having a sequence that is at least 95% identical to that of the corresponding heavy chain variable region and a light chain variable region having a sequence that is at least 95% identical to the corresponding light chain variable region.

In some embodiments, at least 1 or 2 of the substitutions are conservative substitutions; at least 50% of the substitutions are conservative substitutions; or all of the substitutions are conservative substitutions.

In some embodiments, the antibody is a bispecific antibody or a multispecific antibody.

In some embodiments, the immunoglobulin (Ig) class of the isolated antibody is IgG or IgM.

In some embodiments, the Fc subclass of the isolated antibody is selected from the group consisting of hIgG1, hlgG2, and hlgG4.

In some embodiments, the present technology comprises a recombinant nucleic acid encoding an antibody of the present technology.

In some embodiments, the present technology comprises an expression vector comprising a polynucleotide encoding the V_{H} region and/or the V_{L} region of an antibody of the present technology.

In some embodiments, the present technology comprises a host cell comprising an expression vector of the present technology.

In some embodiments, the present technology comprises a polypeptide comprising
(1) a V_{H} sequence having at least 70% amino acid sequence identity to SEQ ID NO:1 and/or
(2) a V_{L} sequence having at least 70% amino acid sequence identity to SEQ ID NO:21.

In some embodiments, the present technology comprises a polypeptide comprising
(1) a V_{H} sequence having at least 70% amino acid sequence identity to SEQ ID NO:68 and/or
(2) a V_{L} sequence having at least 70% amino acid sequence identity to SEQ ID NO:70.

In some embodiments, the present technology comprises a polypeptide comprising
(1) a V_{H} sequence having at least 70% amino acid sequence identity to SEQ ID NO:69 and/or
(2) a V_{L} sequence having at least 70% amino acid sequence identity to SEQ ID NO:70.

In some embodiments, the present technology comprises a pharmaceutical composition comprising an isolated antibody of the present technology and a pharmaceutically acceptable carrier.

In some embodiments, the pharmaceutical composition comprises two or more different antibodies.

In some embodiments, the present technology comprises a method of inducing an immune response and/or treating a hantavirus infectious disease, the method comprising administering an effective amount of a pharmaceutical composition of the present technology to a subject in need thereof.

In some embodiments, the infectious disease is selected from hantavirus cardiopulmonary syndrome (HCPS) and hemorrhagic fever with renal syndrome (HFRS).

### BRIEF DESCRIPTION OF THE DRAWINGS

FIGS. 1A-1E show isolation and characterization of anti-Gn/Gc mAbs from PUUV-experienced donors in accordance with the present technology. FIG. 1A is a phylogenetic tree of the M segment-encoded GPC sequences from Old World and New World hantaviruses (OWH/NWH) based on amino acid (aa) sequences (OWH aa sequences: HTNV, SEOV, DOBV, PUUV, TULV, PHV; NWH aa sequences: LGNW, ANDV, MPRLV, CHOV, BCCV, BAYV, SNV). ANDV, Andes virus; BAYV, Bayou virus; BCCV, Black Creek Canal virus; CHOV, Choclo virus; DOBV, Dobrava-Belgrade virus; HTNV, Hantaan virus; LGNV, Laguna Negra virus; MPRLV, Maporal virus; PHV, Prospect Hill virus; PUUV, Puumala virus; SEOV, Seoul virus; SNV, Sin Nombre virus; TULV, Tula virus. Scale bar represents 0.07 aa substitutions per site. FIG. 1B shows frequency of hantavirus Gn/Gc-reactive B cells isolated with rVSV-mNG particles bearing PUUV or ANDV Gn/Gc from PUUV-naïve and PUUV-experienced donors. Fluorescence-activated cell sorting plots are gated on CD19⁺/CD20⁺/IgD⁻/IgM⁻/virus⁺ B cells. FIG. 1C shows somatic hypermutation (SHM) load as determined by the number of V_{H} and V_{L} nucleotide substitutions away from the predicted V_{H} and V_{L} germline. Bar indicates median. FIG. 1D shows binding reactivity of 180 isolated mAbs (25 nM) to rVSV-PUUV-Gn/Gc, as determined by ELISA. A₄₅₀, absorbance at 450 nm. The dashed line indicates the threshold for designating binders (A₄₅₀=0.3). Bar indicates median. Averages for PUUV GnGc binders (n=4) from two experiments. FIG. 1E shows binding cross-reactivity of 135 isolated mAbs (25 nM) to rVSVs bearing Gn/Gc from the indicated viruses, as determined by ELISA. The dashed line indicates the threshold for designating binders (A₄₅₀=0.3). ANDV, SNV: averages (n=2) from one experiment. Other viruses, averages (n=4) from two experiments.

FIGS. 2A-2D show neutralization activity of PUUV Gn/Gc-reactive monoclonal antibodies (mAbs) in accordance with the present technology. FIG. 2A shows the binding activity of mAbs (25 nM) to rVSV-PUUV-Gn/Gc, as determined by ELISA (see FIG. 1D), plotted against IC₅₀ values calculated from rVSV-PUUV-Gn/Gc neutralization curves displayed as averages (n≥4) from at least two experiments. Data points are shaded and/or patterned according to neutralization activity. Antibodies with IC₅₀ values >100 nM are designated non-neutralizers (N.N.). Dashed lines indicate the threshold for PUUV Gn/Gc binders (A₄₅₀=0.3; x-axis) and potent neutralizers (IC₅₀=1 nM; y-axis). FIG. 2B shows the frequency of mAbs grouped according to their neutralization activity. The number in the center of the pie chart denotes the total number of mAbs analyzed. FIG. 2C shows the frequency of prioritized mAbs grouped according to their neutralization activity towards rVSVs bearing divergent OWH and NWH Gn/Gc. The number in the pie chart's center indicates the total number of mAbs analyzed. Each group is represented as a segment proportional to the group size. FIG. 2D shows the neutralizing activity of 50 prioritized mAbs (IC₅₀ values) against the indicated rVSVs. mAbs with IC₅₀>10 nM are designated non-neutralizers (N.N.), and mAbs leaving an un-neutralized virus fraction are depicted as striped squares. An un-neutralized virus fraction is a residual normalized virus infection of >5% at the highest mAb concentration tested—averages of IC₅₀ values (n≥6) from at least two experiments.

FIGS. 3A-3C depict epitope binning of nAbs by competition analysis in accordance with the present technology. FIG. 3A is a heatmap of competitive pairwise mAb binding studies to rVSV-PUUV-Gn/Gc. Percent mAb competition by the second mAb (columns) are normalized to the binding ELISA value for un-competed binding by the first mAb (rows). Averages (n=2-4) from one to two experiments. FIG. 3B shows proportion of mAbs competing with ADI-42898 or ADI-42098 was determined in a high-throughput yeast competition assay by binding of IgG-displaying yeast cells to soluble PUUV Gn^{H}/Gc pre-complexed with F(ab)s of ADI-42898 or ADI-42098. Number in the center of the pie chart denotes the total number of mAbs analyzed. Each group (I-IV) is represented as a segment proportional to the group size. FIG. 3C is a heatmap showing the capacity of selected mAbs to compete with ADI-42898 or ADI-42098 for binding to PUUV Gn^{H}/Gc from the experiments in panel B. mAbs marked in bold were tested in both competition-binding studies.

FIGS. 4A-4F depict epitope mapping of nAbs by escape mutant analysis in accordance with the present technology. FIG. 4A is a schematic representation of the organization of lattices of Gn/Gc tetramers in viral particles (top, en face view; bottom, side view).The approximate position of the viral membrane is indicated with a black line. FIG. 4B is a ribbon representation of the ANDV Gn^{H}/Gc pre-fusion dimer (modeled from its monomer, PDB: 6Y5W;) with the two-fold molecular axis of the dimeric spike indicated (“2”). Residues exchanged in the neutralization-escape mutants are shown as Gn or Gc spheres. FIGS. 4C and 4D show the Gn^{H}:Gc and Gc:Gc interfaces in panel B (dotted rectangles) were enlarged for clarity and rotated as indicated to show neutralization-escape mutants in the Gn capping loop (FIG. 4C) and Gc (FIG. 4D). In panel C, the od fusion loop in Gc associated with the capping loop in Gn is shown. FIG. 4E shows the PUUV Gc post-fusion trimer with the three-fold molecular axis indicated (“3”) (PDB: 5J81; (22)). Labeling of Gc domains and escape mutations as in panel B.

FIG. 4F is a heatmap showing the capacity of mAbs to neutralize rVSV-PUUV-Gn/Gc bearing neutralization-escape Gn/Gc mutants. mAbs were grouped according to competition Groups I-IV (see FIG. 3). Group I=ADI-42898 competitors; Group II=ADI-42098 competitors; Group III=ADI-42898/-42098 non-competitors; Group IV=undefined/Gn^{H}/Gc non-binders—neutralization averages (n=6) from two experiments.

FIGS. 5A-5C show negative-stain electron microscopy (nsEM) of scFv:PUUV Gn^{H}/Gc complexes in accordance with the present technology. FIG. 5A shows exemplary nsEM 2D-classes of PUUV Gn^{H}/Gc bound to scFvs of the indicated mAbs. FIG. 5B shows 3D reconstructions of scFv:Gn^{H}/Gc complexes are shown in transparent surface representations with the structure of ANDV Gn^{H}/Gc docked into the density and pseudo-colored as described in panel A. Corresponding neutralization-escape mutations are shown in the closeups. FIG. 5C depicts modeled interactions of the indicated scFvs with tetrameric PUUV Gn^{H}/Gc complexes are shown in surface-shaded representation as in panel A. En face and side views are shown.

FIGS. 6A-6D show neutralization potency of mAbs against authentic Old-World hantaviruses in accordance with the present technology. Potency of mAbs to block OWH PUUV (FIG. 6A), HTNV (FIG. 6B), and DOBV (FIG. 6C) infection of VeroE6 cells (mAb concentrations of 100, 4, and 0.04 nM). Data points are shaded/patterned according to the mAb's competition group assignments defined in FIG. 3: Averages ±standard deviation (s.d.), n=2-10 from one to five experiments (PUUV); n=4 from two experiments (HTNV); n=4-6 from two to three experiments (DOBV). (FIG. 6D) Heatmap of IC₅₀ values isolated from PUUV, HTNV, and DOBV dose-response neutralization curves (FIGS. 6A-6C) derived by non-linear regression analysis. Data points are shaded/patterned according to the mAb's neutralization potency. mAbs with IC₅₀ values >5 nM are designated non-neutralizing mAbs (N.N.); mAbs whose neutralization curves did not adhere to a sigmoidal dose-response fit and for which an IC₅₀ could not be computed are designated as N.C. mAbs leaving an un-neutralized virus fraction are shown as striped data points on the map. An un-neutralized virus fraction is defined as a residual normalized virus infection at the highest mAb concentration tested of >5%. N.D. not determined.

FIGS. 7A and 7B show In vivo protective efficacy of human nAbs in a PUUV bank vole challenge model in accordance with the present technology. FIG. 7A: Bank voles were administered a single 25-mg/kg dose (i.p.) of the indicated nAbs (n=12 from two experiments) or PBS vehicle (n=9 from two experiments) followed by a challenge with PUUV/Suo (500 FFU, s.c.) at 4 h post nAb administration. Bars indicate median.

FIG. 7B: Bank voles were challenged with PUUV/Suo (500 FFU, s.c.) followed by treatment with a single 25-mg/kg dose (i.p.) of the indicated nAbs (n=12 from two experiments) or PBS vehicle (n=11 from two experiments) at 24 h post challenge. Bars indicate median. Untreated versus nAb-treated animals, un-paired Kruskal-Wallis test: *, P=0.0021; **, P=0.0002; **, P<0.0001.

FIG. 8A-8D depict broad pan-hantavirus neutralization and protection by ADI-42898 in accordance with the present technology. FIG. 8A shows potency of ADI-42898 to block infection of Vero cells by rVSVs bearing Gn/Gc from the indicated NWH. Viruses were exposed to a 3-fold mAb dilution series starting at 100 nM. Averages±s.d, n=14 from five experiments (ANDV); n=9 from three experiments (SNV); n=12 from four experiments (CHOV). FIG. 8B shows potency of ADI-42898 to block ANDV (strain Chile-9717869) and SNV (strain CC107) infection of HUVECs with a 3-fold mAb dilution series starting at 300 nM. Averages±s.d, n=4 from two experiments. FIG. 8C shows Syrian golden hamsters were challenged with ANDV (200 PFU, i.m.; strain Chile-9717869) followed by treatment with a single dose of ADI-42898 (~25-mg/kg or ~6-mg/kg, i.p.) at 3 days post-virus exposure. Mortality of hamsters was monitored for 35 days. Averages from two experiments, n=5 (~25-mg/kg dose); n=8 (~6-mg/kg dose), n=10 (vehicle). Untreated versus mAb-treated animals, Mantel-Cox test: **, P=0.0003; **, P<0.0001. FIG. 8D shows serum virus titers of Syrian golden hamsters on day 8 post ANDV challenge were determined by plaque assay. Untreated versus ADI-42898-treated animals, un-paired Kruskal-Wallis test: *, P=0.03; *, P=0.002. L.O.D., limit of detection.

FIG. 9 shows neutralization activity of PUUV convalescent subject sera in accordance with the present technology. Neutralization of rVSV-PUUV, -HTNV, and -ANDV-Gn/Gc or -VSV-G infection in the presence of sera from six PUUV-experienced donors (1:150 dilution). Averages±s.d., n=6 from three experiments (PUUV and HTNV); n=4 from two experiments (ANDV); n=2 from one experiment (VSV).

FIG. 10 shows a flow gating strategy for isolation of Gn/Gc-reactive memory B cells in accordance with the present technology. Representative gating strategy to sort single B cells specific for hantaviral Gn/Gc. SSC-A: Side scattered light; FSC-A: Forward scattered light.

FIGS. 11A-11C are curves for selected mAbs with different neutralization patterns in accordance with the present technology. Neutralization of rVSV-HTNV-Gn/Gc by (FIG. 11A) ADI-39044, a neutralizing mAb; (FIG. 11B) ADI-43852, a mAb leaving a non-neutralized virus fraction; (FIG. 11C) ADI-42089, a mAb lacking neutralization activity. Averages±s.d., n=6 for two experiments.

FIG. 12 depicts Domain organization of PUUV Gn/Gc, Gn^{H}/Gc and Gn^{H}. (Top) Diagram of PUUV GPC encoded by a single open reading frame of the genomic RNA segment M in accordance with the present technology. Cleavage sites are marked by arrows. Dashed line indicates the viral membrane. (Middle) Schematic of the single-chain Gn^{H}/Gc construct spanning the Gn head domain, Gn^{H} (aa 20-381) and Gc (aa 659-1107) and bypassing the trans-membrane segments in the precursor polyprotein by a soluble, 43-aa long, flexible linker. A Twin Strep-tag needed for protein purification was encoded in frame at the C-terminal end of the Gc sequence. (Bottom) Diagram of the single Gn^{H} domain (aa 20-381), also containing a Twin Strep-tag connected to its C-terminus.

FIGS. 13A and 13B illustrate characterization of rVSV-PUUV-Gn/Gc neutralization-escape mutants in accordance with the present technology. Capacity of mAbs to neutralize rVSV-PUUV-Gn/Gc bearing neutralization-escape variants in its Gn (FIG. 13A) or Gc (FIG. 13B) subunit. Averages±s.d., n=6-12 from two to four experiments.

FIGS. 14A-14D show locations of viral neutralization escape mutations in PUUV Gn/Gc in accordance with the present technology. Aa sequence alignment of regions of OWH and NWH Gn/Gc encompassing escape mutations are shown (PUUV Gn/Gc numbering). Escape mutations are found in the Gn (FIG. 14A) and Gc (FIGS. 14B-14D) subunit. Gray non-underlined residues indicate conserved residues. Underlined residues indicate divergent residues. Positions of escape mutations are boxed in a solid line, and the residue changes are indicated in dashed unshaded boxes. The location of the Gn capping loop is shown in the shaded box.

FIG. 15 shows binding activity of mAbs to PUUV Gn/Gc subdomain constructs in accordance with the present technology. Heatmap presents binding activity of mAbs to PUUV Gn^{H}, Gn^{H}/Gc and rVSV-PUUV-Gn/Gc (see FIG. 12).

FIGS. 16A and 16B show establishment of fusion-infection assay with rVSV-PUUV-Gn/Gc in accordance with the present technology. (FIG. 16A) Identification of optimal pH for PUUV Gn/Gc-induced fusion-infection. rVSV particles were bound to pre-chilled HUVECs, incubated with media adjusted to pH 7.0-4.0, followed by fusion-infection. rVSV-ANDV-Gn/Gc particles served as positive control. (Top) Averages±s.d., n=8 from three experiments. (Bottom) Representative images of infected eGFP+ or mNG+ cells detected by fluorescence microscopy. (FIG. 16B) Capacity of ADI-37219 and ADI-37236 to neutralize rVSV-PUUV-Gn/Gc. Averages±s.d, n=6 from two experiments (ADI-37219) and n=9 from three experiments (ADI-37236).

FIGS. 17A and 17B illustrate mechanistic basis of mAb-dependent inhibition of virus infection in accordance with the present technology. (FIG. 17A) Capacity of mAbs to block PUUV Gn/Gc-mediated fusion-infection. Data points are shaded/patterned according to the mAb's competition group assignments defined in FIG. 3. Averages±s.d., n=6-18 from two to six experiments. (FIG. 17B) Capacity of mAbs to block attachment of virus particles to host cells. Data points are shaded/patterned according to the mAb's competition group assignments defined in FIG. 3. Averages±s.d., n=two to three experiments.

FIGS. 18A-18D show activity of Group II mAbs against NWH Gn/Gc-mediated infection in accordance with the present technology. (FIGS. 18A and 18C) Capacity of Group II nAbs, ADI-39044 and -42871 to block infection by rVSV-ANDV, —SNV and -CHOV-GnGc on Vero cells. Averages±s.d., n=6 from two experiments. (FIGS. 18B and 18D) Capacity of Group II nAbs, ADI-39044 and -42871 to inhibit infection by authentic NWH, ANDV and SNV on HUVECs. Averages±s.d., n=2 from one experiment (SNV), and n=4 from two experiments (ANDV).

FIG. 19 shows polyreactivity of mAbs isolated from PUUV-experienced donors in accordance with the present technology. Thresholds for high, medium, and low polyreactivity are indicated by dashed lines. Polyspecificity scores for 137 clinical stage mAbs are shown for comparison. Bar indicates median. mAbs tested in bank vole PUUV challenges are marked: 1=ADI-42898; 2=ADI-42877; 3=ADI-38919; 4=ADI-42098.

FIGS. 20A and 20B depict GPC amino acid sequence analyses related to FIG. 1A in accordance with the present technology. FIG. 20A shows GPC amino acid sequences used for the construction of FIG. 1A. FIG. 20B shows pairwise amino acid sequence comparison of orthohantavirus GPCs shown in (FIG. 20A).

FIGS. 21A and 21B show demographic and laboratory characteristics of PUUV cohort donors during acute phase of disease in accordance with the present technology. FIG. 21A shows PUUV subjects displayed typical symptoms for HFRS. The maximum or minimum values during the acute phase of the disease are presented. FIG. 21B shows metadata from six PUUV convalescent subjects prioritized for isolation of memory B cells reactive to hantaviral Gn/Gc. APTT, Activated Partial Thromboplastin Time; PT INR, Prothrombin Time—International Normalized Ratio. “N.D.”—not determined.

FIGS. 22A-22E show ADI-42898 binding a pH-sensitive quaternary epitope at the Gn/Gc interface in accordance with the present technology. FIG. 22A shows mechanism of hantavirus class II membrane fusion. TMIR, target membrane-interacting region. FIG. 22B shows ribbon representation of x-ray structure of PUUV Gn^{H}/Gc in complex with an scFv fragment of ADI-42898. ADI-42898's epitope is shaped by three loops: Gn capping loop, Gc bc loop, Gc cd loop. FIG. 22C shows the ADI-42898:Gn^{H}/Gc interface in FIG. 22B was enlarged for clarity to depict amino acid residues involved in the interaction. FIG. 22D shows surface representations of the Gn^{H}/Gc complex shaded according to sequence conservation (left panel) with the side chains involved in binding to ADI-42898 indicated or shaded by domains (right panel), as indicated. The footprint of the antibody epitope is outlined. FIG. 22E shows analysis of Gn^{H}/Gc heterodimer binding to biolayer interferometry (BLI) sensors coated with ADI-42898 and -42885 at indicated pH values. Averages±SD, n=3 from three independent experiments.

FIGS. 23A-23G show mechanistic basis of ADI-42898's blockade of viral entry in accordance with the present technology. (FIG. 23A) Capacity of Gn/Gc-specific mAbs to block rVSV ANDV-Gn/Gc capture by sEC1-2 measured by ELISA. Averages±SD, n=6 from three independent experiments. (FIG. 23B) Capacity of ADI-42898 (or as a control convalescent serum from a Syrian hamster infected with rVSV ANDV-Gn/Gc) to block rVSV ANDV-Gn/Gc attachment to human umbilical vein endothelial cells (HUVEC). (FIG. 23C) Capacity of ADI-42898 to inhibit PUUV or ANDV Gn/Gc-mediated fusion-infection. Averages±SD, n=6-12 from two to four independent experiments. (FIG. 23D) Heatmap of EC₅₀ (isolated from PUUV and ANDV Gn/Gc VLP dose-response ELISA curves) and IC₅₀ (isolated from PUUV and ANDV dose-response neutralization curves) values derived by nonlinear regression analysis. Data points are shaded/patterned according to the IgGs or Fabs binding and neutralization potency. IgGs or Fabs with EC₅₀ or IC₅₀ values of >100 nM is designated non-binding or non-neutralizing (NB and NN, respectively). (FIG. 23E) Modeled interactions of bivalent ADI-42898 to a local (Gn/Gc)₄ lattice patch based on the PUUV Gn/Gc&:ADI-42898 structure in FIG. 22 are shown in orthogonal (top) and en face (bottom) views. (FIG. 23F) BLI sensorgrams for association of PUUV (top) and ANDV (bottom) Gn^{H}/Gc heterodimers to ADI-42898 at pH 7, followed by dissociation of Gn^{H}/Gc at pH 7 and 5.5 (indicated by dotted line). (FIG. 23G) Capacity of ADI-42898 to bind VLPs bearing PUUV (top) and ANDV (bottom) Gn/Gc in different pH regimes measured by ELISA. Averages±SD, n=4 from 2 independent experiments.

FIGS. 24A-24G show characterization of affinity-matured mAbs, ADI-65533 and -65534 in accordance with the present technology. (FIG. 24A) Sequence alignment of regions in ADI-42898 and its affinity-matured variants encompassing introduced mutations (shaded light; Kabat numbering). (FIG. 24B) The ADI-42898:Gn^{H}/Gc interface was enlarged for clarity to depict amino acid residues mutated during the affinity maturation campaign and outlined in FIG. 24A (left). Potential interactions between introduced residue changes in CDR-H1 (T28E) and CDR-H3 (G100V or “G100aV”) with residues in the bc and cd loops in PUUV and ANDV Gc are shown (right). (FIG. 24C) Graphical representation of association (Kₒₙ at pH 7.0) and dissociation rate (K_{off} at pH 5.5) constants for mAb interactions with PUUV and ANDV Gn^{H}/Gc. averages±SD, n=4 from four independent experiments are shown. (FIG. 24D) Capacity of mAbs to bind ANDV Gn/Gc-decorated VLPs in different pH regimes as indicated and measured by ELISA. Averages±SD, n=6 from three independent experiments. (FIG. 24E) Capacity of Gn/Gc-specific mAbs to block rVSV ANDV-Gn/Gc capture by sEC1-2 measured by ELISA. Averages±SD, n=6 from three independent experiments. (FIG. 24F) Capacity of ADI-42898, -65533, and -65534 to inhibit ANDV Gn/Gc-mediated fusion-infection. rVSV ANDV-Gn/Gc particles were pre-incubated with decreasing amounts of mAb (100 nM to 0.01 nM), followed by fusion-infection of HUVECs. Averages±SD, n=12 from four independent experiments.

FIGS. 25A-25C show broad hantavirus neutralization and protection in Syrian hamsters by affinity-matured mAbs in accordance with the present technology. (FIG. 25A) Potency of ADI-42898, -65533, and -65534 against ANDV infection of HUVEC cells. Viruses were exposed to a 3-fold mAb dilution series starting at 300 nM. Averages±SD, n=14 from seven independent experiments are shown. (FIG. 25B) Heatmap of IC₅₀ from ANDV and PUUV dose-response neutralization curves (FIG. 25A) derived by nonlinear regression analysis. Data points are shaded/patterned according to mAb neutralization potency. ^{#}, data from focus-reduction neutralization assays; *, data from microneutralization assays. (FIG. 25C) Syrian golden hamsters were challenged with ANDV [200 PFU, intramuscular (i.m.)], followed by treatment with a single dose of mAb [2 mg/kg or 0.5 mg/kg, intraperitoneal (i.p.)]3 days after virus exposure. Averages from one experiment, n=6 per group.

FIG. 26 shows epitope of ADI-42898 in hantaviral Gn/Gc in accordance with the present technology. Annotated amino acid sequence alignment of OWH and NWH Gn/Gc (PUUV [strain Astrup], GenBank AJC50718; HTNV [strain 76-118], GenBank Y00386.1; SEOV [strain SR-11], GenBank M34882.1; DOBV [strain Ano-Poroia/Af19], GenBank NP_942554.1; ANDV [strain Chile-9717869], GenBank MT956623.1; SNV [strain NM H10], GenBank KT885045.1; CHOV [strain 588] GenBank KT983772.1; LGNV [strain 510B], AF005728.1) (PUUV Gn/Gc numbering). Gray non-underlined residues indicate conserved residues; underlined residues indicate divergent residues. Position of PUUV^{Q98} and equivalent residues in divergent OWH and NWH Gn/Gcs are boxed. ADI-42898 paratope contact sites (distance <5 Å, determined with PyMol) are indicated and shaded/patterned according to contact CDRs.

FIGS. 27A-27C show characterization of the ADI-42898 paratope in accordance with the present technology. (FIG. 27A and FIG. 27B) Capacity of mAbs carrying alanine substitutions in Gn/Gc contact sites identified in FIGS. 22B and 22C to bind rVSV-PUUV-Gn/Gc (FIG. 27A) or -ANDV-Gn/Gc (FIG. 27B) was measured by ELISA. Averages±SD, n=4 from two independent experiments. (FIG. 27C) Heatmap of EC₅₀ values isolated from PUUV and ANDV Gn/Gc dose-response binding curves (FIGS. 27A and 27B) derived by nonlinear regression analysis. Data points are shaded/patterned according to the mAb's binding potency. mAbs with EC₅₀ values of >100 nM were designated as non-binding mAbs (NB). HC, heavy chain. LC, light chain.

FIGS. 28A-28E show germline origin of ADI-42898 and sequence determinants of its binding activity in accordance with the present technology. (FIG. 28A) Alignment of mature V_{H} and V_{L} sequences (ADI-42898^{WT}) with the reconstruction of an inferred germline ancestor (ADI-42898^{IGL}) bearing the closest human germline V and J gene segments and mature CDR-H3 (Kabat numbering). Amino acid residues divergent from the inferred germline sequence are shown in a lighter shade. (FIGS. 28B and 28C) Capacity of mAb variants carrying inferred germline ancestor sequences to bind rVSV-PUUV-Gn/Gc (left panels) or -ANDV-Gn/Gc (right panels) was measured by ELISA. Averages±SD, n=4 from two independent experiments. (FIG. 28D) Ribbon representation of ADI-42898:PUUV Gn^{H}/Gc contact surface with three discontinuous loops (Gn capping loop, Gc bc loop, and Gc cd loop) engaging the mAb's HC and LC. SHM Y49S located in FR2 of the LC is highlighted (see FIG. 28A). The spatial organization of the CDR-H3 proximal to Y49 in ADI-42898^{IGL} was enlarged for clarity (dashed rectangle). (FIG. 28E) Heatmap of EC₅₀ values isolated from PUUV and ANDV Gn/Gc dose-response binding curves (FIGS. 28B and 28C) derived by nonlinear regression analysis. Data points are shaded/patterned according to the mAb's binding potency. mAbs with EC₅₀ values of >100 nM are designated as non-binding mAbs (NB). HC, heavy chain. LC, light chain.

FIGS. 29A-29D show ADI-42898 neutralization determinants in accordance with the present technology. (FIG. 29A) Amino acid sequence alignment of OWH and NWH Gn/Gc capping loops (PUUV [strain Astrup], GenBank AJC50718; HTNV [strain 76-118], GenBank Y00386.1; SEOV [strain SR-11], GenBank M34882.1; DOBV [strain Ano-Poroia/Af19], GenBank NP_942554.1; ANDV [strain Chile-9717869], GenBank MT956623.1; SNV [strain NM H10], GenBank KT885045.1; CHOV [strain 588]GenBank KT983772.1; LGNV [strain 510B], AF005728.1) (PUUV Gn/Gc numbering). Gray non-underlined residues indicate conserved residues; underlined residues indicate divergent residues. Position of PUUV^{Q98} and equivalent residues in divergent Gn/Gc complexes are boxed. (FIG. 29B) ADI-42898 neutralization profiles of rVSVs bearing OWH Gn/Gc. Averages±SD, n=9-12 from three to four independent experiments. (FIG. 29C) ADI-42898 neutralization profiles of rVSV bearing NWH Gn/Gc. Averages±SD, n=9-12 from three to four independent experiments. (FIG. 29D) Heatmap of IC₅₀ values isolated from rVSV dose-response neutralization curves (FIGS. 29B and 29C) derived by nonlinear regression analysis. Data points are colored according to ADI-42898's neutralization potency.

FIGS. 30A and 30B show characterization of rVSV-ANDV- and -SNV-Gn/Gc mutants resistant to ADI-42898 neutralization in accordance with the present technology. (FIG. 30A) Amino acid sequence alignment of Gn of ANDV and ANDV^{A96T} (left with ANDV Gn/Gc numbering) as well as SNV, SNV^{S88N} and SNV^{T90N} (right with SNV Gn/Gc numbering). Underlined residues indicate viral neutralization escape mutations. Positions of sequons (N-X-S/T) introduced in escape variants are boxed. (FIG. 30B) Capacity of ADI-42898 to neutralize rVSV-ANDV- or -SNV-Gn/Gc bearing neutralization-escape variants in their Gn capping loop. Averages±SD, n=6-12 from two to four independent experiments.

FIGS. 31A and 31B show binding properties of ADI-42898 in accordance with the present technology. (FIG. 31A) BLI sensorgrams for ADI-42898 Fab binding to PUUV and ANDV Gn^{H}/Gc. Experimental curves were fit using a 1:1 binding model. (FIG. 31B) BLI sensorgrams for ADI-42898 IgG binding to PUUV and ANDV Gn^{H}/Gc. Experimental curves were fit using a 1:1 binding model. Results from a representative experiment are shown.

FIGS. 32A and 32B show significance of ADI-42898 valency for its binding and neutralization profiles in accordance with the present technology. (FIG. 32A) Capacity of IgGs and monovalent Fabs to bind ANDV and PUUV Gn/Gc-decorated VLPs as measured by ELISA. Averages±SD, n=6 from three independent experiments. (FIG. 32B) Capacity of IgGs and monovalent Fabs to inhibit PUUV (strain Kazan) and ANDV (strain Chile-9717869) infection. Averages±SD, n=4-6 from two independent experiments.

FIGS. 33A-33D show the engineering of ADI-42898 for improved affinity towards ANDV Gn/Gc in accordance with the present technology. (FIG. 33A) Graphical representation of dissociation rate constants (k_{off}) of Fab fragments of ADI-42898 and 12 progeny variants bearing single-mutant CDRs bound to ANDV and PUUV Gn^{H}/Gc, as assessed by BLI. (FIG. 33B) Kinetic constants of 26 Fabs carrying combinatorial mutations for binding to ANDV and PUUV Gn^{H}/Gc are shown, as determined by BLI. The shaded area indicates clones selected for testing against rVSV-ANDV-Gn/Gc infection. (FIG. 33C) Heatmap of IC₅₀ values from rVSV-PUUV, -HTNV, and -ANDV Gn/Gc dose-response neutralization curves derived by nonlinear regression analysis. Data points are patterned according to mAb neutralization potency. mAbs leaving an un-neutralized virus fraction are shown as striped data points on the map. Averages±SD, n=6 from two independent experiments are shown. (FIG. 33D) Polyreactivity scores. Dashed lines indicate thresholds for high, medium, and low polyreactivity.

FIGS. 34A and 34B show kinetic binding studies of ADI-42898 and affinity-matured mAbs to Gn^{H}/Gc in accordance with the present technology. (FIG. 34A) BLI sensorgrams showing binding kinetics of PUUV (left panel) and ANDV (right panel) Gn^{H}/Gc to ADI-42898, -65533, and -65534 at pH 7.0. (FIG. 34B) BLI sensorgrams for the association of PUUV (left panel) and ANDV (right panel) Gn^{H}/Gc to mAbs at pH 7.0, followed by dissociation of Gn^{H}/Gc at pH 5.5 (pH shift indicated by dotted line). Experimental curves were fitted using a 1:1 binding model using GraphPad Prism. n=4 from four independent experiments are shown.

FIG. 35 shows blocking of PUUV Gn/Gc-mediated fusion-infection and capacity of ADI-42898, -65533, and -65534 to inhibit PUUV Gn/Gc-mediated fusion-infection in accordance with the present technology. Averages±SD, n=6 from two independent experiments.

FIGS. 36A and 36B show potency of ADI-42898, -65533, and -65534 to block ANDV and PUUV infection in accordance with the present technology. (FIG. 36A) ANDV (strain Chile-7913) was exposed to 3-fold mAb serial dilutions starting at 300 nM; infection levels on VeroE6 cells were determined by focus-reduction neutralization assay. Averages±SD, n=2 from one experiment are shown. (FIG. 36B) PUUV (strain Kazan) was exposed to 5-fold mAb serial dilutions starting at 100 nM; infection levels on Vero cells were determined by microneutralization assay. Averages±SD, n=4-6 from two to three experiments are shown.

FIG. 37 shows in vivo protective efficacy of human mAbs in a PUUV bank vole challenge model in accordance with the present technology. Bars indicate the median. Untreated vs. nAb-treated animals, unpaired Kruskal-Wallis test: *, P=0.0021; **, P=0.0002.

FIG. 38 shows crystallographic statistics in accordance with the present technology. ^{a} data for the last resolution shell is displayed in parenthesis throughout the table; ^{b}, R_{sym}=Σ|Iᵢ−<Iᵢ>|/ΣIᵢ, where Iᵢ is the observed intensity and <Iᵢ> is the average intensity obtained from multiple observations of symmetry-related reflections; ^{c}, R_{work}=Σ∥Fobs(hkl)∥−∥Fcalc(hkl)|/Σ|Fobs(hkl)|.

### DETAILED DESCRIPTION

As used herein, the singular forms “a,” “an,” and “the” include plural referents unless the content dictates otherwise. Thus, for example, reference to “an antibody” optionally includes a combination of two or more such molecules and the like.

The term “about,” as used herein, refers to the usual error range for the respective value readily known to the skilled person in this technical field. For example, ±20%, ±10%, ±5%, ±1%, ±0.5%, or ±0.1%, may be within the intended meaning of the recited value, provided that when there are two or more recited values, such a margin of error does not render the two or more recited values to overlap.

As used herein, “antibody” means an isolated or recombinant binding agent that comprises the necessary variable region sequences to specifically bind an antigenic epitope. Therefore, an “antibody” as used herein is any form of an antibody of any class, subclass, or fragment that exhibits the desired biological activity, e.g., binding a specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment with two antigen combining sites and is still capable of cross-linking antigen.

As used herein, “V-region” refers to an antibody variable region domain comprising the segments of Framework 1, CDR1, Framework 2, CDR2, and Framework 3, including CDR3 and Framework 4. The heavy chain V-region, V_{H}, is a consequence of the rearrangement of a V-gene (HV), a D-gene (HD), and a J-gene (HJ), in what is termed V(D)J recombination during B-cell differentiation. The light chain V-region, V_{L}, is a consequence of the rearrangement of a V-gene (LV) and a J-gene (LJ).

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus and are also identified by the chain in which the CDR is located. Thus, for example, a V_{H} CDR3 (HCDR3) is in the variable domain of the heavy chain of the antibody in which it is found. In contrast, a V_{L} CDR3 (LCDR3) is the CDR3 from the variable domain of the light chain of the antibody in which it is located. “CDR” is used interchangeably with “HVR” in this application when referring to CDR sequences.

The amino acid sequences of the CDRs and framework regions may be determined using various well-known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, the structural repertoire of the human V_{H} segments J. Mol. Biol. 227, 799-817; AI-Lazikani et al., J. Mol. Biol. 1997, 273(4)). Definitions of antigen-combining sites are also described in the following: Ruiz et al., IMGT, and the international ImMunoGeneTics database. Nucleic Acids Res., 28, 219-221 (2000); Lefranc, M.-P. IMGT, the international ImMunoGeneTics database. Nucleic Acids Res. January 1; 29(1):207-9 (2001); MacCallum et al., Antibody-antigen interactions: Contact analysis and binding site topography, J. Mol. Biol., 262 (5), 732-745 (1996); and Martin et al., Proc. Natl Acad. Sci. USA, 86, 9268-9272 (1989); Martin et al., Methods Enzymol., 203, 121-153, (1991); Pedersen et al., Immunomethods, 1, 126, (1992); and Rees et al., In Stenberg M.J.E. (ed.), Protein Structure Prediction. Oxford University Press, Oxford, 141-172 1996). For example, reference to CDRs as determined by Kabat numbering is based on Kabat et al., Sequences of Proteins of Immunological Interest, 5^{th} Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

A “nonlinear epitope” or “conformational epitope” comprises noncontiguous polypeptides (or amino acids) within the antigenic protein to which an antibody specific to the epitope binds. A conformational epitope is typically formed by a three-dimensional interaction of amino acids in the epitope that may not necessarily be contained in a single stretch of amino acids. A quaternary epitope is a conformational epitope whose structure depends upon the arrangement of multiple protomers, is enhanced by them, or may depend on the arrangement of multiple protomers (i.e., quaternary epitopes). A quaternary epitope may be in a single protein of a multimeric complex or span multiple protomers, forming de novo by their interaction.

An “Fc region” refers to the constant region of an antibody, excluding the first constant region immunoglobulin domain. Thus, e.g., for human immunoglobulins, “Fc” refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 and Cγ3 and the hinge between Cγ1 and Cy. It is understood in the art that the boundaries of the Fc region may vary; however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or the context of an antibody or antibody fragment. “Fc region” includes naturally occurring allelic variants of the Fc region and modified Fc regions, e.g., limited to modulating effector function or other properties such as pharmacokinetics, stability, or production properties of an antibody. Fc regions also include variants that do not exhibit alterations in biological function. For example, one or more amino acids may be deleted from the N-terminus or C-terminus of the Fc region of an immunoglobulin without substantial loss of biological function. Such variants may be selected according to general rules known in the art to have minimal effect on activity (see, e.g., Bowie et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibody (see, e.g., Angal et al., Mol Immunol 30:105-108, 1993).

An “IC₅₀.” as used herein in the context of neutralizing antibody activity against a viral Gn/Gc protein (e.g., rVSV-mNG-PUUV-Gn/Gc), refers to the half maximal inhibitory concentration, which is a measure of the potency of the antibody in inhibiting binding to the viral epitopes. IC₅₀ is a quantitative measure that indicates the concentration of an antibody needed to inhibit (e.g., by neutralization) the activity of a Gn/Gc protein by 50% (readouts may include but are not limited to fluorescence or luminescence signals).

As used herein, “neutralizing antibody” (NAb) refers to an antibody, for example, a monoclonal antibody, capable of disrupting a formed viral particle or inhibiting formation of a viral particle or prevention of binding to or infection of mammalian cells by a viral particle. As used herein, “human antibodies” refer to antibodies that were isolated from the B cells of a human or directly from the sequence of serum antibodies.

The term “broad neutralizing activity (bnAbs)” or “broad serum neutralizing antibody” refers to an antibody that is capable of neutralizing epitopes of at least 2, 3, 4, 5, or more hantavirus species. The epitopes may be highly conversed among hantavirus species. For instance, a bnAb has “pan-hantavirus” binding characteristics if it may bind to at least 1, 2, 3, 4, 5 or more epitopes of at least 2, 3, 4, 5, or more hantavirus species. For example, bnAb has broad neutralization and protection properties when it may block infection by at least 2, 3, 4, 5, or more hantavirus species.

The term “tetramer” refers to the basic antibody structural unit. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

The term “cell entry” or “cell entry pathway,” as used herein in the context of viral infection, refers to the process of introducing a viral particle into a cell. For instance, during cell entry of hantavirus, endosomal acid pH triggers dissociation of the square-shaped heterotetrameric (Gn/Gc)₄ complex, allowing Gc subunits to insert into the target membrane and refold into post-fusion homotrimers.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for determining percent amino acid sequence identity may be performed in various methods, including publicly available computer software such as BLAST, BLAST-2, ALIGN, or Megalign (DNASTAR). Examples of algorithms suitable for determining percent sequence identity and sequence similarity are the BLAST 2.0 algorithms, described in Altschul et al., Nuc. Acids Res. 25:3389-3402 (1977) and Altschul et al., J. Mol. Biol. 215:403-410 (1990). Thus, for purposes of the present technology, BLAST 2.0 may be used with the default parameters to determine the percent sequence identity.

The terms “corresponding to,” “determined with reference to,” or “numbered with reference to,” when used in the context of the identification of a given amino acid residue in a polypeptide sequence, refers to the position of the residue of a specified reference sequence when the given amino acid sequence is maximally aligned and compared to the reference sequence. The polypeptide aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and refer to both sense and anti-sense strands of RNA, cDNA, genomic DNA, and synthetic forms and mixed polymers of the above. In particular embodiments, a nucleotide refers to a ribonucleotide, deoxynucleotide, or a modified form of either type of nucleotide and combinations thereof. The terms also include, but is not limited to, single- and double-stranded forms of DNA. In addition, a polynucleotide, e.g., a cDNA or mRNA, may include either naturally occurring and modified nucleotides linked together by naturally occurring and/or non-naturally occurring nucleotide linkages. The nucleic acid molecules may be modified chemically or biochemically or contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term includes any topological conformation, including single-stranded, double-stranded, partially duplexed, triplex, hairpinned, circular, and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure and the vector incorporated into the genome of a host cell into which it has been introduced. A “vector” refers to a recombinant construct in which a nucleic acid sequence of interest is inserted into the vector. Specific vectors may direct the expression of nucleic acids to which they are operatively linked. Such vectors are referred to herein as “expression vectors.”

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy or light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cells and includes the primary transformed cell and progeny derived therefrom without regard to the number of passages.

A polypeptide “variant,” as the term is used herein, is a polypeptide that typically differs from one or more polypeptide sequences of the present technology in one or more substitutions, deletions, additions and/or insertions.

The term “blocking” or “inhibiting” as used herein are interchangeable and refer to interfering with a cellular activity or reducing the binding affinity of an antibody to an epitope (e.g., nAb:Gn/Gc). As an illustration, a hantavirus is blocked from entering a host cell when the binding affinity of nAb:Gn/Gc is reduced by at least 5%, at least 10%, at least 15%, at least 20%, at least 30%, at least 40%, at least 50%, at least 60%, at least 70%, at least 80%, at least 90%, at least 95%, at least 99%, or more.

As used herein, “therapeutic agent” refers to an agent that when administered to a subject suffering from a disease, in a therapeutically effective dose, will cure, or at least partially arrest the symptoms of the disease and complications associated with the disease.

As used herein, the term “sibling antibodies” refer to antibodies derived from the same B-cell clonal lineage. In some embodiments, sibling antibodies share sequence and structural properties as well as epitope specificity.

As used herein, the term “convergent antibodies” refer to that antibodies derived from different B-cell lineages that exhibit similar sequence and structural properties.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit may also mean to effect a cure of one or more diseases, conditions, or symptoms under treatment.

The term “acidic pH” refers to a pH that is less than 7. An acidic pH generally refers to a pH in a range between 1 and 6.8, between 2 and 6.5, between 3 and 6, between 4 and 6, or for example, 5.5.

### Hantavirus

Hantaviruses may display a lipid envelope wrapped by a glycoprotein shell formed by two glycoproteins, Gn and Gc, responsible for orchestrating host cell recognition and entry steps. Gn and Gc may be the primary targets of the neutralizing immune response mediated by the antibodies of the present technology. Gn/Gc may be synthesized as a single polyprotein precursor (GPC). In some embodiments, Gn (SEQ ID NO:42) is a modular protein of about 650 amino acids long composed of two extra-viral regions termed head (Gn^{H}) and base (Gn^{B}) and a transmembrane region followed by an intraviral segment of about 110 amino acids. In some embodiments, a second, C-terminal transmembrane region of Gn ends in a conserved motif cleaved by a host signalase in the endoplasmic reticulum (ER) to give rise to the Gc N-terminus. The fusion protein Gc (SEQ ID NO:43) may comprise an ectodomain of about 450 amino acids long, followed by a single transmembrane region and a small intraviral tail. In some embodiments, there are four N-linked glycosylation sites; two in Gn^{H}, one in Gn^{B}, and one in Gc, which are conserved in all hantaviruses. Some hantaviruses, like Dobrava, Hantaan, and Thottapalayam viruses, may display additional N-glycosylation motifs. The first two conserved sugar chains may mediate contacts between Gn^{H} and Gc in the Gn/Gc heterodimer. Their removal may give rise to defects in the intracellular transport of the glycoprotein complex. The other two glycans may stabilize the (Gn/Gc)₄ spike. Their ablation may result in Gn/Gc complexes that reach the Golgi apparatus (GA) may not be recognized by certain conformational antibodies.

The N-terminal region of the Gn ectodomain may exhibit a mixed α/β fold. In some embodiments, the mixed α/β fold locates to the membrane-distal region of the tetrameric spike and is linked to the virion envelope by a C-terminal stalk region. In some embodiments, Gn is membrane distal and/or shields hydrophobic fusion loops on the Gc. The Gc may adopt a three-domain (domains I-III) class II fusion protein architecture and may associate with the Gn, linking adjacent Gn tetramers. In some embodiments, the Gn shields the fusion loop resident in domain II of the Gc.

In some embodiments, a host cell may bind and/or endocytosis the virus. In some embodiments, acidification in endosomal compartments results in at least partial dissolution of the Gn-Gc lattice. Endosomal escape may be mediated by the Gc in a process that involves engagement of the target membrane by the Gc-encoded fusion loop, followed by upward ‘zippering action’ conformational rearrangements of the transmembrane-anchored Gc domain III, which may bring together host and viral membranes.

The hantavirus genome may comprise a medium (M) segment encoding a glycoprotein precursor (GPC), which is cotranslationally cleaved to generate N-terminal (Gn) and C-terminal (Gc) subunits that associate to form square-shaped heterotetrameric (Gn/Gc)₄ spikes. Following cleavage of the GPC at a WAASA recognition sequence during protein biosynthesis, the Gn and Gc may form a lattice of mature spike complexes. A globular membrane-distal N-terminal Gn domain exposed at the viral surface may contain the receptor-binding site.

Gn and Gc glycoproteins may be targets of the neutralizing humoral immune response. Several cellular factors, including integrins, decay-accelerating factor, and complement receptor gC1qR-p32, may mediate hantavirus entry into host cells. Following receptor recognition, hantaviruses undergo clathrin-mediated endocytosis, and the viral genome is delivered into the cytoplasm following Gc-mediated fusion of viral and host membranes, a process triggered by the acidic pH of late endosomes or lysosomes.

During cell entry, endosomal acid pH triggers Gn/Gc dissociation, allowing Gc subunits to insert into the target membrane and refold into post-fusion homotrimers, thereby driving viral membrane fusion and cytoplasmic escape (17, 20-23). Gn/Gc spikes thus provide the primary target for antibodies that mediate their neutralizing activity by blocking one or more steps in the hantavirus entry pathway.

### Hantavirus Antibodies

### Heavy Chain Sequences

In some embodiments, the present technology comprises hantavirus antibodies. The hantavirus antibodies may have a heavy chain having a variable heavy (V_{H}) region and/or a complementary determining region sequence (e.g., HCDR1, HCDR2, HDCR3) of any one of ADI-42898, ADI-42877, ADI-42098, ADI-42885, ADI-37236, ADI-65533, or ADI-65534 as provided in Table 1.

In some embodiments, the hantavirus antibodies comprise or consist of a heavy chain having an amino acid sequence about 50%, about 55%, about 60%, about 65%, about 70%, about 75%, about 80%, about 85%, about 90%, about 95%, or about 100% identical to an amino acid sequence in Table 1.

In some embodiments, the hantavirus antibodies comprise or consist of a heavy chain having an amino acid sequence at least 50%, at least 55%, at least 60%, at least 65%, at least 70%, at least 75%, at least 80%, at least 85%, at least 90%, at least 95%, or at least 100% identical to an amino acid sequence in Table 1.

In some embodiments, the hantavirus antibodies comprise or consist of a heavy chain having an amino acid sequence at least about 50%, at least about 55%, at least about 60%, at least about 65%, at least about 70%, at least about 75%, at least about 80%, at least about 85%, at least about 90%, at least about 95%, or at least about 100% identical to an amino acid sequence in Table 1.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 1-5, 68, 69, or 91.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 1-5, 68, 69, or 91.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 1-5, 68, 69, or 91.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 1.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 2.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 3.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 4.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 5.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 68.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 69.

In some embodiments, the hantavirus antibodies comprise or consist of a V_{H} region about 50%, about 55%, about 60%, about 65%, about 70%, about 75%, about 80%, about 85%, about 90%, about 95%, or about 100% identical to the amino acid sequence of SEQ ID NO: 91.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 1.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 2.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 3.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 4.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 5.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 68.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 69.

In some embodiments, the hantavirus antibodies comprise or consist of a V_{H} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 91.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 1.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 2.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 3.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 4.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 5.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 68.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 69.

In some embodiments, the hantavirus antibodies comprise or consist of a V_{H} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 91.

In some embodiments, the hantavirus antibodies comprise or consist of a heavy chain having one or more CDR sequences about 50%, about 55%, about 60%, about 65%, about 70%, about 75%, about 80%, about 85%, about 90%, about 95%, or about 100% identical to the amino acid sequence of SEQ ID NO: 6-20, 72-77, or 106-108.

In some embodiments, the hantavirus antibodies comprise or consist of a heavy chain having one or more CDR sequences at least 50%, at least 55%, at least 60%, at least 65%, at least 70%, at least 75%, at least 80%, at least 85%, at least 90%, at least 95%, or at least 100% identical to the amino acid sequence of SEQ ID NO: 6-20, 72-77, or 106-108.

In some embodiments, the hantavirus antibodies comprise or consist of a heavy chain having one or more CDR sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 6-20, 72-77, or 106-108.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 6.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 7.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 8.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 9.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 10.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 11.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 12.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 13.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 14.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 15.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 16.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 17.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 18.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 19.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 20.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 72.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 73.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 74.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 75.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 76.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 77.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 106.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 107.

In some embodiments, the hantavirus antibodies comprise or consist of a heavy chain CDR sequence about 50%, about 55%, about 60%, about 65%, about 70%, about 75%, about 80%, about 85%, about 90%, about 95%, or about 100% identical to the amino acid sequence of SEQ ID NO: 108.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 6.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 7.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 8.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 9.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 10.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 11.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 12.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 13.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 14.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 15.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 16.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 17.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 18.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 19.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 20.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 72.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 73.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 74.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 75.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 76.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 77.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 106.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 107.

In some embodiments, the hantavirus antibodies comprise or consist of a heavy chain CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 108.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 6.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 7.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 8.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 9.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 10.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 11.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 12.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 13.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 14.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 15.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 16.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 17.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 18.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 19.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 20.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 72.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 73.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 74.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 75.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 76.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 77.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 106.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 107.

In some embodiments, the hantavirus antibodies comprise or consist of a heavy chain CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 108.

### Light Chain Sequences

In some embodiments, the hantavirus antibodies comprise or consist of a light chain having a variable light (V_{L}) region or a CDR sequence (e.g., LCDR1, LCDR2, LCDR3) of any one of ADI-42898, ADI-42877, ADI-42098, ADI-42885, ADI-37236, ADI-65533, or ADI-65534 as provided in Table 2.

In some embodiments, the hantavirus antibodies comprise or consist of a light chain having an amino acid sequence about 50%, about 55%, about 60%, about 65%, about 70%, about 75%, about 80%, about 85%, about 90%, about 95%, or about 100% identical to an amino acid sequence in Table 2.

In some embodiments, the hantavirus antibodies comprise or consist of a light chain having an amino acid sequence at least 50%, at least 55%, at least 60%, at least 65%, at least 70%, at least 75%, at least 80%, at least 85%, at least 90%, at least 95%, or at least 100% identical to an amino acid sequence in Table 2.

In some embodiments, the hantavirus antibodies comprise or consist of a light chain having an amino acid sequence at least about 50%, at least about 55%, at least about 60%, at least about 65%, at least about 70%, at least about 75%, at least about 80%, at least about 85%, at least about 90%, at least about 95%, or at least about 100% identical to an amino acid sequence in Table 2.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 21-25, 70, 81, or 92.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 21-25, 70, 81, or 92.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 21-25, 70, 81, or 92.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 21.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 22.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 23.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 24.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 25.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 70.

In some embodiments, the hantavirus antibodies comprise or consist of a V_{L} region about 50%, about 55%, about 60%, about 65%, about 70%, about 75%, about 80%, about 85%, about 90%, about 95%, or about 100% identical to the amino acid sequence of SEQ ID NO: 81.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 21.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 22.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 23.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 24.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 25.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 70.

In some embodiments, the hantavirus antibodies comprise or consist of a V_{L} region at least 50%, at least 55%, at least 60%, at least 65%, at least 70%, at least 75%, at least 80%, at least 85%, at least 90%, at least 95%, or at least 100% identical to the amino acid sequence of SEQ ID NO: 81.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 21.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 22.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 23.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 24.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 25.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 70.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 81.

In some embodiments, the hantavirus antibodies comprise or consist of a V_{L} region at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 92.

In some embodiments, the hantavirus antibodies comprise or consist of a light chain having one or more CDR sequences about 50%, about 55%, about 60%, about 65%, about 70%, about 75%, about 80%, about 85%, about 90%, about 95%, or about 100% identical to the amino acid sequence of SEQ ID NO: 35-40, 78-80, or 106-111.

In some embodiments, the hantavirus antibodies comprise or consist of a light chain having one or more CDR sequences at least 50%, at least 55%, at least 60%, at least 65%, at least 70%, at least 75%, at least 80%, at least 85%, at least 90%, at least 95%, or at least 100% identical to the amino acid sequence of SEQ ID NO: 35-40, 78-80, or 106-111.

In some embodiments, the hantavirus antibodies comprise or consist of a light chain having one or more CDR sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 35-40, 78-80, or 106-111.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 35.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 36.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 37.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 38.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 39.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 40.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 78.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 79.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 80.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 109.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 110.

In some embodiments, the hantavirus antibodies comprise or consist of a light chain having a CDR sequence about 50%, about 55%, about 60%, about 65%, about 70%, about 75%, about 80%, about 85%, about 90%, about 95%, or about 100% identical to the amino acid sequence of SEQ ID NO: 111.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 35.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 36.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 37.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 38.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 39.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 40.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 78.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 79.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 80.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 109.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 110.

In some embodiments, the hantavirus antibodies comprise or consist of a light chain having a CDR sequence at least 50%, at least 55%, at least 60%, at least 65%, at least 70%, at least 75%, at least 80%, at least 85%, at least 90%, at least 95%, or at least 100% identical to the amino acid sequence of SEQ ID NO: 111.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 35.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 36.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 37.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 38.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 39.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 40.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 78.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 79.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 80.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 109.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 110.

In some embodiments, the hantavirus antibodies comprise or consist of a light chain having a CDR sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NO: 111.

TABLE 1
Antibodies and heavy chain CDR sequences

| Antibody | | | | |
|---|---|---|---|---|
| ID | V_{H} | HCDR1 | HCDR2 | HCDR3 |
| ADI- | QVQLVESGGGVVQPGRS | FTFSSYAM | TWFDVSKK | ARNLIRYS |
| 42898 | LRLSCAASGFTFSSYAMH | H | DYADSVKG | GSYFPVHG |
| | WVRQAPGKGLEWVAVT | (SEQ ID | (SEQ ID | MDV |
| | WFDVSKKDYADSVKGRF | NO: 6) | NO: 11) | (SEQ ID |
| | TISRDNSKNTLYLQMNSL | | | NO: 16) |
| | RAEDTAVYYCARNLIRYS | | | |
| | GSYFPVHGMDVWGQGT | | | |
| | TVTVSS | | | |
| | (SEQ ID NO: 1) | | | |
| ADI- | QVQLQESGPGLVKPSQT | GSLSRGGY | NIFYSGSTY | ARNGRWV |
| 42877 | LSLTCTVSGGSLSRGGYY | YWS | YNPSLKS | TPDY |
| | WSWIRQHPGKGLEWIGNI | (SEQ ID | (SEQ ID | (SEQ ID |
| | FYSGSTYYNPSLKSRVTIS | NO: 7) | NO: 12) | NO: 17) |
| | VDTSTSQFSLKLNSVTAA | | | |
| | DTAVYYCARNGRWVTPD | | | |
| | YWGQGTLVTVSS | | | |
| | (SEQ ID NO: 2) | | | |
| ADI- | QVQLQESGPGLVRPSQT | DSIRNSLYY | RIYNSGSIN | ARGGYYYD |
| 42098 | LSLTCTVSGDSIRNSLYY | WS | YNPSLKS | GSGFFLPY |
| | WSWIRQPAGKGLEWIGRI | (SEQ ID | (SEQ ID | YFDY |
| | YNSGSINYNPSLKSRVTIL | NO: 8) | NO: 13) | (SEQ ID |
| | VDTSKNQFSLKLSSVTAA | | | NO: 18) |
| | DTAVYYCARGGYYYDGS | | | |
| | GFFLPYYFDYWGQGTLV | | | |
| | TVSS | | | |
| | (SEQ ID NO: 3) | | | |
| ADI- | EVQLVESGGGLVQPGGS | FTVSSTYM | VIYSGGNT | ARDLHRSR |
| 42885 | LRLSCAASGFTVSSTYMS | S | YYTDSVKG | PGP |
| | WVRQAPGKGLECVSVIYS | (SEQ ID | (SEQ ID | (SEQ ID |
| | GGNTYYTDSVKGRFTISR | NO: 9) | NO: 14) | NO: 19) |
| | DNSKNTLYLQMNSLRAED | | | |
| | TAVYYCARDLHRSRPGP | | | |
| | WGQGTLVTVSS | | | |
| | (SEQ ID NO: 4) | | | |
| ADI- | QITLKESGPTLVKPTQTLT | FSLSTSGV | LIFWNEDK | ARRVYSDY |
| 37236 | LTCTFSGFSLSTSGVGVG | GVG | RYSPSLKT | GQGAVFDY |
| | WIRQPPGKALEWLALIFW | (SEQ ID | (SEQ ID | (SEQ ID |
| | NEDKRYSPSLKTRLTITKD | NO: 10) | NO: 15) | NO: 20) |
| | TSKNQVVLTMTNMDPVD | | | |
| | TATYYCARRVYSDYGQG | | | |
| | AVFDYWGQGTLVTVSS | | | |
| | (SEQ ID NO: 5) | | | |
| ADI- | QVQLVESGGGVVQPGRS | GFTFSSYA | TWFDVSKK | ARNLIRYSV |
| 65533 | LRLSCAASGFTFSSYAMH | (SEQ ID | (SEQ ID | SYFPVHGM |
| | WVRQAPGKGLEWVAVT | NO: 72) | NO: 73) | DV |
| | WFDVSKKDYADSVKGRF | | | (SEQ ID |
| | TISRDNSKNTLYLQMNSL | | | NO: 74) |
| | RAEDTAVYYCARNLIRYS | | | |
| | VSYFPVHGMDVWGQGTT | | | |
| | VTVSS | | | |
| | (SEQ ID NO: 68) | | | |
| ADI- | QVQLVESGGGVVQPGRS | GFEFSSYA | TWFDVSKK | ARNLIRYSV |
| 65534 | LRLSCAASGFEFSSYAMH | (SEQ ID | (SEQ ID | SYFPVHGM |
| | WVRQAPGKGLEWVAVT | NO: 75) | NO: 76) | DV |
| | WFDVSKKDYADSVKGRF | | | (SEQ ID |
| | TISRDNSKNTLYLQMNSL | | | NO: 77) |
| | RAEDTAVYYCARNLIRYS | | | |
| | VSYFPVHGMDVWGQGTT | | | |
| | VTVSS | | | |
| | (SEQ ID NO: 69) | | | |
| ADI- | QVQLVESGGGVVQPGRS | GFTFSSGA | IWYDGSNK | RNLIRYSG |
| 42898IGL | LRLSCAASGFTFSSGAMH | (SEQ ID | YYADSVK | SYFPVHGM |
| | WVRQAPGKGLEWVAVIW | NO: 106) | (SEQ ID | D (SEQ ID |
| | YDGSNKYYADSVKGRFTI | | NO: 107) | NO: 108) |
| | SRDNSKNTLYLQMNSLRA | | | |
| | EDTAVYYCARNLIRYSGS | | | |
| | YFPVHGMDVWGQGTTVT | | | |
| | VSS (SEQ ID NO: 91) | | | |

TABLE 2
Antibodies and light chain CDR sequences

| Antibody | | | | |
|---|---|---|---|---|
| ID | V_{L} | LCDR1 | LCDR2 | LCDR2 |
| ADI-42898 | DIVMTQSPLSLPVTPGE | RSSQSLLH | LGSYRAS | MQALHPFT |
| | PASISCRSSQSLLHTYG | TYGYNSLD | (SEQ ID | (SEQ ID |
| | YNSLDWYLQRPGQSPQ | (SEQ ID | NO: 31) | NO: 36) |
| | LLISLGSYRASGVPDRF | NO: 26) | | |
| | SGSGSGTDFTLKISRVE | | | |
| | AEDVGVYYCMQALHPF | | | |
| | TFGGGTKVEIK | | | |
| | (SEQ ID NO: 21) | | | |
| | DIVMTQSPLSLPVTPGE | RSSQSLLH | LGSYRAS | MQALHPFT |
| | PASISCRSSQSLLHTYG | TYGYNCLD | (SEQ ID | (SEQ ID |
| | YNCLDWYLQRPGQSPQ | (SEQ ID | NO: 31) | NO: 36) |
| | LLISLGSYRASGVPDRF | NO: 82) | | |
| | SGSGSGTDFTLKISRVE | | | |
| | AEDVGVYYCMQALHPF | | | |
| | TFGGGTKVEIK | | | |
| | (SEQ ID NO: 81) | | | |
| ADI-42877 | QSVLTQPPSASGTPGQ | SGSSSNIGS | SNNQRPS | AAWDESLH |
| | RVTISCSGSSSNIGSNTV | NTVS | (SEQ ID | GPV |
| | SWYQQLPGTAPKLLIYS | (SEQ ID | NO: 32) | (SEQ ID |
| | NNQRPSGVPDRFSGSK | NO: 27) | | NO: 37) |
| | SGTSGSLAISGLQSEDE | | | |
| | ADYYCAAWDESLHGPV | | | |
| | FGGGTKLTVL | | | |
| | (SEQ ID NO: 22) | | | |
| ADI-42098 | EIVLTQSPGTLSLSPGER | RASQSVRN | SASSRAT | QQYGSEP |
| | ATLSCRASQSVRNTYLA | TYLA | (SEQ ID | WT |
| | WYQQKPGQAPRLLIYSA | (SEQ ID | NO: 33) | (SEQ ID |
| | SSRATGIPDRFGGSGSG | NO: 28) | | NO: 38) |
| | TDFILTISRLEPEDFAVY | | | |
| | YCQQYGSEPWTFGQG | | | |
| | TKVEIK | | | |
| | (SEQ ID NO: 23) | | | |
| ADI-42885 | DIQMTQSPSSVSASVGD | RASQGLST | AASSLQS | QQANTFPP |
| | RVTITCRASQGLSTWLA | WLA | (SEQ ID | T |
| | WYQQKPGKAPKLLIYAA | (SEQ ID | NO: 34) | (SEQ ID |
| | SSLQSGVPSRFSGSGS | NO: 29) | | NO: 39) |
| | GTDFTLTISSLQPEDFAT | | | |
| | YYCQQANTFPPTFGGG | | | |
| | TKVEIK | | | |
| | (SEQ ID NO: 24) | | | |
| ADI-37236 | DIQMTQSPSSLSASVGD | RASQGISN | AAFNLHS | QQYNYYPL |
| | RVTITCRASQGISNYLA | YLA | (SEQ ID | T |
| | WFQQKPGKAPKSLIYAA | (SEQ ID | NO: 35) | (SEQ ID |
| | FNLHSGVPSRFSGSGS | NO: 30) | | NO: 40) |
| | GTDFTLTISSLQPEDFAT | | | |
| | YYCQQYNYYPLTFGGG | | | |
| | TKVEIK | | | |
| | (SEQ ID NO: 25) | | | |
| ADI-65533 | DIVMTQSPLSLPVTPGE | QSLLHTYG | LGS | MQALHPFT |
| or ADI- | PASISCRSSQSLLHTYG | YNV | (SEQ ID | (SEQ ID |
| 65534 | YNVLDWYLQRPGQSPQ | (SEQ ID | NO: 79) | NO: 80) |
| | LLISLGSYRASGVPDRF | NO: 78) | | |
| | SGSGSGTDFTLKISRVE | | | |
| | AEDVGVYYCMQALHPF | | | |
| | TFGGGTKVEIK | | | |
| | (SEQ ID NO: 70) | | | |
| ADI- | DIVMTQSPLSLPVTPGE | RSSQSLLH | LGSNRA | MQALHPFT |
| 42898/GL | PASISCRSSQSLLHSNG | SNGYNYL | (SEQ ID | (SEQ ID |
| | YNYLDWYLQKPGQSPQ | (SEQ ID NO: | NO: 110) | NO: 111) |
| | LLIYLGSNRASGVPDRF | 109) | | |
| | SGSGSGTDFTLKISRVE | | | |
| | AEDVGVYYCMQALHPF | | | |
| | TFGGGTKVEIK (SEQ ID | | | |
| | NO: 92) | | | |

TABLE 3
Mutations of ADI-65533 and ADI-65534 vs ADI-42898.

| | Mutations | |
|---|---|---|
| Antibody | vs ADI- | |
| ID | 42898 | Mutation locations in CDRs |
| ADI-42898 | — | HCDR1: FTFSSYAMH (SEQ ID NO: 6) |
| | | HCDR3: ARNLIRYSGSYFPVHGMDV (SEQ ID NO: 16) |
| | | LCDR1: RSSQSLLHTYGYNSLD (SEQ ID NO: 26) |
| | | HCDR1: FTFSSYAMH (SEQ ID NO: 6) |
| | | HCDR3: ARNLIRYSGSYFPVHGMDV (SEQ ID NO: 16) |
| | | LCDR1: RSSQSLLHTYGYNCLD (SEQ ID NO: 82) |
| ADI-65533 | HV: G105V | HCDR3: ARNLIRYSVSYFPVHGMDV (SEQ ID NO: 74) |
| | LV: S37V | LCDR1: QSLLHTYGYNV (SEQ ID NO: 78) |
| ADI-65534 | HV: T28E, | HCDR1: GFEFSSYA (SEQ ID NO: 75) |
| | G105V | HCDR3: ARNLIRYSVSYFPVHGMDV (SEQ ID NO: 77) |
| | LV: S37V | LCDR1: QSLLHTYGYNV (SEQ ID NO: 78) |

In some embodiments, the antibody comprises or consists of: a V_{H} region and a V_{L} region, wherein (a) the V_{H} region has about 10%, about 20%, about 30%, about 40%, about 50%, about 60%, about 70%, about 80%, about 90%, about 95%, about 99%, or more identity to SEQ ID NO: 1; and comprises a CDR1 of SEQ ID NO:6, or the CDR1 of SEQ ID NO: 6 in which 1, 2, 3, 4, or 5 amino acids are substituted; a CDR2 of SEQ ID NO: 11, or the CDR2 of SEQ ID NO: 11 in which 1, 2, 3, 4, or 5 amino acids are substituted; a CDR3 of SEQ ID NO: 16 or the CDR3 of SEQ ID NO: 16 in which 1, 2, 3, 4, or 5 are substituted; and (b) the V_{L} region has about 10%, about 20%, about 30%, about 40%, about 50%, about 60%, about 70%, about 80%, about 90%, about 95%, about 99%, or more identity to SEQ ID NO: 81, 21, and comprises or consists of a CDR1 of SEQ ID NO: 26 or the CDR1 of SEQ ID NO: 26 in which 1, 2, 3, 4, or 5 amino acids are substituted; a CDR2 of SEQ ID NO: 31, or the CDR2 of SEQ ID NO: 31 in which 1, 2, 3, 4, or 5 amino acids are substituted; a CDR3 of SEQ ID NO: 36 or the CDR3 of SEQ ID NO: 36 in which 1, 2, 3, 4, or 5 are substituted.

In some embodiments, the antibody comprises or consists of: a V_{H} region and a V_{L} region, wherein (a) the V_{H} region has at least 10%, at least 20%, at least 30%, at least 40%, at least 50%, at least 60%, at least 70%, at least 80%, at least 90%, at least 95%, at least 99%, or more identity to SEQ ID NO: 1; and comprises a CDR1 of SEQ ID NO:6, or the CDR1 of SEQ ID NO: 6 in which 1, 2, 3, 4, or 5 amino acids are substituted; a CDR2 of SEQ ID NO: 11, or the CDR2 of SEQ ID NO: 11 in which 1, 2, 3, 4, or 5 amino acids are substituted; a CDR3 of SEQ ID NO: 16 or the CDR3 of SEQ ID NO: 16 in which 1, 2, 3, 4, or 5 are substituted; and (b) the V_{L} region has at least 10%, at least 20%, at least 30%, at least 40%, at least 50%, at least 60%, at least 70%, at least 80%, at least 90%, at least 95%, at least 99%, or more identity to SEQ ID NO: 81, 21, and comprises or consists of a CDR1 of SEQ ID NO: 26 or the CDR1 of SEQ ID NO: 26 in which 1, 2, 3, 4, or 5 amino acids are substituted; a CDR2 of SEQ ID NO: 31, or the CDR2 of SEQ ID NO: 31 in which 1, 2, 3, 4, or 5 amino acids are substituted; a CDR3 of SEQ ID NO: 36 or the CDR3 of SEQ ID NO: 36 in which 1, 2, 3, 4, or 5 are substituted.

In some embodiments, the antibody comprises or consists of: a V_{H} region and a V_{L} region, wherein (a) the V_{H} region has at least about 10%, at least about 20%, at least about 30%, at least about 40%, at least about 50%, at least about 60%, at least about 70%, at least about 80%, at least about 90%, at least about 95%, at least about 99%, or more identity to SEQ ID NO: 1; and comprises a CDR1 of SEQ ID NO:6, or the CDR1 of SEQ ID NO: 6 in which 1, 2, 3, 4, or 5 amino acids are substituted; a CDR2 of SEQ ID NO: 11, or the CDR2 of SEQ ID NO: 11 in which 1, 2, 3, 4, or 5 amino acids are substituted; a CDR3 of SEQ ID NO: 16 or the CDR3 of SEQ ID NO: 16 in which 1, 2, 3, 4, or 5 are substituted; and (b) the V_{L} region has at least about 10%, at least about 20%, at least about 30%, at least about 40%, at least about 50%, at least about 60%, at least about 70%, at least about 80%, at least about 90%, at least about 95%, at least about 99%, or more identity to SEQ ID NO: 81, 21, and comprises or consists of a CDR1 of SEQ ID NO: 26 or the CDR1 of SEQ ID NO: 26 in which 1, 2, 3, 4, or 5 amino acids are substituted; a CDR2 of SEQ ID NO: 31, or the CDR2 of SEQ ID NO: 31 in which 1, 2, 3, 4, or 5 amino acids are substituted; a CDR3 of SEQ ID NO: 36 or the CDR3 of SEQ ID NO: 36 in which 1, 2, 3, 4, or 5 are substituted.

In some embodiments, the antibody comprises or consists of: a V_{H} region and a V_{L}, wherein (a) the V_{H} region has about 10% to about 99%, about 20% to about 90%, about 30% to about 80%, about 40% to about 70%, about 50% to about 95%, about 60% to about 85%, or about 70% to about 90%, or about 85% to about 95% identity to SEQ ID NO: 1; and comprises a CDR1 of SEQ ID NO:6, or the CDR1 of SEQ ID NO: 6 in which 1, 2, 3, 4, or 5 amino acids are substituted; a CDR2 of SEQ ID NO: 11, or the CDR2 of SEQ ID NO: 11 in which 1, 2, 3, 4, or 5 amino acids are substituted; a CDR3 of SEQ ID NO: 16 or the CDR3 of SEQ ID NO: 16 in which 1, 2, 3, 4, or 5 are substituted; and (b) the V_{L} region has about 10% to about 99%, about 20% to about 90%, about 30% to about 80%, about 40% to about 70%, about 50% to about 95%, about 60% to about 85%, or about 70% to about 90%, or about 85% to about 95% identity to SEQ ID NO: 81, 21, and comprises or consists of a CDR1 of SEQ ID NO: 26 or the CDR1 of SEQ ID NO: 26 in which 1, 2, 3, 4, or 5 amino acids are substituted; a CDR2 of SEQ ID NO: 31, or the CDR2 of SEQ ID NO: 31 in which 1, 2, 3, 4, or 5 amino acids are substituted; a CDR3 of SEQ ID NO: 36 or the CDR3 of SEQ ID NO: 36 in which 1, 2, 3, 4, or 5 are substituted.

In some embodiments, the antibody comprises or consists of: a V_{H} region and a V_{L} region, wherein (a) the V_{H} region has about 10%, about 20%, about 30%, about 40%, about 50%, about 60%, about 70%, about 80%, about 90%, about 95%, about 99%, or more identity to SEQ ID NO:68; and comprises a CDR1 of SEQ ID NO:72, or the CDR1 of SEQ ID NO:72 in which 1, 2, 3, 4, or 5 amino acids are substituted; a CDR2 of SEQ ID NO:73, or the CDR2 of SEQ ID NO:73 in which 1, 2, 3, 4, or 5 amino acids are substituted; a CDR3 of SEQ ID NO:74 or the CDR3 of SEQ ID NO:74 in which 1, 2, 3, 4, or 5 are substituted; and (b) the V_{L} region has about 10%, about 20%, about 30%, about 40%, about 50%, about 60%, about 70%, about 80%, about 90%, about 95%, about 99%, or more identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a V_{H} region and a V_{L} region, wherein (a) the V_{H} region has at least 10%, at least 20%, at least 30%, at least 40%, at least 50%, at least 60%, at least 70%, at least 80%, at least 90%, at least 95%, at least 99%, or more identity to SEQ ID NO:68; and comprises a CDR1 of SEQ ID NO:72, or the CDR1 of SEQ ID NO:72 in which 1, 2, 3, 4, or 5 amino acids are substituted; a CDR2 of SEQ ID NO:73, or the CDR2 of SEQ ID NO:73 in which 1, 2, 3, 4, or 5 amino acids are substituted; a CDR3 of SEQ ID NO:74 or the CDR3 of SEQ ID NO:74 in which 1, 2, 3, 4, or 5 are substituted; and (b) the V_{L} region has at least 10%, at least 20%, at least 30%, at least 40%, at least 50%, at least 60%, at least 70%, at least 80%, at least 90%, at least 95%, at least 99%, or more identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a V_{H} region and a V_{L} region, wherein (a) the V_{H} region has at least 10%, at least 20%, at least 30%, at least 40%, at least 50%, at least 60%, at least 70%, at least 80%, at least 90%, at least 95%, at least 99%, or more identity to SEQ ID NO:68; and comprises a CDR1 of SEQ ID NO:72, or the CDR1 of SEQ ID NO:72 in which 1, 2, 3, 4, or 5 amino acids are substituted; a CDR2 of SEQ ID NO:73, or the CDR2 of SEQ ID NO:73 in which 1, 2, 3, 4, or 5 amino acids are substituted; a CDR3 of SEQ ID NO:74 or the CDR3 of SEQ ID NO:74 in which 1, 2, 3, 4, or 5 are substituted; and (b) the V_{L} region has at least 10%, at least 20%, at least 30%, at least 40%, at least 50%, at least 60%, at least 70%, at least 80%, at least 90%, at least 95%, at least 99%, or more identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a V_{H} region and a V_{L} region, wherein (a) the V_{H} region has about 10% to about 99%, about 20% to about 90%, about 30% to about 80%, about 40% to about 70%, about 50% to about 95%, about 60% to about 85%, or about 70% to about 90%, or about 85% to about 95% identity to SEQ ID NO:68; and comprises a CDR1 of SEQ ID NO:72, or the CDR1 of SEQ ID NO:72 in which 1, 2, 3, 4, or 5 amino acids are substituted; a CDR2 of SEQ ID NO:73, or the CDR2 of SEQ ID NO:73 in which 1, 2, 3, 4, or 5 amino acids are substituted; a CDR3 of SEQ ID NO:74 or the CDR3 of SEQ ID NO:74 in which 1, 2, 3, 4, or 5 are substituted; and (b) the V_{L} region has about 10% to about 99%, about 20% to about 90%, about 30% to about 80%, about 40% to about 70%, about 50% to about 95%, about 60% to about 85%, or about 70% to about 90%, or about 85% to about 95% identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a V_{H} region and a V_{L} region, wherein (a) the V_{H} region has about 10%, about 20%, about 30%, about 40%, about 50%, about 60%, about 70%, about 80%, about 90%, about 95%, about 99%, or more identity to SEQ ID NO:69; and comprises a CDR1 of SEQ ID NO:75, or the CDR1 of SEQ ID NO:75 in which 1, 2, 3, 4, or 5 amino acids are substituted; a CDR2 of SEQ ID NO:76, or the CDR2 of SEQ ID NO:76 in which 1, 2, 3, 4, or 5 amino acids are substituted; a CDR3 of SEQ ID NO:77 or the CDR3 of SEQ ID NO:77 in which 1, 2, 3, 4, or 5 are substituted; and (b) the V_{L} region has about 10%, about 20%, about 30%, about 40%, about 50%, about 60%, about 70%, about 80%, about 90%, about 95%, about 99%, or more identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a V_{H} region and a V_{L} region, wherein (a) the V_{H} region has at least 10%, at least 20%, at least 30%, at least 40%, at least 50%, at least 60%, at least 70%, at least 80%, at least 90%, at least 95%, at least 99%, or more identity to SEQ ID NO:69; and comprises a CDR1 of SEQ ID NO:75, or the CDR1 of SEQ ID NO:75 in which 1, 2, 3, 4, or 5 amino acids are substituted; a CDR2 of SEQ ID NO:76, or the CDR2 of SEQ ID NO:76 in which 1, 2, 3, 4, or 5 amino acids are substituted; a CDR3 of SEQ ID NO:77 or the CDR3 of SEQ ID NO:77 in which 1, 2, 3, 4, or 5 are substituted; and (b) the V_{L} region has at least 10%, at least 20%, at least 30%, at least 40%, at least 50%, at least 60%, at least 70%, at least 80%, at least 90%, at least 95%, at least 99%, or more identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a V_{H} region and a V_{L} region, wherein (a) the V_{H} region has at least about 10%, at least about 20%, at least about 30%, at least about 40%, at least about 50%, at least about 60%, at least about 70%, at least about 80%, at least about 90%, at least about 95%, at least about 99%, or more identity to SEQ ID NO:69; and comprises a CDR1 of SEQ ID NO:75, or the CDR1 of SEQ ID NO:75 in which 1, 2, 3, 4, or 5 amino acids are substituted; a CDR2 of SEQ ID NO:76, or the CDR2 of SEQ ID NO:76 in which 1, 2, 3, 4, or 5 amino acids are substituted; a CDR3 of SEQ ID NO:77 or the CDR3 of SEQ ID NO:77 in which 1, 2, 3, 4, or 5 are substituted; and (b) the V_{L} region has at least about 10%, at least about 20%, at least about 30%, at least about 40%, at least about 50%, at least about 60%, at least about 70%, at least about 80%, at least about 90%, at least about 95%, at least about 99%, or more identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a heavy chain variable (V_{H}) region and a light chain variable (V_{L}) region, wherein (a) the V_{H} region has about 10% to about 99%, about 20% to about 90%, about 30% to about 80%, about 40% to about 70%, about 50% to about 95%, about 60% to about 85%, or about 70% to about 90%, or about 85% to about 95% identity to SEQ ID NO:69; and comprises a CDR1 of SEQ ID NO:75, or the CDR1 of SEQ ID NO:75 in which 1, 2, 3, 4, or 5 amino acids are substituted; a CDR2 of SEQ ID NO:76, or the CDR2 of SEQ ID NO:76 in which 1, 2, 3, 4, or 5 amino acids are substituted; a CDR3 of SEQ ID NO:77 or the CDR3 of SEQ ID NO:77 in which 1, 2, 3, 4, or 5 are substituted; and (b) the V_{L} region has about 10% to about 99%, about 20% to about 90%, about 30% to about 80%, about 40% to about 70%, about 50% to about 95%, about 60% to about 85%, or about 70% to about 90%, or about 85% to about 95% identity to SEQ ID NO:70, and comprises or consists of a CDR1 of SEQ ID NO:78 or the CDR1 of SEQ ID NO:78 in which 1, 2, 3, 4, or 5 amino acids are substituted; a CDR2 of SEQ ID NO:79, or the CDR2 of SEQ ID NO:79 in which 1, 2, 3, 4, or 5 amino acids are substituted; a CDR3 of SEQ ID NO:80 or the CDR3 of SEQ ID NO:80 in which 1, 2, 3, 4, or 5 are substituted.

In some embodiments, the antibody comprises or consists of: a V_{H} region comprising amino acid sequence SEQ ID NO: 1 and a V_{L} region comprising or consisting of amino acid sequence SEQ ID NO: 81, 21; a V_{H} region comprising or consisting of amino acid sequence SEQ ID NO: 2 and a V_{L} region comprising or consisting of amino acid sequence SEQ ID NO: 22; a V_{H} region comprising or consisting of amino acid sequence SEQ ID NO: 3 and a V_{L} region comprising or consisting of amino acid sequence SEQ ID NO: 23; a V_{H} region comprising or consisting of amino acid sequence SEQ ID NO: 4 and a V_{L} region comprising or consisting of amino acid sequence SEQ ID NO: 24; a V_{H} region comprising or consisting of amino acid sequence SEQ ID NO: 5 and a V_{L} region comprising or consisting of amino acid sequence SEQ ID NO: 25; a V_{H} region comprising or consisting of amino acid sequence SEQ ID NO: 68 and a V_{L} region comprising or consisting of amino acid sequence SEQ ID NO: 70; or a V_{H} region comprising or consisting of amino acid sequence SEQ ID NO: 69 and a V_{L} region comprising or consisting of amino acid sequence SEQ ID NO: 70.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises a heavy chain variable region having a sequence that is about 10%, about 20%, about 30%, about 40%, about 50%, about 60%, about 70%, about 80%, about 90%, about 95%, about 99%, or more identical to that of the corresponding heavy chain variable region and a light chain variable region having a sequence that is about 95% identical to the corresponding light chain variable region.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises or consists of a heavy chain variable region having a sequence that is about 10%, about 20%, about 30%, about 40%, about 50%, about 60%, about 70%, about 80%, about 90%, about 95%, about 99%, or more identical to that of the corresponding heavy chain variable region and a light chain variable region having a sequence that is about 10%, about 20%, about 30%, about 40%, about 50%, about 60%, about 70%, about 80%, about 90%, about 95%, about 99%, or more identical to the corresponding light chain variable region.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises or consists of a heavy chain variable region having a sequence that is at least 10%, at least 20%, at least 30%, at least 40%, at least 50%, at least 60%, at least 70%, at least 80%, at least 90%, at least 95%, at least 99%, or more identical to that of the corresponding heavy chain variable region and a light chain variable region having a sequence that is at least 95% identical to the corresponding light chain variable region.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises or consists of a heavy chain variable region having a sequence that is at least 10%, at least 20%, at least 30%, at least 40%, at least 50%, at least 60%, at least 70%, at least 80%, at least 90%, at least 95%, at least 99%, or more identical to that of the corresponding heavy chain variable region and a light chain variable region having a sequence that is at least 10%, at least 20%, at least 30%, at least 40%, at least 50%, at least 60%, at least 70%, at least 80%, at least 90%, at least 95%, at least 99%, or more identical to the corresponding light chain variable region.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises or consists of a heavy chain variable region having a sequence that is at least about 10%, at least about 20%, at least about 30%, at least about 40%, at least about 50%, at least about 60%, at least about 70%, at least about 80%, at least about 90%, at least about 95%, at least about 99%, or more identical to that of the corresponding heavy chain variable region and a light chain variable region having a sequence that is at least about 95% identical to the corresponding light chain variable region.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises or consists of a heavy chain variable region having a sequence that is at least about 10%, at least about 20%, at least about 30%, at least about 40%, at least about 50%, at least about 60%, at least about 70%, at least about 80%, at least about 90%, at least about 95%, at least about 99%, or more identical to that of the corresponding heavy chain variable region and a light chain variable region having a sequence that is at least about 10%, at least about 20%, at least about 30%, at least about 40%, at least about 50%, at least about 60%, at least about 70%, at least about 80%, at least about 90%, at least about 95%, at least about 99%, or more identical to the corresponding light chain variable region.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises or consists of a heavy chain variable region having a sequence that is about 10% to about 99%, about 20% to about 90%, about 30% to about 80%, about 40% to about 70%, about 50% to about 95%, about 60% to about 85%, or about 70% to about 90%, or about 85% to about 95% identical to that of the corresponding heavy chain variable region and a light chain variable region having a sequence that is at least 95% identical to the corresponding light chain variable region.

In some embodiments, the antibody comprises or consists of a heavy chain variable region and a light chain variable region of an antibody designated as ADI-42898, ADI-42877, ADI-42098, ADI-42885, ADI-37236, ADI-65533, or ADI-65534 or a variant thereof, wherein the variant comprises or consists of a heavy chain variable region having a sequence that is about 10% to about 99%, about 20% to about 90%, about 30% to about 80%, about 40% to about 70%, about 50% to about 95%, about 60% to about 85%, or about 70% to about 90%, or about 85% to about 95% identical to that of the corresponding heavy chain variable region and a light chain variable region having a sequence that is about 10% to about 99%, about 20% to about 90%, about 30% to about 80%, about 40% to about 70%, about 50% to about 95%, about 60% to about 85%, or about 70% to about 90%, or about 85% to about 95% identical to the corresponding light chain variable region.

In some embodiments, wherein at least 1 or 2 of the substitutions are conservative substitutions, about 50% of the substitutions are conservative, or all are conservative substitutions.

In some embodiments, wherein at least 1 or 2 of the substitutions are conservative substitutions, at least 50% of the substitutions are conservative, or all are conservative substitutions.

In some embodiments, wherein at least 1 or 2 of the substitutions are conservative substitutions, at least about 50% of the substitutions are conservative, or all are conservative substitutions.

In some embodiments, wherein about 1 or 2 of the substitutions are conservative substitutions, about 10%, about 20%, about 30%, about 40%, about 50%, about 60%, about 70%, about 80%, about 90%, about 95%, about 99%, or more of the substitutions are conservative substitutions; or all the substitutions are conservative substitutions.

In some embodiments, wherein at least 1 or 2 of the substitutions are conservative substitutions, at least 10%, at least 20%, at least 30%, at least 40%, at least 50%, at least 60%, at least 70%, at least 80%, at least 90%, at least 95%, at least 99%, or more of the substitutions are conservative substitutions; or all the substitutions are conservative substitutions.

In some embodiments, wherein at least about 1 or 2 of the substitutions are conservative substitutions, at least about 10%, at least about 20%, at least about 30%, at least about 40%, at least about 50%, at least about 60%, at least about 70%, at least about 80%, at least about 90%, at least about 95%, at least about 99%, or more of the substitutions are conservative substitutions; or all the substitutions are conservative substitutions.

In some embodiments, wherein at least 1 or 2 of the substitutions are conservative substitutions, about 10% to about 99%, about 20% to about 90%, about 30% to about 80%, about 40% to about 70%, about 50% to about 95%, about 60% to about 85%, or about 70% to about 90%, or about 85% to about 95% of the substitutions are conservative substitutions; or all the substitutions are conservative substitutions.

In some embodiments, wherein at least 1 or 2 of the substitutions are conservative substitutions, not more than 55%, not more than 60%, not more than 70%, not more than 80%, not more than 90%, not more than 95%, not more than 99%, or less of the substitutions are conservative substitutions; or all the substitutions are conservative substitutions.

In some embodiments, the hantavirus antibodies of the present technology are natural antibodies (e.g., isolated from a subject previously exposed to a hantavirus). In some embodiments, the hantavirus antibodies of the present technology are non-natural antibodies.

In some embodiments, the hantavirus antibody is an antibody encoded by a recombinant polypeptide expressed in a vector. In some embodiments, the polypeptide comprises or consists of (1) a V_{H} sequence having about 70% amino acid sequence identity to SEQ ID NO:1 and/or (2) a V_{L} sequence having about 70% amino acid sequence identity to SEQ ID NO: 81, 21. In some embodiments, the polypeptide comprises or consists of (1) a V_{H} sequence having about 70% amino acid sequence identity to SEQ ID NO:68 and/or (2) a V_{L} sequence having about 70% amino acid sequence identity to SEQ ID NO:70. In some embodiments, the polypeptide comprises or consists of (1) a V_{H} sequence having about 70% amino acid sequence identity to SEQ ID NO:69 and/or (2) a V_{L} sequence having about 70% amino acid sequence identity to SEQ ID NO:70.

In some embodiments, the hantavirus antibody is an antibody encoded by a recombinant polypeptide expressed in a vector. In some embodiments, the polypeptide comprises or consists of (1) a V_{H} sequence having at least 70% amino acid sequence identity to SEQ ID NO:1 and/or (2) a V_{L} sequence having at least 70% amino acid sequence identity to SEQ ID NO: 81, 21. In some embodiments, the polypeptide comprises or consists of (1) a V_{H} sequence having at least 70% amino acid sequence identity to SEQ ID NO:68 and/or (2) a V_{L} sequence having at least 70% amino acid sequence identity to SEQ ID NO:70. In some embodiments, the polypeptide comprises or consists of (1) a V_{H} sequence having at least 70% amino acid sequence identity to SEQ ID NO:69 and/or (2) a V_{L} sequence having at least 70% amino acid sequence identity to SEQ ID NO:70.

In some embodiments, the hantavirus antibody is an antibody encoded by a recombinant polypeptide expressed in a vector. In some embodiments, the polypeptide comprises or consists of (1) a V_{H} sequence having at least about 70% amino acid sequence identity to SEQ ID NO:1 and/or (2) a V_{L} sequence having at least about 70% amino acid sequence identity to SEQ ID NO: 81, 21. In some embodiments, the polypeptide comprises or consists of (1) a V_{H} sequence having at least about 70% amino acid sequence identity to SEQ ID NO:68 and/or (2) a V_{L} sequence having at least about 70% amino acid sequence identity to SEQ ID NO:70. In some embodiments, the polypeptide comprises or consists of (1) a V_{H} sequence having at least about 70% amino acid sequence identity to SEQ ID NO:69 and/or (2) a V_{L} sequence having at least about 70% amino acid sequence identity to SEQ ID NO:70.

In some embodiments, the polypeptide comprises or consists of (1) a V_{H} sequence having at least 10%, at least 20%, at least 30%, at least 40%, at least 50%, at least 60%, at least 70%, at least 80%, at least 90%, at least 95%, at least 99%, or more amino acid sequence identity to SEQ ID NO:1 and/or (2) a V_{L} Sequence having at least 10%, at least 20%, at least 30%, at least 40%, at least 50%, at least 60%, at least 70%, at least 80%, at least 90%, at least 95%, at least 99%, or more amino acid sequence identity to SEQ ID NO: 81 or 21.

In some embodiments, the polypeptide comprises or consists of (1) a V_{H} sequence having about 10% to about 99%, about 20% to about 90%, about 30% to about 80%, about 40% to about 70%, about 50% to about 95%, about 60% to about 85%, or about 70% to about 90%, or about 85% to about 95% amino acid sequence identity to SEQ ID NO:1and/or (2) a V_{L} sequence having about 10% to about 99%, about 20% to about 90%, about 30% to about 80%, about 40% to about 70%, about 50% to about 95%, about 60% to about 85%, or about 70% to about 90%, or about 85% to about 95% amino acid sequence identity to SEQ ID NO: 81 or 21.

In some embodiments, the polypeptide comprises or consists of (1) a V_{H} sequence having about 10%, about 20%, about 30%, about 40%, about 50%, about 60%, about 70%, about 80%, about 90%, about 95%, about 99%, or more amino acid sequence identity to any one of SEQ ID NO:68-69 and/or (2) a V_{L} sequence having about 10%, about 20%, about 30%, about 40%, about 50%, about 60%, about 70%, about 80%, about 90%, about 95%, about 99%, or more amino acid sequence identity to SEQ ID NO:70.

In some embodiments, the polypeptide comprises or consists of (1) a V_{H} sequence having at least 10%, at least 20%, at least 30%, at least 40%, at least 50%, at least 60%, at least 70%, at least 80%, at least 90%, at least 95%, at least 99%, or more amino acid sequence identity to any one of SEQ ID NO:68-69 and/or (2) a V_{L} sequence having at least 10%, at least 20%, at least 30%, at least 40%, at least 50%, at least 60%, at least 70%, at least 80%, at least 90%, at least 95%, at least 99%, or more amino acid sequence identity to SEQ ID NO:70.

In some embodiments, the polypeptide comprises or consists of (1) a V_{H} sequence having at least about 10%, at least about 20%, at least about 30%, at least about 40%, at least about 50%, at least about 60%, at least about 70%, at least about 80%, at least about 90%, at least about 95%, at least about 99%, or more amino acid sequence identity to any one of SEQ ID NO:68-69 and/or (2) a V_{L} sequence having at least about 10%, at least about 20%, at least about 30%, at least about 40%, at least about 50%, at least about 60%, at least about 70%, at least about 80%, at least about 90%, at least about 95%, at least about 99%, or more amino acid sequence identity to SEQ ID NO:70.

In some embodiments, the polypeptide comprises or consists of (1) a V_{H} sequence having about 10% to about 99%, about 20% to about 90%, about 30% to about 80%, about 40% to about 70%, about 50% to about 95%, about 60% to about 85%, or about 70% to about 90%, or about 85% to about 95% amino acid sequence identity to any one of SEQ ID NO:68-69 and/or (2) a V_{L} sequence having about 10% to about 99%, about 20% to about 90%, about 30% to about 80%, about 40% to about 70%, about 50% to about 95%, about 60% to about 85%, or about 70% to about 90%, or about 85% to about 95% amino acid sequence identity to any one of SEQ ID NO:70.

Any conventional methods may be used for glycoengineering of antibodies. In some embodiments, the RAMP system is used for glycoengineering of antibodies, antibody fragments, idiotype vaccines, enzymes, and cytokines. The RAMP technology and its use in antibody discovery is disclosed in, e.g., U.S. Pat. No. 10,836,810, the entire content of which is herein incorporated by reference.

In some embodiments, a hantavirus antibody of the present technology targets one or more epitopes located in the Gn protein (e.g., capping loop region) or the Gc protein. In some embodiments, a hantavirus antibody targets one or more epitopes in the Gn capping loop protein at the position (e.g., E88, E95, Q98, or S101). In some embodiments, a hantavirus antibody targets one or more epitopes in the Gc protein (e.g., at the position D682, S830, 1844, P852, D959, or S966).

### Variants

In some embodiments, variants of any of the hantavirus antibodies of the present technology may be generated by introducing mutations to the heavy chain and/or light chain sequences. In some embodiments, the mutation(s) are introduced into one or more of the CDRs of a hantavirus antibody of the present technology. Nonlimiting examples of mutations include a mutation in the Gn^{H} capping loop (e.g., E95K, Q98P, S101N) and/or Gc domain I (e.g., D682N, 1844S, D959N, S966Y).

In some embodiments, the hantavirus antibody of the present technology comprises (1) a V_{H} region of any one of SEQ ID NOS:1-5, or 68-69, and/or (2) a V_{L} region of any one of SEQ ID NOS: 81, 21-25, or 70. In some embodiments, the hantavirus antibody of the present technology comprises (1) a V_{H} region with about 10%, about 20%, about 30%, about 40%, about 50%, about 60%, about 70%, about 80%, about 90%, about 95%, about 99%, or more identity to any one of SEQ ID NOS:1-5, or 68-69, and (2) a V_{L} region having about 10%, about 20%, about 30%, about 40%, about 50%, about 60%, about 70%, about 80%, about 90%, about 95%, about 99%, or more identity to any one of SEQ ID NOS: 81, 21-25, or 70 with variations to the corresponding V_{H} or V_{L} regions present only in Framework regions.

In some embodiments, the hantavirus antibody of the present technology comprises (1) a V_{H} region of any one of SEQ ID NOS:1-5, or 68-69, and/or (2) a V_{L} region of any one of SEQ ID NOS: 81, 21-25, or 70. In some embodiments, the hantavirus antibody of the present technology comprises (1) a V_{H} region with at least 10%, at least 20%, at least 30%, at least 40%, at least 50%, at least 60%, at least 70%, at least 80%, at least 90%, at least 95%, at least 99%, or more identity to any one of SEQ ID NOS:1-5, or 68-69, and (2) a V_{L} region having at least 10%, at least 20%, at least 30%, at least 40%, at least 50%, at least 60%, at least 70%, at least 80%, at least 90%, at least 95%, at least 99%, or more identity to any one of SEQ ID NOS: 81, 21-25, or 70 with variations to the corresponding V_{H} or V_{L} regions present only in Framework regions.

In some embodiments, the hantavirus antibody of the present technology comprises (1) a V_{H} region of any one of SEQ ID NOS:1-5, or 68-69, and/or (2) a V_{L} region of any one of SEQ ID NOS: 81, 21-25, or 70. In some embodiments, the hantavirus antibody of the present technology comprises (1) a V_{H} region with at least about 10%, at least about 20%, at least about 30%, at least about 40%, at least about 50%, at least about 60%, at least about 70%, at least about 80%, at least about 90%, at least about 95%, at least about 99%, or more identity to any one of SEQ ID NOS:1-5, or 68-69, and (2) a V_{L} region having at least about 10%, at least about 20%, at least about 30%, at least about 40%, at least about 50%, at least about 60%, at least about 70%, at least about 80%, at least about 90%, at least about 95%, at least about 99%, or more identity to any one of SEQ ID NOS: 81, 21-25, or 70 with variations to the corresponding V_{H} or V_{L} regions present only in Framework regions.

In some embodiments, a hantavirus antibody of the present technology comprises mutation in the Gn^{H} capping loop of any one of SEQ ID NOS:41, 44-49, or 68-70 (Tables 1 or 2).

In some embodiments, variants of any of the hantavirus antibodies of the present technology may be generated by introducing mutations to the heavy chain and/or light chain sequences. In some embodiments, the mutation(s) are introduced into one or more of the CDRs of a hantavirus antibody of the present technology, e.g., a mutation in the Gn^{H} capping loop (e.g., E95K, Q98P, or S101N) and Gc domain I e.g., (D682N, 1844S, D959N, or S966Y). In some embodiments, the hantavirus antibody of the present technology comprises a V_{H} region of any one of SEQ ID NOS:1-5, or 68-69 and/or a V_{L} region of any one of SEQ ID NOS: 81, 21-25, or 70. In some embodiments, the hantavirus antibody of the present technology comprises a V_{H} region with at least 80% identity to any one of SEQ ID NOS:1-5, or 68-69 and a V_{L} region having at least 80% identity to any one of SEQ ID NOS: 81, 21-25, or 70 with variations to the corresponding V_{H} or V_{L} regions present only in Framework regions. In some embodiments, the hantavirus antibody comprises one or more mutations in the the Gn^{H} capping loop of any one of SEQ ID NOS:41, 44-49, or 68-70 (Tables 1 and 2).

In some embodiments, a hantavirus antibody of the present technology comprises (1) an HCDR1 having an amino acid sequence at least of any one of SEQ ID NOS: 6-10, 72, or 75; (2) an HCDR2 of any one of SEQ ID NOS:11-15, 73, or 76; (3) an HCDR3 of any one of SEQ ID NOS:16-20, 74, or 77; (4) an LCDR1 of any one of SEQ ID NOS:26-30, or 78; (5) an LCDR2 of any one of SEQ ID NOS:31-35, or 79; (6) an LCDR3 of any one of SEQ ID NOS:36-40; or 80, and the Framework regions in the V_{H} region are about 80% identical to the Framework regions present in the V_{H} region of SEQ ID NOS:1-5, 68, or 69, and wherein the Framework regions in the V_{L} region are about 80% identical to the Framework regions present in the V_{L} region of SEQ ID NOS: 81, 21-25, or 70.

In some embodiments, a hantavirus antibody of the present technology comprises (1) an HCDR1 having an amino acid sequence at least of any one of SEQ ID NOS: 6-10, 72, or 75; (2) an HCDR2 of any one of SEQ ID NOS:11-15, 73, or 76; (3) an HCDR3 of any one of SEQ ID NOS:16-20, 74, or 77; (4) an LCDR1 of any one of SEQ ID NOS:26-30, or 78; (5) an LCDR2 of any one of SEQ ID NOS:31-35, or 79; (6) an LCDR3 of any one of SEQ ID NOS:36-40; or 80, and the Framework regions in the V_{H} region are at least 80% identical to the Framework regions present in the V_{H} region of SEQ ID NOS:1-5, 68, or 69, and wherein the Framework regions in the V_{L} region are at least 80% identical to the Framework regions present in the V_{L} region of SEQ ID NOS: 81, 21-25, or 70.

In some embodiments, a hantavirus antibody of the present technology comprises (1) an HCDR1 having an amino acid sequence at least of any one of SEQ ID NOS: 6-10, 72, or 75; (2) an HCDR2 of any one of SEQ ID NOS:11-15, 73, or 76; (3) an HCDR3 of any one of SEQ ID NOS:16-20, 74, or 77; (4) an LCDR1 of any one of SEQ ID NOS:26-30, or 78; (5) an LCDR2 of any one of SEQ ID NOS:31-35, or 79; (6) an LCDR3 of any one of SEQ ID NOS:36-40; or 80, and the Framework regions in the V_{H} region are at least about 80% identical to the Framework regions present in the V_{H} region of SEQ ID NOS:1-5, 68, or 69, and wherein the Framework regions in the V_{L} region are at least about 80% identical to the Framework regions present in the V_{L} region of SEQ ID NOS: 81, 21-25, or 70.

In some embodiments, a variant is engineered to be as much like self as possible to minimize immunogenicity. This may comprise identifying a close germline sequence and mutatating one of the hantavirus antibodies at as many mismatched positions (also known as “germline deviations”) to the germline residue type as possible. In some embodiments, a variant comprises a mutation in the Gn region relative to the heavy chain of ADI-42898 and/or the light chain of ADI-42898 (e.g., E88D, E95K, Q98R, Q98P, or S101N). In some embodiments, a variant comprises a mutation E88D in the Gn subunit relative to the heavy chain of ADI-42898 and/or the light chain of ADI-42898. In some embodiments, a variant comprises a mutation E95K in the Gn subunit relative to the heavy chain of ADI-42898 and/or the light chain of ADI-42898. In some embodiments, a variant comprises a mutation Q98R in the Gn subunit relative to the heavy chain of ADI-42898 and/or the light chain of ADI-42898. In some embodiments, a variant comprises a mutation in the Gc subunit relative to the heavy chain of ADI-42898 and/or the light chain of ADI-42898 (e.g., D682N, S8301, 1844S, P852T, D959N, or S966Y). In some embodiments, a variant comprises a mutation Q98P in the Gn subunit relative to the heavy chain of ADI-42898 and/or the light chain ADI-42898. In some embodiments, a variant comprises a mutation S101N in the Gn subunit relative to the heavy chain of ADI-42898 and/or the light chain of ADI-42898. In some embodiments, a variant comprises a mutation D682N in the Gc subunit relative to the heavy chain of ADI-42898 and/or the light chain of ADI-42898. In some embodiments, a variant comprises a mutation S8301 in the Gc subunit relative to the heavy chain of ADI-42898 and/or the light chain of ADI-42898. In some embodiments, a variant comprises a mutation 1844S in the Gc subunit relative to the heavy chain of ADI-42898 and/or the light chain of ADI-42898. In some embodiments, a variant comprises a mutation P852T in the Gc subunit relative to the heavy chain of ADI-42898 and/or the light chain of ADI-42898. In some embodiments, a variant comprises a mutation D959N in the Gc subunit relative to the heavy chain of ADI-42898 and/or the light chain of ADI-42898. In some embodiments, a variant comprises a mutation S966Y in the Gc subunit relative to the heavy chain of ADI-42898 and/or the light chain of ADI-42898.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is about 95% identical to that of the corresponding heavy chain variable region and a light chain variable region having a sequence that is about 95% identical to the corresponding light chain variable region.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is at least 95% identical to that of the corresponding heavy chain variable region and a light chain variable region having a sequence that is at least 95% identical to the corresponding light chain variable region.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is at least about 95% identical to that of the corresponding heavy chain variable region and a light chain variable region having a sequence that is at least about 95% identical to the corresponding light chain variable region.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is about 10%, about 20%, about 30%, about 40%, about 50%, about 60%, about 70%, about 80%, about 90%, about 95%, about 99%, or more identical to that of the corresponding heavy chain variable region and a light chain variable region having a sequence that is about 95% identical to the corresponding light chain variable region.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is at least about 10%, at least about 20%, at least about 30%, at least about 40%, at least about 50%, at least about 60%, at least about 70%, at least about 80%, at least about 90%, at least about 95%, at least about 99%, or more identical to that of the corresponding heavy chain variable region and a light chain variable region having a sequence that is at least about 95% identical to the corresponding light chain variable region.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is at least 10%, at least 20%, at least 30%, at least 40%, at least 50%, at least 60%, at least 70%, at least 80%, at least 90%, at least 95%, at least 99%, or more identical to that of the corresponding heavy chain variable region and a light chain variable region having a sequence that is at least 95% identical to the corresponding light chain variable region.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is about 10% to about 99%, about 20% to about 90%, about 30% to about 80%, about 40% to about 70%, about 50% to about 95%, about 60% to about 85%, or about 70% to about 90%, or about 85% to about 95% identical to that of the corresponding heavy chain variable region and a light chain variable region having a sequence that is at least 95% identical to the corresponding light chain variable region.

In some embodiments, the antibody comprises a heavy chain variable region and a light chain variable region of an antibody designated as ADI-65533 or ADI-65534 (Tables 1 and 2), or a variant thereof, wherein the variant comprises a heavy chain variable region having a sequence that is about 10% to about 99%, about 20% to about 90%, about 30% to about 80%, about 40% to about 70%, about 50% to about 95%, about 60% to about 85%, or about 70% to about 90%, or about 85% to about 95% identical to that of the corresponding heavy chain variable region and a light chain variable region having a sequence that is about 10% to about 99%, about 20% to about 90%, about 30% to about 80%, about 40% to about 70%, about 50% to about 95%, about 60% to about 85%, or about 70% to about 90%, or about 85% to about 95% identical to the corresponding light chain variable region.

In some embodiments, the antibody comprises (1) a heavy chain variable region of any one of SEQ ID NOS: 68-69 and/or (2) a light chain variable region of SEQ ID NO: 70 as provided in Tables 1 and 2.

In some embodiments, the antibody comprises (1) a heavy chain variable region having at least 10%, at least 20%, at least 30%, at least 40%, at least 50%, at least 60%, at least 70%, at least 80%, at least 90%, at least 95%, at least 99%, or more amino acid sequence identity to any one of SEQ ID NOS:68 or 69 and/or (2) a light chain variable region having at least 10%, at least 20%, at least 30%, at least 40%, at least 50%, at least 60%, at least 70%, at least 80%, at least 90%, at least 95%, at least 99%, or more amino acid sequence identity to SEQ ID NOS:70.

In some embodiments, the antibody comprises (1) a heavy chain variable region having about 10%, about 20%, about 30%, about 40%, about 50%, about 60%, about 70%, about 80%, about 90%, about 95%, about 99%, or more amino acid sequence identity to any one of SEQ ID NOS:68-69 and/or (2) a light chain variable region having about 10%, about 20%, about 30%, about 40%, about 50%, about 60%, about 70%, about 80%, about 90%, about 95%, about 99%, or more amino acid sequence identity to SEQ ID NO:70.

In some embodiments, the antibody comprises (1) a heavy chain variable region having at least about 10%, at least about 20%, at least about 30%, at least about 40%, at least about 50%, at least about 60%, at least about 70%, at least about 80%, at least about 90%, at least about 95%, at least about 99%, or more amino acid sequence identity to any one of SEQ ID NOS:68-69 and/or (2) a light chain variable region having at least about 10%, at least about 20%, at least about 30%, at least about 40%, at least about 50%, at least about 60%, at least about 70%, at least about 80%, at least about 90%, at least about 95%, at least about 99%, or more amino acid sequence identity to SEQ ID NO:70.

In some embodiments, the antibody comprises (1) a heavy chain variable region having about 10% to about 99%, about 20% to about 90%, about 30% to about 80%, about 40% to about 70%, about 50% to about 95%, about 60% to about 85%, or about 70% to about 90%, or about 85% to about 95% amino acid sequence identity to anyone of SEQ ID NOS:68-69 and/or (2) alight chain variable region having about 10% to about 99%, about 20% to about 90%, about 30% to about 80%, about 40% to about 70%, about 50% to about 95%, about 60% to about 85%, or about 70% to about 90%, or about 85% to about 95% amino acid sequence identity to SEQ ID NO:70.

### Structures of the Hantavirus Antibodies

In some embodiments, a hantavirus antibody targets the Gn/Gc spikes and comprises a heavy chain variable region comprising: an HCDR1 comprising a sequence (SEQ ID NOS:6-10, 72, or 75), or a variant HCDR1 in which 1, 2, 3, 4, or 5 amino acids are substituted relative to the sequence; an HCDR2 comprising a sequence (SEQ ID NOS:11-15, 73, or 76), or a variant HCDR2 in which 1, 2, 3, 4, or 5 amino acids are substituted relative to the sequence; and an HCDR3 comprising a sequence (SEQ ID NOS:16-20, 74, or 77), or a variant HCDR3 in which 1, 2, 3, 4, or 5 amino acids are substituted relative to the sequence. In some embodiments, the antibody comprises a light chain variable region comprising: an LCDR1 comprising a sequence (SEQ ID NOS:26-30, or 78) or a variant LCDR1 in which 1, 2, 3, 4, or 5 amino acids are substituted relative to the sequence; an LCDR2 comprising a sequence (SEQ ID NOS:31-35, or 79), or variant LCDR2 in which 1, 2, or 3 amino acids is substituted relative to the sequence; and an LCDR3 comprising a sequence (SEQ ID NOS:36-40, or 80), or a variant LCDR3 in which 1, 2, 3, 4, or 5 amino acids are substituted relative to the sequence.

In some embodiments, a hantavirus antibody that targets the Gn/Gc spikes comprises: a heavy chain variable region comprising: an HCDR1 of any one of SEQ ID NOS:6-10, 72, or 75, or a variant thereof in which 1, 2, 3, 4, or 5 amino acids are substituted relative to the sequence; an HCDR2 of any one of SEQ ID NOS:11-15, 73, or 76 or a variant thereof in which 1, 2, 3, 4, or 5 amino acids are substituted relative to the sequence; and an HCDR3 of any one of SEQ ID NOS:16-20, 74, or 77 or a variant thereof in which 1, 2, 3, 4, or 5 amino acids are substituted relative to the sequence. The antibody also comprises a light chain variable region comprising: an LCDR1 of any one of SEQ ID NOS:26-30, or 78 or a variant thereof in which 1, 2, 3, 4, or 5 amino acids are substituted relative to the sequence; an LCDR2 of any one of SEQ ID NOS:31-35, or 79 or a variant thereof in which 1, 2, or 3 amino acids are substituted relative to the sequence; and an LCDR3 of any one of SEQ ID NOS:36-40, or 80 or a variant thereof in which 1, 2, 3, 4, or 5 amino acids are substituted relative to the sequence.

In some embodiments, a hantavirus antibody targets the Gn/Gc spikes comprises a V_{H} comprising an amino acid sequence having about 95% identity to any one of (SEQ ID NOS:1-5, 68, or 69); and a V_{L} comprising an amino sequence having at 95% identity to any one of (SEQ ID NOS: 81, 21-25, or 70). In some embodiments, a hantavirus antibody that binds to one or more epitopes on the Gn/Gc spikes comprises a V_{H} comprising an amino acid sequence having about 95% identity to SEQ ID NO:1; and a V_{L} comprising an amino sequence having at 95% identity to SEQ ID NO: 81 or 21. In some embodiments, a hantavirus antibody that binds to one or more epitopes on the Gn/Gc spikes comprises a V_{H} comprising an amino acid sequence having about 95% identity to SEQ ID NO:2; and a V_{L} comprising an amino sequence having at 95% identity to SEQ ID NO:22. In some embodiments, a hantavirus antibody that binds to one or more epitopes on the Gn/Gc spikes comprises a V_{H} comprising an amino acid sequence having about 95% identity to SEQ ID NO:3; and a V_{L} comprising an amino sequence having at 95% identity to SEQ ID NO:23. In some embodiments, a hantavirus antibody that binds to one or more epitopes on the Gn/Gc spikes comprises a V_{H} comprising an amino acid sequence having about 95% identity to SEQ ID NO:4; and a V_{L} comprising an amino sequence having at 95% identity to SEQ ID NO:24. In some embodiments, a hantavirus antibody that binds to one or more epitopes on the Gn/Gc spikes comprises a V_{H} comprising an amino acid sequence having about 95% identity to SEQ ID NO:5; and a V_{L} comprising an amino sequence having at 95% identity to SEQ ID NO:25. In some embodiments, a hantavirus antibody that binds to one or more epitopes on the Gn/Gc spikes comprises a V_{H} comprising an amino acid sequence having about 95% identity to SEQ ID NO:68; and a V_{L} comprising an amino sequence having at 95% identity to SEQ ID NO:70. In some embodiments, a hantavirus antibody that binds to one or more epitopes on the Gn/Gc spikes comprises a V_{H} comprising an amino acid sequence having about 95% identity to SEQ ID NO:69; and a V_{L} comprising an amino sequence having at 95% identity to SEQ ID NO:70.

In some embodiments, a hantavirus antibody targets the Gn/Gc spikes comprises a V_{H} comprising an amino acid sequence having at least 95% identity to any one of (SEQ ID NOS:1-5, 68, or 69); and a V_{L} comprising an amino sequence having at 95% identity to any one of (SEQ ID NOS: 81, 21-25, or 70). In some embodiments, a hantavirus antibody that binds to one or more epitopes on the Gn/Gc spikes comprises a V_{H} comprising an amino acid sequence having at least 95% identity to SEQ ID NO:1; and a V_{L} comprising an amino sequence having at 95% identity to SEQ ID NO: 81 or 21. In some embodiments, a hantavirus antibody that binds to one or more epitopes on the Gn/Gc spikes comprises a V_{H} comprising an amino acid sequence having at least 95% identity to SEQ ID NO:2; and a V_{L} comprising an amino sequence having at 95% identity to SEQ ID NO:22. In some embodiments, a hantavirus antibody that binds to one or more epitopes on the Gn/Gc spikes comprises a V_{H} comprising an amino acid sequence having at least 95% identity to SEQ ID NO:3; and a V_{L} comprising an amino sequence having at 95% identity to SEQ ID NO:23. In some embodiments, a hantavirus antibody that binds to one or more epitopes on the Gn/Gc spikes comprises a V_{H} comprising an amino acid sequence having at least 95% identity to SEQ ID NO:4; and a V_{L} comprising an amino sequence having at 95% identity to SEQ ID NO:24. In some embodiments, a hantavirus antibody that binds to one or more epitopes on the Gn/Gc spikes comprises a V_{H} comprising an amino acid sequence having at least 95% identity to SEQ ID NO:5; and a V_{L} comprising an amino sequence having at 95% identity to SEQ ID NO:25. In some embodiments, a hantavirus antibody that binds to one or more epitopes on the Gn/Gc spikes comprises a V_{H} comprising an amino acid sequence having at least 95% identity to SEQ ID NO:68; and a V_{L} comprising an amino sequence having at 95% identity to SEQ ID NO:70. In some embodiments, a hantavirus antibody that binds to one or more epitopes on the Gn/Gc spikes comprises a V_{H} comprising an amino acid sequence having at least 95% identity to SEQ ID NO:69; and a V_{L} comprising an amino sequence having at 95% identity to SEQ ID NO:70.

In some embodiments, a hantavirus antibody targets the Gn/Gc spikes comprises a V_{H} comprising an amino acid sequence having at least about 95% identity to any one of (SEQ ID NOS:1-5, 68, or 69); and a V_{L} comprising an amino sequence having at 95% identity to any one of (SEQ ID NOS: 81, 21-25, or 70). In some embodiments, a hantavirus antibody that binds to one or more epitopes on the Gn/Gc spikes comprises a V_{H} comprising an amino acid sequence having at least about 95% identity to SEQ ID NO:1; and a V_{L} comprising an amino sequence having at 95% identity to SEQ ID NO: 81 or 21. In some embodiments, a hantavirus antibody that binds to one or more epitopes on the Gn/Gc spikes comprises a V_{H} comprising an amino acid sequence having at least about 95% identity to SEQ ID NO:2; and a V_{L} comprising an amino sequence having at 95% identity to SEQ ID NO:22. In some embodiments, a hantavirus antibody that binds to one or more epitopes on the Gn/Gc spikes comprises a V_{H} comprising an amino acid sequence having at least about 95% identity to SEQ ID NO:3; and a V_{L} comprising an amino sequence having at 95% identity to SEQ ID NO:23. In some embodiments, a hantavirus antibody that binds to one or more epitopes on the Gn/Gc spikes comprises a V_{H} comprising an amino acid sequence having at least about 95% identity to SEQ ID NO:4; and a V_{L} comprising an amino sequence having at 95% identity to SEQ ID NO:24. In some embodiments, a hantavirus antibody that binds to one or more epitopes on the Gn/Gc spikes comprises a V_{H} comprising an amino acid sequence having at least about 95% identity to SEQ ID NO:5; and a V_{L} comprising an amino sequence having at 95% identity to SEQ ID NO:25. In some embodiments, a hantavirus antibody that binds to one or more epitopes on the Gn/Gc spikes comprises a V_{H} comprising an amino acid sequence having at least about 95% identity to SEQ ID NO:68; and a V_{L} comprising an amino sequence having at 95% identity to SEQ ID NO:70. In some embodiments, a hantavirus antibody that binds to one or more epitopes on the Gn/Gc spikes comprises a V_{H} comprising an amino acid sequence having at least about 95% identity to SEQ ID NO:69; and a V_{L} comprising an amino sequence having at 95% identity to SEQ ID NO:70.

In some embodiments, a hantavirus antibody targets the Gn/Gc spikes comprises a V_{H} region and a V_{L} region. The V_{H} region has about 70% amino acid sequence identity to SEQ ID NO:1; and comprises a CDR1 of SEQ ID NO:1, or the CDR1 of SEQ ID NO:1 in which 1, 2, 3, 4, or 5 amino acids are substituted; a CDR2 of SEQ ID NO:1, or the CDR1 of SEQ ID NO:1 in which 1, 2, 3, 4, or 5 amino acids are substituted; a CDR3 of SEQ ID NO:1, or the CDR1 of SEQ ID NO:1 in which 1, 2, 3, 4, or 5 amino acids are substituted. The V_{L} region has about 70% amino acid sequence identity to SEQ ID NO: 81, 21 and comprises a CDR1 of SEQ ID NO: 81, 21 or the CDR1 of SEQ ID NO:81, 21 in which 1, 2, 3, 4, or5 amino acids are substituted; a CDR2 of SEQ ID NO:21, or the CDR2 of SEQ ID NO:81, 21 in which 1, 2, 3, 4, or 5 amino acids are substituted; a CDR3 of SEQ ID NO:21 or the CDR3 of SEQ ID NO:81, 21 in which 1, 2, 3, 4, or 5 are substituted.

In some embodiments, a hantavirus antibody targets the Gn/Gc spikes comprises a V_{H} region and a V_{L} region. The V_{H} region has at least 70% amino acid sequence identity to SEQ ID NO:1; and comprises a CDR1 of SEQ ID NO:1, or the CDR1 of SEQ ID NO:1 in which 1, 2, 3, 4, or 5 amino acids are substituted; a CDR2 of SEQ ID NO:1, or the CDR1 of SEQ ID NO:1 in which 1, 2, 3, 4, or 5 amino acids are substituted; a CDR3 of SEQ ID NO:1, or the CDR1 of SEQ ID NO:1 in which 1, 2, 3, 4, or 5 amino acids are substituted. The V_{L} region has at least 70% amino acid sequence identity to SEQ ID NO: 81 or 21 and comprises a CDR1 of SEQ ID NO: 81 or 21 or the CDR1 of SEQ ID NO:81, 21 in which 1, 2, 3, 4, or5 amino acids are substituted; a CDR2 of SEQ ID NO:21, or the CDR2 of SEQ ID NO:81 or 21 in which 1, 2, 3, 4, or 5 amino acids are substituted; a CDR3 of SEQ ID NO:21 or the CDR3 of SEQ ID NO:81, 21 in which 1, 2, 3, 4, or 5 are substituted.

In some embodiments, a hantavirus antibody targets the Gn/Gc spikes comprises a V_{H} region and a V_{L} region. The V_{H} region has about 70% amino acid sequence identity to SEQ ID NO:2; and comprises a CDR1 of SEQ ID NO:2, or the CDR1 of SEQ ID NO:1 in which 1, 2, 3, 4, or 5 amino acids are substituted; a CDR2 of SEQ ID NO:2, or the CDR1 of SEQ ID NO:2 in which 1, 2, 3, 4, or 5 amino acids are substituted; a CDR3 of SEQ ID NO:2, or the CDR1 of SEQ ID NO:2 in which 1, 2, 3, 4, or 5 amino acids are substituted. The V_{L} region has about 70% amino acid sequence identity to SEQ ID NO:22, and comprises a CDR1 of SEQ ID NO:22 or the CDR1 of SEQ ID NO:22 in which 1, 2, 3, 4, or 5 amino acids are substituted; a CDR2 of SEQ ID NO:22, or the CDR2 of SEQ ID NO:22 in which 1, 2, 3, 4, or 5 amino acids are substituted; a CDR3 of SEQ ID NO:22 or the CDR3 of SEQ ID NO:22 in which 1, 2, 3, 4, or 5 are substituted.

In some embodiments, a hantavirus antibody targets the Gn/Gc spikes comprises a V_{H} region and a V_{L} region. The V_{H} region has at least 70% amino acid sequence identity to SEQ ID NO:2; and comprises a CDR1 of SEQ ID NO:2, or the CDR1 of SEQ ID NO:1 in which 1, 2, 3, 4, or 5 amino acids are substituted; a CDR2 of SEQ ID NO:2, or the CDR1 of SEQ ID NO:2 in which 1, 2, 3, 4, or 5 amino acids are substituted; a CDR3 of SEQ ID NO:2, or the CDR1 of SEQ ID NO:2 in which 1, 2, 3, 4, or 5 amino acids are substituted. The V_{L} region has at least 70% amino acid sequence identity to SEQ ID NO:22, and comprises a CDR1 of SEQ ID NO:22 or the CDR1 of SEQ ID NO:22 in which 1, 2, 3, 4, or 5 amino acids are substituted; a CDR2 of SEQ ID NO:22, or the CDR2 of SEQ ID NO:22 in which 1, 2, 3, 4, or 5 amino acids are substituted; a CDR3 of SEQ ID NO:22 or the CDR3 of SEQ ID NO:22 in which 1, 2, 3, 4, or 5 are substituted.

In some embodiments, a hantavirus antibody targets the Gn/Gc spikes comprises a V_{H} region and a V_{L} region. The V_{H} region has at least 70% amino acid sequence identity to SEQ ID NO:3; and comprises a CDR1 of SEQ ID NO:3, or the CDR1 of SEQ ID NO:1 in which 1, 2, 3, 4, or 5 amino acids are substituted; a CDR2 of SEQ ID NO:3, or the CDR1 of SEQ ID NO:3 in which 1, 2, 3, 4, or 5 amino acids are substituted; a CDR3 of SEQ ID NO:3, or the CDR1 of SEQ ID NO:3 in which 1, 2, 3, 4, or 5 amino acids are substituted. The V_{L} region has at least 70% amino acid sequence identity to SEQ ID NO:23, and comprises a CDR1 of SEQ ID NO:23 or the CDR1 of SEQ ID NO:23 in which 1, 2, 3, 4, or 5 amino acids are substituted; a CDR2 of SEQ ID NO:23, or the CDR2 of SEQ ID NO:23 in which 1, 2, 3, 4, or 5 amino acids are substituted; a CDR3 of SEQ ID NO:23 or the CDR3 of SEQ ID NO:23 in which 1, 2, 3, 4, or 5 are substituted.

In some embodiments, a hantavirus antibody targets the Gn/Gc spikes comprises a heavy chain variable (V_{H}) region and a light chain variable (V_{L}) region. The V_{H} region has about 70% amino acid sequence identity to SEQ ID NO:3; and comprises a CDR1 of SEQ ID NO:3, or the CDR1 of SEQ ID NO:1 in which 1, 2, 3, 4, or 5 amino acids are substituted; a CDR2 of SEQ ID NO:3, or the CDR1 of SEQ ID NO:3 in which 1, 2, 3, 4, or 5 amino acids are substituted; a CDR3 of SEQ ID NO:3, or the CDR1 of SEQ ID NO:3 in which 1, 2, 3, 4, or 5 amino acids are substituted. The V_{L} region has about 70% amino acid sequence identity to SEQ ID NO:23, and comprises a CDR1 of SEQ ID NO:23 or the CDR1 of SEQ ID NO:23 in which 1, 2, 3, 4, or 5 amino acids are substituted; a CDR2 of SEQ ID NO:23, or the CDR2 of SEQ ID NO:23 in which 1, 2, 3, 4, or 5 amino acids are substituted; a CDR3 of SEQ ID NO:23 or the CDR3 of SEQ ID NO:23 in which 1, 2, 3, 4, or 5 are substituted.

In some embodiments, a hantavirus antibody targets the Gn/Gc spikes comprises a V_{H} region and a V_{L} region. The V_{H} region has at least 70% amino acid sequence identity to SEQ ID NO:4; and comprises a CDR1 of SEQ ID NO:4, or the CDR1 of SEQ ID NO:1 in which 1, 2, 3, 4, or 5 amino acids are substituted; a CDR2 of SEQ ID NO:4, or the CDR1 of SEQ ID NO:4 in which 1, 2, 3, 4, or 5 amino acids are substituted; a CDR3 of SEQ ID NO:4, or the CDR1 of SEQ ID NO:4 in which 1, 2, 3, 4, or 5 amino acids are substituted. The V_{L} region has at least 70% amino acid sequence identity to SEQ ID NO:24, and comprises a CDR1 of SEQ ID NO:24 or the CDR1 of SEQ ID NO:24 in which 1, 2, 3, 4, or 5 amino acids are substituted; a CDR2 of SEQ ID NO:24, or the CDR2 of SEQ ID NO:24 in which 1, 2, 3, 4, or 5 amino acids are substituted; a CDR3 of SEQ ID NO:24 or the CDR3 of SEQ ID NO:24 in which 1, 2, 3, 4, or 5 are substituted.

In some embodiments, a hantavirus antibody targets the Gn/Gc spikes comprises a V_{H} region and a V_{L} region. The V_{H} region has about 70% amino acid sequence identity to SEQ ID NO:5; and comprises a CDR1 of SEQ ID NO:5, or the CDR1 of SEQ ID NO:1 in which 1, 2, 3, 4, or 5 amino acids are substituted; a CDR2 of SEQ ID NO:5, or the CDR1 of SEQ ID NO:5 in which 1, 2, 3, 4, or 5 amino acids are substituted; a CDR3 of SEQ ID NO:5, or the CDR1 of SEQ ID NO:5 in which 1, 2, 3, 4, or 5 amino acids are substituted. The V_{L} region has about 70% amino acid sequence identity to SEQ ID NO:25 and comprises a CDR1 of SEQ ID NO:25 or the CDR1 of SEQ ID NO:25 in which 1, 2, 3, 4, or 5 amino acids are substituted; a CDR2 of SEQ ID NO:25, or the CDR2 of SEQ ID NO:25 in which 1, 2, 3, 4, or 5 amino acids are substituted; a CDR3 of SEQ ID NO:25 or the CDR3 of SEQ ID NO:25 in which 1, 2, 3, 4, or 5 are substituted.

In some embodiments, a hantavirus antibody targets the Gn/Gc spikes comprises a V_{H} region and a V_{L} region. The V_{H} region has at least 70% amino acid sequence identity to SEQ ID NO:5; and comprises a CDR1 of SEQ ID NO:5, or the CDR1 of SEQ ID NO:1 in which 1, 2, 3, 4, or 5 amino acids are substituted; a CDR2 of SEQ ID NO:5, or the CDR1 of SEQ ID NO:5 in which 1, 2, 3, 4, or 5 amino acids are substituted; a CDR3 of SEQ ID NO:5, or the CDR1 of SEQ ID NO:5 in which 1, 2, 3, 4, or 5 amino acids are substituted. The V_{L} region has at least 70% amino acid sequence identity to SEQ ID NO:25 and comprises a CDR1 of SEQ ID NO:25 or the CDR1 of SEQ ID NO:25 in which 1, 2, 3, 4, or 5 amino acids are substituted; a CDR2 of SEQ ID NO:25, or the CDR2 of SEQ ID NO:25 in which 1, 2, 3, 4, or 5 amino acids are substituted; a CDR3 of SEQ ID NO:25 or the CDR3 of SEQ ID NO:25 in which 1, 2, 3, 4, or 5 are substituted.

In some embodiments, a hantavirus antibody targets the Gn/Gc spikes comprises a V_{H} region and a V_{L} region. The V_{H} region has about 70% amino acid sequence identity to SEQ ID NO:68; and comprises a CDR1 of SEQ ID NO:68, or the CDR1 of SEQ ID NO:1 in which 1, 2, 3, 4, or 5 amino acids are substituted; a CDR2 of SEQ ID NO:68, or the CDR1 of SEQ ID NO:68 in which 1, 2, 3, 4, or 5 amino acids are substituted; a CDR3 of SEQ ID NO:68, or the CDR1 of SEQ ID NO:68 in which 1, 2, 3, 4, or 5 amino acids are substituted. The V_{L} region has about 70% amino acid sequence identity to SEQ ID NO:70 and comprises a CDR1 of SEQ ID NO:70 or the CDR1 of SEQ ID NO:70 in which 1, 2, 3, 4, or 5 amino acids are substituted; a CDR2 of SEQ ID NO:70, or the CDR2 of SEQ ID NO:70 in which 1, 2, 3, 4, or 5 amino acids are substituted; a CDR3 of SEQ ID NO:70 or the CDR3 of SEQ ID NO:70 in which 1, 2, 3, 4, or 5 are substituted.

In some embodiments, a hantavirus antibody targets the Gn/Gc spikes comprises a V_{H} region and a V_{L} region. The V_{H} region has at least 70% amino acid sequence identity to SEQ ID NO:68; and comprises a CDR1 of SEQ ID NO:68, or the CDR1 of SEQ ID NO:1 in which 1, 2, 3, 4, or 5 amino acids are substituted; a CDR2 of SEQ ID NO:68, or the CDR1 of SEQ ID NO:68 in which 1, 2, 3, 4, or 5 amino acids are substituted; a CDR3 of SEQ ID NO:68, or the CDR1 of SEQ ID NO:68 in which 1, 2, 3, 4, or 5 amino acids are substituted. The V_{L} region has at least 70% amino acid sequence identity to SEQ ID NO:70 and comprises a CDR1 of SEQ ID NO:70 or the CDR1 of SEQ ID NO:70 in which 1, 2, 3, 4, or 5 amino acids are substituted; a CDR2 of SEQ ID NO:70, or the CDR2 of SEQ ID NO:70 in which 1, 2, 3, 4, or 5 amino acids are substituted; a CDR3 of SEQ ID NO:70 or the CDR3 of SEQ ID NO:70 in which 1, 2, 3, 4, or 5 are substituted.

In some embodiments, a hantavirus antibody targets the Gn/Gc spikes comprises a V_{H} region and a V_{L} region. The V_{H} region has about 70% amino acid sequence identity to SEQ ID NO:69; and comprises a CDR1 of SEQ ID NO:69, or the CDR1 of SEQ ID NO:1 in which 1, 2, 3, 4, or 5 amino acids are substituted; a CDR2 of SEQ ID NO:69, or the CDR1 of SEQ ID NO:69 in which 1, 2, 3, 4, or 5 amino acids are substituted; a CDR3 of SEQ ID NO:69, or the CDR1 of SEQ ID NO:69 in which 1, 2, 3, 4, or 5 amino acids are substituted. The V_{L} region has about 70% amino acid sequence identity to SEQ ID NO:70, and comprises a CDR1 of SEQ ID NO:70 or the CDR1 of SEQ ID NO:70 in which 1, 2, 3, 4, or 5 amino acids are substituted; a CDR2 of SEQ ID NO:70, or the CDR2 of SEQ ID NO:70 in which 1, 2, 3, 4, or 5 amino acids are substituted; a CDR3 of SEQ ID NO:70 or the CDR3 of SEQ ID NO:70 in which 1, 2, 3, 4, or 5 are substituted.

In some embodiments, a hantavirus antibody targets the Gn/Gc spikes comprises a V_{H} region and a V_{L} region. The V_{H} region has at least 70% amino acid sequence identity to SEQ ID NO:69; and comprises a CDR1 of SEQ ID NO:69, or the CDR1 of SEQ ID NO:1 in which 1, 2, 3, 4, or 5 amino acids are substituted; a CDR2 of SEQ ID NO:69, or the CDR1 of SEQ ID NO:69 in which 1, 2, 3, 4, or 5 amino acids are substituted; a CDR3 of SEQ ID NO:69, or the CDR1 of SEQ ID NO:69 in which 1, 2, 3, 4, or 5 amino acids are substituted. The V_{L} region has at least 70% amino acid sequence identity to SEQ ID NO:70, and comprises a CDR1 of SEQ ID NO:70 or the CDR1 of SEQ ID NO:70 in which 1, 2, 3, 4, or 5 amino acids are substituted; a CDR2 of SEQ ID NO:70, or the CDR2 of SEQ ID NO:70 in which 1, 2, 3, 4, or 5 amino acids are substituted; a CDR3 of SEQ ID NO:70 or the CDR3 of SEQ ID NO:70 in which 1, 2, 3, 4, or 5 are substituted.

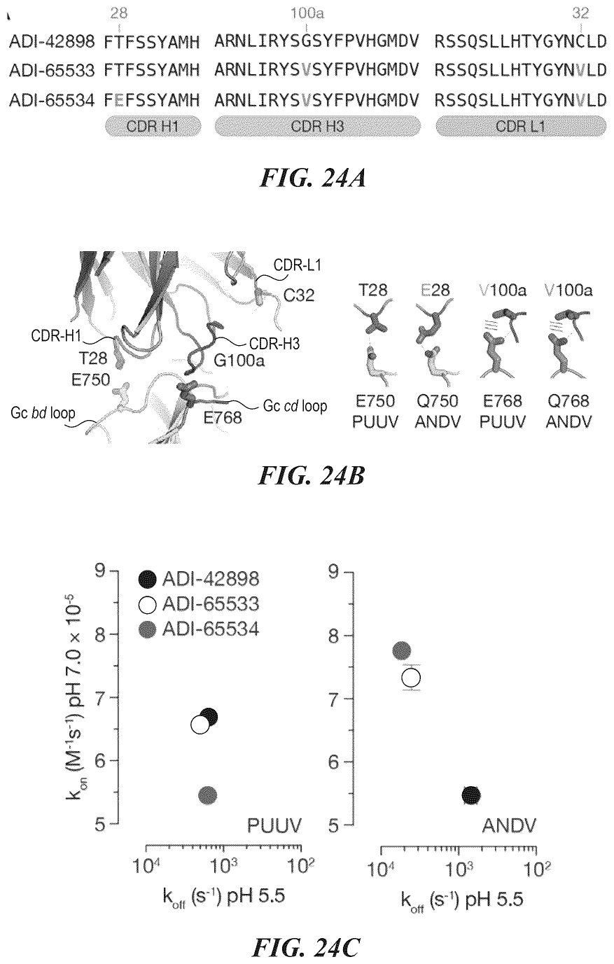

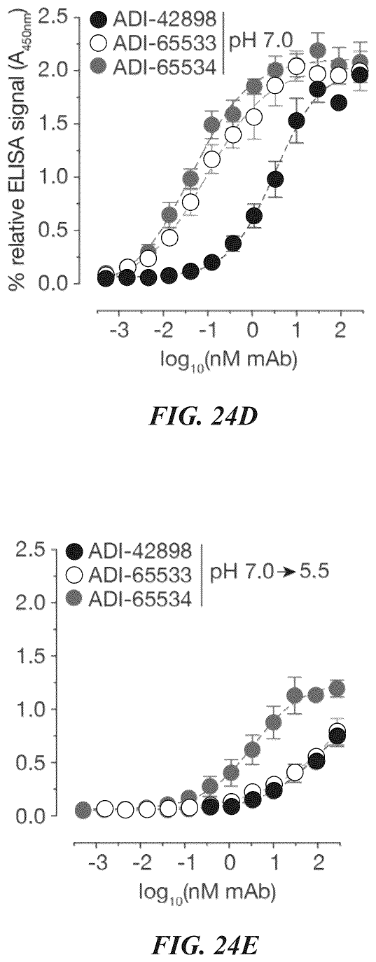

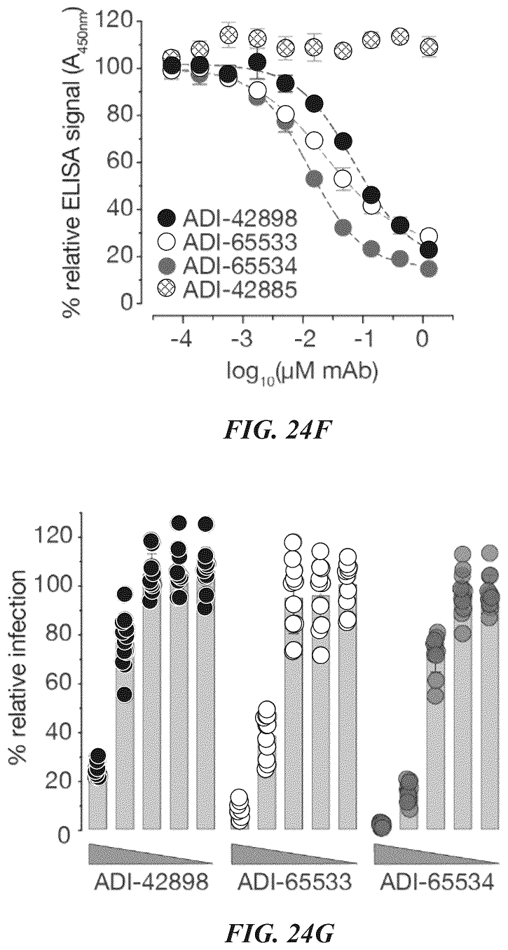

In some embodiments, a hantavirus antibody targets the Gn/Gc spikes of the present technology and has one, two, or three CDRs of a V_{H} sequence in Table 1. In some embodiments, the hantavirus antibody has at least one mutation and no more than 10, 20, 30, 40, or 50 mutations in the V_{H} amino acid sequences compared to a V_{H} sequence set forth in Table 1. In some embodiments, the V_{H} amino acid sequence may comprise 1, 2, 3, 4, 5, 6, 7, 8, 9, or 10 amino acid insertions or deletions compared to a V_{H} sequence set forth in Table 1. In some embodiments, the V_{H} amino acid sequence may comprise a deletion or insertion, e.g., a 1, 2, 3, 4, 5, 6, or 7 amino acid deletion or insertion relative to a CDR sequence shown in Table 1. In some embodiments, the V_{H} region comprises a CDR1 with 1 or 2 substitutions relative to a CDR1 sequence shown in Table 1. In some embodiments, a CDR1 has 3, 4, or 5 substitutions relative to a CDR1 sequence shown in Table 1. In some embodiments, the V_{H} region comprises a CDR2 with 1 or 2, or 1, 2, or 3; substitutions relative to the CDR2 sequence shown in Table 1. In some embodiments, the V_{H} region comprises a CDR3 with 1, 2, or 3; or 1, 2, 3, or 4; substitutions are relative to a CDR3 sequence shown in Table 1. In some embodiments, a hantavirus antibody of the present technology comprises a CDR1, CDR2, and CDR3, each having at least 70% identity to a CDR1, CDR2, and CDR3, as shown in Table 1. In some embodiments, a hantavirus antibody of the present technology comprises a CDR1, CDR2, and CDR3, each having at least 80% identity to a CDR1, CDR2, and CDR3, as shown in Table 1. In some embodiments, a hantavirus of the present technology comprises a CDR1, CDR2, and CDR3, as shown in Table 1. In some embodiments, a hantavirus antibody of the present technology comprises HCDR1, HCDR2, HCDR3, LCDR1, LCDR2, and LCDR3 of an antibody designated as ADI-42898, ADI-42877, ADI-42098, ADI-42885, ADI-37236, ADI-65533, and ADI-65534 in Table 1. In some embodiments, a hantavirus antibody of the present technology comprises a CDR1, CDR2, and CDR3 of the V_{H} of an antibody designated as ADI-42898. In some embodiments, a hantavirus antibody of the present technology comprises a CDR1, CDR2, and CDR3 of the V_{H} of an antibody designated as ADI-65533. In some embodiments, a hantavirus antibody of the present technology comprises a CDR1, CDR2, and CDR3 of the V_{H} of an antibody designated as ADI-65534. ADI-65533 and ADI-65534 are affinity-matured mAbs derived from ADI-42898. The characterization of affinity-matured mAbs, ADI-65533 and ADI-65534 are shown in FIGS. 24A-24E, and the sequence differences compared to ADI-42898 are shown in Table 3.

In some embodiments, a hantavirus antibody targets the Gn/Gc spikes of the present technology and has one, two, or three CDRs of a V_{L} sequence in Table 2. In some embodiments, the hantavirus antibody has at least one mutation and no more than 10, 20, 30, 40, or 50 mutations in the V_{L} amino acid sequences compared to a V_{L} sequence set forth in Table 2. In some embodiments, the V_{L} amino acid sequence may comprise 1, 2, 3, 4, 5, 6, 7, 8, 9, or 10 amino acid insertions or deletions compared to a V_{L} sequence set forth in Table 2. In some embodiments, the V_{L} amino acid sequence may comprise a deletion or insertion, e.g., a 1, 2, 3, 4, 5, 6, or 7 amino acid deletion or insertion relative to a CDR sequence shown in Table 2. In some embodiments, the V_{L} region comprises a CDR1 with 1 or 2 substitutions relative to a CDR1 sequence shown in Table 2. In some embodiments, a CDR1 has 3, 4, or 5 substitutions relative to a CDR1 sequence shown in Table 2. In some embodiments, the V_{L} region comprises a CDR2 with 1 or 2; or 1, 2, or 3; substitutions relative to the CDR2 sequence shown in Table 2. In some embodiments, the V_{L} region comprises a CDR3 with 1, 2, or 3; or 1, 2, 3, or 4; substitutions are relative to a CDR3 sequence shown in Table 2. In some embodiments, a hantavirus antibody of the present technology comprises a CDR1, CDR2, and CDR3, each having at least 70% identity to a CDR1, CDR2, and CDR3, as shown in Table 2. In some embodiments, a hantavirus antibody of the present technology comprises a CDR1, CDR2, and CDR3, each having at least 80% identity to a CDR1, CDR2, and CDR3, as shown in Table 2. In some embodiments, a hantavirus of the present technology comprises a CDR1, CDR2, and CDR3, as shown in Table 2. In some embodiments, a hantavirus antibody of the present technology comprises HCDR1, HCDR2, HCDR3, LCDR1, LCDR2, and LCDR3 of an antibody designated as ADI-42898, ADI-42877, ADI-42098, ADI-42885, ADI-37236, ADI-65533, and ADI-65534 in Table 2. In some embodiments, a hantavirus antibody of the present technology comprises a CDR1, CDR2, and CDR3 of the V_{L} of an antibody designated as ADI-42898. In some embodiments, a hantavirus antibody of the present technology comprises a CDR1, CDR2, and CDR3 of the V_{L} of an antibody designated as ADI-65533. In some embodiments, a hantavirus antibody of the present technology comprises a CDR1, CDR2, and CDR3 of the V_{L} of an antibody designated as ADI-65534.

In some embodiments, the hantavirus antibody of the present technology comprises a heavy chain variable region and a light chain variable region of an antibody designated as ADI-42898, ADI-42877, ADI-42098, ADI-42885, ADI-37236, ADI-65533, ADI-65534 or a variant thereof. The variant comprises a heavy chain variable region with a sequence that is at least 95% identical to that of the corresponding heavy chain variable region and a light chain variable region with a sequence that is at least 95% identical to the corresponding light chain variable region. For example, the variant may comprise a V_{H} that is at least 70%, at least 80%, at least 90%, at least 95%, or at least 99% identical to that of ADI-42898, ADI-42877, ADI-42098, ADI-42885, ADI-37236, ADI-65533, ADI-65534 and/or a V_{L} that is at least 70%, at least 80%, at least 90%, at least 95%, or at least 99% identical to that of ADI-42898, ADI-42877, ADI-42098, ADI-42885, ADI-37236, ADI-65533, ADI-65534.

In some embodiments, the hantavirus antibody of the present technology comprises an HCDR1, HCDR2, HCDR3, LCDR1, LCDR2, and LCDR3 of an antibody designated as ADI-42898, ADI-42877, ADI-42098, ADI-42885, ADI-37236, ADI-65533, ADI-65534 or a variant thereof, and at least one, two, three, four, five, or all six of the CDRs of the variant contain 1 or 2 amino acid substitutions compared to the corresponding CDR. For example, at least one, two, three, four, five, or all six of the CDRs of the variant contain 1 or 2 amino acid substitutions compared to the CDRs of ADI-42898, ADI-42877, ADI-42098, ADI-42885, ADI-37236, ADI-65533, ADI-65534.

In some embodiments, the hantavirus antibody of the present technology comprises an HCDR1, HCDR2, HCDR3, LCDR1, LCDR2, and LCDR3 of an antibody designated as ADI-42898. In some embodiments, the hantavirus antibody of the present technology comprises an HCDR1, HCDR2, HCDR3, LCDR1, LCDR2, and LCDR3 of an antibody designated as ADI-65533. In some embodiments, the hantavirus antibody of the present technology comprises an HCDR1, HCDR2, HCDR3, LCDR1, LCDR2, and LCDR3 of an antibody designated as ADI-65534.

### Linkers

In some embodiments, the heavy chain and the light chain (e.g., the V_{H} region and the V_{L} region) are joined by an amino acid linker. In some embodiments, the amino acid linker is a flexible linker. The majority of the amino acids in the linker may be glycine (G) and/or serine (S) residues.

### Epitopes

A hantavirus antibody of the present technology binds to one or more epitopes of the Gn/Gc spikes (SEQ ID NOS:41-43) as provided in Table 4. In some embodiments, a hantavirus antibody of the present technology binds to an epitope in the Gn subunit (SEQ ID NO:42) and/or the Gc subunit (SEQ ID NO:43) of the Gn/Gc spikes. In some embodiments, a hantavirus antibody of the present technology binds to an epitope in the capping loop of the Gn subunit (SEQ ID NO:41), spanning amino acid (aa) residues 90-103 in Gn^{H}. In some embodiments, a hantavirus antibody of the present technology binds to an epitope in the capping loop of the Gn subunit (SEQ ID NOS:44-49) of the Gn/Gc spike protein.

In some embodiments, the antibodies of the present technology bind to an epitope in a polypeptide having an amino acid sequence about 10%, about 20%, about 30%, about 40%, about 50%, about 60%, about 70%, about 80%, about 90%, about 95%, about 99%, or about 100% identical to one or more of SEQ ID NO: 42-67, 83-90, or 93-105.

In some embodiments, the antibodies of the present technology bind to an epitope in a polypeptide having an amino acid sequence at least 10%, at least 20%, at least 30%, at least 40%, at least 50%, at least 60%, at least 70%, at least 80%, at least 90%, at least 95%, at least 99%, or at least 100% identical to one or more of SEQ ID NO: 42-67, 83-90, or 93-105.

In some embodiments, the antibodies of the present technology bind to an epitope in a polypeptide having an amino acid sequence at least about 10%, at least about 20%, at least about 30%, at least about 40%, at least about 50%, at least about 60%, at least about 70%, at least about 80%, at least about 90%, at least about 95%, at least about 99%, or at least about 100% identical to one or more of SEQ ID NO: 42-67, 83-90, or 93-105.

TABLE 4
Polypeptides and Subunits

| Polypeptide | Sequence |
|---|---|
| Gn Capping | KGDLAENTQASSTS |
| loop | (SEQ ID NO: 41) |
| Gn | ARNLNELKMECPHTIRLGQGLVVGSVELPSLPIQQVETLKLESS |
| | CNFDLHTSTAGQQSFTKWTWEIKGDLAENTQASSTSFQTKSS |
| | EVNLRGLCLIPTLVVETAARMRKTIACYDLSCNQTVCQPTVYLM |
| | GPIQTCITTKSCLLSLGDQRIQVNYEKTYCVSGQLVEGICFNPIH |
| | TMALSQPSHTYDIMTMMVRCFLVIKKVTSGDSMKIEKNFETLVQ |
| | KNGCTANNFQGYYICLIGSSSEPLYVPALDDYRSAEVLSRMAF |
| | APHGEDHDIEKNAVSAMRIAGKVTGKAPSTESSDTVQGIAFSG |
| | SPLYTSTGVLTSKDDPVYIWAPGIIMEGNHSICEKKTLPLTWTG |
| | FISLPGEIEKTTQCTVFCTLAGPGADCEAYSETGIFNISSPTCLIN |
| | RVQRFRGSEQQIKFVCQRVDMDITVYCNGMKKVILTKTLVIGQ |
| | CIYTFTSIFSLIPGVAHSLAVELCVPGLHGWATMLLLLTFCFGWV |
| | LIPTITMILLKILIAFAYLCSKYNTDSKFRILIEKVKREYQKTMGSM |
| | VCEVCQYECETAKELESHRKSCSIGSCPYCLNPSEATTSALQA |
| | HFKVCKLTSRFQENLRKSLTVYEPMQGCYRTLSLFRYRSRFFV |
| | GLVWCVLLVLELIVWAASA |
| | (SEQ ID NO: 42) |
| Gc | ETQNLNAGWTDTAHGSGIIPMKTDLELDFSLPSSASYTYRRQL |
| | QNPANEQEKIPFHLQLSKQVIHAEIQHLGHWMDATFNLKTAFH |
| | CYGSCEKYAYPWQTAGCFIEKDYEYETGWGCNPPDCPGVGT |
| | GCTACGVYLDKLKSVGKVFKIVSLRYTRKVCIQLGTEQTCKTVD |
| | SNDCLITTSVKVCLIGTISKFQPSDTLLFLGPLQQGGLIFKQWCT |
| | TTCQFGDPGDIMSTPTGMKCPELNGSFRKKCAFATTPVCQFD |
| | GNTISGYKRMIATKDSFQSFNVTEPHISTSALEWIDPDSSLRDHI |
| | NVIVSRDLSFQDLSETPCQIDLATASIDGAWGSGVGFNLVCTVS |
| | LTECSAFLTSIKACDAAMCYGSTTANLVRGQNTIHIVGKGGHSG |
| | SKFMCCHDTKCSSTGLVAAAPHLDRVTGYNQADSDKIFDDGA |
| | PECGMSCWFKKSGEWILGVLNGNWMVVAVLVVLLILSILLFTLC |
| | CPRRPSYRKEHKP |
| | (SEQ ID NO: 43) |
| PUUV 1 | PUUVWTWEIKGDLAENTQASSTSFQTKSSEV (SEQ ID NO: 44) |
| HTNV 1 | HTNVVSWRGKADQSQSSQNSFETVSTEV (SEQ ID NO: 45) |
| SEOV 1 | SEOVVIWRKKANQESANQNSFEVVESEV (SEQ ID NO: 46) |
| DOBV 1 | DOBVVSWRKKADKAQAAKDSFETTSSEV (SEQ ID NO: 47) |
| ANDV 1 | ANDVVEWRKKSDTTDTTNAASTTFEAQTKTV (SEQ ID NO: 48) |
| SNV 1 | SNVVDWTKKSSTTESTNAGATTFEAKTKEI (SEQ ID NO: 49) |
| PUUV 2 | PUUVHGSGIIPMKTDLELDFSLPSSSLRDHINVIVSRDLSFQDLS |
| | E (SEQ ID NO: 50) |
| HTNV 2 | HINVHGVGSVPMHTDLELDFSLTSSMLRDHINILVTKDIDFDNLG |
| | E (SEQ ID NO: 51) |
| SEOV 2 | SEOVHGVGSVPMHTDLELDFSLPSSMLRDHINIVISKDIDFENLA |
| | E (SEQ ID NO: 52) |
| DOBV 2 | DOBVHGVGVVPMHTDLELDFSLPSSMLKDHLNILVTKDIDFENL |
| | GE (SEQ ID NO: 53) |
| ANDV 2 | ANDVHGVGEIPMKTDLELDFSLPSSNTRDHVNLVLNRDVSFQD |
| | LSD (SEQ ID NO: 54) |
| SNV 2 | SNVHGVGIIPMKTDLELDFALASSSIKDHINMVLNRDVSFQDLS |
| | D (SEQ ID NO: 55) |
| PUUV 3 | PUUVCKTVDSNDCLITTSVKVCLIGTISKEWIDPDSSLRDHINVIV |
| | SRDL (SEQ ID NO: 56) |
| HTNV 3 | HTNVCKIIDMNDCFVSRHVKVCIIGTVSKEWKDPDGMLRDHINIL |
| | VTKDI (SEQ ID NO: 57) |
| SEOV 3 | SEOVCKTIDMNDCFVTRHAKICIIGTVSKEWRDPDGMLRDHINI |
| | VISKDI (SEQ ID NO: 58) |
| DOBV 3 | DOBVCKTIDMNDCFVTRHVKVCIIGTVSKEWKDPDGMLKDHLN |
| | ILVTKDI (SEQ ID NO: 59) |
| ANDV 3 | ANDVCKHIDANDCLVTPSVKVCIVGTVSKEWIDPDGNTRDHVN |
| | LVLNRDV (SEQ ID NO: 60) |
| SNV 3 | SNVCKHIDVNDCLVTPSVKVCMIGTISKEWIDPDSSIKDHINMVL |
| | NRDV (SEQ ID NO: 61) |
| PUUV 4 | PUUVCLIGTISKFQPSDTLLFLGPL (SEQ ID NO: 62) |
| HTNV 4 | HTNVCIIGTVSKFSQGDTLLFFGPL (SEQ ID NO: 63) |
| SEOV 4 | SEOVCIIGTVSKFSQGDTLLFLGPM (SEQ ID NO: 64) |
| DOBV 4 | DOBVCIIGTVSKFSQGDTLVFLGPM (SEQ ID NO: 65) |
| ANDV 4 | ANDVCIVGTVSKLQPSDTLLFLGPL (SEQ ID NO: 66) |
| SNV 4 | SNVCMIGTISKLQPGDTLLFLGPL (SEQ ID NO: 67) |
| PUUV 5 | PUUVWTWEVKSDLAENTQASSTSFQTKSHCYGSCEKYAYPW |
| | QTAGCFVEKDYEYESGWGCNPADCPGVGTGCTAC (SEQ ID |
| | NO: 83) |
| HTNV 5 | HTNVVSWRGKADQSQSSQNSFETVSHCYGACTKYEYPWHTA |
| | KCHYERDYQYETSWGCNPSDCPGVGTGCTAC (SEQ ID NO: |
| | 84) |
| SEOV 5 | SEOVVIWRKKANQESANQNSFEVVEHCYGACTKYQYPWHTAK |
| | CHFEKDYEYENSWACNPPDCPGVGTGCTAC (SEQ ID NO: 85) |
| DOBV 5 | DOBVVSWRKKADKAQAAKDSFETTSHCYGACTKYEYPWHTA |
| | KCHFERDFEYENNWGCNPADCPGIGTGCTAC (SEQ ID NO: |
| | 86) |
| ANDV 5 | ANDVVEWRKKSDTTDTTNAASTTFEAQTHCYGACQKYSYPW |
| | QTSKCFFEKDYQYETGWGCNPGDCPGVGTGCTAC (SEQ ID |
| | NO: 87) |
| SNV 5 | SNVVDWTKKSSTTESTNAGATTFEAKTHCYGECKKYAYPWQT |
| | AKCFFEKDYQYETSWGCNPPDCPGVGTGCTAC (SEQ ID NO: |
| | 88) |
| CHOV 1 | CHOVVNWAKKSTTTETTNAGATTFEATSHCYGACNKYSYPWQ |
| | TAKCFFEKDYQYETGWACNPGDCPGVGTGCTAC (SEQ ID |
| | NO: 89) |
| LGNV 1 | LGNVLEWRKKAETTDTTQAASTTFEAQTHCYGACKQYSYPWQ |
| | TAKCFFEKDYQYETGWGCNPGDCPGVGTGCTAC (SEQ ID |
| | NO: 90) |
| PUUV 6 | PUUVWTWEVKSDLAENTQASSTSFQTKS (SEQ ID NO: 93) |
| HTNV 6 | HTNVVSWRGKADQSQSSQNSFETVS (SEQ ID NO: 94) |
| SEOV 6 | SEOVVIWRKKANQESANQNSFEVVE (SEQ ID NO: 95) |
| DOBV 6 | DOBVVSWRKKADKAQAAKDSFETTS (SEQ ID NO: 96) |
| ANDV 6 | ANDVVEWRKKSDTTDTTNAASTTFEAQT (SEQ ID NO: 97) |
| SNV 6 | SNVVDWTKKSSTTESTNAGATTFEAKT (SEQ ID NO: 98) |
| CHOV 2 | CHOVVNWAKKSTTTETTNAGATTFEATS (SEQ ID NO: 99) |
| LGNV 2 | LGNVLEWRKKAETTDTTQAASTTFEAQT (SEQ ID NO: 100) |
| ANDV 7 | VEWRKKSDTTDTTNAASTTFEAQT (SEQ ID NO: 101) |
| ANDV 8 | VEWRKKSDTTDTTNATSTTFEAQT (SEQ ID NO: 102) |
| SNV 7 | VDWTKKSSTTESTNAGATTFEAKT (SEQ ID NO: 103) |
| SNV 8 | VDWTKKSNTTESTNAGATTFEAKT (SEQ ID NO: 104) |
| SNV 9 | VDWTKKSSTNESTNAGATTFEAKT (SEQ ID NO: 105) |

In some embodiments, a hantavirus antibody binds to one or more epitopes in the Gn subunit at the position E88, E95, Q98, or S101 with the residue numbering referring to the Gn subunit (SEQ ID NO:42). In some embodiments, a hantavirus antibody targets one or more epitopes in the Gc subunit at the position D682, S830, 1844, P852, or D959, S966 with the residue numbering referring to the GC subunit (SEQ ID NO:43).

In some embodiments, a hantavirus antibody binds to at least one, at least two, at least three, at least four, at least five residues selected from the group consisting of E88, E95, Q98, or S101 with the residue numbering referring to the Gn subunit (SEQ ID NO:42), and/or at least one, at least two, at least three, at least four, at least five residues selected from the group consisting D682, S830, 1844, P852, or D959, S966 with the residue numbering referring to the GC subunit (SEQ ID NO:43).

In some embodiments, a hantavirus antibody binds to the Gn/Gc complex but not to the Gn subunit alone. In some embodiments, a hantavirus antibody binds to the Gn subunit. In some embodiments, a hantavirus antibody binds to the Gc subunit.

In some embodiments, the epitope is a quaternary epitope. In some embodiments, a hantavirus antibody recognizes a quaternary epitope encompassing both Gn and Gc subunits.

In some embodiments, the epitope is a tetrameric spike-dependent quaternary epitope. In some embodiments, a hantavirus antibody binds to the Gn subunit. In some embodiments, a hantavirus antibody binds to the tetrameric spike-dependent quaternary epitope. In some embodiments, a hantavirus antibody binds to the Gn subunit. In some embodiments, a hantavirus antibody does not bind to the tetrameric spike-dependent quaternary epitope.

In some embodiments, the epitope is a tetramer lattice-dependent quaternary epitope. In some embodiments, a hantavirus antibody binds to the Gn subunit. In some embodiments, a hantavirus antibody binds to the tetramer lattice-dependent quaternary epitope. In some embodiments, a hantavirus antibody binds to the Gn subunit. In some embodiments, a hantavirus antibody does not bind to the tetramer lattice-dependent quaternary epitope.

Epitopes for the hantavirus antibodies of the present technology may be determined by any method well known in the art, for example, by conventional immunoassays. In one example, the epitope to which the hantavirus antibody binds may be determined in a systematic screening by using overlapping peptides derived from the hantavirus sequence and determining binding by the antibody. In some cases, during the discovery process, the generation and characterization of antibodies may elucidate information about desirable epitopes. This information may allow screening for antibodies that compete for binding to the same epitope, e.g., an epitope in the Gn or Gc subunit of the Gn/Gc spikes.

### Gn/Gc-Binding Activity

The activity of the hantavirus antibodies of the present technology may be assessed for binding using binding assays. Nonlimiting examples of suitable assays include surface plasmon resonance analysis using plate-based assay such as Enzyme0linked Immunosorbent Assay (ELISA), competition ELISA, or a yeast-based mAb competition assay.

In some embodiments, binding to hantavirus protein is assessed in a competitive assay format with a reference antibody of ADI-42898, ADI-42877, ADI-42098, ADI-42885, ADI-37236, ADI-65533 orADI-65534 or a reference antibody having the variable regions of ADI-42898, ADI-42877, ADI-42098, ADI-42885, ADI-37236, ADI-65533 or ADI-65534.

In some embodiments, a variant hantavirus antibody in accordance with the present technology may block binding of the reference antibody in a competition assay by about 50%, about 60%, about 65%, about 70%, about 75%, about 80%, about 85%, about 90%, about 9% or more. For instance, antibodies with about 75% reduction of binding are in competition with pre-complexed mAbs. Binding may be assessed by any number of means including flow cytometry.

In some embodiments, a variant hantavirus antibody in accordance with the present technology may block binding of the reference antibody in a competition assay by at least 50%, at least 60%, at least 65%, at least 70%, at least 75%, at least 80%, at least 85%, at least 90%, at least 9% or more. For instance, antibodies with at least 75% reduction of binding are in competition with pre-complexed mAbs. Binding may be assessed by any number of means including flow cytometry.

In some embodiments, a variant hantavirus antibody in accordance with the present technology may block binding of the reference antibody in a competition assay by at least about 50%, at least about 60%, at least about 65%, at least about 70%, at least about 75%, at least about 80%, at least about 85%, at least about 90%, at least about 9% or more. For instance, antibodies with at least about 75% reduction of binding are in competition with pre-complexed mAbs. Binding may be assessed by any number of means including flow cytometry.

The antibodies of the present technology (for example, ADI-42898, ADI-65533, and ADI-65534) may bind specifically to the PUUV Gn/Gc spike complex. The antibodies may recognize rVSV particles bearing hantavirus Gn/Gc proteins from both Old World (HTNV, Seoul virus [SEOV], Dobrava-Belgrade virus [DOBV]) and New World (ANDV, SNV) hantaviruses. The antibodies may have potent and broad serum-neutralizing antibody responses against rVSVs bearing Gn/Gc from PUUV, a divergent Old World hantavirus, Hantaan virus (HTNV), and a New World hantavirus, ANDV. Affinity-matured mAbs ADI-65533 and ADI-65534 may bind even better to ANDV Gn^{H}/Gc than their ADI-42898 parent.

In some embodiments, the antibodies have a similar degree of binding cross-reactivity between PUUV and viruses belonging to either Old World or New World hantavirus clades of about 10% to about 99%, about 20% to about 80%, about 30% to about 70%, about 40% to about 60%, about 15% to about 85%, about 5% to about 40%. In some embodiments, the antibodies have a similar degree of binding cross-reactivity between PUUV and viruses belonging to either Old World or New World hantavirus clades of about 14% to about 28%. In some embodiments, the antibodies bind a pH-sensitive quaternary epitope at the Gn/Gc interface. In some embodiments, the antibody is ADI-42898.

### Antibody Neutralizing Activity

In some embodiments, the antibodies of the present technology have potent and broad neutralizing activity against the Gn/Gc spike complex of PUUV and/or of a divergent Gn/Gc glycoproteins from both Old World (HTNV, SEOV, DOBV) and New World hantaviruses (ANDV). In some embodiments, the antibodies comprise neutralization activity against at least three hantaviruses with ten affording cross-clade neutralization.

In some embodiments, the antibodies of the present technology have broad activity recognizing two, three, four, five, or more antigenic sites in the Gn/Gc spike complex. Broad neutralizing antibodies (bnAbs) may be assigned to one of two competition groups, which are designated Groups I and 11. Group I and Group II neutralizing antibodies (nAbs) may recognize overlapping epitopes in the Gn subunit.

In some embodiments, the Group I nAbs recognize the capping loop in the membrane-distal N-terminal head domain of Gn (Gn^{H}). In some embodiments, the Group II nAbs recognize central sequences of the Gc subunit, including epitopes (i) in domain I near the flexible domain 1-111 linker region; (ii) at or near the domain I-II junction; and (iii) the ‘inner sheets’ of domain I, which mediate Gc:Gc contacts between adjacent (Gn/Gc)4 tetramers stabilizing the spike lattice.

In some embodiments, the antibodies of the present technology exhibit similar binding mode. The hantavirus antibodies of the present technology may recognize one or more residues in the Gn subunit (e.g., Gn^{H} capping loop) and the Gc subunit (e.g., Gc oligomerization interface). Gn/Gc recognition may be visualized by performing negative-stain transmission electron microscopy (nsEM) studies of the single-chain variable fragments (scFvs) of the nAbs. In some embodiments, two-dimensional classes of three distinctive elements of the complex, Gn^{H}, Gc, and scFvs of the Gn^{H}/Gc:nAb complexes, are identified. These distinctive elements may be averaged to calculate three-dimensional maps at 20 Å resolution. In some embodiments, all the Group I nAbs interact with the Gn^{H} capping loop. In some embodiments, the Group I nAbs bind apically to well-exposed sequences in pre-fusion spikes at the viral surface.

In some embodiments, the antibodies of the present technology specifically block viral membrane fusion. In some embodiments, the antibodies in Group I and Group II specifically block viral membrane fusion. In some embodiments, the antibodies in Group I and Group II target acid-triggered Gn/Gc rearrangements required for virus-cell membrane fusion. In some embodiments, the antibodies in Group I and Group II prevent the deployment and membrane insertion of the closely associated hydrophobic fusion loops (Group I nAbs). In some embodiments, the antibodies in Group I and Group II prevent the formation of a post-fusion Gc homotrimer (Group II nAbs). The capability of specifically blocking viral membrane fusion may be assessed by a fusion-infection assay.

In some embodiments, the antibodies of the present technology mediate dose dose-dependent reductions in PUUV Gn/Gc-dependent fusion-infection. In some embodiments, all Group I and II nAbs and some Group III and IV antibodies mediate dose dose-dependent reductions in PUUV Gn/Gc-dependent fusion-infection. Gn/Gc-specific polyclonal serum may have none an effect on the attachment of virions to target cells. In some embodiments, the antibodies (nAbs) of the present technology have no effect on the attachment of virions to target cells. In some embodiments, the antibodies directly interfere with Gn/Gc-dependent viral membrane fusion, as also observed for previously isolated nAbs. In some embodiments, the antibodies target one or more steps in the membrane fusion reaction. In some embodiments, the antibodies target Gn:Gc dissociation, Gc target membrane insertion, and/or homotrimerization.

In some embodiments, the antibodies of the present technology neutralize authentic Old World hantaviruses. The antibodies of the present technology may neutralize Old World hantaviruses (e.g., PUUV as shown by neutralization assays on rVSV-mNG-PUUV-Gn/Gc). In some embodiments, Gn- and/or potential Gn/Gc-targeting all Group I nAbs exhibit complete neutralization. In some embodiments, Gn- and/or potential Gn/Gc-targeting Group 111 and Group IV exhibit complete neutralization. In some embodiments, the Gc-targeting nAbs exhibit incomplete neutralization. In some embodiments, all nAbs in Group II exhibit an incomplete neutralization. In some embodiments, nAbs in Group IV exhibit a residual un-neutralized fraction. In some embodiments, all nAbs in Group I and/or some nAbs in Group IV do not neutralize at least 1, at least 2, at least 3, at least 4, at least 5, or more residuals.

In some embodiments, the antibodies of the present technology neutralize authentic HTNV and DOBV. In some embodiments, the antibodies exhibit complete neutralization of authentic HTNV and DOBV. In some embodiments, the antibodies are Gn^{H}/Go-specific bnAb and exhibit complete neutralization of authentic HTNV and DOBV. In some embodiments, the antibody ADI-42898 exhibit complete neutralization of authentic HTNV and DOBV. In some embodiments, the antibodies of the present technology Gc binders in Group II and Group IV have substantial narrowing in neutralization breadth.

In some embodiments, the antibodies of the present technology have recognize epitopes in Gn and Gc, respectively. In some embodiments, some of the antibodies (e.g., ADI-37236) bind to the Gn subunit (Gn^{H}) and the Gn/Gc (Gn^{H}/Gc) spike complex. In some embodiments, some of the antibodies (e.g., ADI-42089 and ADI-42898) only bind to the Gn/Gc (Gn^{H}/Gc) spike complex alone. In some embodiments, some of the antibodies (e.g., ADI-37236) contact more highly conserved sequences in the Gc subunit.

In some embodiments, the antibodies of the present technology have high potent activity against authentic hantaviruses (e.g., PUUV). The protection efficacy of antibodies against hantaviruses may be assessed using the bank vole infection model. some embodiments, the hantavirus nAbs exhibit complete pre-exposure prophylactic protection against viral acquisition in the lung, kidney, spleen, and blood. In some embodiments, the antibodies mediate substantial declines in mean viral RNA levels in the lung, spleen, kidney, and/or in circulation.

In some embodiments, the antibodies of the present technology exhibit cross-clade neutralization and post-exposure protection. In some embodiments, the hantavirus antibodies exhibit complete neutralization of authentic New World hantavirus glycoproteins from ANDV, SNV, and Choclo virus (CHOV). In some embodiments, Group I nAb, ADI-42898 exhibit complete neutralization of ANDV, SNV, and CHOV. In some embodiments, the hantavirus antibodies exhibit little or no cross-clade activity towards authentic New World hantaviruses. Neutralization activity of the antibodies against authentic Old Word hantaviruses and/or an rVSV bearing Gn/Gc from the New World hantavirus such as ANDV, SNV, and CHOV may be evaluated using any conventional methods. In some embodiments, ADI-42898, -65533, and/or 65534 block ANDV and PUUV infection.

### Antibody Formats

In some embodiments, a hantavirus antibody in accordance with the present technology comprises or consists of an antibody fragment (e.g., a Fv, Fab, Fab′, scFv, diabody, or F(ab′)₂ fragment). In some embodiments, the antibody comprises or consists of a substantially full-length antibody (e.g., an IgG antibody or other antibody class or isotype). In some embodiments, the antibody comprises or consists of an antibody fragment disdosed in Hudson et al. Nat. Med. 9: 129-134 (2003), incorporated herein by reference in its entirety. Antibody fragments may be made by various techniques, including but not limited to proteolytic digestion of an intact antibody as well as production by recombinant host cells.

In some embodiments, a hantavirus antibody of the present technology is an IgG of the IgG1 subclass. In some embodiments, such an antibody is an IgG of the IgG2, IgG3, or IgG4 subclass. In some embodiments, such an antibody is an IgM. In some embodiments, such an antibody has a lambda light chain constant region. In some embodiments, such an antibody has a kappa light chain constant region.

In some embodiments, a hantavirus antibody in accordance with the present technology is in a monovalent format. In some embodiments, the hantavirus antibody is in a fragment format (e.g., a Fv, Fab, Fab′, scFv, diabody, or F(ab′)₂ fragment).

In some embodiments, an antibody of the present technology may be chemically modified (e.g., one or more chemical moieties may be attached to the antibody) or be modified, e.g., produced in cell lines and/or in cell culture conditions to alter its glycosylation (e.g., hypofucosylation, afucosylation, or increased sialylation), to alter one or more functional properties of the antibody. For example, the antibody may be linked to one of a variety of polymers, for example, polyethylene glycol. In some embodiments, an antibody may comprise mutations to facilitate linkage to a chemical moiety and/or to alter residues that are subject to post-translational modifications, e.g., glycosylation.

In some embodiments, a hantavirus antibody of the present technology is employed in a bispecific or multi-specific format, e.g., a tri-specific format. For example, in some embodiments, the antibody may be incorporated into a bispecific or multi-specific antibody that comprises a further binding domain that binds to the same or a different antigen.

There are a variety of possible formats that may be used in bispecific or multi-specific antibodies. The formats may vary elements such as the number of binding arms, the format of each binding arm (e.g., Fab, scFv, scFab, or V_{H}-only), the number of antigen binding domains present on the binding arms, the connectivity and geometry of each arm with respect to each other, the presence or absence of an Fc domain, the Ig class (e.g., IgG or IgM), the Fc subclass (e.g., hIgG1, hlgG2, or hIgG4), and any mutations to the Fc (e.g., mutations to reduce or increase effector function or extend serum half-life). The antibodies of the present technology may comprise a bispecific or multispecific format. The bispecific or multispecific format may comprise a format disclosed in Speiss, et al., Alternative Molecular Formats and Therapeutic Applications for Bispecific Antibodies, Mol Immunol, 67, 95-106 (2015), incorporated herein by reference in its entirety.

In some embodiments, the antibody is a bispecific or multispecific antibody construct and the Gn/Gc spike complex comprises all six CDRs (HCDR1, HCDR2, HCDR3, LCDR1, LCDR2, and LCDR3) sequences from any one of antibodies. In some embodiments of the bispecific or multispecific antibody constructs, the binding domain comprises all six CDRs (HCDR1, HCDR2, HCDR3, LCDR1, LCDR2, and LCDR3) sequences from ADI-42898 V_{H}, ADI-42877 V_{H}, ADI-42098 V_{H}, ADI-42885 V_{H}, ADI-37236, ADI-65533, ADI-65534 V_{H} as provided in Table 1, and/or ADI-42898 V_{L}, ADI-42877 V_{L}, ADI-42098 V_{L}, ADI-42885 V_{L}, ADI-37236, ADI-65533, ADI-65534 V_{L} as provided in Table 2.

In some embodiments, the antibody is a bispecific or multispecific antibody construct and the Gn/Gc spike complex comprises the V_{H} and V_{L} sequences any one of ADI-42898, ADI-42877, ADI-42098, ADI-42885, ADI-37236, ADI-65533, and ADI-65534. In some embodiments, a bispecific or multispecific antibody constructs, the Gn/Gc spike complex comprises the V_{H} and V_{L} sequences of ADI-42898. In some embodiments, the antibody is a bispecific or multispecific antibody construct and the Gn/Gc spike complex comprises the V_{H} and V_{L} sequences of ADI-65533. In some embodiments, a bispecific or multispecific antibody constructs, the Gn/Gc spike complex comprises the V_{H} and V_{L} sequences of ADI-65534.

### Generation of Antibodies

Hantavirus antibodies as of the present technology may be produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology, incorporated herein by reference in its entirety). Reagents, cloning vectors, and kits for genetic manipulation are available from commercial vendors. Accordingly, in some embodiments, the present technology comprises isolated nucleic acids encoding a V_{H} and/or V_{L} region, or fragment thereof, of any of the antibodies of the present technology; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the antibody (e.g., the light and/or heavy chains of the antibody). In some embodiments, the host cell contains (1) a vector containing a polynudeotide that encodes the V_{L} amino acid sequence and a polynudeotide that encodes the V_{H} amino acid sequence, or (2) a first vector containing a polynudeotide that encodes the V_{L} amino acid sequence and a second vector containing a polynudeotide that encodes the V_{H} amino acid sequence.

In some embodiments, the present technology comprises methods of making a hantavirus antibody of the present technology. In some embodiments, the method includes culturing a host cell under conditions suitable for expression of the antibody. In some embodiments, the antibody is subsequently recovered from the host cell (or host cell culture medium).

Suitable vectors containing polynucleotides encoding antibodies of the present technology, or fragments thereof, include cloning vectors and expression vectors. While the cloning vector selected may vary according to the host cell intended to be used, useful cloning vectors generally may self-replicate, may possess a single target for a particular restriction endonuclease, and/or may carry genes for a marker that may be used in selecting clones containing the vector. Nonlimiting examples include plasmids and bacterial viruses, e.g., pUC18, pUC19, Bluescript (e.g., pBS SK+) and its derivatives, mpl8, mpl9, pBR322, pMB9, ColE1 plasmids, pCR1, RP4, phage DNAs, and shuttle vectors.

The expression vector may be replicable in the host cells either as episomes or as a part of the chromosomal DNA. Suitable expression vectors include but are not limited to plasmids and viral vectors, including adenoviruses, adeno-associated viruses, retroviruses, and any other vector.

Suitable host cells for expressing a hantavirus antibody of the present technology include both prokaryotic and eukaryotic cells. For example, a hantavirus antibody may be produced in bacteria, when glycosylation and Fc effector function are not needed. After expression, the antibody may be isolated from the bacterial cell paste in a soluble fraction and may be further purified. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures may also be utilized as host cells.

In some embodiments, vertebrate host cells are used for producing a hantavirus antibody of the present technology. For example, mammalian cell lines such as a monkey kidney CV1 line transformed by SV40 (COS-7); human embryonic kidney (HEK) line (e.g., HEK293 or HEK293T cells); baby hamster kidney cells (BHK); mouse sertoli cells (e.g., TM4 cells), 1980 monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells; and FS4 cells may be used to express an antibodies. Other useful mammalian host cell lines include Chinese hamster ovary (CHO) cells, including DHFR-CHO cells (e.g., DHFR_CHO cells disclosed in Urlaub et al., Proc. Natl. Acad. Sci. USA 77:4216, 1980, incorporated herein by reference in its entirety); and myeloma cell lines such as Y0, NS0 and Sp2/0. Host cells of the present technology also include, without limitation, isolated cells, in vitro cultured cells, and ex vivo cultured cells. The host cell may comprise a mammalian host cell lines suitable for antibody production disclosed in Yazaki and Wu, Methods in Molecular Biology, Vol. 248 (B. K. C. Lo, ed., Humana Press, Totowa, NJ), pp. 255-268, 2003, incorporated herein by reference in its entirety.

In some embodiments, a hantavirus antibody of the present technology is produced by a CHO cell line, e.g., the CHO-K1 cell line. One or more expression plasmids may be introduced that encode heavy and light chain sequences. For example, in one embodiment, an expression plasmid encoding a heavy chain of the present technology, e.g., SEQ ID NO:1, and an expression plasmid encoding a light chain of the present technology, e.g., SEQ ID NO: 81 or 21 are transfected into host cells as linearized plasmids at a ratio of 1:1 in the CHO-K1 host cell line using reagents such as Freestyle Max reagent. Fluorescence-activated cell sorting (FACS) coupled with single cell imaging may be used as a cloning method to obtain a production cell line.

A host cell transfected with an expression vector encoding a hantavirus antibody of the present technology, or fragment thereof, may be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, a hantavirus antibody of the present technology may be produced by in vitro synthesis (e.g., Sutro Biopharma biochemical protein synthesis platform).

In some embodiments, the present technology comprises a method of generating variants of a hantavirus antibody as of the present technology. In a nonlimiting example, a construct encoding a variant of a V_{H} CDR3 of the present technology may be modified and the V_{H} region encoded by the modified construct may be tested for binding activity to in vitro (e.g., rVSV-Gn/Gc) neutralizing antibody activity and/or in vivo protection efficacy in the context of a V_{H} region of the present technology, that is paired with a V_{L} region or variant region of the present technology. In another non limiting example, a construct encoding a variant of a V_{L} CDR3 of the present technology may be modified and the V_{L} region encoded by the modified construct may be tested for binding to in vitro (e.g., rVSV-Gn/Gc) neutralizing antibody activity and/or in vivo protection efficacy. Such an analysis may also be performed with other CDRs or framework regions and an antibody having the desired activity may then be selected.

### Engineering Variants

In some embodiments, an antibody or variant thereof of the present technology is modified to have improved developability (i.e., reduced development liabilities), including but not limited to, decreased heterogeneity, increased yield, increased stability, improved net charges to improve pharmacokinetics, and or/reduced immunogenicity. In some embodiments, antibodies having improved developability may be obtained by introducing mutations to reduce or eliminate potential development liabilities. In some embodiments, antibodies having improved developability possess modifications as compared to a reference or control antibody in their amino acid sequence.

In some embodiments, the antibodies or variants thereof of the present technology have improved developability while maintaining comparable or improved binding affinity to the target antigen as compared to a reference or control (unmodified) antibody. In some embodiments, the antibodies or variants thereof of the present technology have improved developability while maintaining activities that are like a reference or control (unmodified) antibody.

In some embodiments, the antibodies or variants thereof have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H₂O₂. Mutations may be considered successful if activity is maintained (or enhanced) while removing or reducing the severity of the liability.

### Antibody Conjugates/Co-Stimulatory Agents

In some embodiments, a hantavirus antibody of the present technology may be conjugated or linked to therapeutic, imaging/detectable moieties, or enzymes. For example, the antibody may be conjugated to a detectable marker, a cytotoxic agent, an immunomodulating agent, an imaging agent, a therapeutic agent, an oligonucleotide, or an enzyme. The antibodies may be conjugated or linked to a desired molecule using any method well known in the art. The moiety may be linked to the antibody covalently or by non-covalent linkages.

In some embodiments, the antibody is conjugated, either directly or via a cleavable or non-cleavable linker, to a cytotoxic moiety or other moiety that inhibits cell proliferation. In some embodiments, the antibody is conjugated to a cytotoxic agent including, but not limited to, e.g., ricin A chain, doxorubicin, daunorubicin, a maytansinoid, taxol, ethidium bromide, mitomycin, etoposide, tenoposide, vincristine, vinblastine, colchicine, dihydroxy anthracin dione, methotrexact, actinomycin, a diphtheria toxin, extotoxin A from Pseudomonas, Pseudomonas exotoxin40, abrin, abrin A chain, modeccin A chain, alpha sarcin, gelonin, mitogellin, restrictocin, cobran venom factor, a ribonuclease, engineered Shiga toxin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a proliferation inhibitor, a lytic agent, a DNA or RNA synthesis inhibitors, a membrane permeability modifier, a DNA metabolite, a dichloroethylsulfide derivative, a protein production inhibitor, a ribosome inhibitor, or an inducer of apoptosis. In some embodiments, the antibody is conjugated to a drug such as a topoisomeriase inhibitor (e.g., a topoisomeraise I inhibitor).

In some embodiments, a hantavirus antibody of the present technology is joined to a molecule that facilitates transport of the antibody across a biological membrane, e.g., by enhancing penetration of the membrane, facilitating protein translocation across membranes. In a nonlimiting example, the antibody may be linked to a cell penetration agent, such as a cell-penetrating peptide. Nonlimiting examples of cell penetrating peptides include TAT, penetrating, polyarginine molecules, Kunitz domain-derived peptides, e.g., angiopep-2, SynB, buforin, transportan, amphiphathic peptides and others. In some embodiments, the antibody may be conjugated with a cationic molecule such as a polyamine. In some embodiments, the antibody may be conjugated to an agent that facilitates transport across the blood brain barrier, e.g., transcytosis. In a nonlimiting example, the antibody may be conjugated to an agent that binds to endothelial cell receptors that are internalized, e.g., transferrin receptor, insulin receptor, insulin-like growth factor receptor, or a low-density lipoprotein receptor, and the like. In some embodiments, the antibody may be conjugated to a toxin facilitating entry of the antibody into the cytoplasm, e.g., Shiga toxin. In some embodiments, a hantavirus antibody of the present technology may be conjugated to an engineered toxin body (ETBs) to facilitate internalization of the antibody into a cell.

In some embodiments, a hantavirus antibody of the present technology is conjugated or administered with a polypeptide immunomodulating agent, e.g., an adjuvant. Examples of immunomodulating agents include, but are not limited to, cytokines (e.g., transforming growth factor-β (TGFβ)), growth factors, lymphotoxins, tumor necrosis factor (TNF), hematopoietic factors, interleukins (e.g., interleukin-1 (IL-1), IL-2, IL-3, IL-6, IL-10, IL-12, IL-15, an IL-15/IL-15Rα, e.g., sushi domain, complex, IL-18, and IL-21), colony stimulating factors (e.g., granulocyte-colony stimulating factor (G-CSF) and granulocyte macrophage-colony stimulating factor (GM-CSF), interferons (e.g., interferon-α, -β or -γ, erythropoietin and thrombopoietin, or a combination thereof. In some embodiments, the antibody is linked or administered with a compound that stimulates the innate immune system, such as an adjuvant, a Toll-like receptor (TLR) agonist, a C-type lectin receptor (CLR) agonist, a retinoic acid-inducible gene I-like receptor (RLR) agonist, a saponin, a polysaccharide such as chitin, chitosan, β-glucan, an ISCOM, QS-21, or a stimulator of interferon genes (STING) agonist.

In some embodiments, the antibody may be linked to a radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

In one embodiment of any of the above constructs, the hantavirus antibody is any one of ADI-42898, ADI-42877, ADI-42098, ADI-42885, ADI-37236, ADI-65533, or ADI-65534. In one embodiment of any of the above constructs, the binding domain comprises the V_{H} and V_{L} sequences of ADI-42898.

### Pharmaceutical Compositions and Formulations

In some embodiments, the hantavirus antibodies of the present technology, or variants thereof, are present in a pharmaceutical composition. The pharmaceutical composition may be formulated for use in a variety of drug delivery systems. One or more physiologically acceptable excipients or carriers may also be included in the compositions for proper formulation. Suitable formulations for use in the present technology may comprise those disclosed in Remington: The Science and Practice of Pharmacy, 21st Edition, Philadelphia, PA. Lippincott Williams & Wilkins, 2005, incorporated herein by reference in its entirety.

In some embodiments, the pharmaceutical compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising antibodies of the present technology further comprise a suitable infusion media.

In some embodiments, the pharmaceutical composition may have various formulations, for example, injectable formulations, lyophilized formulations, liquid formulations, oral formulations, depending on the suitable routes of administration. In some embodiments, the pharmaceutical compositions may have various formulations for injection and/or infusion. Non-limiting examples of formulations for injection and/or infusion include intravenous injection, intraperitoneal injection, intertumoral injection, bone marrow injection, lymph node injection, subcutaneous injection, and cerebrospinal fluid injection.

The antibodies of the present technology may be provided in a solution suitable for administration to the subject, such as a sterile isotonic aqueous solution for injection. The antibodies may be dissolved or suspended at a suitable concentration in an acceptable carrier. In some embodiments the carrier is an aqueous carrier. Nonlimiting examples of aqueous carriers include water, saline, phosphate buffered saline, and the like. The compositions may contain auxiliary pharmaceutical substances as required to approximate physiological conditions, such as pH adjusting and buffering agents, tonicity adjusting agents, and the like.

The solution may comprise agents such as buffering agents and stabilizing agents. For example, in some embodiments, a buffering agent such as histidine is included to maintain a formulation pH of about 5.5. Additional reagents such as sucrose or alternatives may be added to prevent aggregation and fragmentation in solution and during freezing and thawing. Agents such as polysorbate 80 or an alternative may be included to lower surface tension and stabilizes the antibody against agitation-induced denaturation and air-liquid and ice-liquid surface denaturation

In some embodiments, the formulation pH is about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, or about 6.5.

In some embodiments, the formulation pH is at least 4.5, at least 4.6, at least 4.7, at least 4.8, at least 4.9, at least 5.0, at least 5.1, at least 5.2, at least 5.3, at least 5.4, at least 5.5, at least 5.6, at least 5.7, at least 5.8, at least 5.9, at least 6.0, at least 6.1, at least 6.2, at least 6.3, at least 6.4, or at least 6.5.

In some embodiments, the formulation pH is at least about 4.5, at least about 4.6, at least about 4.7, at least about 4.8, at least about 4.9, at least about 5.0, at least about 5.1, at least about 5.2, at least about 5.3, at least about 5.4, at least about 5.5, at least about 5.6, at least about 5.7, at least about 5.8, at least about 5.9, at least about 6.0, at least about 6.1, at least about 6.2, at least about 6.3, at least about 6.4, or at least about 6.5.

### Associated Methods

### Methods of Inducing an Immune Response

In some embodiments, the present technology comprises methods of inducing an immune response by administering a hantavirus antibody of the present technology to a subject in need thereof.

### Treatment of Hantavirus Infection & Hantavirus-Associated Diseases

In some embodiments, a hantavirus antibody or a variant thereof of the present technology, may be used and a therapeutic agent to treat, prevent, reduce, or otherwise ameliorate one or more hantavirus-associated diseases caused by one or more hantavirus species.

A hantavirus antibody of the present technology may be used to treat several different hantavirus-associated diseases or symptoms caused by a hantavirus infection. In some embodiments, a subject (e.g., a mammal, a human) who may benefit from the treatment of the hantavirus antibody has HCPS. In some embodiments, a subject (e.g., a mammal, a human) who may benefit from the treatment of the hantavirus antibody has HFRS. In some embodiments, a subject (e.g., a mammal, a human) who may benefit from the treatment of the hantavirus antibody has nephropathia epidemica (NE). In some embodiments, a subject (e.g., a mammal, a human) who may benefit from the treatment of the hantavirus antibody has one or more symptoms including fatigue, fever, muscle aches, headaches, dizziness, chills, abdominal problems, nausea, vomiting, diarrhea, abdominal pain, coughing, and shortness of breath.

In some embodiments, the hantavirus antibody or variants thereof treat, prevent, reduce, or otherwise ameliorate a hantavirus infection.

In some embodiments, administration of an antibody or a variant thereof of the present technology induces about 50%, or about 60%, or about 70%, about 80%, about 90%, about 95%, or greater protective efficacy against infection of a hantavirus in a subject in need thereof relative to a control.

In some embodiments, administration of an antibody or a variant thereof of the present technology induces at least 50%, or at least 60%, or at least 70%, at least 80%, at least 90%, at least 95%, or greater protective efficacy against infection of a hantavirus a subject in need thereof relative to a control.

In some embodiments, administration of an antibody or a variant thereof of the present technology induces at least about 50%, or at least about 60%, or at least about 70%, at least about 80%, at least about 90%, at least about 95%, or greater protective efficacy against infection of a hantavirus in a subject in need thereof relative to a control.

In some embodiments, administration of an antibody or a variant of the present technology reduces hantavirus infection by about 50%, or about 60%, or about 70%, about 80%, about 90%, about 95%, or greater in a subject in need thereof relative to a control.

In some embodiments, administration of an antibody or a variant of the present technology reduces hantavirus infection by at least 50%, or at least 60%, or at least 70%, at least 80%, at least 90%, at least 95%, or greater in a subject in need thereof relative to a control.

In some embodiments, administration of an antibody or a variant of the present technology reduces hantavirus infection by at least about 50%, or at least about 60%, or at least about 70%, at least about 80%, at least about 90%, at least about 95%, or greater in a subject in need thereof relative to a control.

In some embodiments, a hantavirus antibody of the present technology may be used to treat, prevent, reduce, or otherwise ameliorate one or more hantavirus-associated diseases or symptoms caused by an infection of a hantavirus in the family Hantaviridae, in a subject in need thereof. In some embodiments, a hantavirus antibody of the present technology may be used to treat, prevent, reduce, or otherwise ameliorate infection of a hantavirus in the family Hantaviidae in a subject in need thereof.

In some the hantavirus is selected from the group consisting of Andes orthohantavirus, Asama orthohantavirus, Asikkala orthohantavirus, Bayou orthohantavirus, Black Creek Canal orthohantavirus, Bowe orthohantavirus, Bruges orthohantavirus, Cano Delgadito orthohantavirus, Cao Bang orthohantavirus, Choclo orthohantavirus, Dabieshan orthohantavirus, Dobrava-Belgrade orthohantavirus, El Moro Canyon orthohantavirus, Fugong orthohantavirus, Fusong orthohantavirus, Hantaan orthohantavirus, Jeju orthohantavirus, Kenkeme orthohantavirus, Khabarovsk orthohantavirus, Laguna Negra orthohantavirus, Luxi orthohantavirus, Maporal orthohantavirus, Montano orthohantavirus, Necocli orthohantavirus, Oxbow orthohantavirus, Prospect Hill orthohantavirus, Puumala orthohantavirus, Robina orthohantavirus, Rockport orthohantavirus, Sangassou orthohantavirus, Seewis orhtohantavirus, Seoul orthohantavirus, Sin Nombre orthohantavirus, Tatenale orthohantavirus, Thailand orthohantavirus, Tigray orthohantavirus, Tula orthohantavirus, and Yakeshi orthohantavirus.

In some the hantavirus is selected from the group consisting of Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV).

In some embodiments, the antibodies of the present technology at least partially or completely neutralize one or more hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV).

In some embodiments, the antibodies of the present technology neutralize two or more hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Chodo virus (CHOV), Laguna Negra virus (LGNV), Maporal virus (MPRLV), Prospect Hill virus (PHV), and Tula virus (TULV). In some embodiments, the isolated antibody neutralizes at least three hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Chodo virus (CHOV), Laguna Negra virus (LGNV), Maporal virus (MPRLV), Prospect Hill virus (PHV), and Tula virus (TULV). In some embodiments, the isolated antibody neutralizes at least four hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV). In some embodiments, the isolated antibody neutralizes at least five hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV). In some embodiments, the isolated antibody neutralizes at least six hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV). In some embodiments, the isolated antibody neutralizes at least seven hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV).

In some embodiments, the isolated antibody neutralizes Puumala virus (PUUV) and at least one hantavirus selected from Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV). In some embodiments, the isolated antibody neutralizes Puumala virus (PUUV) and Hantaan virus (HTNV). In some embodiments, the isolated antibody neutralizes Puumala virus (PUUV) and Dobrava-Belgrade virus (DOBV). In some embodiments, the isolated antibody neutralizes Puumala virus (PUUV) and Choclo virus (CHOV). In some embodiments, the isolated antibody neutralizes Puumala virus (PUUV) and Andes virus (ANDV). In some embodiments, the isolated antibody neutralizes Puumala virus (PUUV) and Sin Nombre virus (SNV). In some embodiments, the isolated antibody neutralizes Puumala virus (PUUV), Hantaan virus (HTNV) and Dobrava-Belgrade virus (DOBV). In some embodiments, the isolated antibody neutralizes Puumala virus (PUUV), Andes virus (ANDV), Sin Nombre virus (SNV), and Choclo virus (CHOV).

In some embodiments, the isolated antibody neutralizes Hantaan virus (HTNV) and Dobrava-Belgrade virus (DOBV). In some embodiments, the isolated antibody neutralizes Andes virus (ANDV), Sin Nombre virus (SNV), and Choclo virus (CHOV).

In some embodiments, the isolated antibody neutralizes Puumala virus (PUUV). In some embodiments, the isolated antibody neutralizes Hantaan virus (HTNV). In some embodiments, the isolated antibody neutralizes Dobrava-Belgrade virus (DOBV). In some embodiments, the isolated antibody neutralizes Andes virus (ANDV). In some embodiments, the isolated antibody neutralizes Sin Nombre virus (SNV). In some embodiments, the isolated antibody neutralizes Choclo virus (CHOV). In some embodiments, the isolated antibody neutralizes Seoul virus (SEOV).

In some embodiments, the hantavirus is a hantavirus carried by rodents such as Old-World mice and rats (Murinae), New World mice and rats (Neotominae and Sigmodontinae), and voles (Arvicolinae).

### Dosing & Administration

The antibodies and/or the pharmaceutical compositions of the present technology may be administered to a subject (e.g., a mammal, a human) in an amount sufficient to cure or at least partially arrest the disease or symptoms of the disease and its complications. An amount adequate to accomplish this is defined as a “therapeutically effective dose.” A therapeutically effective dose is determined by monitoring a subject's response to therapy. Typical benchmarks indicative of a therapeutically effective dose includes the amelioration of symptoms of the disease in the subject. Amounts effective for this use will depend upon the severity of the disease and the general state of the subjects health, including other factors such as age, weight, gender, administration route, and the like single or multiple administrations of the antibody may be administered depending on the dosage and frequency as required and tolerated by the subject. In any event, the methods provide a sufficient quantity of hantavirus epitope targeting antibody to effectively treat the subject.

A hantavirus antibody and/or pharmaceutical composition of the present technology may be administered by any suitable means, including, for example, parenteral, intrapulmonary, and intranasal, administration, as well as local administration. Parenteral infusions include intramuscular, intravenous, intraarterial, intraperitoneal, or subcutaneous administration. In some embodiments, the antibody or the pharmaceutical composition may be administered by insufflation. In some embodiments, the antibody or pharmaceutical composition may be stored at 10 mg/ml in sterile isotonic aqueous saline solution for injection at 4° C. and is diluted in either 100 ml or 200 ml 0.9% sodium chloride for injection prior to administration to the subject. In some embodiments, the antibody or the pharmaceutical composition is administered by intravenous infusion over the course of about 1 hour at a dose of between about 0.01 and about 25 mg/kg. In other embodiments, the antibody or the pharmaceutical composition is administered by intravenous infusion over a period of between about 15 minutes and about 2 hours. In still other embodiments, the administration procedure is via sub-cutaneous bolus injection.

The dose of antibody or the pharmaceutical composition is chosen to provide effective therapy for the subject and is in the range of less than about 0.01 mg/kg body weight to about 25 mg/kg body weight or in the range 1 mg-2 g per subject. In some embodiments, the dose is in the range of about 0.1-about 10 mg/kg or approximately about 50 mg-about 1000 mg/subject. The dose may be repeated at an appropriate frequency which may be in the range once per day to once every three months, or every six months, depending on the pharmacokinetics of the antibody (e.g., half-life of the antibody in the circulation) or the pharmaceutical composition and the pharmacodynamic response (e.g., the duration of the therapeutic effect of the antibody). In some embodiments, the in vivo half-life of between about 7 and about 25 days and antibody or pharmaceutical composition dosing is repeated between once per week and once every 3 months or once every 6 months. In other embodiments, the antibody or the pharmaceutical composition is administered approximately once per month.

In some embodiments, the antibody or the pharmaceutical composition may be stored at about 10 mg/ml or about 20 mg/ml in a sterile isotonic aqueous solution. In some embodiments, the solution for injection is stored at about 4° C. and is diluted in either about 100 ml or about 200 ml 0.9% sodium chloride for injection prior to administration to the subject.

In some embodiments, the antibody or the pharmaceutical composition for intravenous administration is formulated at a target concentration of about 20 mg/mL in about 20 mM histidine buffer, about 8% (w/v) sucrose and about 0.02% (w/v) polysorbate 80, about pH 5.5.

In some embodiments, the subject is administered the antibody or the pharmaceutical composition of the present technology at a dose of about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10 mg/kg, about 12 mg/kg, about 14 mg/kg, about 15 mg/kg, about 16 mg/kg, about 18 mg/kg, about 20 mg/kg, about 22 mg/kg, about 24 mg/kg, about 25 mg/kg, about 26 mg/kg, about 28 mg/kg, about 30 mg/kg, about 35 mg/kg, about 40 mg/kg, about 50 mg/kg, about 60 mg/kg, about 70 mg/kg, about 80 mg/kg, or about 100 mg/kg.

In some embodiments, the subject is administered the antibody or the pharmaceutical composition of the present technology at a dose of at least 0.5 mg/kg, at least 1.0 mg/kg, at least 1.5 mg/kg, at least 2.0 mg/kg, at least 2.5 mg/kg, at least 3.0 mg/kg, at least 3.5 mg/kg, at least 4.0 mg/kg, at least 4.5 mg/kg, at least 5.0 mg/kg, at least 5.5 mg/kg, at least 6.0 mg/kg, at least 6.5 mg/kg, at least 7.0 mg/kg, at least 7.5 mg/kg, at least 8.0 mg/kg, at least 8.5 mg/kg, at least 9.0 mg/kg, at least 9.5 mg/kg, at least 10 mg/kg, at least 12 mg/kg, at least 14 mg/kg, at least 15 mg/kg, at least 16 mg/kg, at least 18 mg/kg, at least 20 mg/kg, at least 22 mg/kg, at least 24 mg/kg, at least 25 mg/kg, at least 26 mg/kg, at least 28 mg/kg, at least 30 mg/kg, at least 35 mg/kg, at least 40 mg/kg, at least 50 mg/kg, at least 60 mg/kg, at least 70 mg/kg, at least 80 mg/kg, or at least 100 mg/kg.

In some embodiments, the subject is administered the antibody or the pharmaceutical composition of the present technology at a dose of at least about 0.5 mg/kg, at least about 1.0 mg/kg, at least about 1.5 mg/kg, at least about 2.0 mg/kg, at least about 2.5 mg/kg, at least about 3.0 mg/kg, at least about 3.5 mg/kg, at least about 4.0 mg/kg, at least about 4.5 mg/kg, at least about 5.0 mg/kg, at least about 5.5 mg/kg, at least about 6.0 mg/kg, at least about 6.5 mg/kg, at least about 7.0 mg/kg, at least about 7.5 mg/kg, at least about 8.0 mg/kg, at least about 8.5 mg/kg, at least about 9.0 mg/kg, at least about 9.5 mg/kg, at least about 10 mg/kg, at least about 12 mg/kg, at least about 14 mg/kg, at least about 15 mg/kg, at least about 16 mg/kg, at least about 18 mg/kg, at least about 20 mg/kg, at least about 22 mg/kg, at least about 24 mg/kg, at least about 25 mg/kg, at least about 26 mg/kg, at least about 28 mg/kg, at least about 30 mg/kg, at least about 35 mg/kg, at least about 40 mg/kg, at least about 50 mg/kg, at least about 60 mg/kg, at least about 70 mg/kg, at least about 80 mg/kg, or at least about 100 mg/kg.

In some embodiments, the antibodies or the pharmaceutical compositions of the present technology are administered once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years.

In some embodiments, the antibodies or the pharmaceutical compositions of the present technology are administered once a day, twice a day, three times a day, or four times a day for a period of at least 3 days, at least 5 days, at least 7 days, at least 10 days, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 1.25 years, at least 1.5 years, at least 1.75 years, at least 2 years, at least 2.25 years, at least 2.5 years, at least 2.75 years, at least 3 years, at least 3.25 years, at least 3.5 years, at least 3.75 years, at least 4 years, at least 4.25 years, at least 4.5 years, at least 4.75 years, at least 5 years, or more than at least 5 years.

In some embodiments, the antibodies or the pharmaceutical compositions of the present technology are administered once a day, twice a day, three times a day, or four times a day for a period of at least about 3 days, at least about 5 days, at least about 7 days, at least about 10 days, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 1.25 years, at least about 1.5 years, at least about 1.75 years, at least about 2 years, at least about 2.25 years, at least about 2.5 years, at least about 2.75 years, at least about 3 years, at least about 3.25 years, at least about 3.5 years, at least about 3.75 years, at least about 4 years, at least about 4.25 years, at least about 4.5 years, at least about 4.75 years, at least about 5 years, or more than at least about 5 years.

In some embodiments, the antibodies or the pharmaceutical compositions of the present technology are administered every day, every other day, every third day, weekly, biweekly (i.e., every other week), every third week, monthly, every other month, every third month, every fourth month, every fifth month, every sixth month, every ninth month, every year, every 18 months, or every 2 years.

### Combination Therapy

A hantavirus antibody of the present technology may be administered with one or more additional antibodies. In some embodiments, a hantavirus antibody of the present technology (e.g., ADI-42898) is administered sequentially or simultaneously with one or more additional antibodies (e.g., ADI-42877, ADI-42098, ADI-42885, ADI-37236, ADI-65533, or ADI-65534). In some embodiments, a hantavirus antibody of the present technology (e.g., ADI-65533) is administered sequentially or simultaneously with one or more additional antibodies (e.g., ADI-42898, ADI-42877, ADI-42098, ADI-42885, ADI-37236, or ADI-65534). In some embodiments, a hantavirus antibody of the present technology (e.g., ADI-65534) is administered sequentially or simultaneously with one or more additional antibodies (e.g., ADI-42898, ADI-42877, ADI-42098, ADI-42885, ADI-37236, or ADI-65533).

In some embodiments, the hantavirus antibody of the present technology is administered about 30 seconds, about 1 minute, about 2 minutes, about 3 minutes, about 4 minutes, about 5 minutes, about 10 minutes, about 20 minutes, about 30 minutes, about 45 minutes, about 60 minutes, about 90 minutes, about 2 hours, about 3 hours, about 4 hours, about 6 hours, about 8 hours, about 10 hours, about 12 hours, about 14 hours, about 16 hours, about 18 hours, about 20 hours, about 1 day, about 2 days, about 3 days, about 4 days, about 5 days, about 6 days, about 8 days, about 10 days, about 12 days, about 14 days, about 3 weeks, about 1 month, about 2 months, about 3 months, about 4 months, about 5 months, about 6 months, about 8 months, about 10 months, or about 1 year before or after administration of the one or more additional antibodies.

In some embodiments, the hantavirus antibody of the present technology is administered at least 30 seconds, at least 1 minute, at least 2 minutes, at least 3 minutes, at least 4 minutes, at least 5 minutes, at least 10 minutes, at least 20 minutes, at least 30 minutes, at least 45 minutes, at least 60 minutes, at least 90 minutes, at least 2 hours, at least 3 hours, at least 4 hours, at least 6 hours, at least 8 hours, at least 10 hours, at least 12 hours, at least 14 hours, at least 16 hours, at least 18 hours, at least 20 hours, at least 1 day, at least 2 days, at least 3 days, at least 4 days, at least 5 days, at least 6 days, at least 8 days, at least 10 days, at least 12 days, at least 14 days, at least 3 weeks, at least 1 month, at least 2 months, at least 3 months, at least 4 months, at least 5 months, at least 6 months, at least 8 months, at least 10 months, or at least 1 year before or after administration of the one or more additional antibodies.

In some embodiments, the hantavirus antibody of the present technology is administered at least about 30 seconds, at least about 1 minute, at least about 2 minutes, at least about 3 minutes, at least about 4 minutes, at least about 5 minutes, at least about 10 minutes, at least about 20 minutes, at least about 30 minutes, at least about 45 minutes, at least about 60 minutes, at least about 90 minutes, at least about 2 hours, at least about 3 hours, at least about 4 hours, at least about 6 hours, at least about 8 hours, at least about 10 hours, at least about 12 hours, at least about 14 hours, at least about 16 hours, at least about 18 hours, at least about 20 hours, at least about 1 day, at least about 2 days, at least about 3 days, at least about 4 days, at least about 5 days, at least about 6 days, at least about 8 days, at least about 10 days, at least about 12 days, at least about 14 days, at least about 3 weeks, at least about 1 month, at least about 2 months, at least about 3 months, at least about 4 months, at least about 5 months, at least about 6 months, at least about 8 months, at least about 10 months, or at least about 1 year before or after administration of the one or more additional antibodies.

A hantavirus antibody of may be administered with one or more additional therapeutic agents (e.g., an immunostimulatory agent or an immunotherapeutic agent).

In some embodiments, a hantavirus antibody may be administered in conjunction with a therapeutic agent that targets an immune checkpoint antigen. In some embodiments, the therapeutic agent is a biologic therapeutic or a small molecule. In some embodiments, the therapeutic agent is a monoclonal antibody, a humanized antibody, a human antibody, a fusion protein or a combination thereof. In some embodiments, the therapeutic agents inhibit, e.g., by blocking ligand binding to receptor, a checkpoint antigen that may be PD1, PDL1, CTLA-4, ICOS, PDL2, IDO1, IDO2, B7-H3, B7-H4, BTLA, HVEM, TIM3, GAL9, GITR, HAVCR2, LAG3, KIR, LAIR1, LIGHT, MARCO, OX-40, SLAM, 2B4, CD2, CD27, CD28, CD30, CD40, CD70, CD80, CD86, CD137 (4-1BB), CD160, CD39, VISTA, TIGIT, a SIGLEC, CGEN-15049, 2B4, CHK1, CHK2, A2aR, B-7 family ligands or their receptors, or a combination thereof. In some embodiments, the therapeutic agent targets PD-1, e.g., an antibody that blocks PD-L1 binding to PD-1 or otherwise inhibits PD-1. In some embodiments, the therapeutic agent targets CTLA-4. In some embodiments, the therapeutic agent targets LAG3. In some embodiments, the therapeutic agent targets TIM3. In some embodiments, the therapeutic agents target ICOS.

In some embodiments, a hantavirus antibody of the present technology may be administered with a therapeutic agent, e.g., a corticosteroid, that mitigates side-effects resulting from stimulation of the immune system.

In some embodiments, the hantavirus antibody of the present technology is administered about 30 seconds, about 1 minute, about 2 minutes, about 3 minutes, about 4 minutes, about 5 minutes, about 10 minutes, about 20 minutes, about 30 minutes, about 45 minutes, about 60 minutes, about 90 minutes, about 2 hours, about 3 hours, about 4 hours, about 6 hours, about 8 hours, about 10 hours, about 12 hours, about 14 hours, about 16 hours, about 18 hours, about 20 hours, about 1 day, about 2 days, about 3 days, about 4 days, about 5 days, about 6 days, about 8 days, about 10 days, about 12 days, about 14 days, about 3 weeks, about 1 month, about 2 months, about 3 months, about 4 months, about 5 months, about 6 months, about 8 months, about 10 months, or about 1 year before or after administration of the therapeutic agent.

In some embodiments, the hantavirus antibody of the present technology is administered at least 30 seconds, at least 1 minute, at least 2 minutes, at least 3 minutes, at least 4 minutes, at least 5 minutes, at least 10 minutes, at least 20 minutes, at least 30 minutes, at least 45 minutes, at least 60 minutes, at least 90 minutes, at least 2 hours, at least 3 hours, at least 4 hours, at least 6 hours, at least 8 hours, at least 10 hours, at least 12 hours, at least 14 hours, at least 16 hours, at least 18 hours, at least 20 hours, at least 1 day, at least 2 days, at least 3 days, at least 4 days, at least 5 days, at least 6 days, at least 8 days, at least 10 days, at least 12 days, at least 14 days, at least 3 weeks, at least 1 month, at least 2 months, at least 3 months, at least 4 months, at least 5 months, at least 6 months, at least 8 months, at least 10 months, or at least 1 year before or after administration of the therapeutic agent.

In some embodiments, the hantavirus antibody of the present technology is administered at least about 30 seconds, at least about 1 minute, at least about 2 minutes, at least about 3 minutes, at least about 4 minutes, at least about 5 minutes, at least about 10 minutes, at least about 20 minutes, at least about 30 minutes, at least about 45 minutes, at least about 60 minutes, at least about 90 minutes, at least about 2 hours, at least about 3 hours, at least about 4 hours, at least about 6 hours, at least about 8 hours, at least about 10 hours, at least about 12 hours, at least about 14 hours, at least about 16 hours, at least about 18 hours, at least about 20 hours, at least about 1 day, at least about 2 days, at least about 3 days, at least about 4 days, at least about 5 days, at least about 6 days, at least about 8 days, at least about 10 days, at least about 12 days, at least about 14 days, at least about 3 weeks, at least about 1 month, at least about 2 months, at least about 3 months, at least about 4 months, at least about 5 months, at least about 6 months, at least about 8 months, at least about 10 months, or at least about 1 year before or after administration of the therapeutic agent.

### Functional Assays

The present technology comprises functional assays that may be used to determine the ability of the antibodies of the present technology to mediate neutralizing antibody activity. In some embodiments, the assay measures binding affinity to the Gn/Gc spike complex of a hantavirus, degree of blocking of viral membrane fusion, ability to provide pre- and post-exposure protection in a bank vole model, or ability to exhibit cross-clade neutralization against epitopes from different hantavirus species.

### In Vivo Assays

In some embodiments, the present technology comprises methods for determining the activity of the antibodies of the present technology. In some embodiments, the activity of the antibodies is evaluated in vivo in an animal model (e.g., a Syrian hamster model of uniformly lethal ANDV challenge). Hantavirus epitope targeting activity of these antibodies in vivo may be assessed by using assays, including but not limited to, using flow cytometry to analyze the immune profiling of the blood, measuring displayed half-maximal inhibitory concentration (IC₅₀), monitoring Gn:Gc dissociation, Gc target membrane insertion, and/or homotorizerization.

In some embodiments, the present technology comprises antibodies that exhibit inhibitory effects on viral entry into the host cell and/orviral membrane fusion, including blocking Gn:Gc dissociation, Gc target membrane insertion, and/or homotorizerization. Such antibodies exhibit hantavirus epitope targeting effects in vivo, e.g., when administered to a subject that has been infected with a hantavirus or exhibits one or more symptoms associated with a hantavirus infection.

### Diagnosis of Infection

The hantavirus antibodies of the present technology may also be used for diagnosing a subject. In some embodiments, the diagnosis is a cancer diagnosis or a diagnosis of viral infection. In some embodiments, the method comprises (1) contacting a biological sample (e.g., whole blood, serum, plasma) from a subject (e.g., a mammal, a human) with an antibody of the present technology, (2) detecting binding of the antibody to one or more epitopes of the present technology in the biological sample, and (3) diagnosing the subject as having a cancer or a viral infection if the antibody in (2) binds to the one or more epitopes in the biological sample. In some embodiments, the amount of antibody binding in (2) is quantified and compared to a control. The difference in the amount of binding in the control compared to the biological sample may be compared to a threshold value. When the amount of binding exceeds the threshold value, the subject is diagnosed with the cancer or the viral infection.

The antibody in (1) comprises a V_{H} and V_{L} regions having one or more CDR sequences provided in Tables 1 and 2, which confer binding to the one or more epitopes. In a nonlimiting example, the antibody in (1) may comprise one or more of (a) a heavy chain having a V_{H} region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 1-5, 68, or 69; (b) a light chain having a V_{L} region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 21-25, 70, or 81; and (c) binding capabilities to one or more epitopes of the present technology. In some embodiments, the one or more epitopes comprise a viral spike protein (e.g., a hantavirus spike protein) epitope. In some embodiments, the one or more epitopes are present in a Gn/Gc spike complex or a nAb:Gn/Gc complex.

In some cases, the antibodies are conjugated to a detectable label that produces fluorescent, luminescent, or colorimetric signals, and detecting the signal from the label indicates a subject is suitable for treatment with a hantavirus antibody of the present technology. In some cases, after the antibodies are contacted with the biological sample, a labeled secondary antibody is added to the biological sample that have been contacted with the antibody of the present technology and detecting the signal from the secondary antibody indicates that the binding of the antibody to a Gn/Gc spike complex or presence of a nAb:Gn/Gc complex.

### Screening Antibodies

The present technology comprises methods for screening and/or selecting antibodies for use in the methods of the present technology. The method comprises (1) contacting an antibody of the present technology with a polypeptide, (2) contacting the antibody with a biological sample collected from a subject suspected of being infected with a hantavirus, and (3) selecting the antibody if the antibody binds to the polypeptide. In some embodiments, the antibody also binds preferentially to the biological sample from the subject suspected of being infected as compared to biological sample from the subject who has not been exposed to a hantavirus.

In some embodiments, the polypeptide in (1) comprises an amino acid sequence about 10%, about 20%, about 30%, about 40%, about 50%, about 60%, about 70%, about 75%, about 80%, about 85%, about 90%, about 95%, about 99%, or about 100% identical to the amino acid sequence of SEQ ID NOS:1-5, 68, or 69 and/or SEQ ID NOS:81, 21-25, or 70.

In some embodiments, the polypeptide in (1) comprises an amino acid sequence at least 10%, at least 20%, at least 30%, at least 40%, at least 50%, at least 60%, at least 70%, at least 75%, about 80%, at least 85%, at least 90%, at least 95%, at least 99%, or at least 100% identical to the amino acid sequence of SEQ ID NOS:1-5, 68, or 69 and/or SEQ ID NOS:81, 21-25, or 70.

In some embodiments, the polypeptide in (1) comprises an amino acid sequence at least about 10%, at least about 20%, at least about 30%, at least about 40%, at least about 50%, at least about 60%, at least about 70%, at least about 75%, about 80%, at least about 85%, at least about 90%, at least about 95%, at least about 99%, or at least about 100% identical to the amino acid sequence of SEQ ID NOS:1-5, 68, or 69 and/or SEQ ID NOS:81, 21-25, or 70.

### Subjects

In some embodiments, the subject of the present technology has or is at risk of developing one or more of a cancer, hemorrhagic fever with renal syndrome (HFRS) (e.g., acute HFRS), or hantavirus cardiopulmonary syndrome (HCPS).

In some embodiments, the subject of the present technology is infected with a hantavirus or is at risk of being infected with a hantavirus. In some embodiments, the subject has active infection or has been actively infected with the hantavirus in one or more of the lung, kidney, spleen, or blood.

In some embodiments, the subject has or is at risk of developing a disease associated with hantavirus infection. In some embodiments, the disease manifests in or affects one or more of the lung, kidney, spleen, or blood.

In some embodiments, the subject has one or more symptoms associated with hantavirus infection. Nonlimiting examples of symptoms include fever, myalgia, head pain, and abdominal pain. In some embodiments, the subject has or is at risk of developing thrombocytopenia. In some embodiments, the subject has or is at risk of developing renal failure (e.g., transient renal failure). In some embodiments, the symptoms are characterized by acute onset.

In some embodiments, the subject has been infected with the hantavirus for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 25 days, about 30 days, about 35 days, about 40 days, about 45 days, about 50 days, about 55 days, about 60 days, about 65 days, about 70 days, about 75 days, about 78 days, about 80 days, about 90 days, about 100 days, about 200 days, or about 1 year.

In some embodiments, the subject has been infected with the hantavirus for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 78 days, at least 80 days, at least 90 days, at least 100 days, at least 200 days, or at least 1 year.

In some embodiments, the subject has been infected with the hantavirus for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 25 days, at least about 30 days, at least about 35 days, at least about 40 days, at least about 45 days, at least about 50 days, at least about 55 days, at least about 60 days, at least about 65 days, at least about 70 days, at least about 75 days, at least about 78 days, at least about 80 days, at least about 90 days, at least about 100 days, at least about 200 days, or at least about 1 year.

In some embodiments, the subject has had a disease associated with hantavirus infection for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 25 days, about 30 days, about 35 days, about 40 days, about 45 days, about 50 days, about 55 days, about 60 days, about 65 days, about 70 days, about 75 days, about 78 days, about 80 days, about 90 days, about 100 days, about 200 days, or about 1 year.

In some embodiments, the subject has had a disease associated with hantavirus infection at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 78 days, at least 80 days, at least 90 days, at least 100 days, at least 200 days, or at least 1 year.

In some embodiments, the subject has had a disease associated with hantavirus infection for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 25 days, at least about 30 days, at least about 35 days, at least about 40 days, at least about 45 days, at least about 50 days, at least about 55 days, at least about 60 days, at least about 65 days, at least about 70 days, at least about 75 days, at least about 78 days, at least about 80 days, at least about 90 days, at least about 100 days, at least about 200 days, or at least about 1 year.

The following examples are offered for illustrative purposes and are not intended to limit the present technology. Those of skill in the art will readily recognize a variety of non-critical parameters that may be changed or modified to yield essentially the same results.

### EXAMPLES

### Example 1: Recognition of and Protection Against Orthohantaviruses Using Antibodies of the Present Technology

This example illustrates human antibody recognizing a quaternary epitope in the Puumala virus glycoprotein provides broad protection against orthohantaviruses.

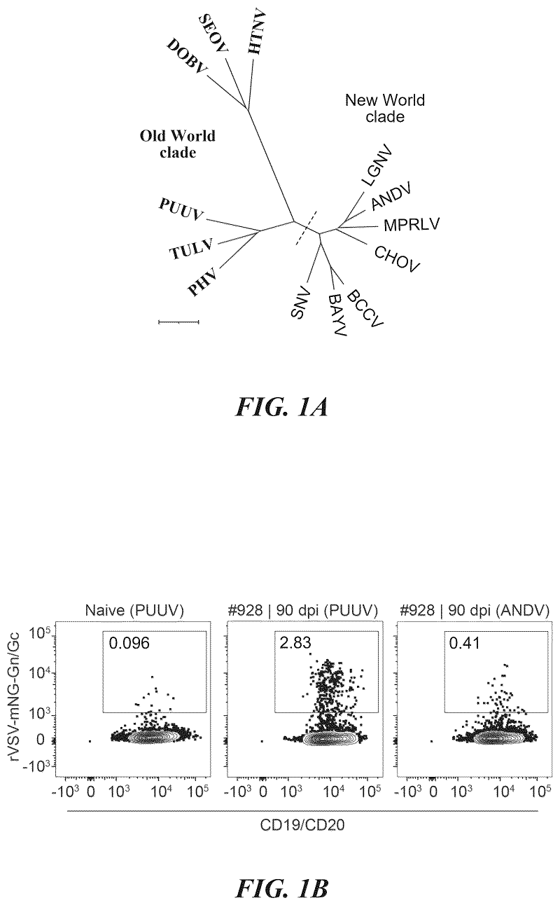

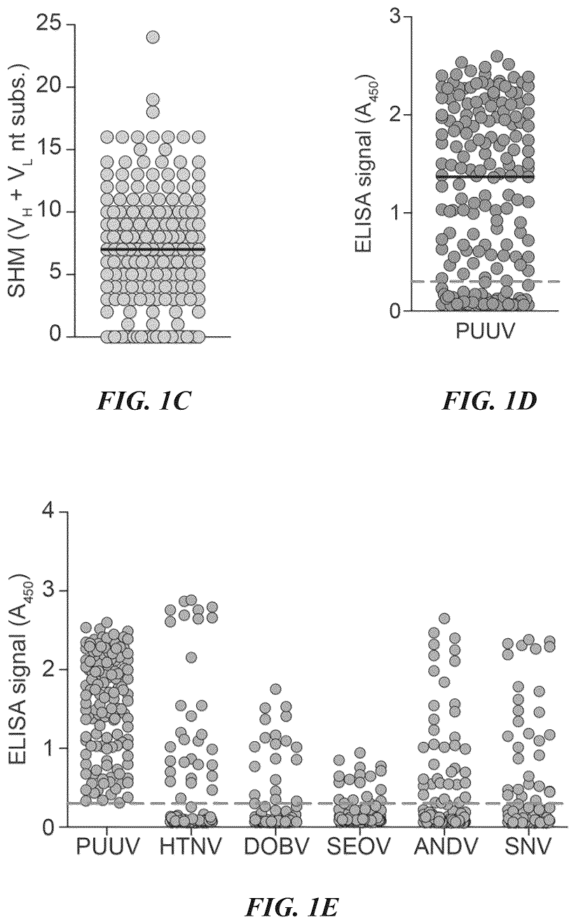

Recent FDA approval of monoclonal antibody (mAb) countermeasures against Ebola virus and emergency use authorization (EUA) approval of mAbs for COVID-19 validate passive immunization as a strategy for viral diseases. Although most of the protective lead mAbs may recognize and/or neutralize Gn/Gc in a virus-specific manner, several nAbs isolated from SNV-experienced donors with activity against members of both Old World and New World clades have been identified (hereafter, cross-clade activity; see FIG. 1A). Two of these nAbs may partially protect Syrian hamsters from lethal ANDV challenge, but their in vivo protective efficacy against HFRS-causing hantaviruses has not been evaluated. Further, their epitopes have not been determined, and thus, the molecular bases of cross-clade Gn/Gc binding, neutralization, and in vivo protection by these nAbs remain unknown.

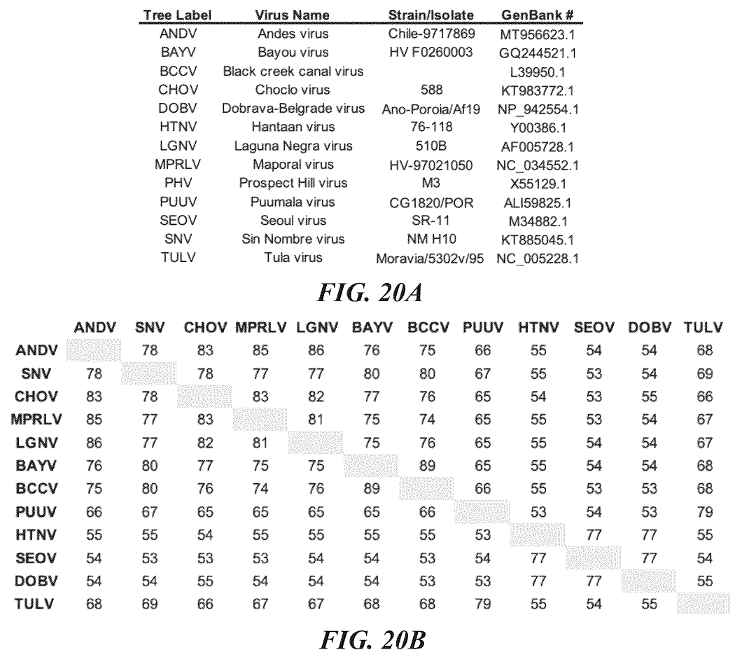

The exclusive focus on New World hantavirus-experienced donors in these previous studies may have limited the discovery of nAbs that could also target Old World hantaviruses. Interestingly, a Gn/Gc sequence alignment revealed that the Old World hantavirus, PUUV, genetically bisects other Old World and New World hantaviruses (FIG. 1A and FIGS. 20A-20B), raising the possibility that human exposure to PUUV may engender distinct, and likely more cross-reactive, antibody specificities.

To test this possibility, mAbs from PUUV-experienced donors were isolated from a Swedish HFRS-convalescent cohort. Briefly, human neutralizing monoclonal antibodies (nAbs) against tetrameric Gn/Gc glycoprotein spikes from Puumala virus (PUUV)-experienced donors were isolated. A new, potentially dominant class of nAbs recognizing the ‘capping loop’ of Gn that masks the hydrophobic fusion loops in Gc was defined. 135 mAbs with varying degrees of cross-clade reactivity and neutralizing activity that bound diverse epitopes in Gn/Gc were identified. A subset of nAbs in this class, including ADI-42898, bound Gn/Gc complexes but not Gn alone, suggests that they recognize a quaternary epitope encompassing both Gn and Gc. ADI-42898 blocked cell entry by seven HCPS- and HFRS-associated hantaviruses. Single doses of this nAb may protect Syrian hamsters and bank voles challenged with the highly virulent HCPS-causing Andes virus and HFRS-causing PUUV, respectively. A single mAb, ADI-42898, was found to have exceptional cross-clade (FIG. 1A) neutralization breadth and recognized a previously unknown epitope at a GnGc intersubunit interface. ADI-42898 may protect against both ANDV in Syrian hamsters and PUUV in a newly established bank vole challenge model. These results suggest that ADI-42898 may be a promising candidate for clinical development as a countermeasure for HCPS and HFRS. Its unique Gn/Gc recognition mode may inform the development of broadly protective hantavirus vaccines.

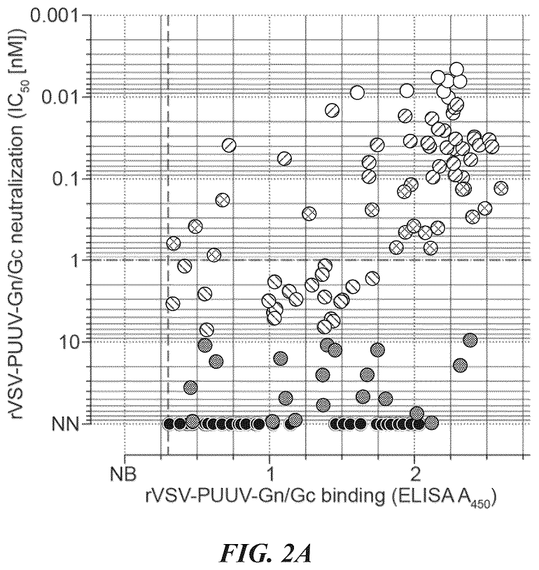

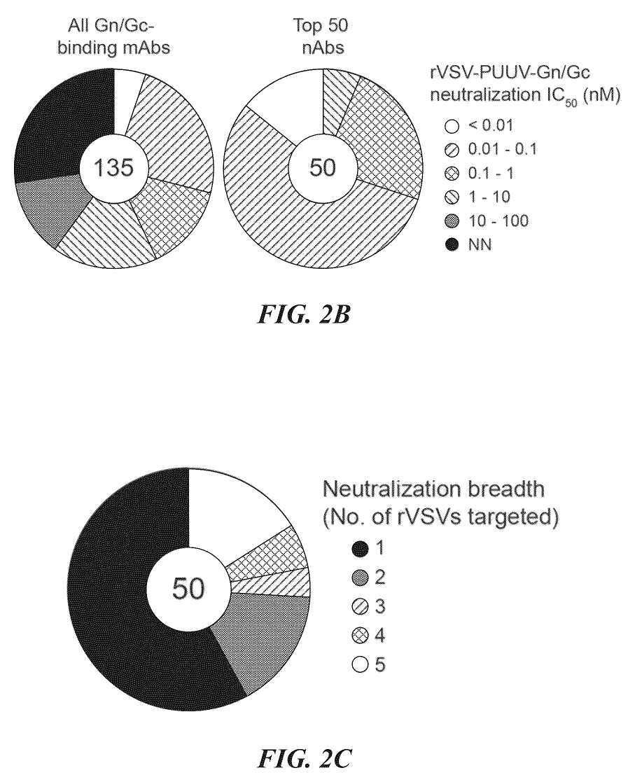

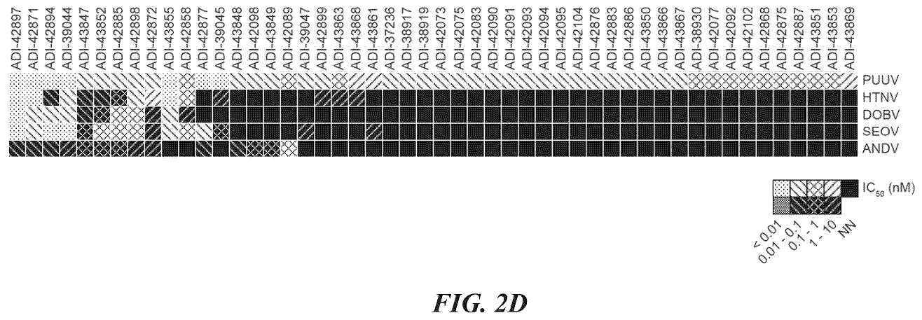

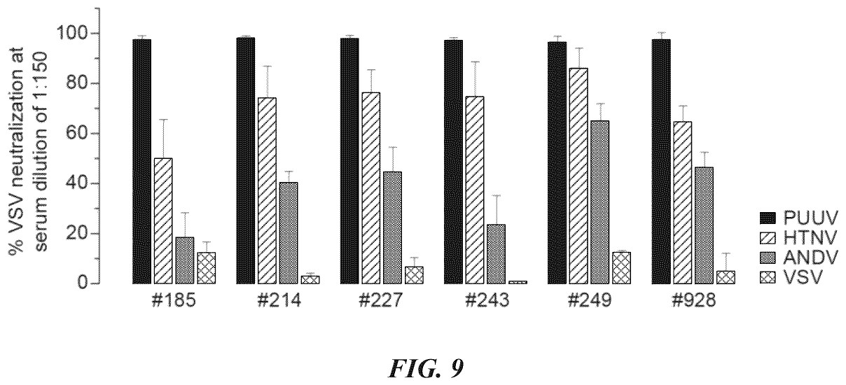

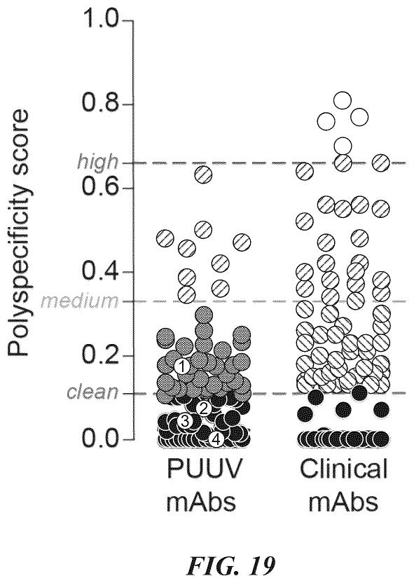

Three strategies were adopted to enrich for bnAbs targeting conserved epitopes in native Gn/Gc tetramers. First, PUUV was chosen as the target virus because of the genetically intermediate position of its M segment sequence (encoding Gn/Gc) relative to those of the Old World and New World hantaviruses (FIG. 1A). Second, many sera from PUUV-experienced donors in the Swedish cohort was screened to identify source samples with a high degree of cross-clade serological reactivity (FIG. 9). Third, to improve the chances of retrieving nAbs targeting quaternary Gn/Gc epitopes, memory B cells were panned with fluorescently labeled rVSV particles bearing membrane-bound Gn/Gc, instead of recombinant proteins that fail to fully recapitulate the structure of the Gn/Gc tetramer. Of the 180 IgG V_{H}/V_{L} sequence pairs captured from antigen-sorted memory B cells, 135 recognized rVSV-PUUV-Gn/Gc, attesting to the specificity of the sorting approach. Moreover, some degree of cross-reactivity against Gn/Gc from five other hantaviruses was observed and—concordant with the hypothesis—comparable cross-reactivities against both Old World and New World agents (FIGS. 1E and 2D). Importantly, mAbs with broad binding activity displayed low levels of polyreactivity (FIG. 19), demonstrating that their cross-binding is not due to nonspecific cross-reactivity (42). Interestingly, a recent study found mAbs from SNV-experienced donors but not ANDV-experienced donors to display a greater degree of cross-clade reactivity, providing additional evidence that the nature of the primary antigenic exposure may influence the breadth of the antibody response against hantaviruses in humans.

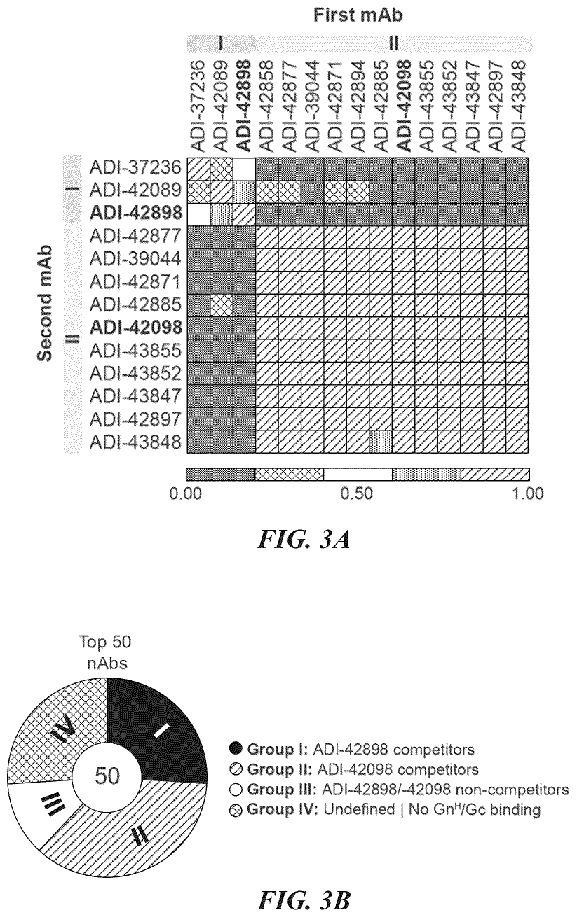

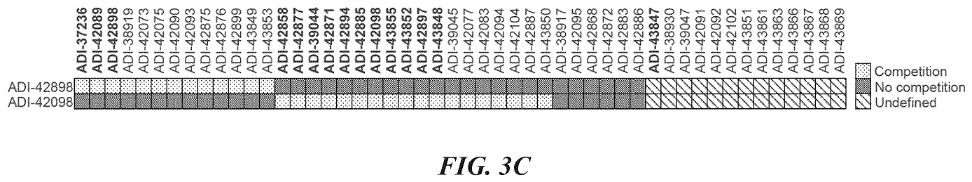

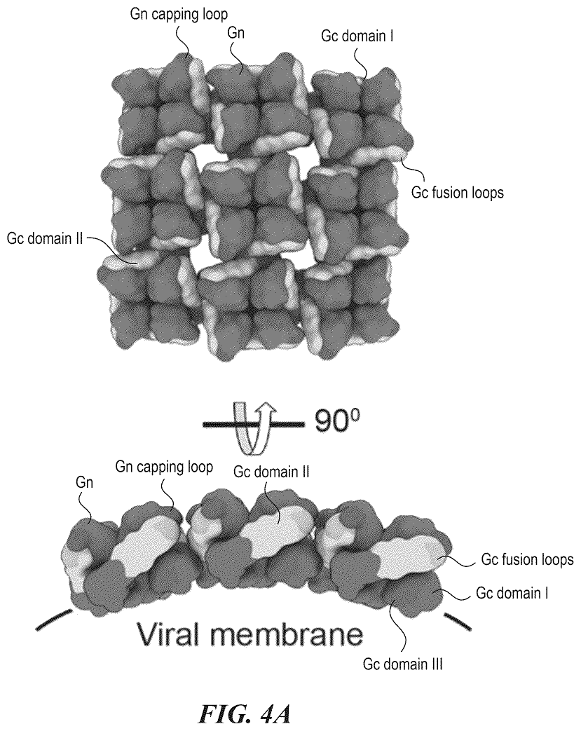

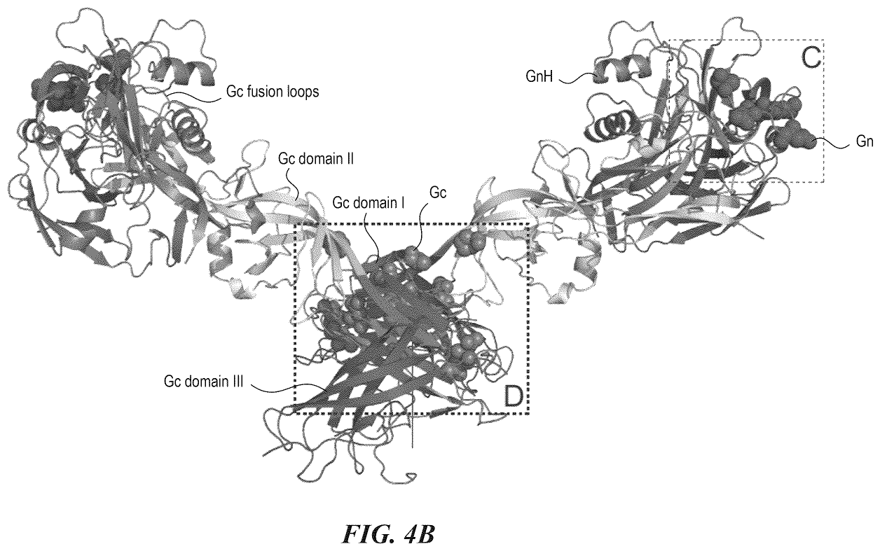

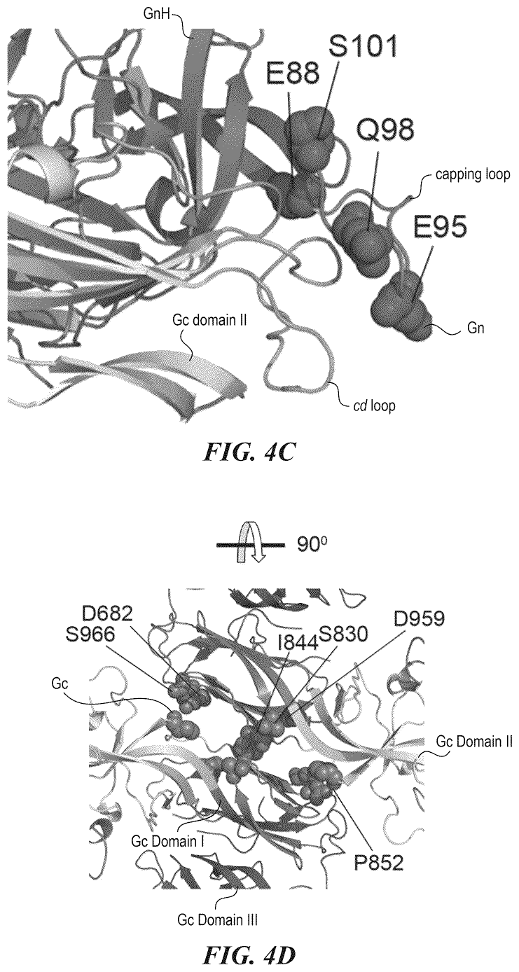

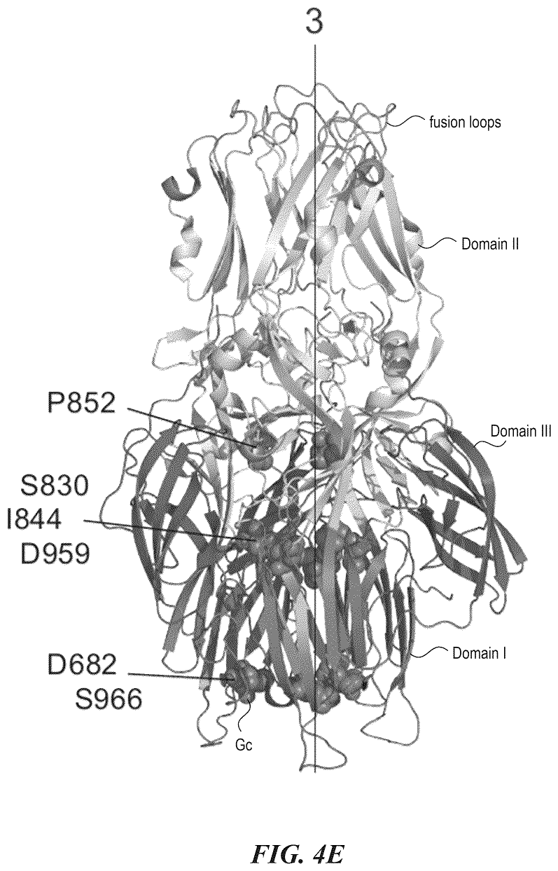

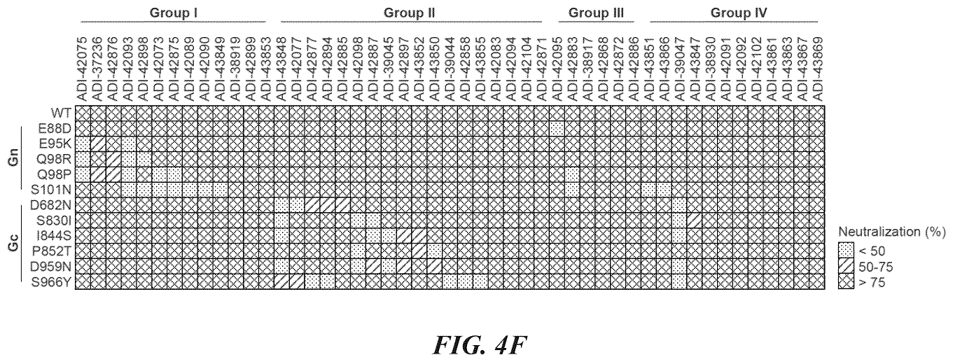

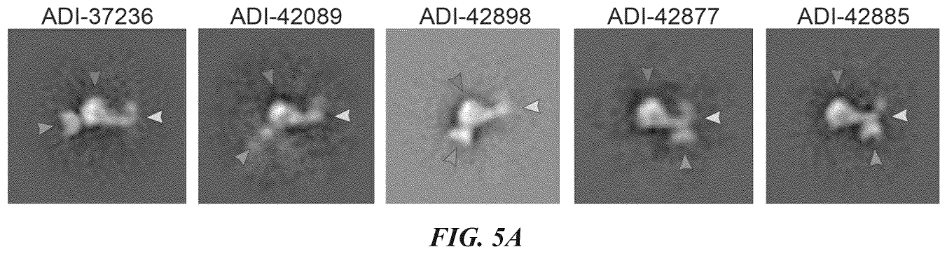

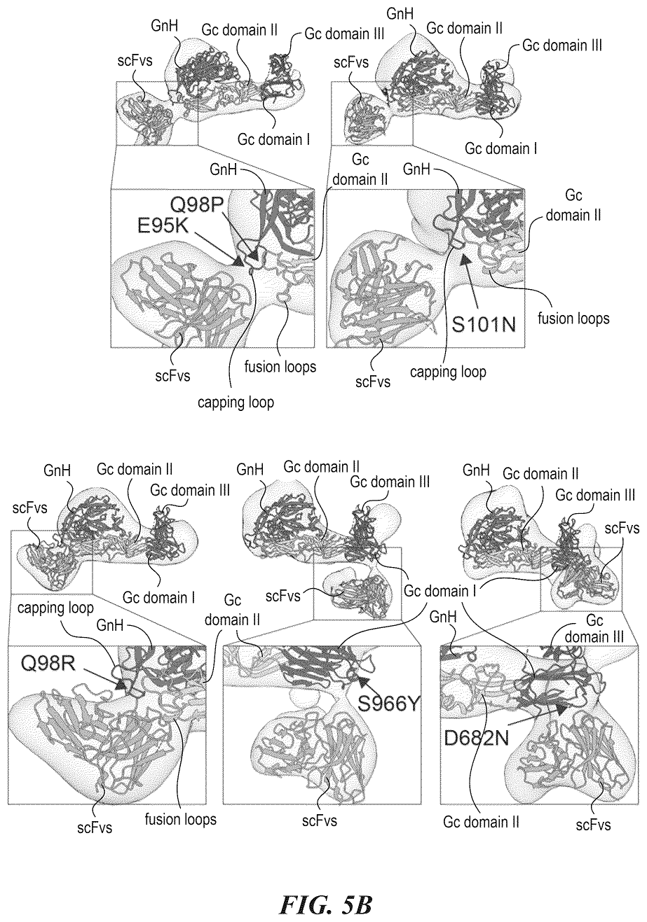

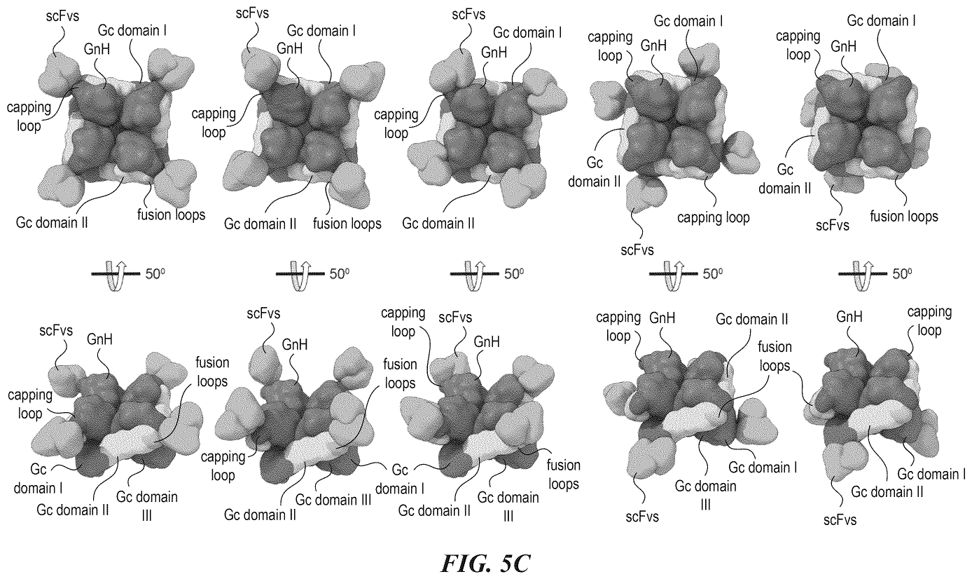

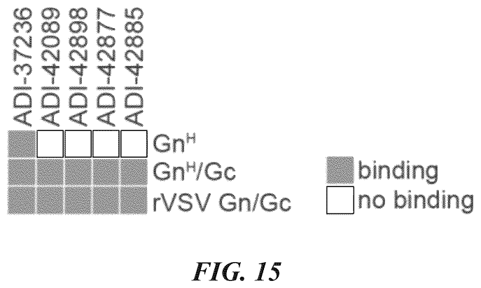

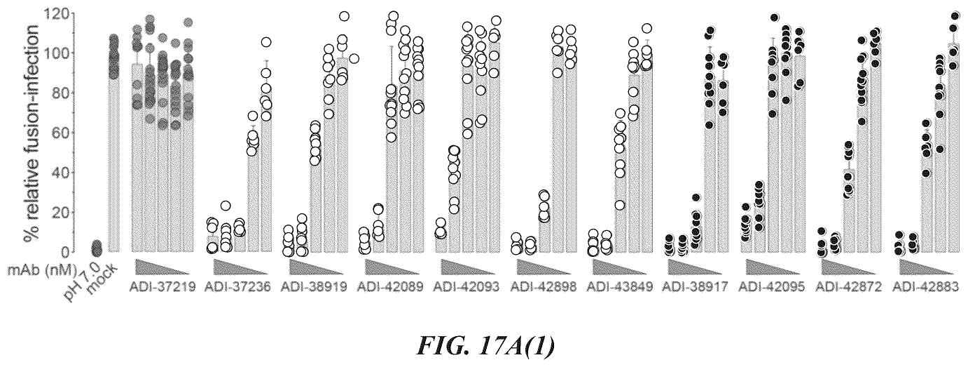

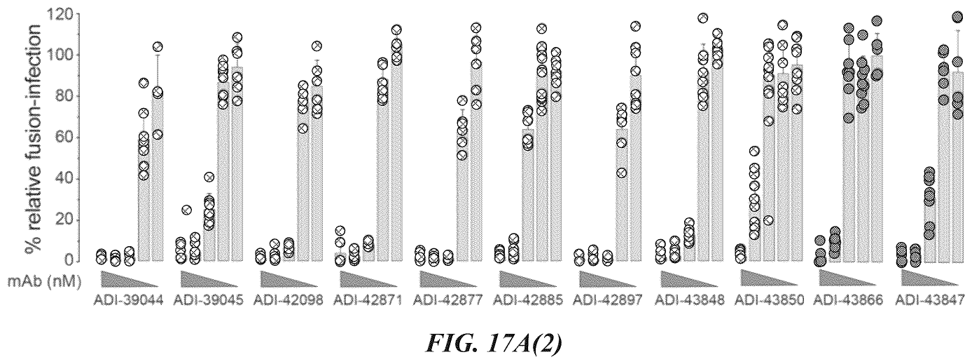

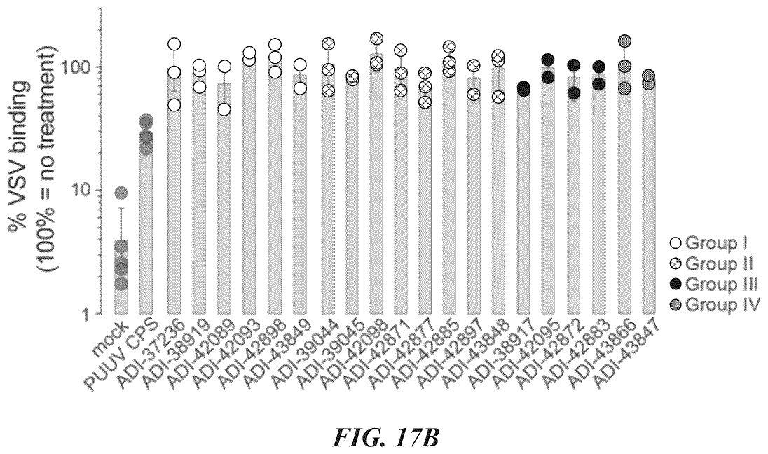

The epitope-binning studies on a prioritized subset of potent nAbs uncovered at least four distinct antigenic sites in Gn/Gc that fine-mapped through rVSV-based selections of neutralization escape mutants (FIGS. 3A-3C, 4A-4F). Antibodies were clustered according to their Pearson correlation-generated relatedness scores using Morpheus software (Broad Institute, Cambridge, USA). A major class of nAbs (Group 1) whose members engage the capping loop in the membrane-distal N-terminal head domain of Gn (Gn^{H}) was identified (FIGS. 4A-4F, 5A-5C). The preponderance of Group I nAbs in the panel, together with recent descriptions of nAbs elicited against HTNV Gn/Gc that recognize overlapping or adjacent sequences in Gn^{H}, suggest that this region of Gn, well-exposed on viral particles is a major target of the antibody response to Gn/Gc in mammals. However, it has not been described in earlier work aimed at identifying neutralizing epitopes. Here, new evidence was that such mAbs exhibit at least two distinct binding modes, exemplified by ADI-37236 and previously reported mAbs on the one hand and ADI-42898 on the other presented. Binding of mAbs to rVSV-PUUV-Gn/Gc was determined in a single concentration mAb spot ELISA; mAbs crossing an ELISA signal threshold of 0.3 were defined as binders. Binding of mAbs to PUUV GnH and GnH/Gc was determined by BLI. mAbs presenting with binding responses >0.1 nm were defined as binders. Despite escaping binding and neutralization via similar mutations in the capping loop, ADI-37236 could bind both monomeric Gn^{H} and heterodimeric Gn^{H}/Gc proteins, whereas ADI-42898 could only bind Gn^{H}/Gc (FIG. 15). The former nAbs may exclusively contact sequences in Gn^{H}, whereas the latter contact both Gn^{H} and residues in the closely associated Gc cd loop that forms part of Gc's membrane-interacting fusion loops. This hypothesis rationalizes the observed relationship between nAb binding and breadth: ADI-37236 and other “pure” Gn^{H} binders that recognize the highly variable capping loop are virus-specific nAbs, whereas ADI-42898 and other Gn^{H}/Go-binders whose epitopes include more conserved Gc residues are bnAbs (FIGS. 2D, 4C and 4F). The failure to obtain escape mutations in Gc with ADI-42898 may reflect the possibility that such amino acid exchanges have higher costs on viral fitness or that the energetics of mAb binding is driven by its contacts with Gn and not Gc. Finally, both Gc-dependent and Gc-independent capping-loop binders could act downstream of virus-cell attachment to block viral membrane fusion (FIGS. 17A-17B), indicating that the direct engagement of Gc by Group I nAbs may not be required to prevent fusion loop deployment and/or Gc rearrangement during membrane fusion.

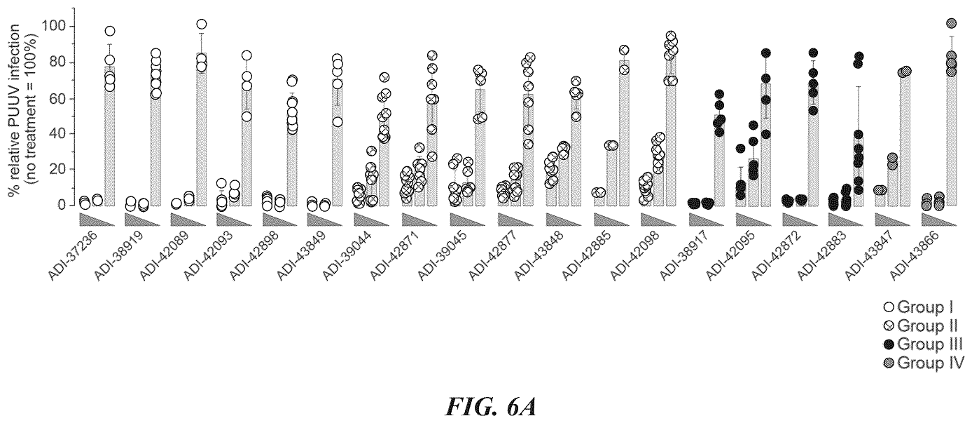

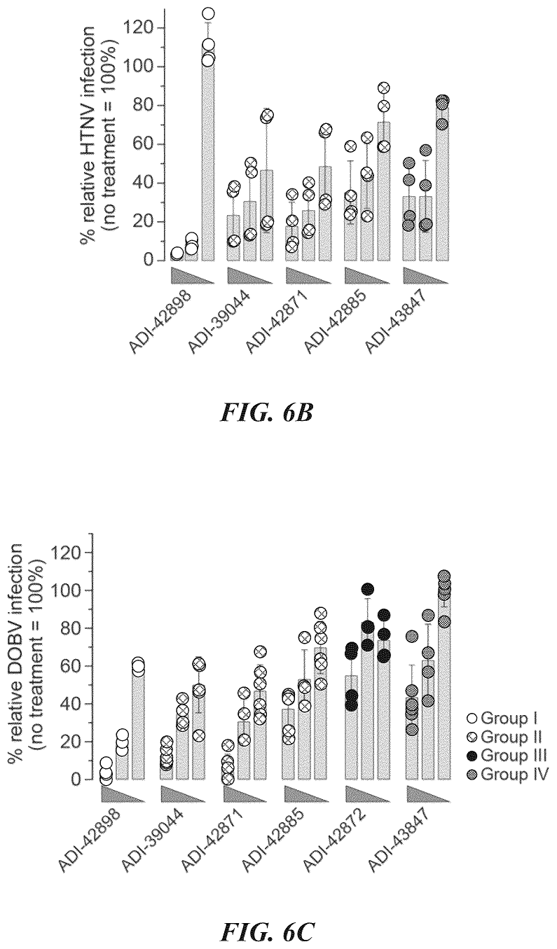

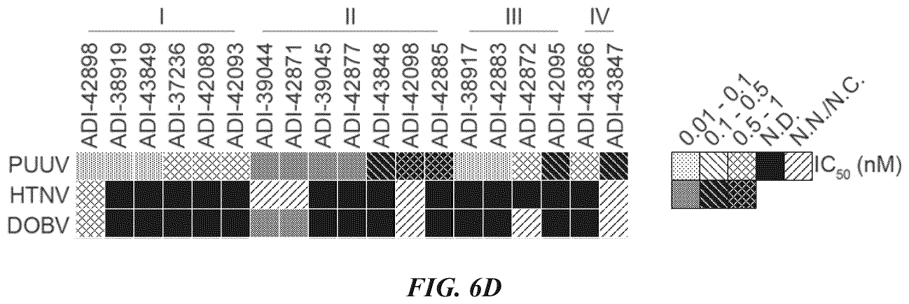

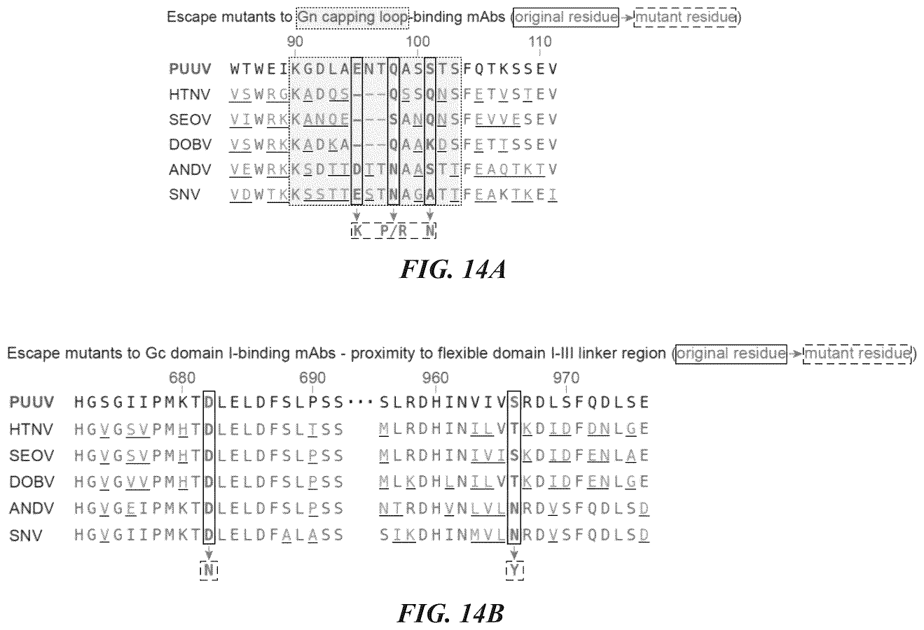

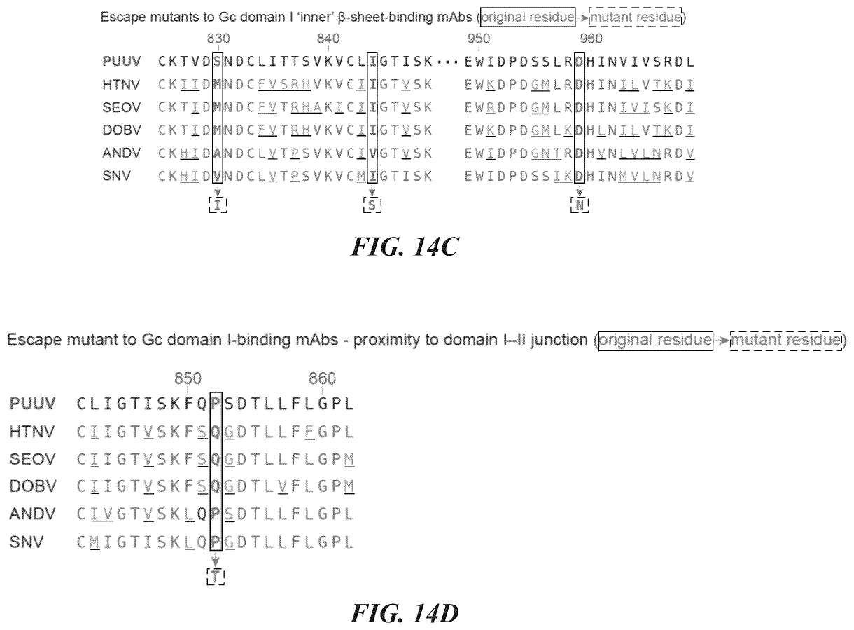

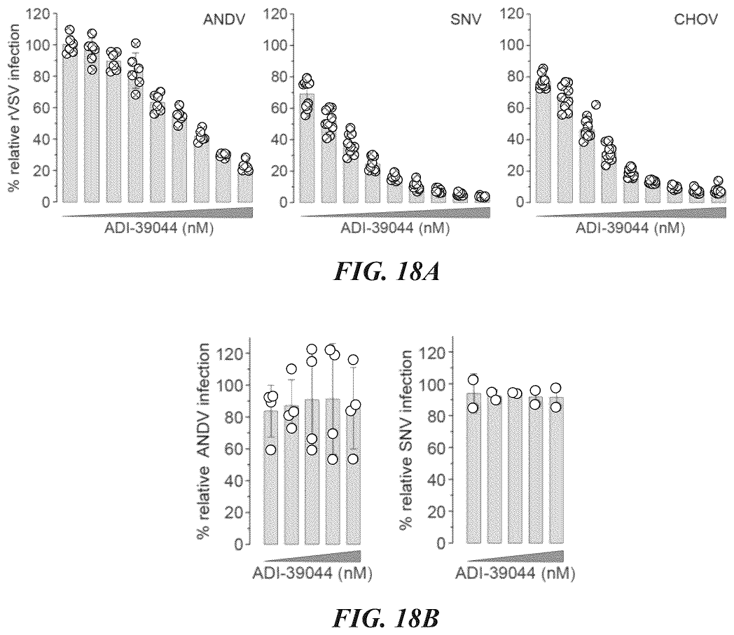

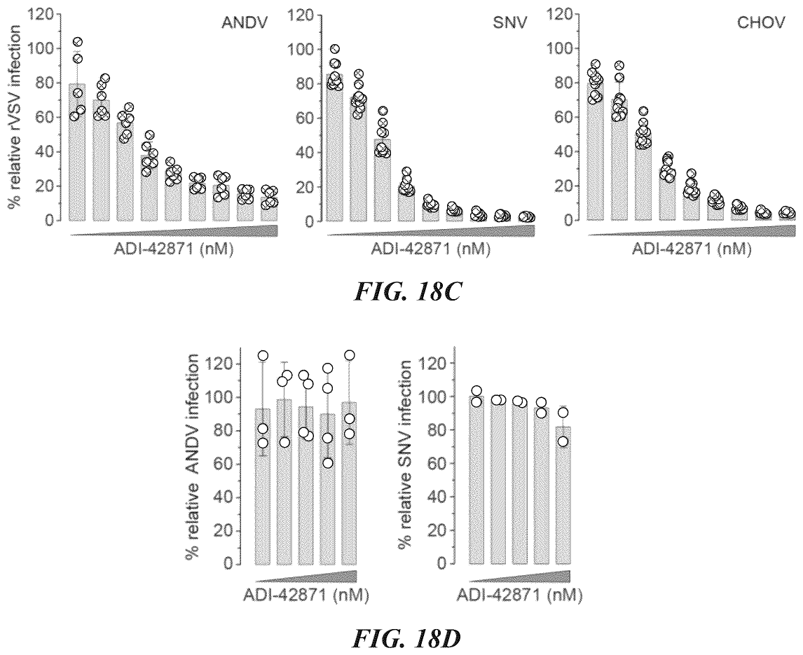

Members of the second major class of nAbs identified and mapped in this study (Group II) recognize central sequences of the Gc subunit, including epitopes (i) in domain I near the flexible domain 1-111 linker region; (ii) at or near the domain 1-II junction; and (iii) the ‘inner sheets’ of domain I, which mediate Gc:Gc contacts between adjacent (Gn/Gc)₄ tetramers stabilizing the spike lattice (FIGS. 4A-4F, 5A-5C). Similar nAbs, elicited through a variety of approaches for different hantaviruses, have been described previously, suggesting that these Gc sequences may be immunodominant despite their reduced accessibility to mAbs in intact viral particles (see below). The behavior of Group II nAbs in neutralization assays was consistent with their mode of Gn/Gc recognition. First, it is found that they tended to exhibit greater neutralization breadth than did Group I nAbs (FIG. 2D), in accordance with the greater intra-clade and cross-clade conservation of their target sequences (FIGS. 14A-14D). Second, Group II nAbs often failed to completely neutralize authentic viral infection (FIGS. 6A-6D and 18A-18D), as also reported recently for ANDV- and SNV-targeted Gc binders. Cryo-EM tomographic analysis of a Gc domain I/II-targeting bank vole mAb bound to PUUV virus-like particles provides a likely structural explanation for the un-neutralized fraction: the close apposition of glycoprotein tetramers at Gc:Gc two-fold axes of symmetry permits mAb binding only at lattice-free sites in virions. Together with differences in their intrinsic binding affinities for Gc from different hantaviruses, this observation may also explain the discordant behavior of Group II nAbs against rVSVs and authentic viruses (FIGS. 2D, 6A-6D and 18A-18D); lattice-free binding sites may be more available in rVSV particles due to their distinct curvature, lower levels of Gn/Gc incorporation, or both.

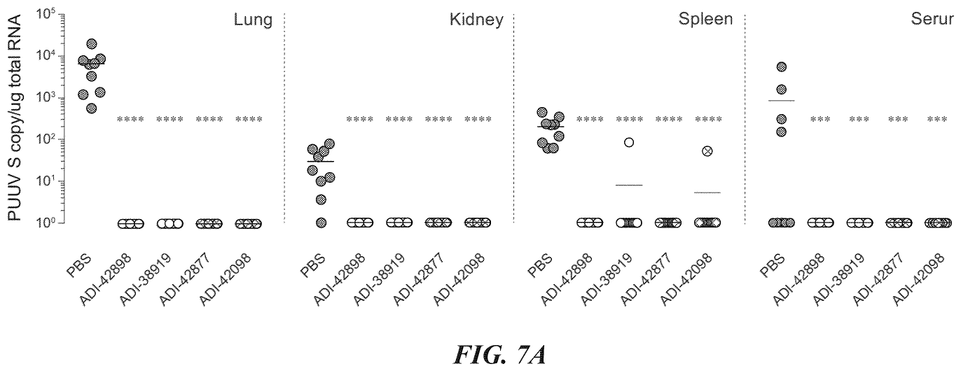

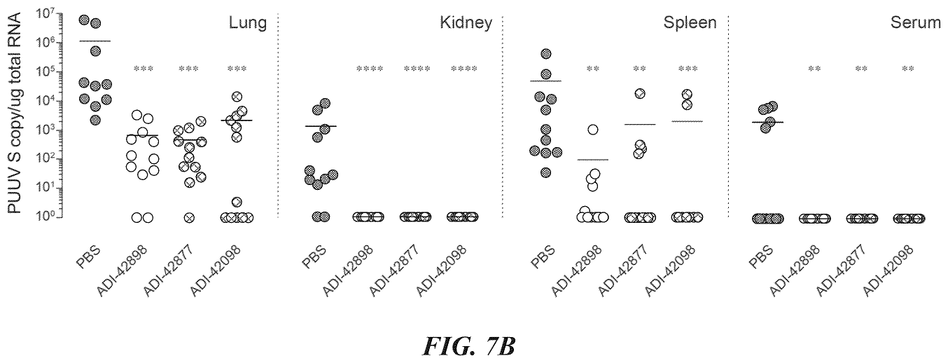

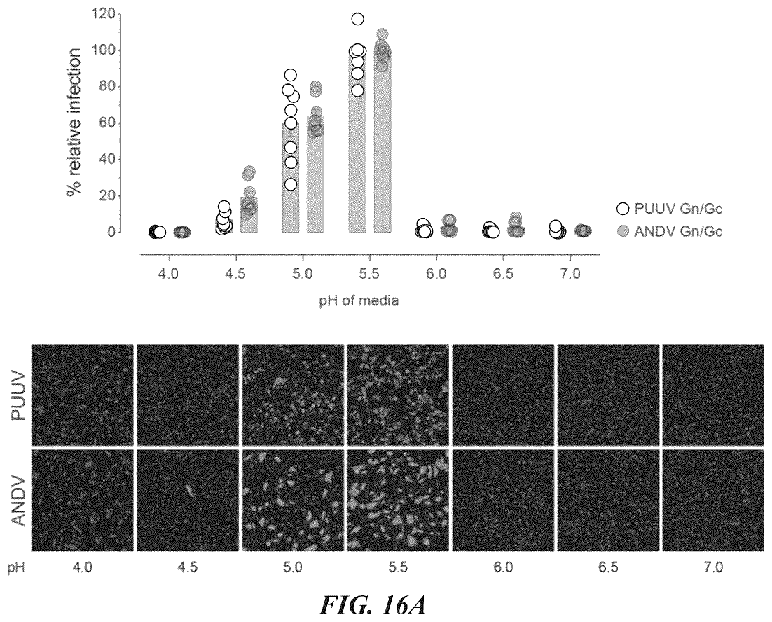

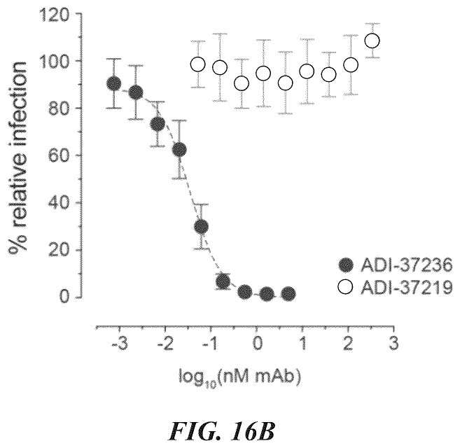

rVSV-PUUV-Gn/Gc particles were pre-incubated with decreasing amounts of indicated mAbs (50 nM to 0.005 nM), followed by fusion-infection of HUVECs. ADI-37219, a non-neutralizing mAb, was included as a negative control (also see FIG. 16B) and pre-titrated amounts of rVSV-mNG-P-PUUV-Gn/Gc were incubated with mAbs at 25 nM (or PUUV convalescent patient serum (CPS) at a 1:100 dilution). Binding of rVSV:mAb complexes to U20S cells was determined by flow cytometry. Gc-specific nAbs tested afforded the potent inhibition of viral membrane fusion (FIGS. 17A and 17B), most likely through the prevention of Gc homotrimerization and/or domain III foldback onto domain 1-11 trimers. The capacity of domain I inner sheet binders to block formation of the Gc:Gc interface, at least in principle, also raises the intriguing possibility that some Gc binders may act in part by interfering with glycoprotein lattice formation during Old World hantavirus assembly. Finally, despite their failure to fully neutralize viral infectivity, two Group II nAbs, ADI-42877 and -42098, afforded complete protection against PUUV challenge in naïve bank voles (FIG. 7). Therefore, it is conceivable that Gc-targeting mAbs contribute to viral clearance through mechanisms other than entry neutralization, including Fc-mediated effector functions, as previously suggested. Given their non-competing epitopes and potentially distinct mechanisms of neutralization and protection, it is possible that specific combinations of Group II and Group I nAbs may afford enhanced protection and/or robustness to variations in Gn/Gc sequence and viral neutralization escape.

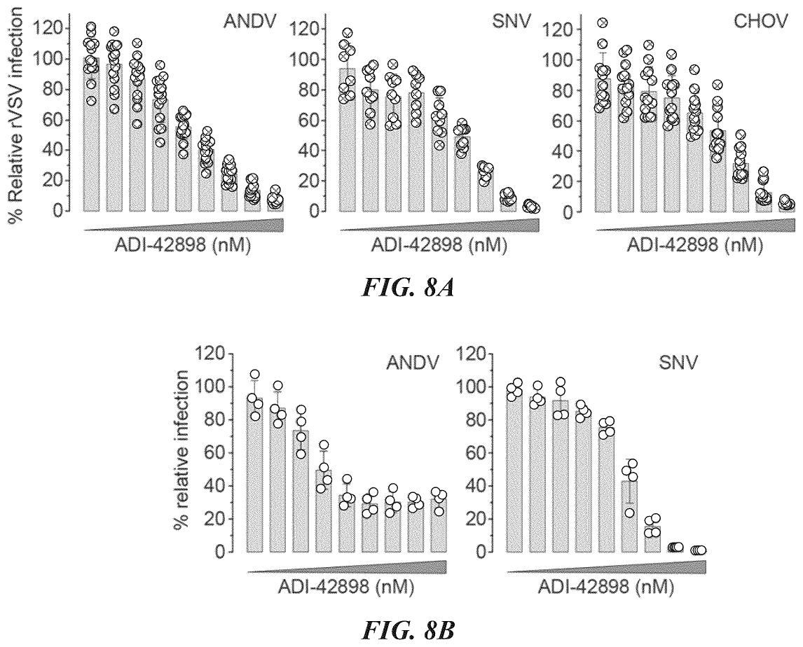

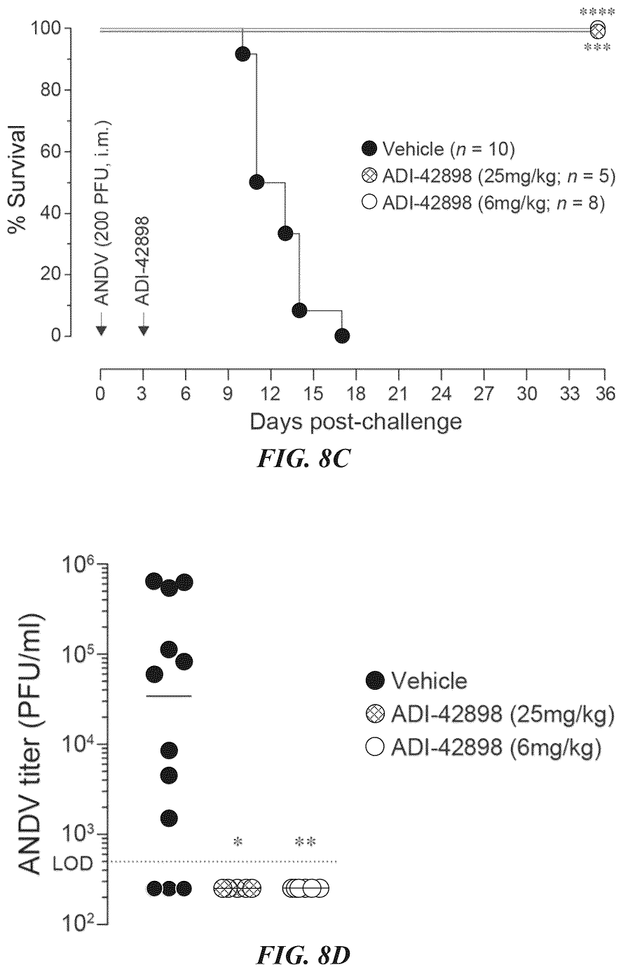

A single Group I nAb, ADI-42898, with broad neutralizing activity against virulent hantaviruses from both Old World and New World clades (FIGS. 6A-6D and 18A-18D) was identified and successfully evaluated it in both pre- and post-exposure settings in a recently described bank vole model of PUUV challenge (FIGS. 7A-7B). ADI-42898 also afforded complete protection of Syrian hamsters from lethal ANDV challenge when administered as single 25-mg/kg or 6-mg/kg doses at 3 days post-virus challenge (FIGS. 8A-8D), raising the possibility that it could be dosed down further (below 6-mg/kg) and still retain protective activity. By contrast, ANDV-specific nAbs previously reported to be fully protective in this model were either tested with two 5-mg/kg doses per animal or with a substantially higher two-dose regimen (25-50-mg/kg). Moreover, recently described bnAbs only afforded partial protection against ANDV challenge, even when animals were dosed twice, and these bnAbs were not evaluated against Old World hantaviruses in vivo.

ADI-42898 is the first antibody-based treatment demonstrated to protect against both Old World and New World hantaviruses and is a promising candidate for clinical development as a broad countermeasure against HFRS- and HCPS-causing hantaviruses. The ongoing and future work is aimed at extending these studies of protective breadth to additional rodent and nonhuman primate models of PUUV, HTNV, ANDV, and SNV challenge. The studies in nonhuman primates would greatly facilitate the identification of key correlates of protection that could be used to design human clinical trials and to seek drug licensure under the FDA's Animal Rule. Finally, the properties of ADI-42898, and those of the Group I nAbs as a whole, suggest strategies for the design of broadly protective vaccines that preferentially elicit bnAbs targeting the Gn/Gc interface at the expense of virus-specific nAbs targeting only Gn.

Accordingly, these results suggest that ADI-42898 is a promising candidate for clinical development as a broad countermeasure against HCPS and HFRS.

### Example 2: Isolation of Monoclonal Antibodies from PUUV-Experienced Donors

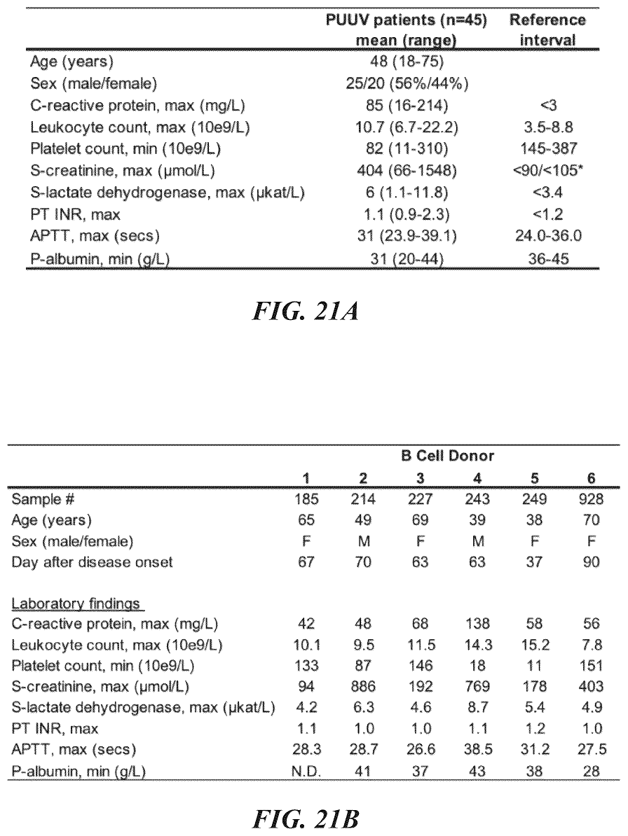

To identify donors for single B-cell sorting, sera from a cohort of 45 Swedish subjects with clinically documented PUUV exposure was screened (FIG. 21A). Signs of thrombocytopenia and transient renal failure were frequent. Serum neutralizing antibody titers were determined with recombinant vesicular stomatitis viruses (rVSV) displaying hantavirus Gn/Gc (1-3). Six donors with potent and broad serum neutralizing antibody responses against rVSVs bearing Gn/Gc from PUUV, a divergent Old World hantavirus, Hantaan virus (HTNV), and a New World hantavirus, ANDV, were identified (FIGS. 9 and 21B).

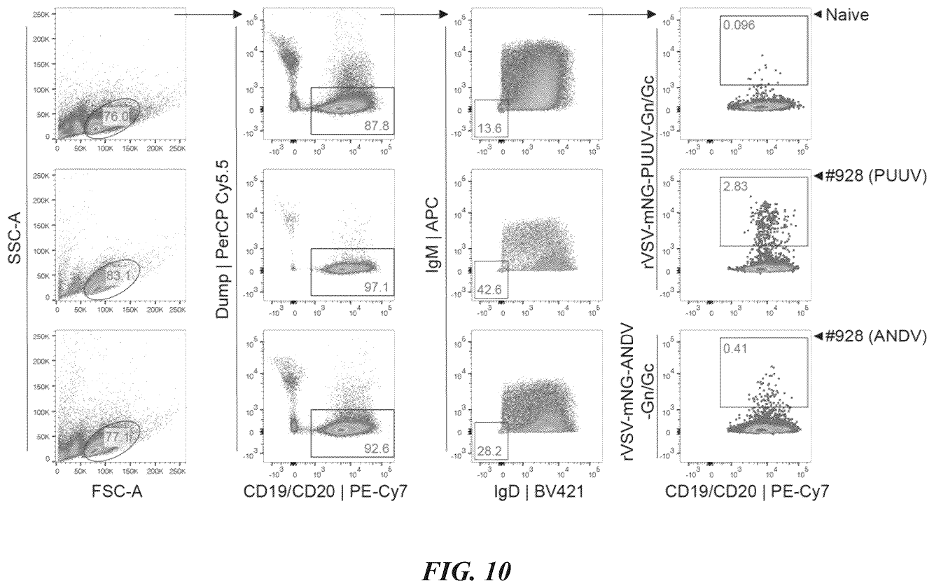

To sample antibody specificities recognizing a diversity of epitopes, including conformational and quaternary epitopes, entry-competent rVSV-Gn/Gc particles bearing fluorescent mNeonGreen-labeled cores (rVSV-mNG-Gn/Gc) (1, 4) were used as baits to single-cell sort class-switched (swIg⁺), antigen specific B cells (CD19⁺/IgM⁻/IgD⁻/virus⁺) from peripheral blood. Across donors, up to 2.8% of swIg⁺ B cells specifically bound rVSV-mNG-PUUV-Gn/Gc, whereas a smaller fraction (~0.4%) was reactive to rVSV-mNG-ANDV-Gn/Gc, concordant with the donors' exposure history, the sequence divergence between these glycoproteins and the lower but detectable cross-neutralizing serum activity against ANDV Gn/Gc (FIGS. 1B, 9, 10, and 20B). Cognate antibody heavy- and light-chain pairs were recovered from 180 individual hantavirus Gn/Gc-reactive B cells and found to contain somatic hypermutations (SHMs), consistent with their memory B cell origin (FIG. 1C). Full-length immunoglobulin G subclass I (IgG1) molecules bearing these variable region sequences were expressed in an engineered strain of Saccharomyces cerevisiae (36) and screened by an enzyme-linked immunosorbent assay (ELISA) to identify 135 PUUV Gn/Gc-specific binders (FIG. 1D). The mAbs were further evaluated for their capacity to recognize rVSV particles bearing hantavirus Gn/Gc proteins from both Old World (HTNV, Seoul virus [SEOV], Dobrava-Belgrade virus [DOBV]), and New World (ANDV, SNV) hantaviruses by ELISA (1-3). Consistent with the serum activity studies (FIG. 9) and the PUUV M segments genetically intermediate position in Orthohantavirus, a similar degree of binding cross-reactivity between PUUV and viruses belonging to either Old World or New World hantavirus clades was observed (FIG. 1E; 14-28%).

### Example 3: Identification of Monoclonal Antibodies Having Neutralizing Activity Against Orthohantaviruses

This example illustrates identification of mAbs with potent and broad neutralizing activity.

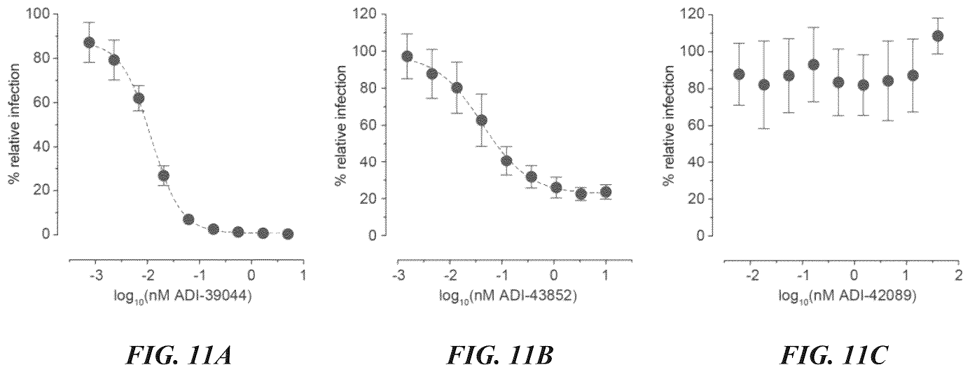

135 PUUV Gn/Gc-reactive mAbs were screened for neutralizing activity against rVSV-mNG-PUUV-Gn/Gc (FIGS. 2A and 2B). 58 of these mAbs displayed half-maximal inhibitory concentration (IC₅₀) values of <1 nM and most bound efficiently to rVSV-PUUV-Gn/Gc particles (FIG. 2A). Next, the 50 most potent nAbs were evaluated against rVSVs bearing divergent Gn/Gc glycoproteins from both Old World (HTNV, SEOV, DOBV) and New World hantaviruses (ANDV) (FIGS. 2C and 2D). Although most of the nAbs (28/50) were PUUV-specific neutralizers, the remaining exhibited varying levels of neutralization breadth. 13 nAbs displayed neutralization activity against at least three hantaviruses with ten affording cross-clade neutralization, indicating that they recognize highly conserved epitopes. It is noted, however, the existence of an infectivity fraction resistant to neutralization (‘un-neutralized fraction’ >5%, FIGS. 2D and FIGS. 11A-11C) that persisted even at the highest tested concentrations for some nAbs, suggesting either reduced nAb:Gn/Gc binding affinities and/or the existence of Gn/Gc subpopulations in viral particles with cryptic epitopes unavailable for antibody binding.

### Example 4: Recognition of Orthohantavirus Antigenic Sites Using Neutralizing Antibodies

This example illustrates nAbs with broad activity predominantly recognize two antigenic sites in Gn/Gc.

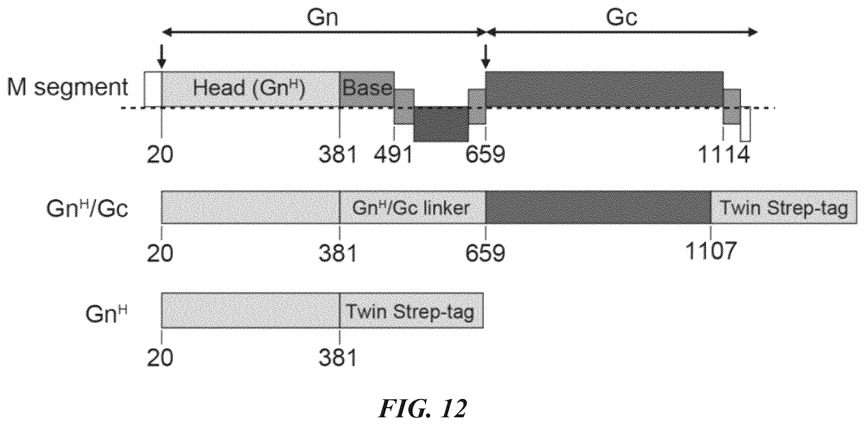

To assess antigenic sites recognized by the nAbs, a subset with broad neutralizing activity (bnAbs) was screened in a pairwise competition ELISA (cELISA) that employed rVSV-mNG-PUUV-Gn/Gc as antigen (FIG. 3A). Most tested bnAbs could be assigned to one of two competition groups, which were designated as Groups I and 11. Group I bnAbs also competed with the PUUV-specific nAb ADI-37236, which was previously isolated from a PUUV-experienced donor by sorting with the recombinant soluble N-terminal head domain of PUUV Gn (Gn^{H}; FIG. 12), providing initial evidence that Group I nAbs recognize overlapping epitopes in the Gn subunit. The top 50 nAbs were binned against selected Group I and Group II nAbs (ADI-42898 and ADI-42098, respectively) in a high-throughput yeast display-based competition assay with a recombinant PUUV Gn^{H}/Gc heterodimer as antigen (FIGS. 3B, 3C, and FIG. 12). This second competition-binning approach largely corroborated the results from the cELISA and showed that the majority of the top 50 nAbs could be assigned to Groups I and 11. Group III nAbs did not compete with ADI-42898 or ADI-42098, indicating that they bind distinct epitopes in Gn/Gc. Group IV nAbs failed to bind to soluble Gn^{H}/Gc, raising the possibility that they recognize tetrameric spike- or tetramer lattice-dependent quaternary Gn/Gc epitopes in viral particles (see FIG. 4A). Because most nAbs from Groups III-IV lacked broad neutralizing activity, nAbs from Groups I-II were assessed for further characterization.

### Example 5: Recognition of Viral Epitopes Using Group I and Group II Neutralizing Antibodies

This example illustrates Group I and II nAbs recognize epitopes in Gn and Gc, respectively.

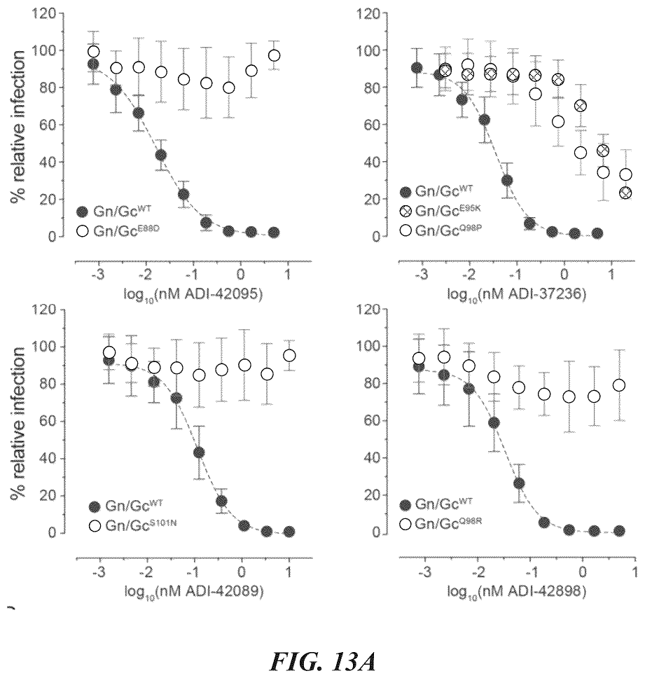

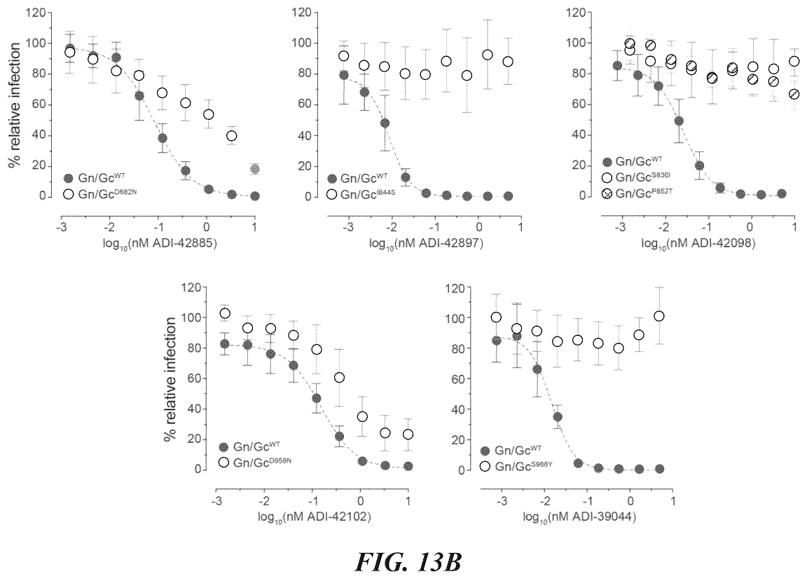

To identify Gn/Gc residues critical for viral recognition by Group I and II nAbs, rVSV-mNG-PUUV-Gn/Gc mutants resistant to neutralization by ADI-42898 or ADI-42098 were selected and the glycoprotein genes of plaque-isolated viral clones to identify escape mutations were sequenced. ADI-42898 elicited an escape mutant bearing a Q98R substitution in the Gn ‘capping loop’ (SEQ ID NO:41), spanning amino acid (aa) residues 90-103 in Gn^{H} (FIGS. 4B, 4C, and 13A). The capping loop associates closely with and caps the cd and bc membrane-interacting fusion loops in Gc domain II in the Gn^{H}/Gc pre-fusion heterodimer. Escape mutants to ADI-42098 bore two independent substitutions, S8301 and P852T, in Gc's central domain I and domain II close to the domain I-II junction, respectively, which form part of the oligomerization interfaces between adjacent pre-fusion Gn/Gc tetramers in the viral lattice and between adjacent Gc monomers in the post-fusion homotrimer (FIGS. 4A, 4B, 4D, 4E and 13B). Concordantly, escape mutants selected with other Group I and II nAbs pinpointed additional residues in the Gn^{H} capping loop (E95K, Q98P, S101N) and Gc domain I (D682N, 1844S, D959N, S966Y), respectively (FIGS. 4A-4F and 13A-13B).

Neutralization screens of prioritized nAbs against this panel of 11 escape mutants further corroborated the competition mapping and escape mutant analyses above (FIG. 4F). Although, as expected, Group III-IV nAbs were largely resistant to this escape mutant panel, substitution E88D, located N-terminal to the capping loop, afforded escape by one of the Group III nAb ADI-42095 (FIGS. 4C, 4F, and 13A). Further, substitution S101N, which introduces an N-linked glycosylation (N-X-S/T sequence) at aa residue 101 in the capping loop (FIG. 14A), afforded viral escape to three of the Group III-IV nAbs (FIG. 4F), possibly through N-glycan-mediated steric blockade of nAb binding to capping loop-proximal epitopes. Taken together, these findings establish the Gn^{H} capping loop and the Gc oligomerization interface as critical sites for viral recognition and neutralization by the majority of the nAbs, including most of the bnAbs isolated herein. However, they also point to the existence of additional unmapped sites of vulnerability in hantavirus Gn/Gc, including some that only appear to be available in Gn/Gc conformers present in viral particles (Group IV).

### Example 6: Visualization of Viral Epitope Recognition Using Group I and Group II Neutralizing Antibodies

This example illustrates visualization of Gn/Gc:nAb complexes and implications for nAb breadth.

To visualize Gn/Gc recognition by Group I and II nAbs, negative-stain transmission electron microscopy (nsEM) studies of the single-chain variable fragments (scFvs) of selected nAbs from Group I (ADI-37236, -42089, -42898) and Group II (ADI-42877, -42885) bound to monomeric PUUV Gn^{H}/Gc was performed. Two-dimensional classes in which the three distinctive elements of the complex, Gn^{H}, Gc, and scFvs, could be readily identified (FIGS. 5A and 5B). These class averages were used to calculate three-dimensional maps at 20 Å resolution (FIG. 5B).

In agreement with epitope assignments, all the Group I nAbs appeared to interact with the Gn^{H} capping loop. Moreover, tetramer models of the Gn^{H}/Gc:nAb complexes suggested that these nAbs bind apically to well-exposed sequences in pre-fusion spikes at the viral surface (FIG. 5C). Although all three Group I nAbs exhibited similar binding modes at this resolution, they differ in two key functional aspects. First, ADI-37236 appears to be a ‘pure Gn binder’ that recognizes both PUUV Gn^{H} and Gn^{H}/Gc, whereas ADI-42089 and ADI-42898 only bind to the latter complex (FIG. 15). Second, ADI-37236 is PUUV-specific, whereas ADI-42089 and ADI-42898 are bnAbs (FIG. 2D). The high sequence variability of the Gn^{H} capping loop (FIG. 14A) and its proximity to the Gc fusion loops (FIG. 4B) raises the possibility that ADI-37236 contacts residues in the variable regions of Gn^{H} only, whereas ADI-42089 and ADI-42898 also make contacts with more highly conserved sequences in Gc. Reconstructions of the Group II nAbs ADI-42885 and ADI-42877 confirmed the epitope mapping to sequences in Gc domain 1, near the domain 1-111 linker region, and in proximity to Gc:Gc homodimer interfaces between neighboring Gn/Gc tetramers (FIG. 5B). The tetramer models of these Gn^{H}/Gc:nAb complexes indicate that the epitopes of Group II nAbs are more proximal to the viral membrane than those of the Group I nAbs (FIG. 5C), suggesting that the former may be less accessible in viral particles than the latter, especially in regions with a closely packed lattice of Gn/Gc tetramers. Cryo-electron tomography of PUUV virus-like particles bound to the F(ab) fragment of a bank vole-derived mAb that recognizes a similar site in Gc supports this hypothesis.

### Example 7: Prevention of Viral Membrane Fusion Using Group I and Group II Neutralizing Antibodies

This example illustrates Group I and II nAbs specifically block viral membrane fusion.

Based on the above epitope definition and structural studies, it is postulated that nAbs from both Groups I and II target acid-triggered Gn/Gc rearrangements required for virus-cell membrane fusion, by preventing either the deployment and membrane insertion of the closely associated hydrophobic fusion loops (Group I nAbs) or the formation of a post-fusion Gc homotrimer (Group II nAbs). To evaluate this hypothesis, a ‘fusion-infection’ assay was established in which rVSV-PUUV-Gn/Gc particles were bound to cells at 4° C. and then subjected to an extracellular acidic pulse (pH 5.5 at 37° C.) to trigger virus-cell membrane fusion at the plasma membrane instead of in endosomal compartments (FIG. 16A).

All Group I and II nAbs tested in this assay mediated dose-dependent reductions in PUUV Gn/G-dependent fusion-infection, as also did several nAbs from Groups III and IV (FIG. 17A). Importantly, and in contrast to a Gn/Ga-specific polyclonal serum, none of these nAbs had any effect on a preceding step in viral entry, the attachment of virions to target cells (FIG. 17B). Taken together, these results indicate that many of the nAbs selected for neutralization potency and breadth directly interfere with Gn/Ga-dependent viral membrane fusion, as also observed for previously isolated nAbs, suggesting that they act by targeting one or more steps in the membrane fusion reaction, including Gn:Gc dissociation, and Gc target membrane insertion and homotrimerization.

### Example 8: Neutralization of Old World Hantaviruses Using Group I and Group II Antibodies

This example illustrates that Group I/II nAbs are potent and broad neutralizers of authentic Old World hantaviruses.

The most potent nAbs were used to test against authentic Old World hantaviruses (FIG. 6A-6D). PUUV neutralization assays largely corroborated the findings with rVSV-mNG-PUUV-Gn/Gc. However, whereas the Gn- and/or potential Gn/Ga-targeting Group I nAbs afforded complete neutralization (all nAbs in Group I and some from Groups III-IV), the G-targeting nAbs (all nAbs in Group II and some from Group IV) left a residual un-neutralized fraction (FIGS. 6A and 6D). The much greater propensity of Group II nAbs to mediate incomplete neutralization in the authentic neutralization assay relative to the rVSV assay (FIG. 2D), rises the possibility that differences between authentic virions and rVSVs in the organization of the (Gn/Gc)₄ spike lattice account for their distinct neutralization phenotypes with Gc binders (also see Discussion).

Next, a subset of these nAbs with at least some neutralization breadth against Old World hantavirus Gn/Gc in the rVSV assay (FIG. 2D) was selected and evaluated with authentic HTNV and DOBV (FIGS. 6B-6D). Concordant with the findings with authentic PUUV above, a substantial narrowing in the breadth of all the Gc binders tested from Groups II and IV was observed. Only a single Gn^{H}/Go-specific bnAb from Group I, ADI-42898, afforded complete neutralization of HTNV and DOBV in a manner that stent with its exceptional breadth of activity against the corresponding rVSVs (FIGS. 2D and 6B-6D).

### Example 9: Protective Effects of Group I and Group II Neutralizing Antibodies Before and After Viral Exposure

This example illustrates that Group I/II nAbs provide pre- and post-exposure protection in a bank vole model of PUUV challenge.

Two Group I (ADI-38919, -42898) and two Group II (ADI-42098, -42877) nAbs with potent activity against authentic PUUV (FIG. 6A) were selected and assessed their protective efficacy against the PUUV strain ‘Suonenjoki’ (PUUV/Suo) in a recently described bank vole infection model (FIGS. 7A-7B). PUUV/Suo establishes a persistent infection in bank voles within three days after the virus challenge and resembles a natural infection in these rodents in terms of viral load and tissue tropism. Virus-naïve bank voles from a laboratory-bred colony were first administered single 25-mg/kg doses of nAb intraperitoneally (i.p.) at 4 h pre-infection, then challenged with PUUV/Suo subcutaneously (s.c.). Viral loads in tissues previously associated with active PUUV replication were determined at 3 ys post-infection. Animals were sacrificed at 3 days post challenge and serum and organ viral RNA loads were determined by RT-qPCR detecting the PUUV S segment. All four nAbs afforded essentially complete pre-exposure prophylactic protection against viral acquisition in the lung, kidney, spleen, and blood (FIG. 7A).

Next, the nAbs as above were retested in a more stringent post-exposure setting (nAb treatment 24 h post-virus challenge). Both Group I and II nAbs mediated substantial (~1,000-fold) declines in mean viral RNA levels in the lung. This organ displayed the highest levels of viral replication, with reductions to undetectable levels in some animals. Animals were sacrificed at 3 days post challenge, and viral RNA loads were determined. Viral RNA levels in the spleen also declined in treated animals and were uniformly reduced to undetectable levels in the kidney and in circulation (FIG. 7B).

### Example 10: Cross-Clade Neutralization Effects of Antibodies of the Present Technology

This example illustrates that bnAb ADI-42898 affords cross-clade neutralization and post-exposure protection.

To identify bnAbs with cross-clade potential, nAbs with activity against multiple authentic Old World hantaviruses (FIGS. 6A-6D), and an rVSV bearing Gn/Gc from the New World hantavirus, ANDV (FIG. 2D) were prioritized. This set of nAbs, comprising a single Group I nAb, ADI-42898, and two Group II nAbs, ADI-39044 and -42871, was evaluated for neutralizing activity against rVSVs bearing New World hantavirus glycoproteins from ANDV, SNV, and Choclo virus (CHOV) as well as against authentic ANDV and SNV (FIGS. 8A and 8B and 18A-18D). ADI-42898 completely neutralized all three rVSV-Gn/Gc and authentic SNV, and it neutralized authentic ANDV with an un-neutralized virus fraction (FIGS. 8A and 8B). By contrast, the two Group II nAbs had little or no cross-clade activity towards authentic New World hantaviruses (FIGS. 18B and 18D), providing another example of the discordances between the rVSV and authentic virus assays for Group II nAbs.

ADI-42898's protective efficacy was evaluated in the Syrian hamster model of uniformly lethal ANDV challenge. Hamsters were exposed to the Chile variant of ANDV (ANDV/Chile-9717869) and then administered a single dose of ADI-42898 (4 mg; ~25 mg/kg) on day 3. All nAb-treated animals survived treatment, whereas all animals in the vehicle arm succumbed to infection (FIG. 8C). ADI-429898-mediated protection was associated with a complete reversal of viremia (FIG. 8D). This nAb fully retained its protective efficacy even at a fourfold lower dose (1 mg; ~6 mg/kg) (FIGS. 8C and 8D). ADI-42898, a fully human, Gn^{H}/Go-specific nAb with broad cross-clade activity against the major disease-associated hantaviruses, is the first mAb with demonstrated in vivo post-exposure protective efficacy against both Old World and New World rodent-borne hantaviruses.

### Material and Methods

### Study Design

This study was initiated to identify, isolate, and characterize human mAbs broadly reactive to hantaviral Gn/Gc, with the potential to neutralize Old World and New World hantavirus infections and prevent HCPS/HFRS. Sera from a cohort of 45 PUUV-experienced subjects for PUUV, HTNV and ANDV Gn/Gc-specific neutralizing antibody titers were screened and identified six donors presenting potent and broad neutralization activity. PUUV and ANDV Gn/Gc-reactive memory B cells were isolated by single-cell sorting; antibody heavy- and light-chain variable regions were cloned as full-length human IgG1 and expressed in S. cerevisiae. 135 mAbs for their cross-clade binding and neutralization activity as well as epitope specificity were characterized. Lastly, the protective efficacy of mAbs was evaluated in two animal models: a bank vole PUUV infection model for HFRS as well as a Syrian hamster ANDV challenge model for lethal HCPS.

Peripheral blood mononuclear cells (PBMC) and matching serum and clinical parameters from PUUV-exposed individuals were collected 19-78 days after disease onset. Subjects that had been laboratory-confirmed with PUUV infection during acute HFRS were recruited for a prospective and longitudinal study of HFRS.

Bank vole (Myodes glareolus) 5-8 months old were housed in groups of 3-5 animals and were provided standard rodent pellets and water ad libitum. During infection studies, group sizes for each condition were six animals with a 50/50 gender distribution. Using a one-tailed Fishers exact test to compare efficacy rates between the control and treated groups, the experiment requires a sample size of six animals per group for adequate (>80%) power. This sample size may permit detection of a minimum efficacy rate of 88% (56 protected) in the treated group compared to 0% (06 not protected) in the control group at a 95% confidence level. Randomization and blinding were not a part of this study. All bank voles from two independent experiments were included in the data analysis; no outliers were excluded. Upon completion of the experiment, 72 h post-virus inoculation, bank voles were euthanized by cervical dislocation.

Syrian golden hamster (Mesocricetus auratus) were used for experimentation. During challenge studies, group sizes were six 5-10 months old female animals. The group size was determined using statistical power analysis referred to in the bank vole infection study section. Randomization and blinding were not a part of this study. From two independent experiments several hamsters which were deemed untreated were excluded from the dataset (as described in the ‘Syrian hamster challenge studies’ section).

### Single B Cell Sorting

B cells were first purified from PBMCs using a MACS Human B Cells isolation kit (Miltenyi Biotec) and subsequently incubated for 30 minutes (min) on ice, with pre-titrated amounts of rVSV-mNG-P-PUUV-Gn/Gc or -ANDV-Gn/Gc to label antigen-specific B cells. The amounts of viral particles giving specific labeling of immune PBMC samples (PUUV-experienced donors) and non-specific labeling of non-immune PBMC samples (PUUV-naïve donors) were determined for each rVSV stock before sorting experiments. Following antigen labeling, cells were washed once with ice-cold MACS buffer, centrifuged for 10 min at 300×g at 4° C., and then stained using a panel of markers to aid identification of antigen-specific memory (class-switched) B cells for sorting: anti-human CD19 (PE-Cy7, Biolegend Cat #302216), CD3 (PerCP-Cy5.5, Biolegend Cat #30040), CD8 (PerCP-Cy5.5, Biolegend Cat #344710), CD14 (PerCP-Cy5.5, Invitrogen Cat #45-0149-42), CD16 (PerCP-Cy5.5, Biolegend Cat #360712), propidium iodide, IgM (APC, BD Biosciences Cat #314510), IgD (BV421, Biolegend Cat #348226) for 20 min on ice in the dark. Class-switched B cells, defined as CD19⁺/CD3⁻/CD8⁺/CD14⁺/CD16⁺/IgM⁺/IgD⁺ cells, showing over-background reactivity to either rVSV-mNG-P-PUUV-Gn/Gc or -ANDV-Gn/Gc were single-cell sorted using BD FACS Aria II (BD Biosciences) into 96-well polystyrene microplates (Corning) containing 20 μl/well of lysis buffer (5 μl of 5× first strand Super Script IV cDNA buffer [Invitrogen], 1.25 μl dithiothreitol [Invitrogen], 0.625 μl of NP-40 [Thermo Scientific], 0.25 μl RNaseOUT [Invitrogen], and 12.8 μl dH₂O). Plates were immediately stored at −80° C. Flow cytometry data were analyzed using FlowJo software.

### Bank Vole Challenge Studies

The neutralizing potential of human mAbs against PUUV/Suo challenge in vivo was investigated in an established experimental PUUV reservoir host model system (5). The initially introduced bank voles were tested for pathogens. Bank voles were challenged with 500 FFU of PUUV/Suo by s.c. inoculation. Group sizes for each condition were six animals with a 50/50 gender distribution. In a prophylactic treatment regimen, voles were inoculated i.p. with ~25-mg/kg of mAbs (or PBS as vehicle control) 4 h prior to challenge; in a post-exposure treatment scheme, voles were inoculated with ~25-mg/kg of mAbs 24 h post virus inoculation. The animals were euthanized 72 h post virus inoculation by cervical dislocation, after which RNA was isolated from lungs, spleen, kidneys, and serum; virus RNA loads were assessed by PUUV S-segment RT-qPCR.

### Syrian Hamster Challenge Studies

Groups (n=6) of Syrian golden hamsters, 100-200 grams, were exposed i.m. to 200 plaque-forming units (PFU) of ANDV strain Chile-9717869 diluted in 0.1 ml PBS. On day 3 post-exposure, hamsters were treated i.p. with indicated amounts (~25- or ~6-mg/kg) of mAb diluted in 0.8 ml of PBS. Hamsters were monitored daily for clinical signs of disease, morbidity, and mortality for 35 days. Moribund animals, defined as those experiencing severe respiratory distress or showing unresponsiveness, were humanely euthanized. On day 8 post-exposure, blood samples were collected by venipuncture of the anterior vena cava and the serum fraction was collected following centrifugation.

Viral titers of serum collected at day 8 post-exposure were determined by plaque assay. Serial log dilutions of serum were prepared in MEM media supplemented with 5% FBS, 2 mM L-glutamine and 1% gentamicin, and confluent monolayers of VeroE6 cells on 6-well plates were inoculated. After adsorption for 1 h at 37° C., monolayers were overlaid with Eagle basal medium (EBME) supplemented with 0.5% agarose (Seakem), 30 mM Hepes buffer, and 5% FBS. After incubation at 37° C. for 8 days, a second overlay, supplemented with 5% Neutral red, was added. Plaques were counted the following day and titers were expressed as PFU/ml.

Human IgG concentrations in hamster serum collected at day 8 post-exposure were determined by quantitative capture ELISA in order to exclude any hamsters that remained human IgG negative following antibody treatment, a phenomenon that has been described previously (54). ELISA plates were coated with sheep anti-human IgG capture antibody at 0.1 pg per well and blocked with Casein buffer (G Biosciences) at 4° C. overnight. Coated plates were washed with PBS/0.1% Tween 20 and blocked with Casein buffer for 1 h at room temperature on a shaker. A standard curve for ADI-42898 was generated by 2-fold dilutions from 500 ng/ml to 15.6 ng/ml of mAb in assay buffer (Casein buffer/10% normal hamster serum). Day 8 serum samples were diluted 1:50 in assay buffer. Standards and diluted samples were added to coated and washed wells and incubated at room temperature for 1 h. Antibodies were detected by adding goat anti-human IgG-HRP conjugate for 1 h. Absorbance values were measured using a SpectraMax 5 (Molecular Devices) and standard curves were generated using GraphPad Prism software and nonlinear regression analysis (sigmoidal, 4PL). Absorbance values from samples with unknown human antibody concentrations were applied to the standard curve to determine the concentration of human IgG. Hamsters that were human IgG negative at day 8 were deemed untreated and removed from the dataset.

### Statistical Analysis

Statistical details for each experiment may be found in the respective figure legends. These include the number of replicates (n), measures of precision, and the statistical test used. Dose-response neutralization curves were subjected to nonlinear regression analysis to derive IC₅₀ values (4-parameter, variable slope sigmoidal dose-response equation constraining bottom parameters to 0-100). Viral load in animal tissue or serum (FIGS. 7 and 8) was analyzed using a Kruskal-Wallis test with Dunn's correction for multiple comparisons. Survival curves from the hamster challenge study (FIG. 8) were compared by a Mantel-Cox (log-rank) test. All statistical analyses were carried out in GraphPad Prism (alpha, 0.05).

### Cell Lines

African green monkey kidney Vero cells (ATCC Cat #CCL-81) and VeroE6 cells (ATCC Cat #CRL-1586) were cultured in a humidified 37° C., 5% CO₂ incubator in high-glucose Dulbecco's modified Eagle medium (DMEM, Life Technologies) supplemented with 2% fetal bovine serum (FBS, Atlanta Biologicals), 1% GlutaMAX, and 1% penicillin-streptomycin (both Life Technologies). Human hepatocarcinoma Huh.7.5.1 cells (a generous gift of Jan Carette, Stanford University, CA) were maintained in high-glucose DMEM (Life Technologies) supplemented with 10% FBS (Atlanta Biologicals), 1% GlutaMAX (Life Technologies), 1% non-essential amino acids (Life Technologies) and 1% penicillin-streptomycin (Life Technologies). Chinese hamster ovary (FREESTYLE™ CHO-S) cells (Gibco, Cat #R80007ATCC) were cultured in a humidified 37° C., 5% CO₂ incubator in high-glucose Dulbecco's modified Eagle medium (DMEM, Life Technologies) supplemented with 10% fetal bovine serum (FBS, Atlanta Biologicals), 1% GlutaMAX, and 1% penicillin-streptomycin (both Life Technologies). Human osteosarcoma U20S cells (ATCC Cat #HTB-96) were similarly maintained in modified McCoy's 5A medium (Life Technologies) supplemented with the above-named reagents. Human umbilical vein endothelial cells (HUVECs, Lonza Cat #C2517A) were maintained in endothelial cell growth medium (EGM) supplemented with EGM-SingleQuots (Lonza) at 37° C. with 5% CO₂. Mygla.Rec.B bank vole epithelial cells were cultured as described previously (5). Drosophila melanogaster S2 cells (Thermo Fisher Cat #R69007) were maintained in serum-free insect cell medium (HyClone, GE HealthCare) supplemented with 1% penicillin-streptomycin in spinner flasks in a 28° C., 150 rotation per minute (rpm) humidified shaking incubator. ExpiCHO cells (Thermo Fisher Cat #A29127) carrying a FUT8-knockout mutation (Mapp Biopharmaceuticals Inc.) were maintained in their designated growth medium (Thermo Fisher) and cultured in a 37° C., 8% CO₂, 125 rpm humidified shaking incubator.

Recombinant Vesicular Stomatitis Viruses (rVSVs)

rVSVs expressing enhanced green fluorescent protein (eGFP) and bearing Gn/Gc from HTNV, SEOV, DOBV, ANDV, SNV, CHOV and VSV G were engineered, rescued and propagated following published protocols (2, 3, 6-8). rVSV carrying a phosphoprotein P fused to mNeonGreen (mNG) and bearing PUUV Gn/Gc has been described previously (31). Generation of viruses with an equivalent mNG-P backbone decorated with ANDV Gn/Gc (Chilean field strain R123, GenBank accession number NP_604472.1), SNV Gn/Gc (human field isolate, GenBank accession number NP_941974.1), and HTNV Gn/Gc (cell culture-adapted strain 76-118, GenBank accession number NP_941978.1) was performed by using standard molecular biology techniques. The identity and sequence of each virus-encoded Gn/Gc was determined by Sanger sequencing of rVSV genomic RNA-derived cDNAs. All viruses were propagated on Vero cells except rVSV-mNG-P PUUV-Gn/Gc, which was amplified on Huh.7.5.1 cells.

### VSV Infectivity Measurements and Neutralization Assays

Confluent Vero cells were inoculated with pre-titrated amounts of pseudotyped rVSV particles bearing either PUUV, HTNV, SEOV, DOBV, ANDV, SNV, and CHOV Gn/Gc or, when indicated, rVSV-PUUV-Gn/Gc neutralization-escape mutants. Prior to infection, rVSVs were diluted in corresponding media, and infected cells were maintained at 37° C. for 14 to 16 h post-infection before automated counting of eGFP+ and mNG+ cells using a Cytation 5 cell imaging multi-mode reader (BioTek Instruments) or a Cell-Insight CX5 imager (Thermo Fisher) including onboard software. For neutralization experiments, rVSV particles were incubated with increasing concentrations of mAbs or, when indicated, human serum diluted in corresponding media at room temperature for 1 h prior to addition to cell monolayers. Virus neutralization data were subjected to nonlinear regression analysis to derive IC₅₀ values (4-parameter, variable slope sigmoidal dose-response equation constraining bottom parameters to 0-100; GraphPad Prism).

### Authentic Hantavirus Infections and Microneutralization Assays

PUUV strain Suo (PUUV/Suo) was propagated in Mygla.Rec.B bank vole epithelial cells (5), whereas PUUV strain Kazan (9) and DOBV strain Ano-Poroia/AF19 (10) were grown in VeroE6 cells as described. VeroE6 cells were exposed to PUUV Kazan or DOBV at a multiplicity of infection (MOI) of 0.1-1 of fluorescent focus-forming units (FFU) following a pre-incubation with indicated mAb concentrations. Viral infectivity was calculated by immunostaining of formaldehyde-fixed cells at 20 h (PUUV) or 48 h (DOBV) post-infection using polyclonal rabbit serum targeting the PUUV nucleocapsid protein N (NR-9675, BEI Resources) or a broadly cross-reactive hantavirus N-specific antiserum (5). Plates were imaged with either the Plate RUNNER HD (Trophos) or the Opera Phenix High-Content Screening System (PerkinElmer) at the FIMM high content imaging and analysis. Images were analyzed and individual infected cells counted using the system's onboard software.

ANDV strain Chile-9717869, SNV strain CC107, and HTNV strain 76-118 used for all in vitro assays were propagated and viral titers determined using VeroE6 cells as previously described (2, 11-13). For neutralization assays using authentic virus, pre-titrated amounts of virus were mixed with serially diluted mAbs for 1 h before adding virus/mAb mixture to HUVECs at a MOI of 1 (ANDV and HTNV) or MOI of 2 (SNV). Viral infectivity was determined by immunostaining of formaldehyde-fixed and permeabilized cells at 48 h post-infection with polyclonal rabbit sera specific for ANDV, HTNV or SNV N (NR-9673, NR-9674, and NR-12152; BEI Resources). Images were acquired on an Operetta high-content imaging device (PerkinElmer). Images were analyzed with a customized scheme built from image-analysis functions present in the Harmonia software, and the percentage of infected cells was determined using the analysis functions. Virus neutralization data were subjected to nonlinear regression analysis to derive IC₅₀ values (4-parameter, variable slope sigmoidal dose-response equation constraining bottom parameters to 0-100; GraphPad Prism).

Generation of Recombinant PUUV Gn^{H} and Gn^{H}/Gc Antioen

To obtain the head domain of PUUV Gn (Gn^{H}, aa 20-381) and the PUUV Gn^{H}/Gc complex (both strain Mu/07/1219, GenBank code: AJC50718) the approach described previously was used (14). In short, a codon optimized sequence (GenScript) encoding Gn^{H} alone, and a single-chain construct of Gn^{H} in frame with the Gc ectodomain (aa 659-1093) in which the amphipathic C-terminal membrane proximal external region was removed was generated. To prevent complex dissociation during purification, both subunits were joined using a flexible linker of 43-aa long (GGSGLVPRGSGGSGGGSWSHPQFEKGGGTGGGTLVPRSGTGG) (SEQ ID NO:71) containing two thrombin cleavage sites (underlined) at either end or a Strep-tag sequence (in bold) in the middle. Both Gn^{H} and Gn^{H}/Gc encoding sequences were cloned into a modified pMT/BiP plasmid (Invitrogen) expressing them in frame with a C-terminal double Strep-tag. This plasmid was used to generate stable Drosophila melanogaster S2 cells, which were selected and maintained in serum-free insect cell medium containing 7 μg/ml puromycin. Protein expression was induced by adding 5 μM CdCl₂ and cell supernatant was harvested 5 days post induction, supplemented with 10 μg/mL avidin and 0.1 M Tris-HCl (pH 8.0). Proteins were purified via strep-tactin affinity chromatography followed by size exclusion chromatography (SEC) using a Superdex 200 10/30 column (GE Healthcare) in a buffer composed of 10 mM Tris-HCl (pH 8), 150 mM NaCl, and 1 mM EDTA.

### Amplification and Cloning of Antibody Variable Genes

Human antibody variable gene transcripts (V_{H}, V_{κ}, V_{λ}) were amplified by RT-PCR using the Super Script IV (Invitrogen) followed by nested PCRs using cocktails of IgM-, IgD-, IgA- and IgG-specific primers and the HotStarTaq Plus DNA Polymerase (Qiagen), as described previously (15). To allow cloning by homologous recombination into S. cerevisiae, the primers used in the second round of nested PCR contained 40 base pairs of 5′ and 3′ homology to the digested yeast expression vectors. Amplified variable gene transcripts were transformed into yeast using the lithium acetate method for chemical transformation (16). 200 μl of the transformed yeast cell suspension was plated on selective media and the plates were incubated at 30° C. for 48 h. Following the growth of transformants on the plates, individual yeast colonies were picked for sequencing.

Expression and Purification of IgGs, scFv and F(Ab) Fragments

IgGs were expressed in S. cerevisiae cultures grown in 24-well plates, as described previously (17). Briefly, after 6 days, the cultures were harvested by centrifugation and IgGs were purified by Protein A affinity chromatography. The bound antibodies were eluted (200 mM acetic acid/50 mM NaCl [pH 3.5]) into ⅛th volume of 2 M Hepes buffer (pH 8.0) and buffer-exchanged into phosphate buffered saline (PBS, pH 7.0).

F(ab) fragments were generated by digesting IgGs with papain for 2 h at 30° C. The digestion was terminated by the addition of iodoacetamide, and the F(ab) and Fc mixtures were passed over Protein A agarose to remove Fc fragments and undigested IgG. The flow-through of the Protein A resin was then passed over CAPTURESELECT™ IgG-CH1 affinity resin (Thermo Fisher) and eluted (200 mM acetic acid/50 mM NaCl [pH 3.5]) into ⅛th volume of 2 M Hepes buffer (pH 8.0). F(ab) fragments then were buffer exchanged into PBS (pH 7.0).

Single chain Fv fragments (scFv) coding sequences of ADI-37236, -42089, -42898, -42877 and -42885 were generated by joining together the sequences encoding the V_{H} and V_{L} chains by a flexible linker and cloned into a modified pMT/BiP plasmid (Invitrogen). The antibody fragments were purified following the protocol described above in the ‘Generation of recombinant PUUV Gn^{H} and Gn^{H}/Gc antigen’ section.

### ELISA for Gn/Gc:mAb Binding

All mAbs cloned from antigen-specific B cells were tested for their reactivity to rVSV particles displaying the Gn/Gcs of PUUV, HTNV, DOBV, SEOV, ANDV and SNV in a single concentration spot ELISA. Viral particles were diluted in PBS (pH 7.4) and captured directly onto high binding half-area 96-well plates (Corning) overnight at 4° C. in amounts determined using a calibration assay (designed to ensure equivalent particle loading across the antigenically distinct rVSVs). Plates were then washed with PBS and blocked with a blocking/binding buffer (PBS containing 5% non-fat dried milk) for 1 h. Blocked plates were exposed to antibodies, in duplicate, at 25 nM mAb concentration for 1 h at 37° C. Plates were washed with PBS and mAb binding was detected with anti-human IgG secondary antibody conjugated to horseradish peroxidase (HRP, Thermo Fisher) for 1 h at 37° C. Plates were washed, 1-STEP™ Ultra TMB-ELISA Substrate Solution (Thermo Fisher) was added and quenched with the addition of 2 M sulfuric acid, per manufacturer recommendations. Absorbance was read at 450 nm and antibodies producing signal >0.3 were categorized as binders.

### Competition ELISA

High-protein-binding 96-well ELISA plates (Corning) were coated with purified human mAbs (40 ng per well, ‘first mAb’) at 37° C. and blocked with 3% (w/v) bovine serum albumin (BSA; Thermo Fisher) in PBS. The membranes of rVSV-PUUV-Gn/Gc particles were labelled with a short-chain phospholipid probe, functional-component spacer diacyl lipid conjugated to biotin (FSL-biotin, Sigma-Aldrich), as described (2). Subsequently, pre-titrated amounts of biotin-labeled rVSVs were incubated with purified human mAb (100 nM, ‘second mAb’) for 1 h at 37° C. before their addition to mAb-coated plates. Bound rVSV-PUUV-Gn/Gc was detected by incubation with a streptavidin-HRP conjugate (Thermo Scientific). Competition levels were determined by reduction of rVSV binding in presence of a competitor mAb compared to rVSV binding in the absence of competition (rVSV complexed with human IgG1 [Sigma-Aldrich]). Antibodies with >75% reduction of rVSV binding were in competition with pre-complexed mAbs.

### Yeast-Based mAb Competition Assay

Antibody competition assays were performed as previously described (18). Antibody competition was measured by the ability of a competitor Group I (ADI-42898) or Group II (ADI-42098) F(ab) to inhibit binding of yeast surface-expressed anti-Gn/Gc IgGs to recombinant PUUV Gn^{H}/Gc. 50 nM biotinylated Gn^{H}/Gc was pre-incubated with 1 μM competitor F(ab) for 30 min at room temperature and then added to a suspension of yeast expressing anti-Gn/Gc IgGs in a 96-well format. Unbound antigen was removed by washing with PBS containing 0.1% BSA. After washing, bound antigen was detected using streptavidin-Alexa Fluor 633 conjugate (Life Technologies) and analyzed by flow cytometry using a FACSCanto II (BD Biosciences). The level of competition was assessed by measuring the fold reduction in PUUV Gn^{H}/Gc binding in the presence of competitor F(ab) relative to an antigen-only control. Antibodies were considered competitors when a greater than five-fold reduction was observed in the presence of control F(ab) relative to an antigen-only control.

Selection of rVSV Neutralization-Escape Mutants

Selection of escape mutants was performed as described previously (19). In short, rVSV-PUUV-Gn/Gc was serially passaged after rVSV pre-incubation with a concentration of mAb corresponding to its IC₅₀ value derived from neutralization assays, and then added to confluent monolayers of Huh.7.5.1 cells. Infection was allowed to proceed to completion (>90% cell death by eye), supernatants were harvested and following several passages under mAb selection, supernatants were tested for viral neutralization escape. If resistance was evident, individual viral clones were plaque-purified on Huh.7.5.1 cells, and their Gn/Gc-encoding sequences were determined.

### Negative-Stain Electron Microscopy

The PUUV Gn^{H}/Gc:scFv complexes were assembled in vitro by co-incubating the heterodimer Gn^{H}/Gc with a 1.5-fold excess of scFv for 1 h at 4° C. prior to purification over a Superdex 200 gel filtration column (GE Healthcare). Complexes were diluted to a final concentration of 40 nM, deposited onto glow-discharged carbon grids (CF300, EMS), and negatively stained in 2% (w/v) uranyl acetate. The negatively stained specimens were mounted onto a transmission electron microscope holder and examined with a Tecnai F20 electron microscope operating at a voltage of 200 kV. Images were collected with a Falcon II direct electron detector (FEI) with a pixel size of 2 Å per pixel and a total accumulated dose of 20 electrons per Å². All image processing and analyses was carried out using cryoSPARC (20). In short, the images were CTF—(contrast transfer function) corrected, the particles automatically picked, and then 2D-classified. The best 2D classes were used to generate the initial 3D model, which was further refined and filtered to 20 Å.

### Production of Human IgGs for Animal Studies

Antibody V_{H} and V_{L} coding sequences were ordered as gBlocks (Integrated DNA Technologies) with a 15 base pair 5′ overlap to a mouse IgKVIlI secretion signal coding sequence and a 15 base pair 3′ overlap to the appropriate sequences encoding for a constant region (human kappa, human lambda or human IgG1). The variable regions coding sequences were cloned into a pcDNA3.4 (Thermo Fisher) expression vector modified to contain coding sequences of a mouse IgKVIII signal sequence and the appropriate constant regions. The gBlocks were then inserted into the vector between the secretion signal and the constant region coding sequences using In-Fusion enzyme (Takara Bio). Antibodies were transiently expressed in ExpiCHO cells (Thermo Fisher) carrying a FUT8-knockout mutation (Mapp Biopharmaceuticals Inc.) utilizing the high titer protocol for CHO Expifectamine (Thermo Fisher). Approximately 10 days post transfection, cell culture supernatants were clarified by spinning at 3,200×g for 10 min, filtered and loaded onto a HiTrap MabSelect SuRe affinity column (Cytiva) using an ÅKTA pure 25 fast protein liquid chromatography (FPLC) system. Once loaded, the column was washed with 10 column volumes of PBS (pH 7.2), followed by elution of the target antibody with Pierce IgG elution buffer (Thermo Fisher). Elution fractions containing the target antibody were pooled and neutralized to ~pH 7 with 1.0 M Tris, pH 7.8.

### Quantitative Real-Time PCR (RT-QPCR) Assay

Following euthanasia and dissections, RNA was extracted from bank vole tissues using Trizol Reagent (Thermo Fisher) according to the manufacturer's instructions. In case of serum, RNA was isolated using the QIAamp Viral RNA Mini Kit (Qiagen). For RT-qPCR, RNA was either analyzed with the qPCRBIO SyGreen 1-Step Go Lo-Rox kit (PCR Biosystems), or reverse transcribed using RevertAid reverse transcriptase (Thermo Fisher) and random hexamer primers (Thermo Fisher) followed by PCR analysis with Maxima SYBR Green/ROX qPCR Master Mix (Thermo Fisher). Protocol, primers and standard for PUUV S-segment RT-qPCR analyses were carried out as previously described (21) and the analyses were performed using AriaMx instrumentation (Agilent). The results were normalized to total RNA concentration of the samples and presented as S segment copy number/μg total RNA.

### Polyreactivity Assay

Antibody polyreactivity was measured as described previously (22). Briefly, soluble membrane protein (SMP) and cytosolic protein (SCP) fractions obtained from CHO cells were biotinylated using NHS-LC-Biotin (Thermo Fisher). Yeast presenting IgGs on their surface were incubated with biotinylated CHO cell preparations on ice. Cells were then washed twice with ice-cold PBSF (PBS containing 0.1% w/v BSA) and incubated with a secondary labelling mix containing ExtrAvidin-R-PE (Sigma-Aldrich Cat #E4011), anti-human LC-FITC (Southern Biotech Cat #2062-02) and propidium iodide for 15 min. The cells were washed twice with PBSF, resuspended and run on a FACSCanto II (BD Biosciences). The mean fluorescence intensity of binding was normalized using control antibodies that display low, medium or high polyreactivity to assess the non-specific binding.

Biolayer Interferometry Binding Analysis with sGn and Gn^{H}/Gc

For monovalent apparent dissociation constant (K_{D}) determination, IgG binding to recombinant sGn and Gn^{H}/Gc antigen was measured by biolayer interferometry (BLI) using a ForteBio Octet HTX instrument (Molecular Devices). For measurements with PUUV Gn^{H}/Gc, the IgGs were captured (response level 0.5-1.5 nm) to anti-human IgG capture (AHC) biosensors (Molecular Devices) and allowed to stand in PBSF for a minimum of 30 min. After a short (60 s) baseline step in PBSF, the IgG-loaded biosensor tips were exposed (180 s, 1000 rpm of orbital shaking) to the test antigen (100 nM in PBSF) and then dipped (180 s, 1000 rpm of orbital shaking) into PBSF to measure any dissociation of the antigen from the biosensor tip surface. For measurements with sGn, biotinylated antigen (100 nM) was bound to streptavidin capture sensors (Molecular Devices). Binding analysis of IgGs (100 nM) in solution to sGn was performed as described for Gn^{H}/Gc antigen. Data for which binding responses were 0.1 nm or greater were aligned, inter-step corrected (to the association step) and fit to a 1:1 binding model using the ForteBio Data Analysis Software, version 11.1.

### Virus Attachment Assay

This assay was performed following a previously published protocol (32). In short, rVSV-PUUV-Gn/Gc particles bearing mNG-labeled nucleocapsids were incubated with mAb (25 nM) or serum from PUUV-experienced donors at room temperature for 1 h. These rVSV:mAb complexes were spin-occulated onto prechilled U20S cells at 1,000 rpm and 4° C. for 10 min followed by an incubation on a cooling plate at 6° C. for 30 min. Unbound virus was removed by washing with cold PBS. Cells were harvested in PBS, fixed with 4% paraformaldehyde on ice and surface-bound virus was detected by flow cytometry by gating on mNG using an LSRII flow cytometer (BD Biosciences) at the Flow Cytometry Core Facility of the Albert Einstein College of Medicine.

### Fusion Infection Assay

rVSV-PUUV-Gn/Gc or -ANDV-Gn/Gc particles were bound to prechilled HUVECs by centrifugation at 2,500 rpm and 4° C. for 1 h. Cells were placed on ice and unbound virus washed away with cold PBS. To establish the ideal pH for PUUV Gn/Gc-mediated fusion at the plasma membrane, PBS was replaced with DMEM-F12 media adjusted to pH values ranging from 4.0 to 7.0. Fusion was initiated by incubating HUVECs at 37° C. for 1 min, followed by shifting cells back to 4° C. Further rounds of infection were prevented by the exchange of media supplemented with 20 mM NH₄Cl, and infected cells were maintained at 37° C. for 12 to 14 h post-infection before automated counting of eGFP+ and mNG+ cells using a Cytation 5 cell imaging multi-mode reader (BioTek Instruments) and a Cell-Insight CX5 imager (Thermo Fisher) including onboard software. To evaluate the ability of mAbs to inhibit fusion, rVSV-PUUV-Gn/Gc were pre-incubated with increasing amounts of mAb (0.005 to 5.0 nM) for 1 h at 4° C. prior to centrifugation onto HUVECs. Fusion was initiated with an optimal pH of 5.5 and viral infectivity was scored as described above.

### Example 11: Assessing Neutralization Effects Using Affinity Matured Variants of Antibodies of the Present Technology

This example illustrates two antibodies, ADI-65533 and ADI-65534, that showed cross-clade neutralization and post-exposure protection. ADI-65533 and ADI-65534 are affinity matured variants of ADI-42898, and the mutations relative to ADI-42898 are shown in Table 3.

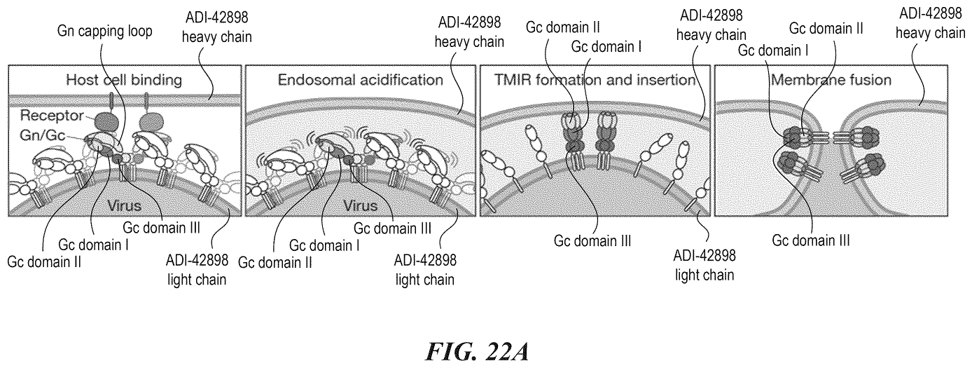

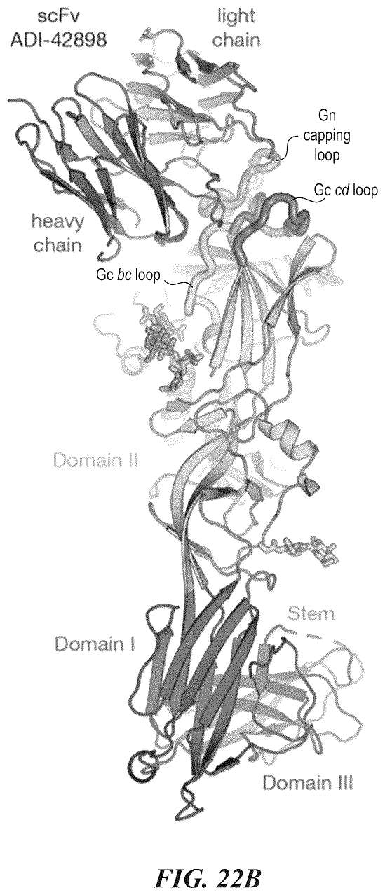

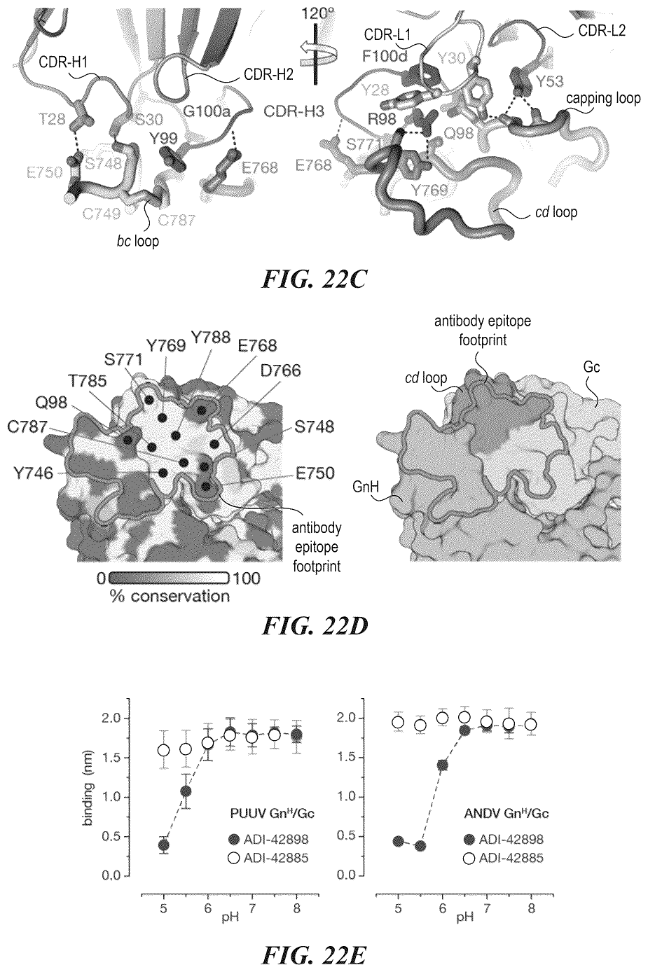

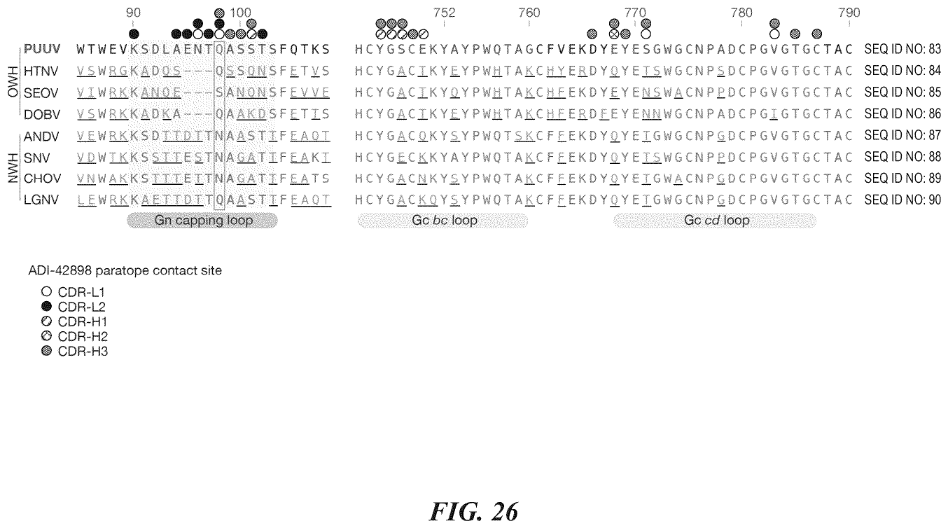

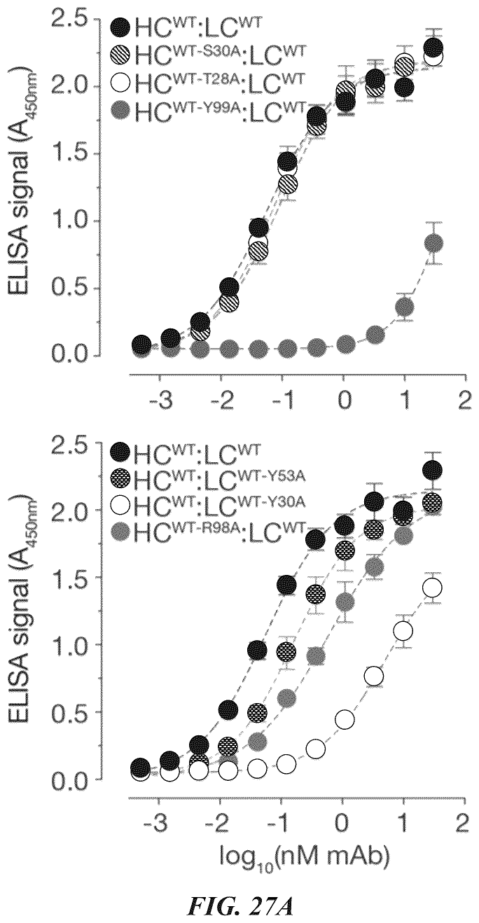

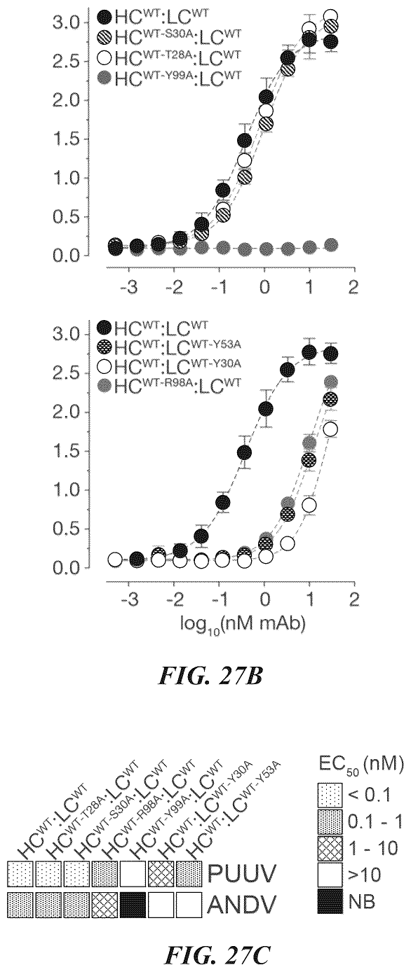

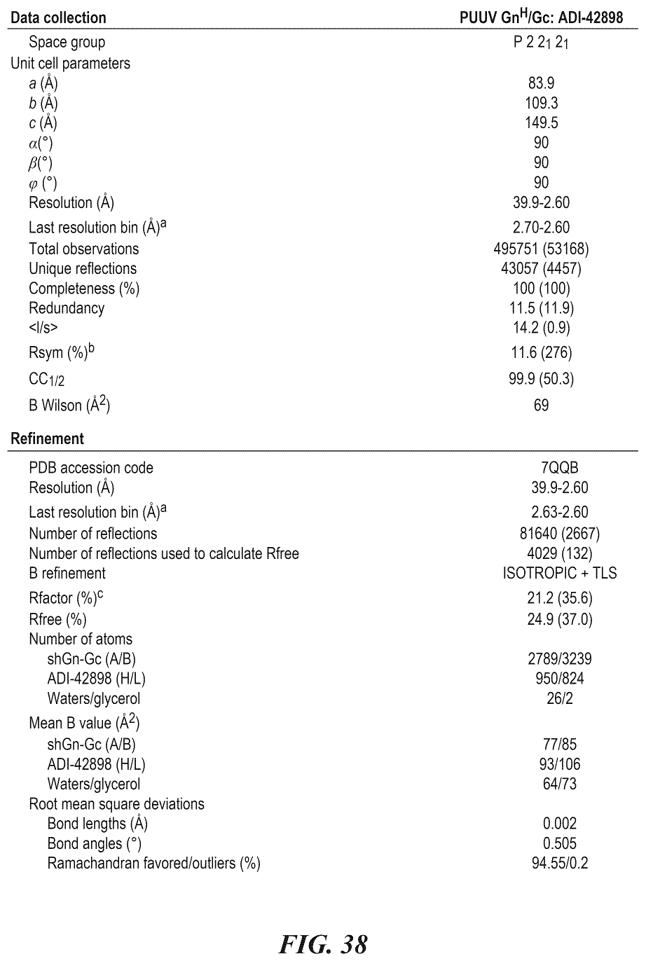

To uncover the structural basis of ADI-42898:Gn/Gc recognition, a complex formed by a single-chain, variable domain fragment of the antibody (scFv) and the PUUV glycoprotein complex was crystallized. The latter was generated by joining the Gn head (Gn^{H}, amino acid residues 20-381) and the Gc ectodomain (residues 659-1093) with a linker, as previously reported for ANDV and Maporal virus (MPRLV) (FIG. 38). In the crystal structure, PUUV Gn^{H}/Gc forms a pre-fusion complex resembling those of ANDV and MPRLV, with Gn^{H} making extensive contacts with Gc at the tip of domain II (FIGS. 22B, 22C, and 26). ADI-42898 simultaneously binds both glycoprotein subunits and engages three discontinuous regions: the capping loop in Gn^{H}, and the bc and od loops in Gc domain II. The total surface area buried by the antibody is 745 Å², distributed in a 40:60 ratio between Gn and Gc (FIG. 22D). In line with the quaternary nature of the epitope, dissociation of the Gn^{H}/Gc complex at acidic pH abrogated binding by ADI-42898, but not by a Gc binding monoclonal antibody (mAb), ADI-42885 (FIG. 22E). The Gc and Gn portions of the ADI-42898 epitope are framed by the complementary-determining regions (CDRs) H1 and H3 of the antibody's heavy chain (Gc subunit), and by CDRs L1 and L2 of its light chain (Gn subunit), such that (i) T28^{H1} and S30^{H1} are hydrogen-bonded with side chain atoms of the bc loop; (ii) R98^{H3} and Y99^{H3} interact with a cleft between the bc and cd loops; and (iii) Y30^{L1} and Y53^{L2} contact main chain atoms in the capping loop (FIG. 22C). Each of these contact residues were exchanged for alanine and measured the variants' binding activities to recombinant vesicular stomatitis virus (rVSV) pseudotypes bearing PUUV or ANDV Gn/Gc via enzyme-linked immunosorbent assays (ELISA) (FIG. 27). While substitution of T28^{H1} and S30^{H1} had no effect on Gn/Gc engagement, mutation of Y53^{L3}, R98^{H3}, and Y30^{L1} each significantly reduced binding, with Y30^{L1} having the strongest impact [reduction by ~70-fold (ANDV) and ~90-fold (PUUV)]. Exchange of Y99^{H3} rendered the antibody completely inactive. The contacts between CDR-H3 and the cd loop in Gc, and between CDRs-L1 and -L2 and the Gn capping loop may drive ADI-42898:Gn/Gc recognition.

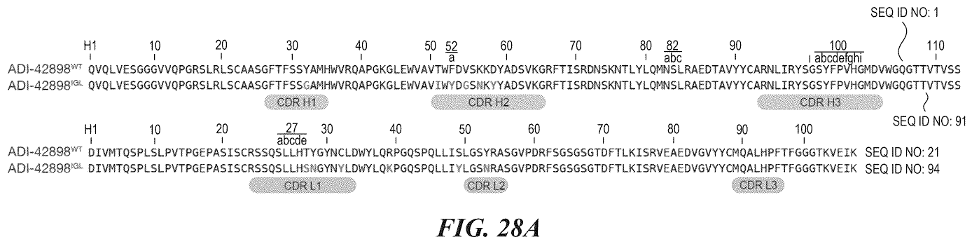

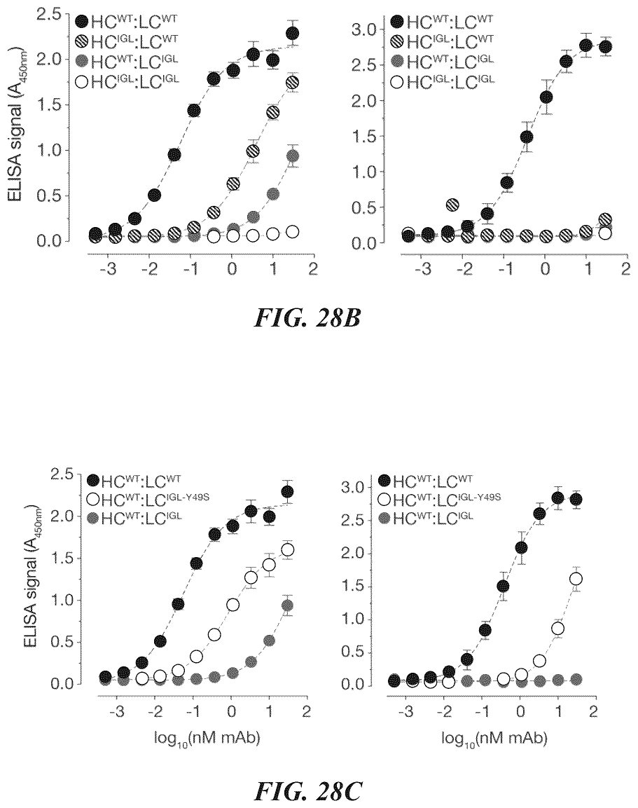

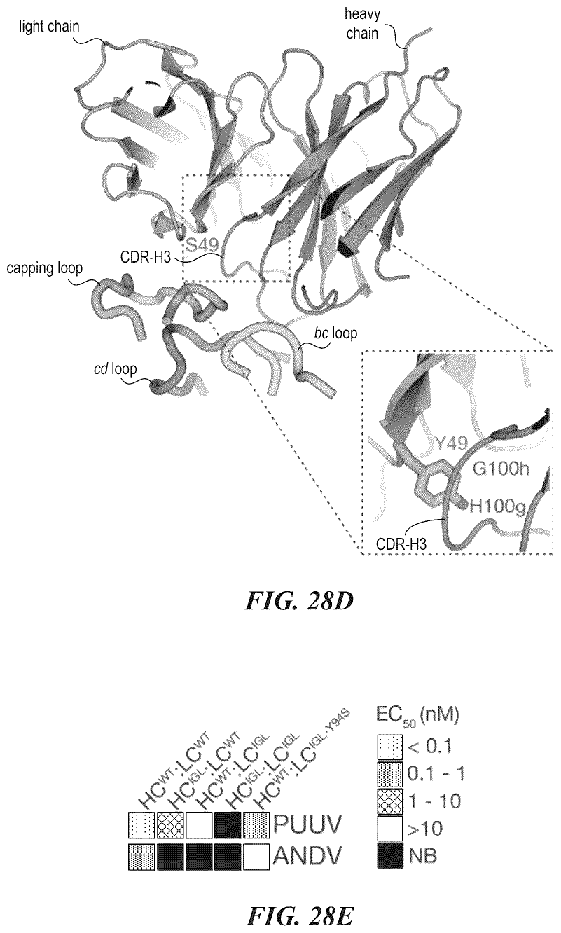

Of the above six contact residues in the ADI-42898 paratope, only Y53^{L3} resulted from a somatic hypermutation (SHM) (FIG. 28A). To determine if any other SHMs contribute to ADI-42898 activity, the heavy (HC) and light (LC) chains were reverted to their inferred germline (IGL) ancestors (FIG. 28), but retained the mature CDR-H3, since its germline sequence could not be predicted with confidence. Three antibodies, the IGL ancestor (HC^{IGL}:LC^{IGL}) and two chimeras containing the inferred germline of one chain and the mature (wild-type, WT) version of the other (HC^{IGLL}:LC^{WT} and HC^{WT}:LC^{IGL}) were generated and assessed their binding properties as above (FIGS. 28B, 28C and 28E). Concordant with the findings indicating a key role for the LC in Gn/Gc binding, only the chimera containing the mature LC (HC^{IGL}:LC^{WT}) and CDR-H3 retained partial binding activity. A closer examination of the structure suggests that the SHM Y49S (FIG. 28D) located in the framework region 2 (FR2) of the LC might be required to pre-organize H3 into a conformation favorable for epitope recognition. Consistent with this hypothesis, introduction of the SHM Y49S into HC^{WT}:LC^{IGL} rescued ADI-42898's binding activity (FIGS. 28C-28E).

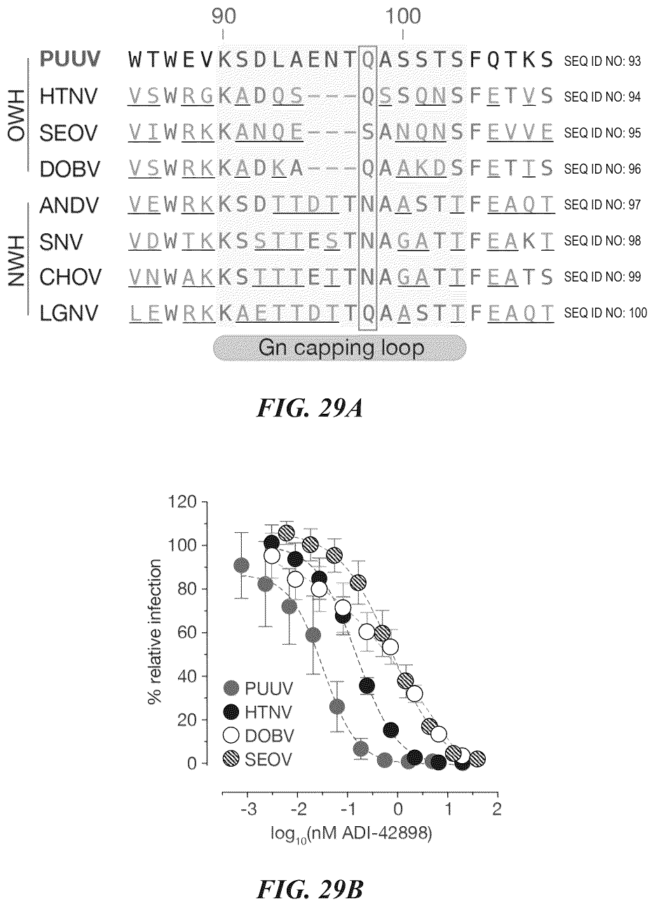

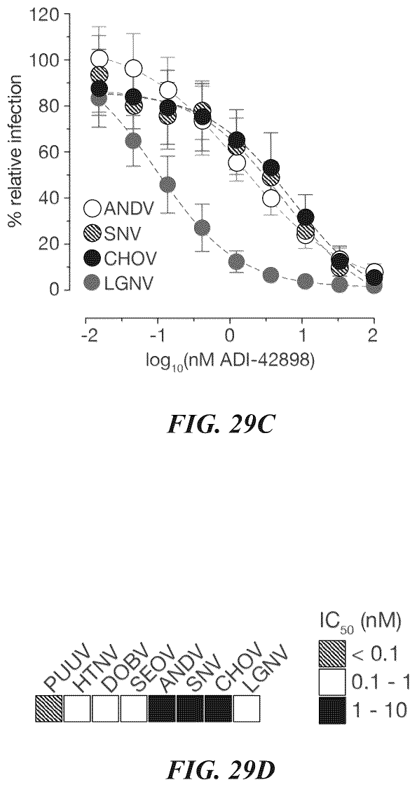

To identify critical binding determinants in ADI-42898's quaternary Gn/Gc-spanning epitope, the effects of natural sequence variations in Gn/Gc on antibody neutralization activity were assessed. Specifically, rVSVs bearing Gn/Gc derived from eight divergent, rodent-borne hantaviruses were surveyed (FIG. 29). Although rVSVs bearing NWH Gn/Gc were generally less susceptible to ADI-42898 neutralization than their OWH counterparts, rVSV bearing the Laguna Negra virus (LGNV) Gn/Gc was a notable exception to this trend. The enhanced susceptibility of rVSV-LGNV-Gn/Gc may be tentatively attributed to residue 98^{Gn} (Q in OWH and LGNV, N in NWH), the only residue in the capping loop whose side chain is directly contacted by ADI-42898 (FIGS. 26 and 29). The key role for 98^{Gn} in susceptibility to ADI-42898 neutralization, regardless of viral clade and the length of the capping loop, is also supported by previous observation that rVSV-PUUV-Gn/Gc^{Q98R} escapes ADI-42898 neutralization.

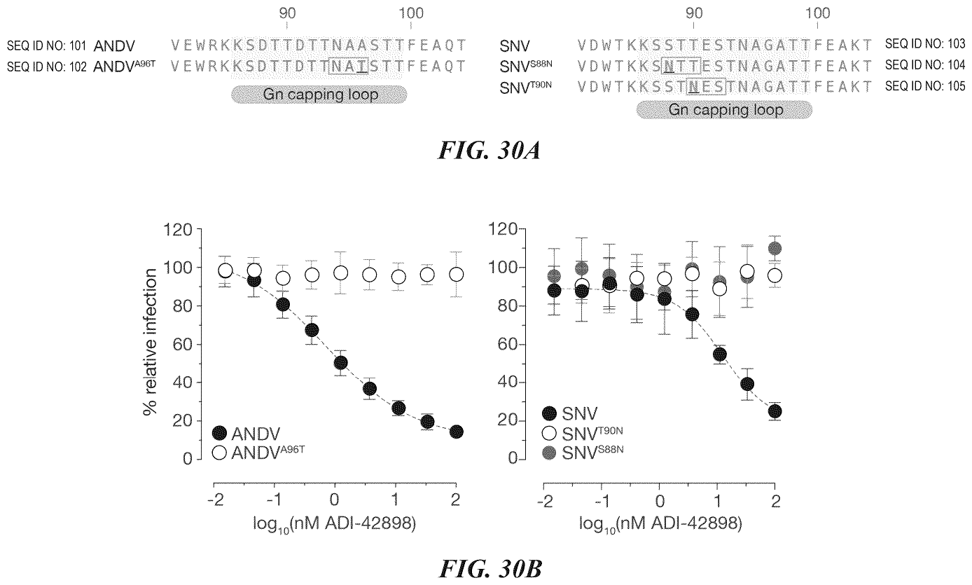

To uncover additional Gn/Gc residues important for ADI-42898 recognition, escape mutants with rVSV-ANDV- and -SNV-Gn/Gc were selected, both of which bear an asparagine at residue 98^{Gn}. Three mutations at distinct sites in the capping loop -A96T^{Gn} in ANDV Gn/Gc, and S88N^{Gn} and T90N^{Gn} in SNV Gn/Gc—afforded resistance to ADI-42898 (FIG. 30). Strikingly, all variations introduced an N-glycosylation motif (sequon; N-X-S/T) into the capping loop (FIG. 30A). rVSV-ANDV- and -SNV-Gn/Gc thus appear to “resurface” their capping loops to sterically occlude antibody recognition. These findings reinforce the conclusion that contacts with the polypeptide backbone of the highly variable Gn capping loop in divergent hantaviruses drive ADI-42898 recognition. Furthermore, the failure to isolate ADI-42898-mediated escape mutants located in Gc, which accounts for most of the buried surface area at the epitope-paratope interface (FIG. 22D), may reflect the viral fitness cost exacted by such mutations. N-glycosylation sequons have not been observed in the capping loop sequences of hantavirus isolates, perhaps because N-linked glycans at the vertices of the Gn/Gc tetramer are incompatible with lattice formation and virion budding. Such loop-resurfacing escape mutations may be deleterious for virus morphogenesis and thus unlikely to arise in response to ADI-42898 treatment of authentic hantaviruses.

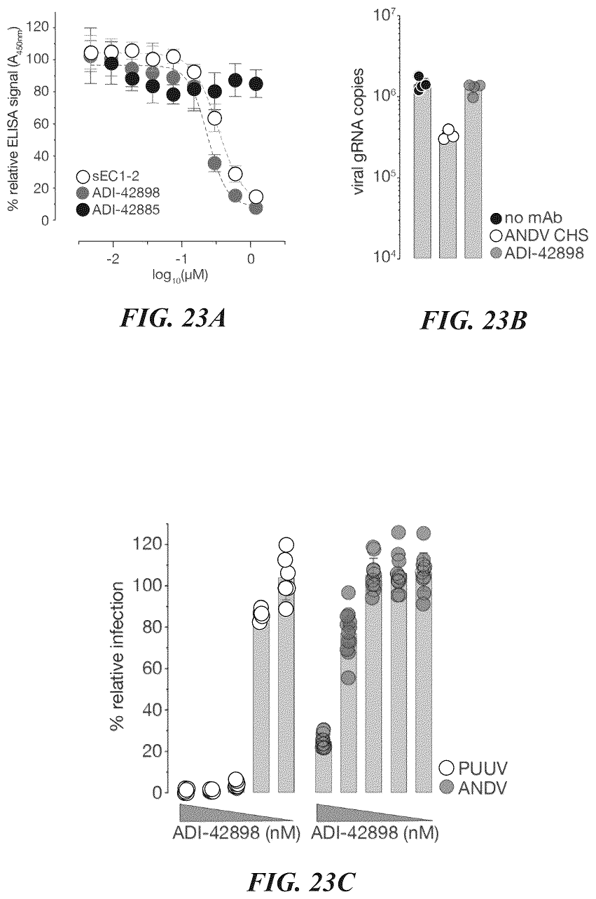

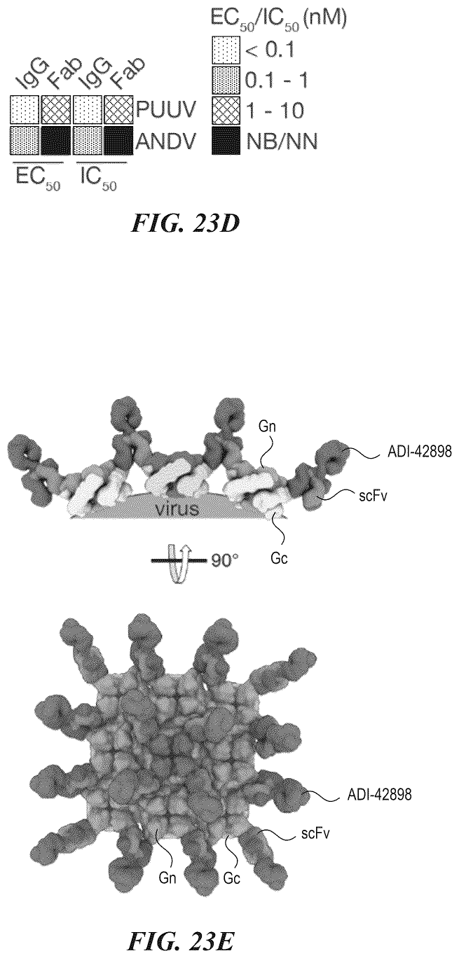

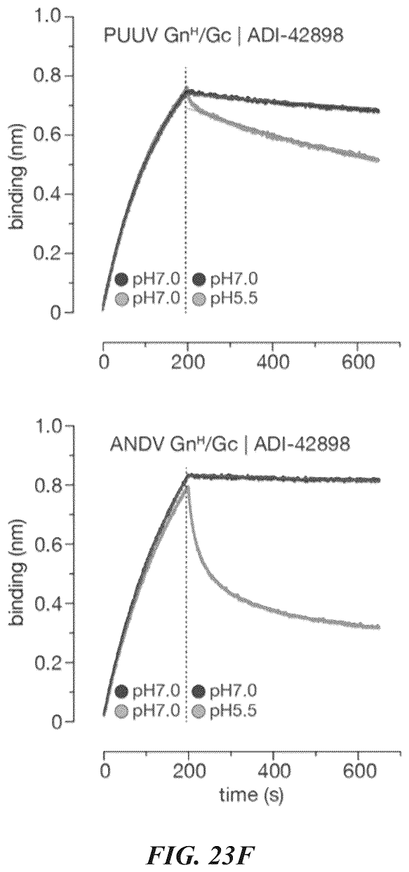

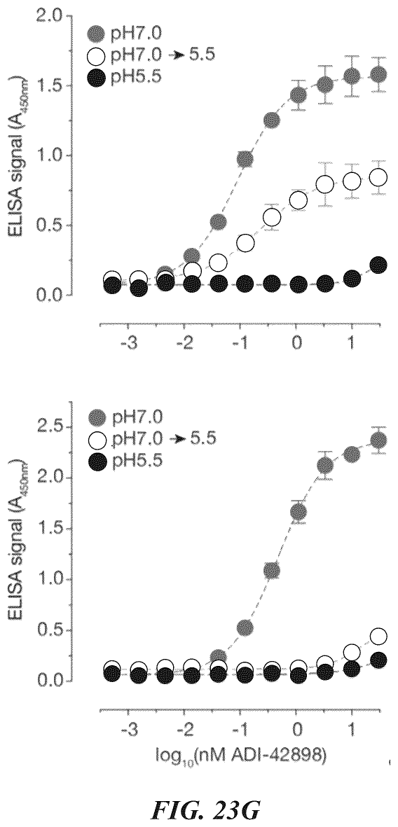

These structural studies suggest multiple non-mutually exclusive mechanisms by which ADI-42898 could block hantavirus entry. First, the apical location of its epitope in the Gn/Gc tetramer raised the possibility that ADI-42898 could affect virus-receptor binding. Consistent with this hypothesis, ADI-42898 prevented capture of rVSV-ANDV-Gn/Gc by a soluble form of the NWH-clade specific hantavirus receptor, protocadherin-1 (PCDH1) in a dose-dependent manner, whereas a broadly neutralizing Gc-targeting mAb (ADI-42885) had no effect (FIG. 23A). Despite this activity, ADI-42898 could not prevent viral attachment to PCDH1-bearing endothelial cells, likely because virions may exploit alternative attachment factors in cultured cells, as observed previously (FIG. 23B). Bound viral particles were enumerated by quantitative reverse transcription PCR (qPCR) detecting the open-reading frame encoding the VSV matrix protein M. CHS, convalescent hamster serum. Thus, the inhibition of virus-receptor interaction cannot fully explain the potent neutralizing activity of ADI-42898, at least in cell culture.

A second mechanism of action was considered: inhibition of the acid-induced Gn/Gc rearrangements required for viral membrane fusion through ADI-42898's recognition and stabilization of the GnGc pre-fusion complex (FIGS. 22A-22D). To test this hypothesis, the impact of ADI-42898 in a “fusion-infection” assay was measured, in which rVSV-Gn/Gc particles were forced to infect cells by fusing their viral membranes with the plasma membranes of endothelial cells and rVSV PUUV- or ANDV-Gn/Gc particles were pre-incubated with decreasing amounts of ADI-42898 (100 nM to 0.01 nM), followed by fusion-infection of HUVECs (FIG. 23C). ADI-42898 inhibited fusion-infection by rVSVs bearing both OWH and NWH Gn/Gc in a dose-dependent manner. This suggests that viral neutralization by ADI-42898 is primarily driven by its capacity to lock the Gn/Gc complex in a pre-fusion conformation, thereby preventing its acid-triggered dissociation, formation of the TMIR, and insertion of the TMIR into the endosomal membrane (FIG. 22A).

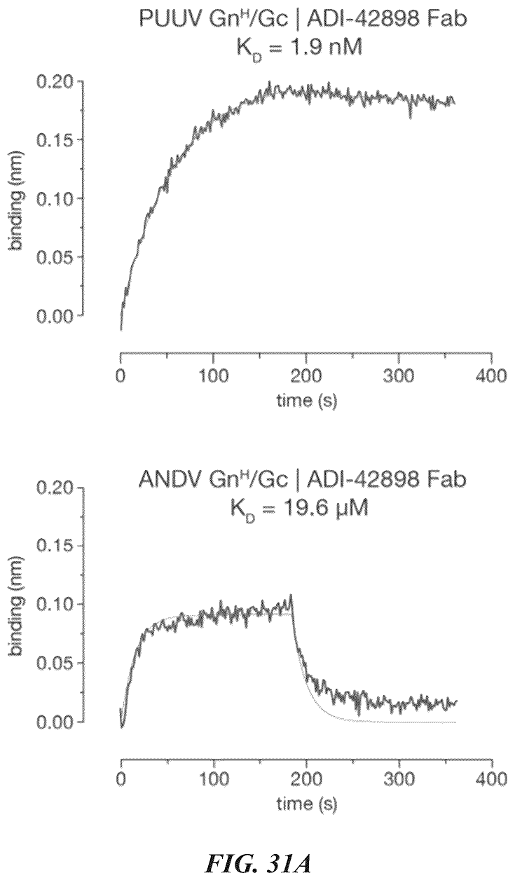

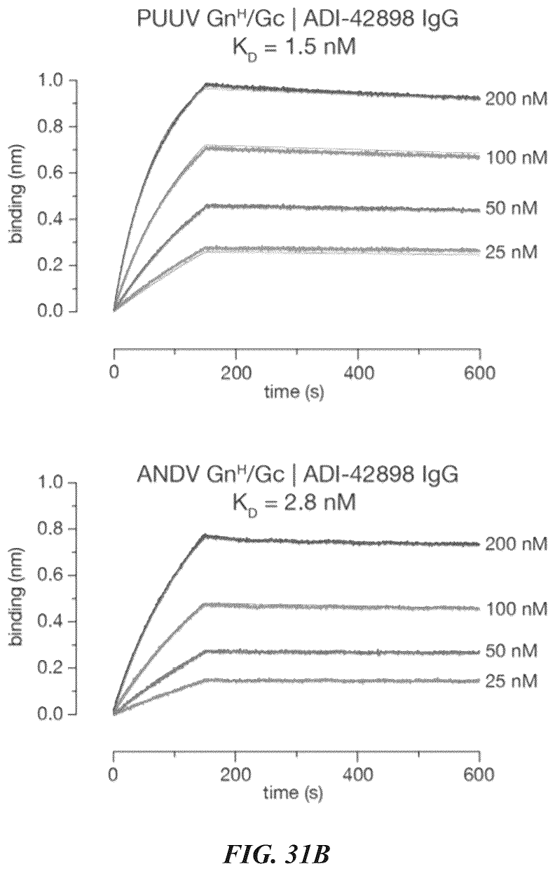

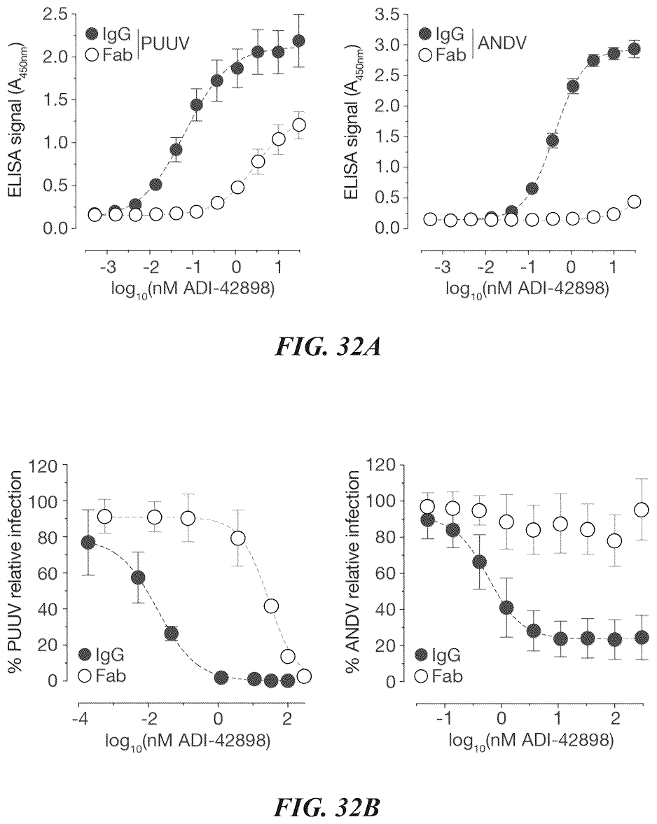

Although ADI-42898 was broadly active against hantavirus Gn/Gc in the preceding fusion-infection experiments, it was much more potent against PUUV than against ANDV Gn/Gc-initiated fusion (FIG. 23C), consistent with its relative neutralizing activities against viruses bearing these glycoproteins (FIG. 29). These differences may arise from the reduced binding affinity of ADI-42898 for ANDV Gn/Gc. Consistent with this hypothesis, an ADI-42898 monovalent antibody-binding fragment (Fab) bound much less well to ANDV Gn^{H}/Gc and ANDV virus-like particles (VLPs) than to its PUUV counterparts in BLI (FIGS. 31A and 31B) and ELISA-based assays. Biotinylated GnH/Gc was loaded onto probes and dipped into solutions of ADI-42898 Fab (100 nM for PUUV GnH/Gc, 500 nM for ANDV GnH/Gc (FIG. 31A) and ADI-42898 was loaded onto probes, which were dipped in solutions of GnH/Gc at the indicated concentration (FIG. 31B). More significant losses in neutralizing activity against ANDV than against PUUV were observed (FIGS. 23D, 32A, and 32B). Comparisons of Fabs to IgGs in these experiments reinforced the conclusion that bivalent binding is critical to the activity of ADI-42898 against both PUUV and ANDV, especially against the latter. Modeling of ADI-42898 IgG molecules bound to Gn/Gc tetramers (PDB: 6ZJM) and to lattices of tetramers (EMDB: 11236) suggested that ADI-42898 cannot bind bivalently to a single tetramer and must instead crosslink neighboring tetramers in the virion lattice (FIG. 23E).

The observations that ADI-42898's reduced binding affinity for ANDV Gn/Gc could be largely overcome through IgG-dependent avidity suggested that binding differences at the neutral pH of the extracellular milieu cannot alone explain ADI-42898's reduced neutralization potency against ANDV. Accordingly, it was next considered that ADI-42898:Gn/Gc immunocomplexes might be differentially labile at the acidic pH of late endosomes (~pH 5.5) where hantavirus membrane fusion is triggered and where ADI-42898 presumably deploys its fusion-blocking activity. To investigate this possibility, ADI-42898-coated BLI sensors were dipped into PUUV or ANDV Gn^{H}/Gc solutions at pH 7.0 to form immunocomplexes and then monitored their dissociation at pH 5.5 (FIG. 23F). Although both PUUV and ANDV Gn^{H}/Gc dissociated more rapidly from ADI-42898 at pH 5.5 than at pH 7.0, the ANDV Gn^{H}/Gc:ADI-42898 complex was markedly less stable at acidic pH than its PUUV counterpart (tin of 519 s vs. 1083 s for ANDV and PUUV Gn^{H}/Gc, respectively; FIG. 23F), possibly as a function of its lower binding affinity for ANDV Gn/Gc. Qualitatively similar results were obtained in a distinct assay format in which the pH-dependent release of ADI-42898 from immobilized ANDV and PUUV VLPs was assessed by ELISA (FIG. 23G). These findings suggest the following model for ADI-42898's mechanism of action: IgGs engage PUUV and ANDV virions efficiently in the extracellular space and transit with them into late endosomes. IgGs that remain bound to Gn/Gc upon endosomal acidification effect virus neutralization by locking the viral glycoprotein spikes in their pre-fusion conformation. The potency of ADI-42898 against ANDV is greatly diminished relative to PUUV because a more significant proportion of the antibody molecules bound to ANDV virions are released from their Gn/Gc targets in endosomes, thereby allowing more Gn/Gc spikes to undergo acid-triggered, fusion-driving conformational changes.

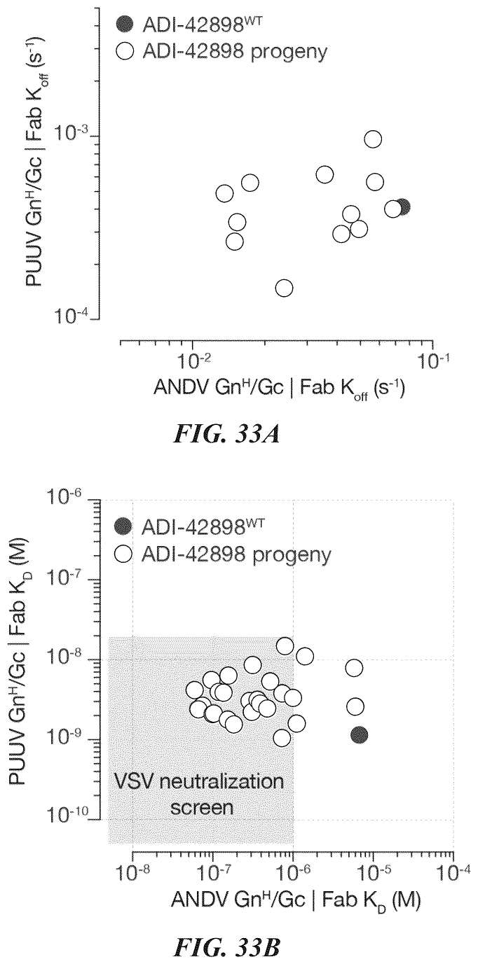

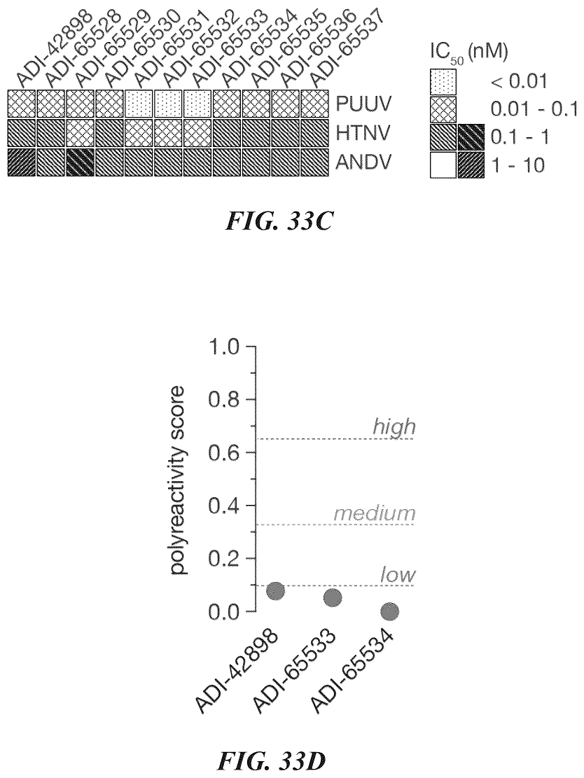

To enhance ADI-42898's potency against ANDV without compromising its cross-hantavirus clade breadth, affinity-matured ADI-42898 against ANDV Gn/Gc was assessed (see Methods and FIG. 33 for details). A panel of 1,020 Fabs bearing single CDR mutations was generated, which were screened for improved binding to ANDV Gn^{H}/Gc, and counter-screened for retention of binding to PUUV Gn^{H}/Gc. Twelve clones bearing eight distinct mutations showed improved off-rates to ANDV Gn^{H}/Gc and improved or similar off-rates to PUUV Gn^{H}/Gc (FIG. 33A). These mutations were then screened in combination, yielding 26 progeny Fabs with improved and parent-like binding affinity for ANDV and PUUV Gn^{H}/Gc, respectively (FIG. 33B). Of these, two affinity-matured antibodies, ADI-65533 and ADI-65534 were prioritized as they displayed great enhancements over ADI-42898^{WT} in neutralization potency against VSV bearing NWH Gn/Gc (ANDV) with no attendant loss in neutralization against their OWH counterparts (PUUV and HTNV).Twenty ADI-42898 progeny variants were tested for their neutralization activity against rVSV-ANDV-Gn/Gc (data not shown); 10 mAbs presenting the highest increase in neutralization potency were advanced for testing of their activity against divergent Gn/Gc proteins. An un-neutralized virus fraction is a residual normalized virus infection of >5% at the highest mAb concentration tested. (FIG. 33C).

Lastly, because in vitro engineering may lead to polyreactivity with potential risks off-target binding and accelerated clearance in vivo, ADI-65533 and ADI-65534 were evaluated in a polyreactivity assay that is predictive of serum half-life in humans. Both mAbs lacked polyreactivity in this assay, indicating a low risk for poor pharmacokinetic behavior (FIG. 33D). Accordingly, these affinity-matured variants of ADI-42898 were selected for further analysis.

ADI-65533 and ADI-65534 share two amino acid exchanges, G100V (mutation in the HCDR3) and C32V (mutation in LCDR1) (FIG. 24A). The polypeptide backbone at position G100 forms H bonds with the side chain of E768 in PUUV Gc (Q768 in ANDV Gc). The introduction of a V at this position might allow the formation of additional van der Waals interactions with residue Q768 in ANDV Gn/Gc (represented as lines in FIG. 24B, right panels). Residue C32 does not contact Gn/Gc but is part of a cluster of hydrophobic amino acids that includes F100d in CDR-H3 and thus may indirectly stabilize the conformation of that loop. ADI-65534 contains one additional substitution, T28E in CDR-H1. The side chains of T28 and E750 in PUUV Gc (Q750 in ANDV Gc) form a hydrogen bond; therefore, the mutation of T28 to the negatively charged E may afford the formation of two hydrogen bonds with Q750 and thereby strengthen ADI-65534's interaction with ANDV Gn/Gc. However, this may come at the expense of charge repulsion between ADI-65534 (E28) and PUUV Gn/Gc (E750) (FIG. 24B, middle panels).

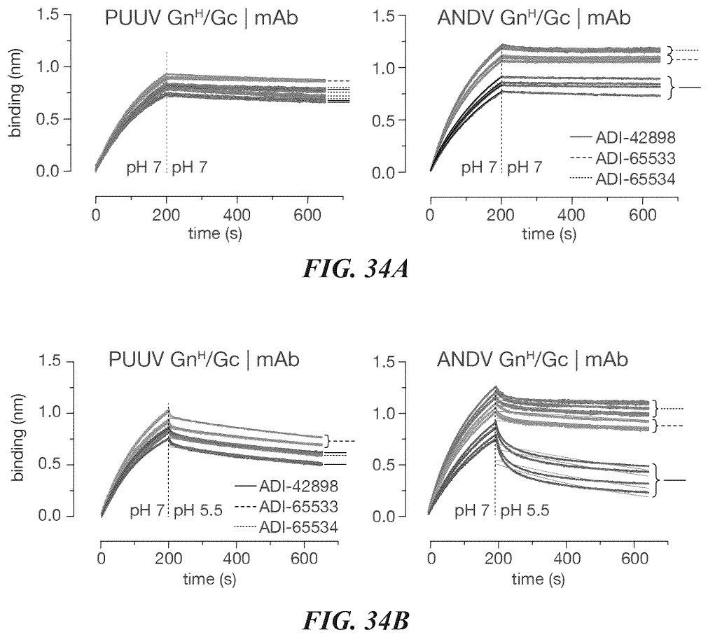

To investigate the impact of these amino acid substitutions on the antibodies' mechanisms of action, a kinetic map for mAb:Gn^{H}/Gc binding was generated by plotting association (kₒₙ at pH 7.0) and dissociation (k_{off} at pH 5.5) rates measured by BLI, where rates were isolated from BLI sensorgrams (FIGS. 24C and 34). Both affinity-matured mAbs bound better to ANDV Gn^{H}/Gc than did their ADI-42898 parent by virtue of their enhanced association at neutral pH and ~10-fold reduction in their dissociation at acidic pH (FIG. 24C, right panel). ADI-65533 bound to PUUV Gn^{H}/Gc as well as ADI-42898, but ADI-65534 bound less well at neutral pH, likely due to the repulsive effect of the E28/E750 pair (FIG. 24C, left panel).

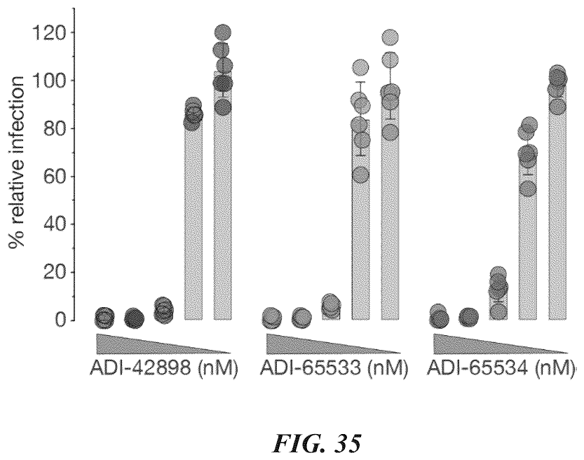

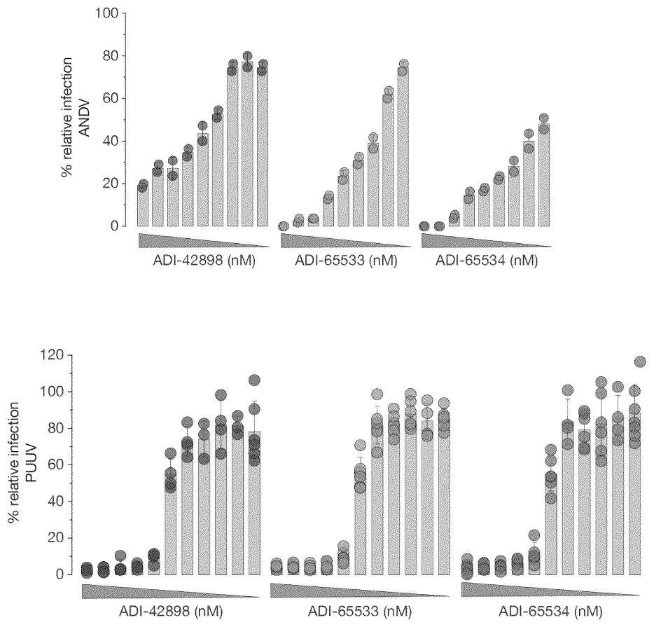

These findings were recapitulated in assays with viral particles: both affinity-matured variants bound better to ANDV VLPs than their ADI-42898 parent in a direct-binding ELISA at neutral pH and outcompeted ADI-42898 for rVSV-ANDV-Gn/Gc binding in a competition ELISA with PCDH1 (FIGS. 24D-24F). ADI-65534 also showed reduced acid pH-dependent release from immobilized ANDV VLPs (FIG. 24E). However, despite its slower dissociation from ANDV Gn^{H}/Gc in the BLI assay, ADI-65533 resembled ADI-42898 in its capacity to remain bound to ANDV VLPs at acidic pH, suggesting that additional variables associated with membrane-bound tetramers and tetramer lattices in viral particles influence the binding and dissociation of ADI-42898 and its variants. In line with its increased stability at neutral and low pH, ADI-65534 was superior to both ADI-65533 and ADI-42898 in its capacity to block ANDV Gn/Gc-mediated fusion-infection (IC₅₀: 15.4 nM vs. 2.3 nM for ADI-42898 and ADI-65534, respectively; FIG. 24G), while the affinity-matured mAbs had no advantage over ADI-42898 in blocking PUUV Gn/Gc-mediated fusion-infection (FIG. 35) (rVSV-PUUV-Gn/Gc particles were pre-incubated with mAb (100 nM to 0.01 nM), followed by fusion-infection of HUVECs). Together, these findings support the hypothesis that the mutations in the ADI-65534 variant increase its propensity to stay bound to and lock ANDV Gn/Gc in its pre-fusion conformation after viral internalization and endosomal acidification, thereby allowing for more efficient viral neutralization in endosomal compartments.

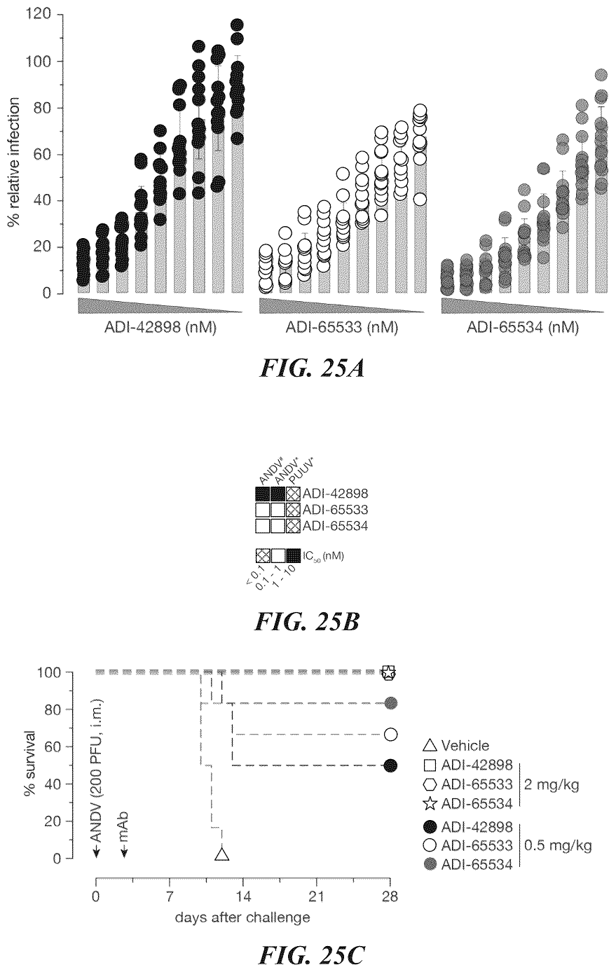

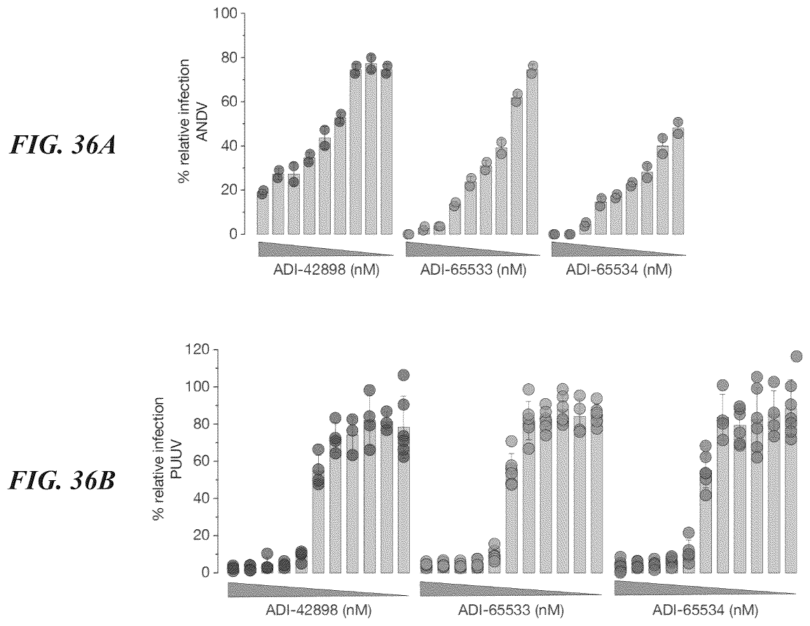

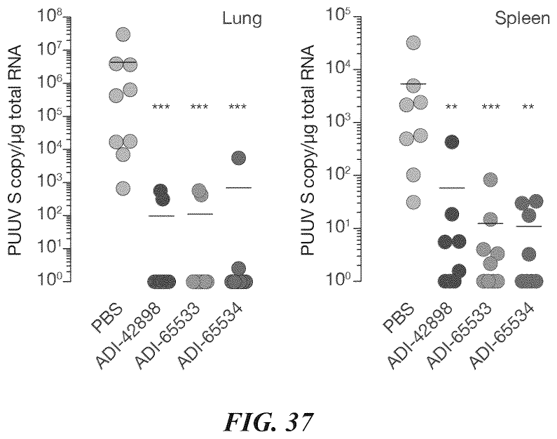

Both affinity-matured mAbs, ADI-65533 and ADI-65534, were tested for their cross-clade neutralization potency against authentic ANDV and PUUV (FIGS. 25A-25B and FIG. 36). NWH infection was blocked with higher efficiency by both affinity-matured mAbs than by their parent ADI-42898 variant as determined by two distinct in vitro neutralization assays. At the same time, affinity maturation did not impact the mAb's activity towards PUUV. The capacity of ADI-42898 and both affinity-matured variants ADI-65533 and ADI-65534 to protect Syrian hamsters against lethal ANDV challenge was assessed (FIG. 25C) (mortality of hamsters was monitored for 28 days). It was previously demonstrated that a single 6 mg/kg dose of ADI-42898 was highly protective when administered 3 days post-virus exposure. Here, all three antibodies were fully protective even at a lower 2 mg/kg dose, a testament to their cross-clade potency. However, by dosing down further, a breakthrough condition—0.5 mg/kg per animal—at which ADI-42898 provided only 50% protection but ADI-65534 afforded -85% protection. Significantly, affinity maturation of ADI-42898 to ADI-65534 did not compromise efficacy against PUUV since both antibodies afforded equivalent protection in a pre-exposure setting in a bank vole model of PUUV challenge (FIG. 37). Bank voles were administered a single 5 mg/kg dose (i.p.) of the indicated mAbs or PBS vehicle (n =8 from two independent experiments), followed by a challenge with PUUV/Suo [500 FFU, subcutaneous (s.c.)] at 4 h after mAb administration. Animals were euthanized 3 days after challenge, and organ viral RNA loads were determined by RT-qPCR detecting the PUUV S segment.

This illustrates at atomic resolution how a unique hantavirus cross-clade neutralizing antibody from a PUUV-experienced human donor, ADI-42898, recognizes a quaternary epitope in the tetrameric viral spike. Mechanistic studies fueled by these structural insights showed that ADI-42898 blocks viral membrane fusion by stapling together the spike's Gn and Gc subunits, which must rearrange for Gc to insert into the target membrane at acidic pH. These experiments also identify a limit of ADI-42898's activity against ANDV: it dissociates rapidly from the viral spike at the acidic pH of late endosomes where membrane fusion takes place. Correcting this liability through high-throughput antibody engineering yielded an optimized antibody variant, ADI-65534, with enhanced cross-clade neutralization potency and demonstrated in vivo efficacy that is unprecedented in the literature. ADI-65534 is a promising candidate for prophylaxis and therapy of HFRS and HCPS and represents an important step towards preparedness for future hantavirus outbreaks caused by known and novel hantaviruses.

### Materials and Methods

Cell lines. African green monkey kidney Vero and VeroE6 cells were cultured in a humidified 37° C., 5% CO₂ incubator in high-glucose Dulbecco's modified Eagle medium (DMEM, Life Technologies) supplemented with 2% fetal bovine serum (FBS, Atlanta Biologicals), 1% GlutaMAX, and 1% penicillin-streptomycin (both Life Technologies). Human hepatocarcinoma Huh.7.5.1 cells (a generous gift of J. Carette, Stanford University, CA) were maintained in high-glucose DMEM (Life Technologies) supplemented with 10% FBS (Atlanta Biologicals), 1% GlutaMAX (Life Technologies), 1% non-essential amino acids (Life Technologies) and 1% penicillin-streptomycin (Life Technologies). Human umbilical vein endothelial cells (HUVECs, Invitrogen, Cat #C0035C) were maintained in Human Large Vessel Endothelial (LVE) Cell Basal Medium supplemented with LVE supplements (Invitrogen) at 37° C. with 5% CO₂. Alternatively, HUVECs (ATCC, Cat #CRL-1730) were maintained in Endothelial cell Growth Medium (Lonza, Cat #CC-3156) supplemented with Endothelial cell growth medium-2 SingleQuots (Lonza, Cat #CC-4176) at 37° C. with 5% CO₂. FREESTYLE™ 293-F cells (Thermo Fisher Scientific, Cat #R79007) were grown in FREESTYLE™ 293 expression media, at 37° C. with 8% CO₂ in a shaking incubator. Mygla.REC.B bank vole epithelial cells (a gift of I. Eckerle, Geneva Centre for Emerging Viral Diseases, Switzerland) were cultured as described previously (23). Drosophila melanogaster S2 cells (Thermo Fisher Scientific, Cat #R69007) were maintained in serum-free insect cell medium (HyClone, GE Healthcare) supplemented with 1% penicillin-streptomycin in spinner flasks in a 28° C. humidified shaking incubator. Chinese hamster ovary (FREESTYLE™ CHO-S) cells (Gibco, Cat #R80007) were cultured in a humidified 37° C., 5% CO₂ incubator in CHOgro expression media (Mirus Bio, Cat #MIR 6200).

Recombinant Vesicular Stomatitis Viruses (rVSVs). rVSVs expressing enhanced green fluorescent protein (eGFP) and bearing Gn/Gc from HTNV, SEOV, DOBV, ANDV, SNV, and CHOV were engineered, rescued and propagated following published protocols (24-28). rVSV carrying a phosphoprotein P fused to mNeonGreen (mNG) and bearing PUUV GnGc has been described previously. Generation of viruses with an equivalent eGFP backbone decorated with LGNV Gn/Gc (GenBank code: AF005728) was performed by using standard molecular biology techniques. The identity and sequence of each virus-encoded Gn/Gc was determined by Sanger sequencing of rVSV genomic RNA-derived cDNAs. All viruses were propagated on Vero cells except rVSV-PUUV-Gn/Gc, which was amplified on Huh.7.5.1 cells.

VSV infectivity measurements and neutralization assays. Infectivity measurements and neutralization assays on confluent Vero cells with pre-titrated amounts of pseudotyped rVSV particles bearing either PUUV, HTNV, SEOV, DOBV, ANDV, SNV, CHOV, and LGNV Gn/Gc or, when indicated, rVSV-ANDV, and -SNV-Gn/Gc neutralization-escape mutants were performed as described previously (29). Virus neutralization data were subjected to nonlinear regression analysis to derive IC₅₀ values (4-parameter, variable slope sigmoidal dose-response equation constraining bottom parameters to 0-100; GraphPad Prism).

Authentic hantavirus infections and in vitro neutralization assays. PUUV strain Suonenjoki (PUUV/Suo) was propagated in Mygla.REC.B bank vole epithelial cells, whereas PUUV strain Kazan was grown in VeroE6 cells as described (30). VeroE6 cells were exposed to PUUV Kazan at a multiplicity of infection (MOI) of 0.1 to 1 of fluorescent focus-forming units (FFU) after a preincubation with indicated mAb concentrations. Viral infectivity was calculated by immunostaining of formaldehyde-fixed cells at 24 h after infection using polyclonal rabbit serum targeting the PUUV nucleocapsid protein N (NR-9675, BEI Resources). Plates were imaged with the Plate RUNNER HD (Trophos) and images were analyzed, and individual infected cells counted using the system's onboard software.

ANDV strain Chile-9717869 used for in vitro microneutralization assays was propagated and viral titers determined using VeroE6 cells as previously described (25, 31). For neutralization assays, pretitrated amounts of virus were mixed with serially diluted mAbs for 1 h before adding virus/mAb mixture to HUVECs at an MOI of 0.5. Viral infectivity was determined by immunostaining of formaldehyde-fixed and permeabilized cells at 48 h after infection with polyclonal rabbit sera specific for ANDV N (NR-9673; BEI Resources). Images were acquired on an Operetta high-content imaging device (PerkinElmer). Images were analyzed with a customized scheme built from image analysis functions present in the Harmonia software, and the percentage of infected cells was determined using the analysis functions.

For in vitro focus-reduction neutralization assays, 50-100 FFUs of ANDV strain Chile-7913 were mixed with serially diluted mAbs for 1 h before adding virus/mAb mixture to VeroE6 cells. Mixtures were removed after an incubation of 2 h at 37° C., cells washed with PBS, covered with an overlay media [equal volumes of 2.4% methylcellulose and 2× Eagle's Minimum Essential Medium (Gibco) supplemented with 5% FBS and 2× penicillin-streptomycin (Life Technologies)] and incubated at 37° C. for 7 days. Overlay was removed and viral infectivity was determined by immunostaining of methanol-fixed cells with polyclonal rabbit sera specific for ANDV N. Number of foci are counted by hand on a CKX53 microscope (Olympus LS). Virus neutralization data were subjected to nonlinear regression analysis to derive IC₅₀ values (four-parameter, variable slope sigmoidal dose-response equation constraining bottom parameters to 0 to 100; GraphPad Prism).

Preparation of virus-like particles (VLPs). Open-reading frames encoding C-terminally flag-tagged ANDV Gn/Gc or PUUV Gn/Gc were cloned into an expression plasmid (pCAGGS) under the control of a cytomegalovirus (CMV) promoter. Plasmids were transfected into FREESTYLE™ 293-F cells with polyethylenimine (Polysciences) and cells were grown for 72 h. Cell supernatant was clarified and passed through an 0.45-μm pore filter. VLPs were purified by ultracentrifugation at 23,600 rpm for 75 min in a SW28 rotor, resuspended in NT buffer (10 mM Tris-HCl [pH 7.5], 135 mM NaCl) and stored at −80° C.

Expression and purification of recombinant PUUV Gn^{H}/Gc. To obtain the PUUV Gn^{H}/Gc complex (strain Mu/07/1219, GenBank code: AJC50718, the approach described previously was followed (32). In short, a DNA fragment codon-optimized for expression in Drosophila melanogaster S2 cells (GenScript) encoding Gn^{H} (amino acid residues [aa]20 to 381) in frame with the Gc ectodomain (aa 659 to 1093) was generated. Both subunits were joined using a linker (GGSGLVPRGSGGGSGGGSWSHPQFEKGGGTGGGTLVPRGSGTGG) containing two thrombin cleavage sites (underlined) at either end and a Strep-tag sequence in the middle (bold). Gn^{H}/Gc encoding sequences were cloned into a modified pMT/BiP plasmid (Invitrogen) expressing them in frame with a C-terminal double Strep-tag. This plasmid was used to generate stable Drosophila melanogaster S2 cells, which were selected and maintained in spinner-flasks in serum-free insect cell medium containing puromycin (7 pg/ml). Protein expression was induced by adding 5 pM CdCl₂ and cell supernatant was harvested 5 days after induction, supplemented with avidin (10 pg/ml) and 0.1 M Tris-HCl (pH 8.0). Proteins were purified via Strep-Tactin affinity chromatography, followed by size exclusion chromatography (SEC) using a Superdex 200 10/30 column (GE Healthcare) in a buffer composed of 10 mM Tris-HCl (pH 8), 150 mM NaCl, and 1 mM EDTA.

Expression and purification of IgGs, scFv and Fabs. Full-length IgG1 proteins used for initial in vitro assays as well as germline-reverted (IGL) mAb constructs, WT:IGL chimeras and point mutants of ADI-42898 were expressed in S. cerevisiae cultures, as previously described (33). Briefly, yeast cultures were grown in 24-well plates in a shaking incubator at 30° C. After 6 days, the culture supernatants were harvested by centrifugation, and IgGs were purified by protein A affinity chromatography. Bound IgGs were eluted (200 mM acetic acid, 50 mM NaCl [pH 3.5]) and buffer-exchanged into phosphate-buffered saline (PBS, pH 7.0). Fab fragments were generated by digesting IgGs with papain for 2 h at 30° C., the reaction was terminated by the addition of iodoacetamide and passed over protein A agarose to remove Fc fragments and undigested IgG. The flow-through of the protein A agarose was passed over KappaSelect resin (Cytiva) and Fabs were eluted (200 mM acetic acid, 50 mM NaCl [pH 5.2]) into % volume of 2 M Hepes buffer (pH 8.0). To generate scFv fragments of ADI-42898, sequences encoding the V_{H} and V_{L} chains were joined together by a flexible (GS) linker and cloned into a modified pMT/BiP plasmid (Invitrogen). The antibody fragments were purified following the protocol described above in the “Expression and purification of recombinant PUUV Gn^{H}/Gc” section.

For authentic hantavirus neutralization assays, in vivo protection studies, and antibody biophysical characterizations, mAbs were produced as full-length IgG1 proteins in FREESTYLE™ CHO-S cells. Briefly, the V_{H}- and V_{L}-encoding genes were subcloned into vectors encoding human HC and LC constant regions and transiently transfected into CHO cells. After 6 days, the IgG-containing supernatants were harvested by centrifugation and purified by protein A-affinity chromatography. Bound IgGs were eluted, further purified by SEC to at least 95% purity and buffer-exchanged into 150 mM NaCl supplemented with 20 mM histidine (pH 6.0).

Crystallization and structure determination. The complex between PUUV Gn^{H}/Gc and the ADI-42898 scFv fragment was formed by incubation of Gn^{H}/Gc with a 1.5 molar excess of the scFv fragment for 1 h at 4° C. The complex was purified by SEC and eluted fractions corresponding to a monomeric and monodisperse complex were concentrated to 5-7 mg/ml using a Vivaspin centricon in 10 mM Tris-HCl (pH 8), 150 mM NaCl (buffer TN). The flexible linker joining Gn^{H} and Gc as well as the purification tag at the C-terminus of Gc were removed by adding 1 unit of biotinylated thrombin (Novagen) per mg of protein, and the mixture was incubated at 4° C. until the digestion was completed as judged by SDS-PAGE. The sample was used without further purification in crystallization trials at 18° C. using the sitting-drop vapor diffusion method. Crystals grew within 5-10 days in the presence of 25% (w/v) PEG 1000, 0.1 M Hepes buffer (pH 7.5) and were flash-frozen by immersion into a cryo-protectant containing the crystallization solution supplemented with 20% (v/v) glycerol, followed by rapid transfer into liquid nitrogen.

The structure of the complex was solved by molecular replacement (MR) with Phaser from the suite PHENIX using ANDV Gn^{H}/Gc (PDB code: 6Y5) and the variable domains of an unrelated antibody (PDB code: 4UT7) as search models. The final model was built by combining real space model building in Coot with reciprocal space refinement with phenix.refine to a final R(Rfree) of 21.2 (24.9%). The final model, which was validated with Molprobity (34), contained aa 22 to 377 of Gn and 652 to 1086 of Gc with two disordered regions: aa 196-199 and 1064-1075. Molecular graphic images were generated using PyMOL (Schrödinger).

Structural representation of Gn/Gc sequence conservation. To graphically represent the degree of sequence conservation of ADI-42898's epitope, 69 hantavirus Gn/Gc-encoding sequences were extracted and aligned with Clustal Omega (35). The GenBank codes for the sequences used in the analysis are: JX465398, JX465397, JX465402, JX465399, JX465400, JX465401, JF784178, NC_010708, EF641799, EF641806, HQ834696, FJ539167, EU929073, EU929074, HM015219, EF543526, JX465390, JX465391, JX465392, JX465393, JX465394, JX465395, JX465403, Y00386, AF143675, AB620029, AY675353, AB027115, GF796031, AY168577, AJ410616, GQ205412, AY961616, JQ082301, AF288298, GU592827, EF990916, L08756, GQ274938, HM756287, AB297666, AB433850, EF198413, AJ011648, EU072489, AJ011647, EU072488, Z69993, X55129, L36930, DQ177347, L39950, DQ284451, DQ285047, AB620104, AB620107, U26828, AB620101, U36801, U36802, AF030551, L33474, L37903, AF291703, AF028024, AF005728, FJ608550, AY363179, AF307323. This multiple sequence alignment was submitted to Espript (39) to calculate a conservation score at each amino acid position.

ELISA for Gn/Gc:antibody binding. VSV or VLP bearing ANDV or PUUV Gn/Gc were diluted in PBS (pH 7.4) and captured onto high binding half-area 96-well plates (Corning) in pre-titrated amounts. Plates were then washed with PBS and blocked with a blocking/binding buffer (PBS containing 3% bovine serum albumin [BSA]) for 1-2 h at 37° C. Blocked plates were exposed to a 3-fold dilution series of mAb or Fab fragment starting at 30 nM diluted in blocking/binding buffer for 1 h at 37° C. Plates were washed with PBS and mAb binding was detected with anti-human IgG conjugated to horseradish peroxidase (HRP, Thermo Fisher) for 1 h at 37° C. Fab fragment binding was detected with a biotin-conjugated CAPTURESELECT™ anti-human IgG-CH1 nanobody (Thermo Fisher) followed by an incubation with Streptavidin-HRP. Plates were washed, 1-STEP™ Ultra TMB-ELISA Substrate Solution (Thermo Fisher) was added and quenched with 0.5 M H₂SO₄. Absorbance was read at 450 nm on a Cytation 5 cell imaging multi-mode reader (BioTek).

VLP binding data were subjected to nonlinear regression analysis to derive EC₅₀ values (variable slope sigmoidal dose-response equation; GraphPad Prism). To measure the impact of acidic pH on Gn/Gc:mAb binding, VLPs were captured on 96-well plates and blocked as indicated above. When indicated, VLPs were exposed to a wash with PBS (pH 5.5) for 10 min at 37° C. After an incubation with a 3-fold dilution series of ADI-42898 at pH 7 for 1 h at 37° C., plates were washed with PBS, and VLPs (when indicated) were exposed to a wash with PBS (pH 5.5) for 10 min at 37° C. Following steps were all performed at neutral pH and as described above.

Selection of rVSV neutralization-escape mutants. Selection of escape mutants was performed as described previously (36). In short, rVSV-ANDV- and -SNV-Gn/Gc were serially passaged after rVSV pre-incubation with a concentration of ADI-42898 corresponding to its IC₉₀ value derived from neutralization assays, and then added to confluent monolayers of Vero cells. Supernatants were harvested and following several passages under mAb selection, supernatants were tested for viral neutralization escape. If resistance was evident, individual viral clones were plaque-purified on Vero cells, and their Gn/Gc-encoding sequences were determined.

Biolayer interferometry (BLI) assays. To determine binding properties of IgGs and Fabs to ANDV or PUUV Gn^{H}/Gc the OCTET RED™ system (ForteBio) was used. To measure the binding kinetics of Gn^{H}/Gc:mAb complexes, IgGs were captured on anti-human Fc biosensors (Sartorius) by dipping them into a mAb solution (10 μg/ml mAb in PBS) for 300 sec followed by a quenching step (0.2 mg/ml BSA in PBS) for 150 sec. IgG-loaded biosensors were exposed to PUUV or ANDV Gn^{H}/Gc (400 nM in buffer containing 100 mM Tr-HCl, 50 mM MES, 50 mM sodium acetate adjusted to pH 8.0, 7.5, 7.0, 6.5, 6.0, 5.5 or 5.0) for 500 sec; the values for the last 50 sec of the association phase were averaged and plotted using GraphPad Prism. To determine the binding affinity (K_{D}) of ANDV and PUUV Gn^{H}/Gc to ADI-42898, IgG-loaded biosensors were dipped into Gn^{H}/Gc solutions (200 nM to 25 nM) for 300 sec followed by a dissociation step. Experimental curves were fitted using a 1:1 binding model using GraphPad Prism.

To determine off-rates of Gn^{H}/Gc from Gn^{H}/Gc:mAb complexes formed on the biosensor tip surface, mAbs were immobilized on anti-human Fc biosensors and quenched as above. Next, biosensors were dipped into a Gn^{H}/Gc solution (100 nM diluted in PBS supplemented with BSA [0.2 mg/ml]) for 200 sec to monitor formation of the Gn^{H}/Gc:mAb complex. Dissociation of the complex was monitored by moving the sensors into a pH-adjusted buffer (20 mM MES, 150 mM NaCl, 0.2 mg/ml BSA at pH 7.0 or 5.5) for 400 sec. To account for potential nonspecific interactions, a sensor lacking mAb was dipped into Gn^{H}/Gc solutions and to account for dissociation of the mAb from the sensor over the course of the experiment, the functionalized sensor was dipped into buffer lacking analyte followed by double subtractions of the signals using the OCTET RED™ Analysis Studio software. BLI data was subjected to dissociation kinetic analysis to derive mAb dissociation rates at pH 5.5 (setting the non-specific binding term at infinite time to 0; GraphPad Prism). To derive association and dissociation rates at pH 7.0, BLI data was subjected to association-then-dissociation kinetic analysis (setting the non-specific binding term at infinite time to 0; GraphPad Prism).

To measure Fab fragment binding affinities, streptavidin capture biosensors (Sartorius) were used to immobilize biotinylated ANDV or PUUV Gn^{H}/Gc (100 nM) followed by an incubation with PBSF (1×PBS supplemented with 0.1% [w/v] BSA). The antigen-loaded biosensors were exposed to the Fabs at 100 nM for PUUV Gn^{H}/Gc and 500 nM for ANDV Gn^{H}/Gc. Dissociation of Fabs from the biosensor surface was determined by transferring them into PBSF. For binding responses >0.1 nm, experimental data were aligned, interstep corrected (to the association step) (colored traces) and fitted to a 1:1 binding model (gray traces) using the ForteBio Data Analysis Software, version 11.1 or Scrubber, version 2.0c (BioLogic Software).

Competition ELISA. Competition assays monitoring the capacity of ANDV Gn/Gc-specific mAbs or soluble EC1-2 protein to inhibit rVSV:PCDH1 binding were performed as previously described (24, 29). In brief, high-protein-binding 96-well ELISA plates (Corning) were coated with purified sEC1-2 protein (100 ng per well) overnight at 4° C. and blocked with blocking buffer (5% nonfat dry milk in PBS). The membranes of rVSV-ANDV-Gn/Gc particles were labeled with a short-chain phospholipid probe, functional-component spacer diacyl lipid conjugated to biotin (FSL-biotin; Sigma-Aldrich), as described (25). Biotinylated rVSV particles were incubated with the indicated dilutions of mAb, or sEC1-2 protein as a control in PBS for 1 h at room temperature before their addition to sEC1-2-coated plates. Bound rVSV was detected by incubation with a streptavidin-HRP conjugate diluted in binding buffer (PBS supplemented with 3% bovine serum albumin [BSA]). Plates were washed, 1-STEP™ Ultra TMB-ELISA Substrate Solution (Thermo Fisher) was added and quenched with 0.5 M H₂SO₄. Absorbance was read at 450 nm on a Cytation 5 cell imaging multi-mode reader (BioTek).

Virus attachment assay. rVSV-ANDV-Gn/Gc particles were incubated with mAb (300 nM) or serum from rVSV-ANDV-Gn/Gc-infected Syrian hamsters (a generous gift of D. Safronetz, Public Health Agency of Canada) at room temperature for 1 h. These rVSV:mAb complexes were spin-occulated onto prechilled HUVEC cells at 1,000 rpm and 4° C. for 10 min followed by an incubation on a cooling plate at 6° C. for 30 min. Unbound virus was removed by washing with cold PBS. Cells were harvested in PBS, RNA was isolated using PureLink RNA Mini Kit (Invitrogen) according to manufacturer's protocols and cell-bound virus was quantified by quantitative reverse transcription PCR (RT-qPCR) detecting the open-reading frame encoding the VSV matrix protein M. For the one-step RT-qPCR, the isolated RNA was mixed with VSV M-specific probe (10 μmol, 56-FAM/TGT GGC AGC/ZEN/CGC TGT ATC CCA TT/31ABkFQ), forward primer (25 μmol, AAT CGT CCG TTC AGA ACA TAC), reverse primer (25 μmol, GAA GAA CCC AAA AAA GCC AAG) and TAQPATH™ 1-Step RT-qPCR Master Mix, CG (Life Technologies, #A15299). All samples were run and analyzed using the QUANTSTUDIO™ 3 Real-Time PCR System device (Applied Biosystem). Serial ten-fold dilutions of a plasmid encoding the full-length genomic cDNA of rVSV-ANDV-Gn/Gc were used to generate a standard curve for quantification.

Fusion infection assay. VSV particles bearing ANDV or PUUV Gn/Gc were bound to pre-chilled HUVEC cells and fusion-infection assays were performed as previously described (29).

Generation and selection of affinity matured ADI-42898 variants. Single-position mutant affinity maturation variants were generated by introducing mutations into each position of the complementary determining regions (CDRs) 1, 2, and 3 of the V_{H} and V_{L} genes of ADI-42898 via overlap extension PCR. Briefly, forward or reverse-priming oligos (IDT) encoding ‘NNK’ or ‘MNN’ degenerate codons at a single site were used to amplify overlapping fragments. The fragments were assembled into full-length V_{H} or V_{L} regions via a second nested PCR with flanking primers which contained 40 base pairs of 5′ and 3′ homology to linearized yeast expression vectors to allow cloning by homologous recombination. The resulting 76 single-position libraries were then transformed into S. cerevisiae following the lithium acetate method for chemical transformation. Individual colonies from single-position libraries were validated by Sanger sequencing and ~13 individual yeast colonies from each single-position library were picked for production of 1,020 Fab fragments of ADI-42898 sequence variants.

Eight Fab variants carrying single-point mutations displayed improved ANDV Gn^{H}/Gc binding affinities; these beneficial mutations were combined to generate 47 combinatorial mutation variants which were produced as full-length IgGs in S. cerevisiae following established protocols (33). Resulting ADI-42898 variants were screened for further improvements in binding activity via BLI assays (ForteBio).

Polyreactivity assay. Antibody polyreactivity was measured as described previously (37). Briefly, soluble membrane protein and soluble cytosolic protein fractions obtained from CHO cells were biotinylated using NHS-LC-Biotin reagent (Thermo Fisher). Yeast cells presenting IgGs on their surface were incubated with biotinylated CHO cell preparations on ice, followed by two washes with ice-cold PBSF and incubated with a secondary labeling mix containing ExtrAvidin-R-PE (Sigma-Aldrich), anti-human LC-FITC (Southern Biotech), and propidium iodide (Invitrogen) for 15 min. Cells were washed with PBSF, resuspended, and analyzed by flow cytometry on a FACS Canto II (BD Biosciences). The mean fluorescence intensity of binding was normalized using control antibodies that display low, medium, or high polyreactivity to assess the non-specific binding.

Quantitative real-time PCR assay. After euthanasia and dissections, RNA was extracted from bank vole tissues using TRIzol Reagent (Thermo Fisher Scientific) according to the manufacturers instructions. RT-qPCR was performed as previously described (38). The results were normalized to the total RNA concentration of the samples and presented as S-segment copy number per microgram of total RNA.

Bank vole challenge studies. The neutralizing potential of human mAbs against PUUV/Suo challenge in vivo was investigated in an established experimental PUUV reservoir host model system (23). Adult bank voles (Myodes glareolus) were challenged with 500 FFU of PUUV/Suo by subcutaneous inoculation. In a prophylactic treatment regimen, bank voles were inoculated intraperitoneally with mAbs (~5 mg/kg) (or PBS as vehicle control) 4 h before challenge. The animals were euthanized 72 h after virus inoculation by cervical dislocation, after which RNA was isolated from lungs and spleen; virus RNA loads were assessed by PUUV S-segment RT-qPCR.

Syrian hamster challenge study. Syrian golden hamster (Mesocricetus auratus) studies were conducted under IACUC-approved protocols in compliance with the Animal Welfare Act, PHS Policy, and other applicable federal statutes and regulations elating to animals and experiments involving animals. The facility where these studies was conducted (USAMRIID) is accredited by the Association for Assessment and Accreditation of Laboratory Animal Care, International (AAALAC), and adheres to principles stated in the Guide for the Care and Use of Laboratory Animals, National Research Council, 2011.

Syrian golden hamsters (Envigo, catalog no. HsdHan AURA 089), 100 to 120 g, were exposed intramuscularly to 200 plaque-forming units (PFU) of ANDV strain Chile-9717869 diluted in 0.1 ml of PBS. On day 3 post-exposure, hamsters were treated intraperitoneally with indicated amounts (2 or 0.5 mg/kg) of mAb diluted in 0.8 ml of PBS. Hamsters were monitored daily for clinical signs of disease, morbidity, and mortality for 28 days. Moribund animals, defined as those experiencing severe respiratory distress or showing unresponsiveness, were humanely euthanized according to IACUC-approved criteria.

Statistical analysis. Statistical details for each experiment may be found in the respective figure legends. These include the number of replicates (n), measures of precision, and the statistical test used. Dose-response neutralization curves were subjected to nonlinear regression analysis to derive IC₅₀ values (four-parameter, variable slope sigmoidal dose-response equation constraining bottom parameters to 0 to 100). Dose-response binding curves were subjected to nonlinear regression analysis to derive EC₅₀ values (variable slope sigmoidal dose-response equation). Viral RNA load in animal tissue (FIG. 37) was analyzed using a Kruskal-Wallis test with Dunn's correction for multiple comparisons. All statistical analyses were carried out in GraphPad Prism.

Each and every publication and patent document referred to in the present technology is incorporated herein by reference in its entirety for all purposes to the same extent as if each such publication or document was specifically and individually indicated to be incorporated herein by reference.

While the present technology has been illustrated with reference to the specific examples and illustrations, changes may be made and equivalents may be substituted to adapt to a particular context or intended use as a matter of routine development and optimization and within the purview of one of ordinary skill in the art, thereby achieving benefits of the present technology without departing from the scope of what is claimed and their equivalents.

### Additional Embodiments

Various embodiments of the present technology are set forth below in paragraphs [0656] to [0713]:

1. An isolated antibody that binds to a spike protein of a hantavirus.

2. The isolated antibody of embodiment 1, wherein the isolated antibody comprises a heavy chain having a variable heavy (V_{H}) region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 1-5, 68, or 69.

3. The isolated antibody of embodiment 2, wherein the heavy chain comprises one or more complementarity determining regions (CDRs) (HCDRs) comprising or consisting of an amino acid sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NOS: 6-20 or 72-77.

4. The isolated antibody of any one of embodiments 1-3, wherein the isolated antibody comprises a light chain having a variable light (V_{L}) region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 21-25, 70, or 81.

5. The isolated antibody of embodiment 4, wherein the light chain comprises one or more CDRs (LCDRs) comprising or consisting of an amino acid sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NOS: 35-40 or 78-80.

6. The isolated antibody of embodiment 3, wherein the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 6, 11, and 16.

7. The isolated antibody of embodiment 3, wherein the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 7, 12, and 17.

8. The isolated antibody of embodiment 3, wherein the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 8, 13, and 18.

9. The isolated antibody of embodiment 3, wherein the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 9, 14, and 19.

10. The isolated antibody of embodiment 3, wherein the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 10, 15, and 20.

11. The isolated antibody of embodiment 3, wherein the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 72, 73, and 74.

12. The isolated antibody of embodiment 3, wherein the HCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 75, 76, and 77.

13. The isolated antibody of embodiment 5, wherein the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 26, 31, and 36.

14. The isolated antibody of embodiment 5, wherein the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 82, 31, and 36.

15. The isolated antibody of embodiment 5, wherein the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 27, 32, and 37.

16. The isolated antibody of embodiment 5, wherein the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 28, 33, and 38.

17. The isolated antibody of embodiment 5, wherein the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 29, 34, and 39.

18. The isolated antibody of embodiment 5, wherein the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 30, 35, and 40.

19. The isolated antibody of embodiment 5, wherein the LCDRs comprise or consist of amino acid sequences at least about 50%, at least about 55%, at least about 60%, at least about 65%, at least about 70%, at least about 75%, at least about 80%, at least about 85%, at least about 90%, at least about 95%, or at least about 100% identical to the amino acid sequences of SEQ ID NOS: 78, 79, and 80.

20. The isolated antibody of any one of embodiments 1-19, wherein the isolated antibody binds to an epitope located in an N-terminal (Gn) subunit (SEQ ID NO: 42) and/or a C-terminal (Gc) subunit (SEQ ID NO: 43) of the spike protein.

21. The isolated antibody of any one of embodiments 1-20, wherein the antibody binds to an epitope located in the capping loop of Gn (SEQ ID NO: 41).

22. The isolated antibody of any one of embodiments 1-21, wherein the antibody binds to a quaternary epitope encompassing both the Gn subunit and the Gc subunit.

23. The isolated antibody of any one of embodiments 1-22, wherein the epitope comprises one or more of E95, Q98 or S101 in the Gn subunit.

24. The isolated antibody of any one of embodiments 1-23, wherein the epitope comprises one or more of D682, S830, 1844, P852, D959, or S966 in the Gc subunit.

25. The isolated antibody of embodiment 22, wherein the antibody does not bind to Gn in the absence of Gc.

26. The isolated antibody of any one of embodiments 20-25, wherein the epitope comprises one or both of E88 and S101 of the Gn subunit.

27. The isolated antibody of any one of embodiments 1-26, wherein the isolated antibody binds to the spike protein of a hantavirus or an epitope of the hantavirus in an environment with an acidic pH.

28. The isolated antibody of any one of embodiments 1-26, wherein the isolated antibody neutralizes one or more hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV).

29. The isolated antibody of any one of embodiments 1 or 20-28, wherein the isolated antibody comprises:
a HCDR comprising:
   an HCDR1 comprising a sequence of any one of SEQ ID NOS: 6-10, 72, 75 or a variant HCDR1 in which 1, 2, 3, 4, or 5 amino acids are substituted relative to the sequence;
   an HCDR2 comprising a sequence of any one of SEQ ID NOS: 11-15, 73, 76 or a variant HCDR2 in which 1, 2, 3, 4, or 5 amino acids are substituted relative to the sequence; and an HCDR3 comprising a sequence of any one of SEQ ID NOS: 16-20, 74, 77, or a variant HCDR3 in which 1, 2, 3, 4, or 5 amino acids are substituted relative to the sequence; and
a LCDR comprising:
   an LCDR1 comprising a sequence of any one of SEQ ID NOS: 82, 26-30, 78, or a variant LCDR1 in which 1, 2, 3, 4, or 5 amino acids are substituted relative to the sequence;
   an LCDR2 comprising a sequence of any one of SEQ ID NOS: 31-35, 79 or a variant LCDR2 in which 1, 2, 3, 4, or 5 amino acids are substituted relative to the sequence; and
   an LCDR3 comprising a sequence of any one of SEQ ID NOS: 36-40, 80 or a variant LCDR3 in which 1, 2, 3, 4, or 5 amino acids are substituted relative to the sequence.

30. The isolated antibody of embodiment 29, wherein the isolated antibody comprises:
a HCDR comprising:
   an HCDR1 comprising a sequence of SEQ ID NOS: 72 or a variant HCDR1 in which 1, 2, 3, 4, or 5 amino acids are substituted relative to the sequence;
   an HCDR2 comprising a sequence of SEQ ID NOS: 73 or a variant HCDR2 in which 1, 2, 3, 4, or 5 amino acids are substituted relative to the sequence; and
   an HCDR3 comprising a sequence of SEQ ID NOS: 74, or a variant HCDR3 in which 1, 2, 3, 4, or 5 amino acids are substituted relative to the sequence; and
a LCDR comprising:
   an LCDR1 comprising a sequence of SEQ ID NOS: 78, or a variant LCDR1 in which 1, 2, 3, 4, or 5 amino acids are substituted relative to the sequence;
   an LCDR2 comprising a sequence of SEQ ID NOS: 79, or a variant LCDR2 in which 1, 2, 3, 4, or 5 amino acids are substituted relative to the sequence; and
   an LCDR3 comprising a sequence of SEQ ID NOS: 80, or a variant LCDR3 in which 1, 2, 3, 4, or 5 amino acids are substituted relative to the sequence.

31. The isolated antibody of embodiment 29, wherein the isolated antibody comprises:
a heavy chain complementarity-determining region (HCDR) comprising:
   an HCDR1 comprising a sequence of SEQ ID NOS: 75 or a variant HCDR1 in which 1, 2, 3, 4, or 5 amino acids are substituted relative to the sequence;
   an HCDR2 comprising a sequence of SEQ ID NOS: 76 or a variant HCDR2 in which 1, 2, 3, 4, or 5 amino acids are substituted relative to the sequence; and
   an HCDR3 comprising a sequence of SEQ ID NOS: 77, or a variant HCDR3 in which 1, 2, 3, 4, or 5 amino acids are substituted relative to the sequence; and
a light chain complementarity-determining region (LCDR) comprising:
   an LCDR1 comprising a sequence of SEQ ID NOS: 78, or a variant LCDR1 in which 1, 2, 3, 4, or 5 amino acids are substituted relative to the sequence;
   an LCDR2 comprising a sequence of SEQ ID NOS: 79, or a variant LCDR2 in which 1, 2, 3, 4, or 5 amino acids are substituted relative to the sequence; and
   an LCDR3 comprising a sequence of SEQ ID NOS: 80, or a variant LCDR3 in which 1, 2, 3, 4, or 5 amino acids are substituted relative to the sequence.

32. The isolated antibody of embodiment 1 or 29, wherein the V_{H} comprises an amino acid sequence having at least 95% identity to a V_{H} of any one of SEQ ID NOS: 1-5, and the V_{L} comprises an amino sequence having at 95% identity to a V_{L} of any one of SEQ ID NOS: 21-25; wherein the V_{H} preferably comprises an amino acid sequence having at least 95% identity to a V_{H} of SEQ ID NO: 1, and the V_{L} preferably comprises an amino sequence having at 95% identity to a V_{L} of SEQ ID NO: 21.

33. The isolated antibody of embodiment 1 or 29, wherein the V_{H} comprises an amino acid sequence having at least 95% identity to a V_{H} of SEQ ID NOS: 68 or 69 and the V_{L} comprises an amino sequence having at 95% identity to a V_{L} of SEQ ID NOS: 70.

34. The isolated antibody of embodiment 1 or 29, wherein the antibody comprises:
a V_{H} region comprising amino acid sequence SEQ ID NO: 1 and a V_{L} region comprising amino acid sequence SEQ ID NO: 21;
a V_{H} region comprising amino acid sequence SEQ ID NO: 2 and a V_{L} region comprising amino acid sequence SEQ ID NO: 22;
a V_{H} region comprising amino acid sequence SEQ ID NO: 3 and a V_{L} region comprising amino acid sequence SEQ ID NO: 23;
a V_{H} region comprising amino acid sequence SEQ ID NO: 4 and a V_{L} region comprising amino acid sequence SEQ ID NO: 24;
a V_{H} region comprising amino acid sequence SEQ ID NO: 5 and a V_{L} region comprising amino acid sequence SEQ ID NO: 25;
a V_{H} region comprising amino acid sequence SEQ ID NO: 68 and a V_{L} region comprising amino acid sequence SEQ ID NO: 70; or
a V_{H} region comprising amino acid sequence SEQ ID NO: 69 and a V_{L} region comprising amino acid sequence SEQ ID NO: 70.

35. The isolated antibody of embodiment 1, wherein the antibody comprises:
a heavy chain variable (V_{H}) region and a light chain variable (V_{L}) region, wherein:
   (a) the V_{H} region has at least 70% identity to SEQ ID NO: 1; and comprises a CDR1 of SEQ ID NO:6, or the CDR1 of SEQ ID NO: 6 in which 1, 2, 3, 4, or 5 amino acids are substituted; a CDR2 of SEQ ID NO: 11, or the CDR2 of SEQ ID NO: 11 in which 1, 2, 3, 4, or 5 amino acids are substituted; a CDR3 of SEQ ID NO: 16 or the CDR3 of SEQ ID NO: 16 in which 1, 2, 3, 4, or 5 are substituted; and
   (b) the V_{L} region has at least 70% identity to SEQ ID NO: 21, and comprises a CDR1 of SEQ ID NO: 82 or 26 or the CDR1 of SEQ ID NO: 82 or 26 in which 1, 2, 3, 4, or 5 amino acids are substituted; a CDR2 of SEQ ID NO: 31, or the CDR2 of SEQ ID NO: 31 in which 1, 2, 3, 4, or 5 amino acids are substituted; a CDR3 of SEQ ID NO: 36 or the CDR3 of SEQ ID NO: 36 in which 1, 2, 3, 4, or 5 are substituted.

36. The isolated antibody of embodiment 1, wherein the antibody comprises:
a heavy chain variable (V_{H}) region and a light chain variable (V_{L}) region, wherein:
   (a) the V_{H} region has at least 70% identity to SEQ ID NO: 68; and comprises a CDR1 of SEQ ID NO:72, or the CDR1 of SEQ ID NO: 72 in which 1, 2, 3, 4, or 5 amino acids are substituted; a CDR2 of SEQ ID NO: 73, or the CDR2 of SEQ ID NO: 73 in which 1, 2, 3, 4, or 5 amino acids are substituted; a CDR3 of SEQ ID NO: 74 or the CDR3 of SEQ ID NO: 74 in which 1, 2, 3, 4, or 5 are substituted; and
   (b) the V_{L} region has at least 70% identity to SEQ ID NO: 70, and comprises a CDR1 of SEQ ID NO: 78 or the CDR1 of SEQ ID NO: 78 in which 1, 2, 3, 4, or 5 amino acids are substituted; a CDR2 of SEQ ID NO: 79, or the CDR2 of SEQ ID NO: 79 in which 1, 2, 3, 4, or 5 amino acids are substituted; a CDR3 of SEQ ID NO: 80 or the CDR3 of SEQ ID NO: 80 in which 1, 2, 3, 4, or 5 are substituted.

37. The isolated antibody of embodiment 1, wherein the antibody comprises:
a heavy chain variable (V_{H}) region and a light chain variable (V_{L}) region, wherein:
   (a) the V_{H} region has at least 70% identity to SEQ ID NO: 69; and comprises a CDR1 of SEQ ID NO:75, or the CDR1 of SEQ ID NO: 75 in which 1, 2, 3, 4, or 5 amino acids are substituted; a CDR2 of SEQ ID NO: 76, or the CDR2 of SEQ ID NO: 76 in which 1, 2, 3, 4, or 5 amino acids are substituted; a CDR3 of SEQ ID NO: 77 or the CDR3 of SEQ ID NO: 77 in which 1, 2, 3, 4, or 5 are substituted; and
   (b) the V_{L} region has at least 70% identity to SEQ ID NO: 70, and comprises a CDR1 of SEQ ID NO: 78 or the CDR1 of SEQ ID NO: 78 in which 1, 2, 3, 4, or 5 amino acids are substituted; a CDR2 of SEQ ID NO: 79, or the CDR2 of SEQ ID NO: 79 in which 1, 2, 3, 4, or 5 amino acids are substituted; a CDR3 of SEQ ID NO: 80 or the CDR3 of SEQ ID NO: 80 in which 1, 2, 3, 4, or 5 are substituted.

38. The isolated antibody of any one of embodiments 1-37, wherein the antibody comprises an HCDR1, HCDR2, HCDR3, LCDR1, LCDR2, and LCDR3 of an antibody designated as ADI-42898, ADI-42877, ADI-42098, ADI-42885, ADI-37236, ADI-65533, ADI-65534 or a variant thereof in which at least one, two, three, four, five, or all six of the CDRs contain 1 or 2 amino acid substitutions compared to the corresponding CDR.

39. The isolated antibody of any one of embodiments 1-37, wherein the antibody comprises a heavy chain variable region and a light chain variable region of an antibody designated as ADI-42898, ADI-42877, ADI-42098, ADI-42885, ADI-37236, ADI-65533, ADI-65534 or a variant thereof, wherein the variant comprises a heavy chain variable region having a sequence that is at least 95% identical to that of the corresponding heavy chain variable region and a light chain variable region having a sequence that is at least 95% identical to the corresponding light chain variable region.

40. The isolated antibody of any one of embodiments 1-39, wherein at least 1 or 2 of the substitutions are conservative substitutions; at least 50% of the substitutions are conservative substitutions; or all of the substitutions are conservative substitutions.

41. The isolated antibody of any one of embodiments 1-40, wherein the antibody is a bispecific antibody or a multispecific antibody.

42. The isolated antibody of any one of embodiments 1-41, wherein the immunoglobulin (Ig) class of the isolated antibody is IgG or IgM.

43. The isolated antibody of any one of embodiments 1-42, wherein the Fc subclass of the isolated antibody is selected from the group consisting of hIgG1, hIgG2, and hIgG4.

44. An antibody that binds to the same epitope as the antibody of any one of embodiments 1-40.

45. An antibody that competes for binding of the epitope of any one of embodiments 20-27 with the antibody of any one of embodiments 1-22.

46. The antibody of any one of embodiments 1-45, wherein the antibody is a non-natural antibody.

47. A recombinant nucleic acid encoding an antibody according to any of embodiments 1-46.

48. An expression vector comprising a polynucleotide encoding the V_{H} region and/or the V_{L} region of the antibody of any one of embodiments 1-46.

49. A host cell that comprises an expression vector of embodiment 48.

50. A polypeptide comprising
(1) a V_{H} sequence having at least 70% amino acid sequence identity to SEQ ID NO:1 and/or
(2) a V_{L} sequence having at least 70% amino acid sequence identity to SEQ ID NO:21.

51. A polypeptide comprising
(1) a V_{H} sequence having at least 70% amino acid sequence identity to SEQ ID NO:68 and/or
(2) a V_{L} sequence having at least 70% amino acid sequence identity to SEQ ID NO:70.

52. A polypeptide comprising
(1) a V_{H} sequence having at least 70% amino acid sequence identity to SEQ ID NO:69 and/or
(2) a V_{L} sequence having at least 70% amino acid sequence identity to SEQ ID NO:70.

53. A pharmaceutical composition comprising the isolated antibody of any one of embodiments 1-46 and a pharmaceutically acceptable carrier.

54. The pharmaceutical composition of embodiment 53, wherein the pharmaceutical composition comprises two or more different antibodies of any one of embodiments 1-25.

55. A method of inducing an immune response and/or treating a hantavirus infectious disease, the method comprising administering an effective amount of a pharmaceutical composition of embodiment 53 to a subject in need thereof.

56. The method of embodiment 55, wherein the infectious disease is selected from hantavirus cardiopulmonary syndrome (HCPS) and hemorrhagic fever with renal syndrome (HFRS).

57. Use of the pharmaceutical composition of embodiment 53 or 54 for inducing an immune response and/or treating a hantavirus infectious disease in a subject in need thereof.

58. The use of embodiment 57, wherein the infectious disease is selected from HCPS and HFRS.

### REFERENCES

1. P. F. Kerkman, A. Demstedt, L. Tadala, E. Mittier, M. Dannborg, C. Sundling, K. T. Maleki, J. Tauriainen, A. Tuiskunen-Back, J. Wigren Byström, P. Ocaya, T. Thunberg, R. K. Jangra, G. Romen-Sosa, P. Guardado-Calvo, F. A. Rey, J. Klingström, K. Chandran, A. Puhar, C. Ahlm, M. N. Forsell, Generation of plasma cells and CD27-IgD-B cells during hantavirus infection is associated with distinct pathological findings. Clin. Transl. Immunology. 10, e1313 (2021).
2. L. M. Kleinfelter, R. K. Jangra, L. T. Jae, A. S. Herbert, E. Mittler, K. M. Stiles, A. S. Wirchnianski, M. Kielian, T. R. Brummelkamp, J. M. Dye, K. Chandran, Haploid genetic screen reveals a profound and direct dependence on cholesterol for hantavirus membrane fusion. MBio. 6, e00801 (2015).
3. M. M. Slough, K. Chandran, R. K. Jangra, Two point mutations in old world hantavirus glycoproteins afford the generation of highly infectious recombinant vesicular stomatitis virus vectors. MBio. 10 (2019), doi:10.1128/mBio.02372-18.
4. M. Jia, R. A. Liberatore, Y. Guo, K.-W. Chan, R. Pan, H. Lu, E. Waltari, E. Mittler, K. Chandran, A. Finzi, D. E. Kaufmann, M. S. Seaman, D. D. Ho, L. Shapiro, Z. Sheng, X.-P. Kong, P. D. Bieniasz, X. Wu, VSV-Displayed HIV-1 Envelope Identifies Broadly Neutralizing Antibodies Class-Switched to IgG and IgA. Cell Host Microbe. 27, 963-975.e5 (2020).
5. T. Strandin, T. Smura, P. Ahola, K. Aaltonen, T. Sironen, J. Hepojoki, I. Eckerle, R. G. Ulrich, O. Vapalahti, A. Kipar, K. M. Forbes, Orthohantavirus Isolated in Reservoir Host Cells Displays Minimal Genetic Changes and Retains Wild-Type Infection Properties. Viruses. 12 (2020), doi:10.3390/v12040457.
6. R. K. Jangra, A. S. Herbert, R. Li, L. T. Jae, L. M. Kleinfelter, M. M. Slough, S. L. Barker, P. Guardado-Calvo, G. Romen-Sosa, M. E. Dieterle, A. I. Kuehne, N. A. Muena, A. S. Wirchnianski, E. K. Nyakatura, J. M. Fels, M. Ng, E. Mittler, J. Pan, S. Bharrhan, A. Z. Wec, K. Chandran, Protocadherin-1 is essential for cell entry by New World hantaviruses. Nature. 563, 559-563 (2018).
7. A. C. Wong, R. G. Sandesara, N. Mulherkar, S. P. Whelan, K. Chandran, A forward genetic strategy reveals destabilizing mutations in the Ebolavirus glycoprotein that alter its protease dependence during cell entry. J. Virol. 84, 163-175 (2010).
8. M. E. Dieterle, C. Soli-Riera, C. Ye, S. M. Goodfellow, E. Mittier, E. Kasikci, S. B. Bradfute, J. Klingström, R. K. Jangra, K. Chandran, Genetic depletion studies inform receptor usage by virulent hantaviruses in human endothelial cells. eLife. 10 (2021), doi:10.7554/eLife.69708.
9. A. Lundkvist, Y. Cheng, K. B. Sjölander, B. Niklasson, A. Vaheri, A. Plyusnin, Cell culture adaptation of Puumala hantavirus changes the infectivity for its natural reservoir, Clethrionomys glareolus, and leads to accumulation of mutants with altered genomic RNA S segment. J. Virol. 71, 9515-9523 (1997).
10. A. Papa, K. Nemirov, H. Henttonen, J. Niemimaa, A. Antoniadis, A. Vaheri, A. Plyusnin, O. Vapalahti, Isolation of Dobrava virus from Apodemus flavicollis in Greece. J. Clin. Microbiol. 39, 2291-2293 (2001).
11. J. W. Hooper, T. Larsen, D. M. Custer, C. S. Schmaljohn, A lethal disease model for hantavirus pulmonary syndrome. Virology. 289, 6-14 (2001).
12. H. W. Lee, P. W. Lee, K. M. Johnson, Isolation of the etiologic agent of Korean Hemorrhagic fever. J. Infect. Dis. 137, 298-308 (1978).
13. A. L. Schmaljohn, D. Li, D. L. Negley, D. S. Bressler, M. J. Turell, G. W. Korch, M. S. Ascher, C. S. Schmaljohn, Isolation and initial characterization of a newfound hantavirus from California. Virology. 206, 963-972 (1995).
14. A. Serris, R. Stass, E. A. Bignon, N. A. Muena, J.-C. Manuguerra, R. K. Jangra, S. Li, K. Chandran, N. D. Tischler, J. T. Huiskonen, F. A. Rey, P. Guardado-Calvo, The hantavirus surface glycoprotein lattice and its fusion control mechanism. Cell. 183, 442-456.e16 (2020).
15. T. Tiller, E. Meffre, S. Yurasov, M. Tsuiji, M. C. Nussenzweig, H. Wardemann, Efficient generation of monoclonal antibodies from single human B cells by single cell RT-PCR and expression vector cloning. J. Immunol. Methods. 329, 112-124 (2008).
16. R. D. Gietz, R. A. Woods, Yeast transformation by the LiAc/SS Carrier DNA/PEG method. Methods Mol. Biol. 313, 107-120 (2006).
17. A. Z. Wec, D. Wrapp, A. S. Herbert, D. P. Maurer, D. Hasiwanter, M. Sakharkar, R. K. Jangra, M. E. Dieterle, A. Lilov, D. Huang, L. V. Tse, N. V. Johnson, C.-L. Hsieh, N. Wang, J. H. Nett, E. Champney, I. Bumina, M. Brown, S. Lin, M. Sinclair, L. M. Walker, Broad neutralization of SARS-related viruses by human monoclonal antibodies. Science. 369, 731-736 (2020).
18. Z. A. Bomholdt, H. L. Turner, C. D. Murin, W. Li, D. Sok, C. A. Souders, A. E. Piper, A. Goff, J. D. Shamblin, S. E. Wollen, T. R. Sprague, M. L. Fusco, K. B. J. Pommert, L. A. Cavacini, H. L. Smith, M. Klempner, K. A. Reimann, E. Krauland, T. U. Gerngross, K. D. Wittrup, L. M. Walker, Isolation of potent neutralizing antibodies from a survivor of the 2014 Ebola virus outbreak. Science. 351, 1078-1083 (2016).
19. A. Z. Wec, A. S. Herbert, C. D. Murin, E. K. Nyakatura, D. M. Abelson, J. M. Fels, S. He, R. M. James, M.-A. de La Vega, W. Zhu, R. R. Bakken, E. Goodwin, H. L. Turner, R. K. Jangra, L. Zeitlin, X. Qiu, J. R. Lai, L. M. Walker, A. B. Ward, J. M. Dye, Z. A. Bomholdt, Antibodies from a Human Survivor Define Sites of Vulnerability for Broad Protection against Ebolaviruses. Cell. 169, 878-890.e15 (2017).
20. A. Punjani, J. L. Rubinstein, D. J. Fleet, M. A. Brubaker, cryoSPARC: algorithms for rapid unsupervised cryo-EM structure determination. Nat. Methods. 14, 290-296 (2017).
21. J. Wigren Byström, J. Naslund, F. Trulsson, M. Evander, O. Wesula Lwande, C. Ahlm, G. Bucht, Quantification and kinetics of viral RNA transcripts produced in Orthohantavirus infected cells. Virol. J. 15, 18 (2018).
22. L. Shehata, D. P. Maurer, A. Z. Wec, A. Lilov, E. Champney, T. Sun, K. Archambault, I. Bumina, H. Lynaugh, X. Zhi, Y. Xu, L. M. Walker, Affinity maturation enhances antibody specificity but compromises conformational stability. Cell Rep. 28, 3300-3308.e4 (2019).
23. T. Strandin, T. Smura, P. Ahola, K. Aaltonen, T. Sironen, J. Hepojoki, I. Eckerle, R. G. Ulrich, O. Vapalahti, A. Kipar, K. M. Forbes, Orthohantavirus Isolated in Reservoir Host Cells Displays Minimal Genetic Changes and Retains Wild-Type Infection Properties., Viruses 12 (2020), doi:10.3390/v12040457.
24. R. K. Jangra, A. S. Herbert, R. Li, L. T. Jae, L. M. Kleinfelter, M. M. Slough, S. L. Barker, P. Guardado-Calvo, G. Romen-Sosa, M. E. Dieterle, A. I. Kuehne, N. A. Muena, A. S. Wirchnianski, E. K. Nyakatura, J. M. Fels, M. Ng, E. Mittier, J. Pan, S. Bharrhan, A. Z. Wec, J. R. Lai, S. S. Sidhu, N. D. Tischler, F. A. Rey, J. Moffat, T. R. Brummelkamp, Z. Wang, J. M. Dye, K. Chandran, Protocadherin-1 is essential for cell entry by New World hantaviruses., Nature 563, 559-563 (2018).
25. L. M. Kleinfelter, R. K. Jangra, L. T. Jae, A. S. Herbert, E. Mittler, K. M. Stiles, A. S. Wirchnianski, M. Kielian, T. R. Brummelkamp, J. M. Dye, K. Chandran, Haploid genetic screen reveals a profound and direct dependence on cholesterol for hantavirus membrane fusion., MBio 6, e00801 (2015).
26. M. M. Slough, K. Chandran, R. K. Jangra, Two point mutations in old world hantavirus glycoproteins afford the generation of highly infectious recombinant vesicular stomatitis virus vectors., MBio 10 (2019), doi:10.1128/mBio.02372-18.
27. A. C. Wong, R. G. Sandesara, N. Mulherkar, S. P. Whelan, K. Chandran, A forward genetic strategy reveals destabilizing mutations in the Ebolavirus glycoprotein that alter its protease dependence during cell entry., J. Virol. 84, 163-175 (2010).
28. M. E. Dieterle, C. Solc-Riera, C. Ye, S. M. Goodfellow, E. Mittier, E. Kasikci, S. B. Bradfute, J. Klingström, R. K. Jangra, K. Chandran, Genetic depletion studies inform receptor usage by virulent hantaviruses in human endothelial cells., Elife 10 (2021), doi:10.7554/eLife.69708.
29. E. Mittier, A. Z. Wec, J. Tynell, P. Guardado-Calvo, J. Wigren-Byström, L. C. Polanco, C. M. O'Brien, M. M. Slough, D. M. Abelson, A. Serris, M. Sakharkar, G. Pehau-Amaudet, R. R. Bakken, J. C. Geoghegan, R. K. Jangra, M. Keller, L. Zeitlin, O. Vapalahti, R. G. Ulrich, Z. A. Bomholdt, C. Ahlm, F. A. Rey, J. M. Dye, S. B. Bradfute, T. Strandin, A. S. Herbert, M. N. E. Forsell, L. M. Walker, K. Chandran, Human antibody recognizing a quaternary epitope in the Puumala virus glycoprotein provides broad protection against orthohantaviruses., Sci. Transl. Med. 14, eabl5399 (2022).
30. A. Lundkvist, Y. Cheng, K. B. Sjölander, B. Niklasson, A. Vaheri, A. Plyusnin, Cell culture adaptation of Puumala hantavirus changes the infectivity for its natural reservoir, Clethrionomys glareolus, and leads to accumulation of mutants with altered genomic RNA S segment., J. Virol. 71, 9515-9523 (1997).
31. J. W. Hooper, T. Larsen, D. M. Custer, C. S. Schmaljohn, A lethal disease model for hantavirus pulmonary syndrome., Virology 289, 6-14 (2001).
32. A. Serris, R. Stass, E. A. Bignon, N. A. Muena, J.-C. Manuguerra, R. K. Jangra, S. Li, K. Chandran, N. D. Tischler, J. T. Huiskonen, F. A. Rey, P. Guardado-Calvo, The hantavirus surface glycoprotein lattice and its fusion control mechanism., Cell 183, 442-456.e16 (2020).
33. M. Sakharkar, C. G. Rappazzo, W. F. Wieland-Alter, C.-L. Hsieh, D. Wrapp, E. S. Esterman, C. I. Kaku, A. Z. Wec, J. C. Geoghegan, J. S. McLellan, R. I. Connor, P. F. Wright, L. M. Walker, Prolonged evolution of the human B cell response to SARS-CoV-2 infection., Sci. Immunol. 6 (2021), doi:10.1126/sciimmunol.abg6916.
34. C. J. Williams, J. J. Headd, N. W. Moriarty, M. G. Prisant, L. L. Videau, L. N. Deis, V. Verma, D. A. Keedy, B. J. Hintze, V. B. Chen, S. Jain, S. M. Lewis, W. B. Arendall, J. Snoeyink, P. D. Adams, S. C. Lovell, J. S. Richardson, D. C. Richardson, MolProbity: more and better reference data for improved all-atom structure validation., Protein Sci. 27, 293-315(2018).
35. F. Sievers, A. Wilm, D. Dineen, T. J. Gibson, K. Karplus, W. Li, R. Lopez, H. McWilliam, M. Remmert, J. Söding, J. D. Thompson, D. G. Higgins, Fast, scalable generation of high-quality protein multiple sequence alignments using Clustal Omega., Mol. Syst. Biol. 7, 539 (2011).
36. A. Z. Wec, A. S. Herbert, C. D. Murin, E. K. Nyakatura, D. M. Abelson, J. M. Fels, S. He, R. M. James, M.-A. de La Vega, W. Zhu, R. R. Bakken, E. Goodwin, H. L. Turner, R. K. Jangra, L. Zeitlin, X. Qiu, J. R. Lai, L. M. Walker, A. B. Ward, J. M. Dye, K. Chandran, Z. A. Bomholdt, Antibodies from a Human Survivor Define Sites of Vulnerability for Broad Protection against Ebolaviruses., Cell 169, 878-890.e15 (2017).
37. Y. Xu, W. Roach, T. Sun, T. Jain, B. Prinz, T.-Y. Yu, J. Torrey, J. Thomas, P. Bobrowicz, M. Vásquez, K. D. Wittrup, E. Krauland, Addressing polyspecificity of antibodies selected from an in vitro yeast presentation system: a FACS-based, high-throughput selection and analytical tool., Protein Eng Des Sel 26, 663-670 (2013).
38. J. WIGREN BYSTRÖM, J. NÄSLUND, F. TRULSSON, M. EVANDER, O. WESULA LWANDE, C. AHLM, G. BUCHT, QUANTIFICATION AND KINETICS OF VIRAL RNA TRANSCRIPTS PRODUCED IN ORTHOHANTAVIRUS INFECTED CELLS., VIROL. J. 15, 18 (2018).
39 X. Robert, P. Gouet, Deciphering key features in protein structures with the new ENDscript server., Nucleic Acids Res. 42, W320-W324 (2014).

1. An isolated antibody that binds to a spike protein of a hantavirus.

2. The isolated antibody of claim 1, wherein the isolated antibody comprises a heavy chain having a variable heavy (V_{H}) region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 1-5, 68, or 69.

3. The isolated antibody of claim 2, wherein the heavy chain comprises one or more complementarity determining regions (CDRs) (HCDRs) comprising or consisting of an amino acid sequence at least about 50%, at least about 55%, at least about 60⁰/, at least about 65%, at least about 70%, at least about 75%, at least about 80%, at least about 85%, at least about 90%, at least about 95%, or at least about 100% identical to the amino acid sequence of SEQ ID NOS: 6-20 or 72-77.

4. The isolated antibody of claim 1, wherein the isolated antibody comprises a light chain having a variable light (V_{L}) region comprising or consisting of an amino acid sequence that is at least about 50%, at least about 55%, at least about 60%, at least about 65%, at least about 70%, at least about 75%, at least about 80%, at least about 85%, at least about 90%, at least about 95%, or at least about 100% identical to the amino acid sequence of one or more of SEQ ID NOS: 21-25, 70, or 81.

5. The isolated antibody of claim 4, wherein the light chain comprises one or more CDRs (LCDRs) comprising or consisting of an amino acid sequence at least about 50%, at least about 55%, at least about 60%, at least about 65%, at least about 70%, at least about 75%, at least about 80%, at least about 85%, at least about 90%, at least about 95%, or at least about 100% identical to the amino acid sequence of SEQ ID NOS: 35-40 or 78-80.

6.-19. (canceled)

20. The isolated antibody of claim 1, wherein the isolated antibody binds to an epitope located in an N-terminal (Gn) subunit (SEQ ID NO: 42) and/or a C-terminal (Gc) subunit (SEQ ID NO: 43) of the spike protein.

21. The isolated antibody of claim 1, wherein the antibody binds to an epitope located in the capping loop of Gn (SEQ ID NO: 41).

22. The isolated antibody of claim 1, wherein the antibody binds to a quaternary epitope encompassing both the Gn subunit and the Gc subunit.

23. The isolated antibody of claim 1, wherein the epitope comprises one or more of E95, Q98 or S101 in the Gn subunit.

24. The isolated antibody of claim 1, wherein the epitope comprises one or more of D682, S830, I844, P852, D959, or S966 in the Gc subunit.

25. The isolated antibody of claim 22, wherein the antibody does not bind to Gn in the absence of Gc.

26. The isolated antibody of claim 20, wherein the epitope comprises one or both of E88 and S101 of the Gn subunit.

27. The isolated antibody of claim 1, wherein the isolated antibody binds to the spike protein of a hantavirus or an epitope of the hantavirus in an environment with an acidic pH.

28. The isolated antibody of claim 1, wherein the isolated antibody neutralizes one or more hantaviruses selected from Puumala virus (PUUV), Andes virus (ANDV), Sin Nombre virus (SNV), Hantaan virus (HTNV), Seoul virus (SEOV), Dobrava-Belgrade virus (DOBV); Bayou virus (BAYV); Black Creek Canal virus (BCCV), Choclo virus (CHOV), Laguna Negra virus (LGNV), Maporal virus (MPRLV), Prospect Hill virus (PHV), and Tula virus (TULV).

29. The isolated antibody of claim 1, wherein the isolated antibody comprises:
a HCDR comprising:
an HCDR1 comprising a sequence of any one of SEQ ID NOS: 6-10, 72, 75 or a variant HCDR1 in which 1, 2, 3, 4, or 5 amino acids are substituted relative to the sequence;
an HCDR2 comprising a sequence of any one of SEQ ID NOS: 11-15, 73, 76 or a variant HCDR2 in which 1, 2, 3, 4, or 5 amino acids are substituted relative to the sequence; and
an HCDR3 comprising a sequence of any one of SEQ ID NOS: 16-20, 74, 77, or a variant HCDR3 in which 1, 2, 3, 4, or 5 amino acids are substituted relative to the sequence; and
a LCDR comprising:
an LCDR1 comprising a sequence of any one of SEQ ID NOS: 82, 26-30, 78, or a variant LCDR1 in which 1, 2, 3, 4, or 5 amino acids are substituted relative to the sequence;
an LCDR2 comprising a sequence of any one of SEQ ID NOS: 31-35, 79 or a variant LCDR2 in which 1, 2, 3, 4, or 5 amino acids are substituted relative to the sequence; and
an LCDR3 comprising a sequence of any one of SEQ ID NOS: 36-40, 80 or a variant LCDR3 in which 1, 2, 3, 4, or 5 amino acids are substituted relative to the sequence.

30.-46. (canceled)

47. A recombinant nucleic acid encoding an antibody according to claim 1.

48. An expression vector comprising a polynucleotide encoding the V_{H} region and/or the V_{L} region of the antibody of claim 1.

49. (canceled)

50. A polypeptide comprising
(1) a V_{H} sequence having at least 70% amino acid sequence identity to SEQ ID NO:1 and/or
(2) a V_{L} sequence having at least 70% amino acid sequence identity to SEQ ID NO:21.

51. (canceled)

52. (canceled)

53. A pharmaceutical composition comprising the isolated antibody of claim 1 and a pharmaceutically acceptable carrier.

54. (canceled)

55. A method of inducing an immune response and/or treating a hantavirus infectious disease, the method comprising administering an effective amount of a pharmaceutical composition of claim 53 to a subject in need thereof.

56.-58. (canceled)


SEQUENCE LISTING (111 sequences)

SEQ ID NO: 1 - protein, 126 aa, synthetic construct
QVQLVESGGGVVQPGRSLRLSCAASGFTFSSYAMHWVRQAPGKGLEWVAVTWFDVSKKDYADSVKGRFTISRDNSKNTLYLQMNSLRAEDTAVYYCARNLIRYSGSYFPVHGMDVWGQGTTVTVSS

SEQ ID NO: 2 - protein, 119 aa, synthetic construct
QVQLQESGPGLVKPSQTLSLTCTVSGGSLSRGGYYWSWIRQHPGKGLEWIGNIFYSGSTYYNPSLKSRVTISVDTSTSQFSLKLNSVTAADTAVYYCARNGRWVTPDYWGQGTLVTVSS

SEQ ID NO: 3 - protein, 128 aa, synthetic construct
QVQLQESGPGLVRPSQTLSLTCTVSGDSIRNSLYYWSWIRQPAGKGLEWIGRIYNSGSINYNPSLKSRVTILVDTSKNQFSLKLSSVTAADTAVYYCARGGYYYDGSGFFLPYYFDYWGQGTLVTVSS

SEQ ID NO: 4 - protein, 117 aa, synthetic construct
EVQLVESGGGLVQPGGSLRLSCAASGFTVSSTYMSWVRQAPGKGLECVSVIYSGGNTYYTDSVKGRFTISRDNSKNTLYLQMNSLRAEDTAVYYCARDLHRSRPGPWGQGTLVTVSS

SEQ ID NO: 5 - protein, 124 aa, synthetic construct
QITLKESGPTLVKPTQTLTLTCTFSGFSLSTSGVGVGWIRQPPGKALEWLALIFWNEDKRYSPSLKTRLTITKDTSKNQVVLTMTNMDPVDTATYYCARRVYSDYGQGAVFDYWGQGTLVTVSS

SEQ ID NO: 6 - protein, 9 aa, synthetic construct
FTFSSYAMH

SEQ ID NO: 7 - protein, 11 aa, synthetic construct
GSLSRGGYYWS

SEQ ID NO: 8 - protein, 11 aa, synthetic construct
DSIRNSLYYWS

SEQ ID NO: 9 - protein, 9 aa, synthetic construct
FTVSSTYMS

SEQ ID NO: 10 - protein, 11 aa, synthetic construct
FSLSTSGVGVG

SEQ ID NO: 11 - protein, 16 aa, synthetic construct
TWFDVSKKDYADSVKG

SEQ ID NO: 12 - protein, 16 aa, synthetic construct
NIFYSGSTYYNPSLKS

SEQ ID NO: 13 - protein, 16 aa, synthetic construct
RIYNSGSINYNPSLKS

SEQ ID NO: 14 - protein, 16 aa, synthetic construct
VIYSGGNTYYTDSVKG

SEQ ID NO: 15 - protein, 16 aa, synthetic construct
LIFWNEDKRYSPSLKT

SEQ ID NO: 16 - protein, 19 aa, synthetic construct
ARNLIRYSGSYFPVHGMDV

SEQ ID NO: 17 - protein, 11 aa, synthetic construct
ARNGRWVTPDY

SEQ ID NO: 18 - protein, 20 aa, synthetic construct
ARGGYYYDGSGFFLPYYFDY

SEQ ID NO: 19 - protein, 11 aa, synthetic construct
ARDLHRSRPGP

SEQ ID NO: 20 - protein, 16 aa, synthetic construct
ARRVYSDYGQGAVFDY

SEQ ID NO: 21 - protein, 111 aa, synthetic construct
DIVMTQSPLSLPVTPGEPASISCRSSQSLLHTYGYNSLDWYLQRPGQSPQLLISLGSYRASGVPDRFSGSGSGTDFTLKISRVEAEDVGVYYCMQALHPFTFGGGTKVEIK

SEQ ID NO: 22 - protein, 110 aa, synthetic construct
QSVLTQPPSASGTPGQRVTISCSGSSSNIGSNTVSWYQQLPGTAPKLLIYSNNQRPSGVPDRFSGSKSGTSGSLAISGLQSEDEADYYCAAWDESLHGPVFGGGTKLTVL

SEQ ID NO: 23 - protein, 108 aa, synthetic construct
EIVLTQSPGTLSLSPGERATLSCRASQSVRNTYLAWYQQKPGQAPRLLIYSASSRATGIPDRFGGSGSGTDFILTISRLEPEDFAVYYCQQYGSEPWTFGQGTKVEIK

SEQ ID NO: 24 - protein, 107 aa, synthetic construct
DIQMTQSPSSVSASVGDRVTITCRASQGLSTWLAWYQQKPGKAPKLLIYAASSLQSGVPSRFSGSGSGTDFTLTISSLQPEDFATYYCQQANTFPPTFGGGTKVEIK

SEQ ID NO: 25 - protein, 107 aa, synthetic construct
DIQMTQSPSSLSASVGDRVTITCRASQGISNYLAWFQQKPGKAPKSLIYAAFNLHSGVPSRFSGSGSGTDFTLTISSLQPEDFATYYCQQYNYYPLTFGGGTKVEIK

SEQ ID NO: 26 - protein, 16 aa, synthetic construct
RSSQSLLHTYGYNSLD

SEQ ID NO: 27 - protein, 13 aa, synthetic construct
SGSSSNIGSNTVS

SEQ ID NO: 28 - protein, 12 aa, synthetic construct
RASQSVRNTYLA

SEQ ID NO: 29 - protein, 11 aa, synthetic construct
RASQGLSTWLA

SEQ ID NO: 30 - protein, 11 aa, synthetic construct
RASQGISNYLA

SEQ ID NO: 31 - protein, 7 aa, synthetic construct
LGSYRAS

SEQ ID NO: 32 - protein, 7 aa, synthetic construct
SNNQRPS

SEQ ID NO: 33 - protein, 7 aa, synthetic construct
SASSRAT

SEQ ID NO: 34 - protein, 7 aa, synthetic construct
AASSLQS

SEQ ID NO: 35 - protein, 7 aa, synthetic construct
AAFNLHS

SEQ ID NO: 36 - protein, 8 aa, synthetic construct
MQALHPFT

SEQ ID NO: 37 - protein, 11 aa, synthetic construct
AAWDESLHGPV

SEQ ID NO: 38 - protein, 9 aa, synthetic construct
QQYGSEPWT

SEQ ID NO: 39 - protein, 9 aa, synthetic construct
QQANTFPPT

SEQ ID NO: 40 - protein, 9 aa, synthetic construct
QQYNYYPLT

SEQ ID NO: 41 - protein, 14 aa, synthetic construct
KGDLAENTQASSTS

SEQ ID NO: 42 - protein, 635 aa, synthetic construct
ARNLNELKMECPHTIRLGQGLVVGSVELPSLPIQQVETLKLESSCNFDLHTSTAGQQSFTKWTWEIKGDLAENTQASSTSFQTKSSEVNLRGLCLIPTLVVETAARMRKTIACYDLSCNQTVCQPTVYLMGPIQTCITTKSCLLSLGDQRIQVNYEKTYCVSGQLVEGICFNPIHTMALSQPSHTYDIMTMMVRCFLVIKKVTSGDSMKIEKNFETLVQKNGCTANNFQGYYICLIGSSSEPLYVPALDDYRSAEVLSRMAFAPHGEDHDIEKNAVSAMRIAGKVTGKAPSTESSDTVQGIAFSGSPLYTSTGVLTSKDDPVYIWAPGIIMEGNHSICEKKTLPLTWTGFISLPGEIEKTTQCTVFCTLAGPGADCEAYSETGIFNISSPTCLINRVQRFRGSEQQIKFVCQRVDMDITVYCNGMKKVILTKTLVIGQCIYTFTSIFSLIPGVAHSLAVELCVPGLHGWATMLLLLTFCFGWVLIPTITMILLKILIAFAYLCSKYNTDSKFRILIEKVKREYQKTMGSMVCEVCQYECETAKELESHRKSCSIGSCPYCLNPSEATTSALQAHFKVCKLTSRFQENLRKSLTVYEPMQGCYRTLSLFRYRSRFFVGLVWCVLLVLELIVWAASA

SEQ ID NO: 43 - protein, 490 aa, synthetic construct
ETQNLNAGWTDTAHGSGIIPMKTDLELDFSLPSSASYTYRRQLQNPANEQEKIPFHLQLSKQVIHAEIQHLGHWMDATFNLKTAFHCYGSCEKYAYPWQTAGCFIEKDYEYETGWGCNPPDCPGVGTGCTACGVYLDKLKSVGKVFKIVSLRYTRKVCIQLGTEQTCKTVDSNDCLITTSVKVCLIGTISKFQPSDTLLFLGPLQQGGLIFKQWCTTTCQFGDPGDIMSTPTGMKCPELNGSFRKKCAFATTPVCQFDGNTISGYKRMIATKDSFQSFNVTEPHISTSALEWIDPDSSLRDHINVIVSRDLSFQDLSETPCQIDLATASIDGAWGSGVGFNLVCTVSLTECSAFLTSIKACDAAMCYGSTTANLVRGQNTIHIVGKGGHSGSKFMCCHDTKCSSTGLVAAAPHLDRVTGYNQADSDKIFDDGAPECGMSCWFKKSGEWILGVLNGNWMVVAVLVVLLILSILLFTLCCPRRPSYRKEHKP

SEQ ID NO: 44 - protein, 31 aa, synthetic construct
PUUVWTWEIKGDLAENTQASSTSFQTKSSEV

SEQ ID NO: 45 - protein, 28 aa, synthetic construct
HTNVVSWRGKADQSQSSQNSFETVSTEV

SEQ ID NO: 46 - protein, 28 aa, synthetic construct
SEOVVIWRKKANQESANQNSFEVVESEV

SEQ ID NO: 47 - protein, 28 aa, synthetic construct
DOBVVSWRKKADKAQAAKDSFETTSSEV

SEQ ID NO: 48 - protein, 31 aa, synthetic construct
ANDVVEWRKKSDTTDTTNAASTTFEAQTKTV

SEQ ID NO: 49 - protein, 30 aa, synthetic construct
SNVVDWTKKSSTTESTNAGATTFEAKTKEI

SEQ ID NO: 50 - protein, 46 aa, synthetic construct
PUUVHGSGIIPMKTDLELDFSLPSSSLRDHINVIVSRDLSFQDLSE

SEQ ID NO: 51 - protein, 46 aa, synthetic construct
HINVHGVGSVPMHTDLELDFSLTSSMLRDHINILVTKDIDFDNLGE

SEQ ID NO: 52 - protein, 46 aa, synthetic construct
SEOVHGVGSVPMHTDLELDFSLPSSMLRDHINIVISKDIDFENLAE

SEQ ID NO: 53 - protein, 46 aa, synthetic construct
DOBVHGVGVVPMHTDLELDFSLPSSMLKDHLNILVTKDIDFENLGE

SEQ ID NO: 54 - protein, 46 aa, synthetic construct
ANDVHGVGEIPMKTDLELDFSLPSSNTRDHVNLVLNRDVSFQDLSD

SEQ ID NO: 55 - protein, 45 aa, synthetic construct
SNVHGVGIIPMKTDLELDFALASSSIKDHINMVLNRDVSFQDLSD

SEQ ID NO: 56 - protein, 50 aa, synthetic construct
PUUVCKTVDSNDCLITTSVKVCLIGTISKEWIDPDSSLRDHINVIVSRDL

SEQ ID NO: 57 - protein, 50 aa, synthetic construct
HTNVCKIIDMNDCFVSRHVKVCIIGTVSKEWKDPDGMLRDHINILVTKDI

SEQ ID NO: 58 - protein, 50 aa, synthetic construct
SEOVCKTIDMNDCFVTRHAKICIIGTVSKEWRDPDGMLRDHINIVISKDI

SEQ ID NO: 59 - protein, 50 aa, synthetic construct
DOBVCKTIDMNDCFVTRHVKVCIIGTVSKEWKDPDGMLKDHLNILVTKDI

SEQ ID NO: 60 - protein, 50 aa, synthetic construct
ANDVCKHIDANDCLVTPSVKVCIVGTVSKEWIDPDGNTRDHVNLVLNRDV

SEQ ID NO: 61 - protein, 49 aa, synthetic construct
SNVCKHIDVNDCLVTPSVKVCMIGTISKEWIDPDSSIKDHINMVLNRDV

SEQ ID NO: 62 - protein, 25 aa, synthetic construct
PUUVCLIGTISKFQPSDTLLFLGPL

SEQ ID NO: 63 - protein, 25 aa, synthetic construct
HTNVCIIGTVSKFSQGDTLLFFGPL

SEQ ID NO: 64 - protein, 25 aa, synthetic construct
SEOVCIIGTVSKFSQGDTLLFLGPM

SEQ ID NO: 65 - protein, 25 aa, synthetic construct
DOBVCIIGTVSKFSQGDTLVFLGPM

SEQ ID NO: 66 - protein, 25 aa, synthetic construct
ANDVCIVGTVSKLQPSDTLLFLGPL

SEQ ID NO: 67 - protein, 24 aa, synthetic construct
SNVCMIGTISKLQPGDTLLFLGPL

SEQ ID NO: 68 - protein, 126 aa, synthetic construct
QVQLVESGGGVVQPGRSLRLSCAASGFTFSSYAMHWVRQAPGKGLEWVAVTWFDVSKKDYADSVKGRFTISRDNSKNTLYLQMNSLRAEDTAVYYCARNLIRYSVSYFPVHGMDVWGQGTTVTVSS

SEQ ID NO: 69 - protein, 126 aa, synthetic construct
QVQLVESGGGVVQPGRSLRLSCAASGFEFSSYAMHWVRQAPGKGLEWVAVTWFDVSKKDYADSVKGRFTISRDNSKNTLYLQMNSLRAEDTAVYYCARNLIRYSVSYFPVHGMDVWGQGTTVTVSS

SEQ ID NO: 70 - protein, 111 aa, synthetic construct
DIVMTQSPLSLPVTPGEPASISCRSSQSLLHTYGYNVLDWYLQRPGQSPQLLISLGSYRASGVPDRFSGSGSGTDFTLKISRVEAEDVGVYYCMQALHPFTFGGGTKVEIK

SEQ ID NO: 71 - protein, 44 aa, synthetic construct
GGSGLVPRGSGGGSGGGSWSHPQFEKGGGTGGGTLVPRGSGTGG

SEQ ID NO: 72 - protein, 8 aa, synthetic construct
GFTFSSYA

SEQ ID NO: 73 - protein, 8 aa, synthetic construct
TWFDVSKK

SEQ ID NO: 74 - protein, 19 aa, synthetic construct
ARNLIRYSVSYFPVHGMDV

SEQ ID NO: 75 - protein, 8 aa, synthetic construct
GFEFSSYA

SEQ ID NO: 76 - protein, 8 aa, synthetic construct
TWFDVSKK

SEQ ID NO: 77 - protein, 19 aa, synthetic construct
ARNLIRYSVSYFPVHGMDV

SEQ ID NO: 78 - protein, 11 aa, synthetic construct
QSLLHTYGYNV

SEQ ID NO: 79 - skipped

SEQ ID NO: 80 - protein, 8 aa, synthetic construct
MQALHPFT

SEQ ID NO: 81 - protein, 111 aa, synthetic construct
DIVMTQSPLSLPVTPGEPASISCRSSQSLLHTYGYNCLDWYLQRPGQSPQLLISLGSYRASGVPDRFSGSGSGTDFTLKISRVEAEDVGVYYCMQALHPFTFGGGTKVEIK

SEQ ID NO: 82 - protein, 16 aa, synthetic construct
RSSQSLLHTYGYNCLD

SEQ ID NO: 83 - protein, 75 aa, synthetic construct
PUUVWTWEVKSDLAENTQASSTSFQTKSHCYGSCEKYAYPWQTAGCFVEKDYEYESGWGCNPADCPGVGTGCTAC

SEQ ID NO: 84 - protein, 72 aa, synthetic construct
HTNVVSWRGKADQSQSSQNSFETVSHCYGACTKYEYPWHTAKCHYERDYQYETSWGCNPSDCPGVGTGCTAC

SEQ ID NO: 85 - protein, 72 aa, synthetic construct
SEOVVIWRKKANQESANQNSFEVVEHCYGACTKYQYPWHTAKCHFEKDYEYENSWACNPPDCPGVGTGCTAC

SEQ ID NO: 86 - protein, 72 aa, synthetic construct
DOBVVSWRKKADKAQAAKDSFETTSHCYGACTKYEYPWHTAKCHFERDFEYENNWGCNPADCPGIGTGCTAC

SEQ ID NO: 87 - protein, 75 aa, synthetic construct
ANDVVEWRKKSDTTDTTNAASTTFEAQTHCYGACQKYSYPWQTSKCFFEKDYQYETGWGCNPGDCPGVGTGCTAC

SEQ ID NO: 88 - protein, 74 aa, synthetic construct
SNVVDWTKKSSTTESTNAGATTFEAKTHCYGECKKYAYPWQTAKCFFEKDYQYETSWGCNPPDCPGVGTGCTAC

SEQ ID NO: 89 - protein, 75 aa, synthetic construct
CHOVVNWAKKSTTTETTNAGATTFEATSHCYGACNKYSYPWQTAKCFFEKDYQYETGWACNPGDCPGVGTGCTAC

SEQ ID NO: 90 - protein, 75 aa, synthetic construct
LGNVLEWRKKAETTDTTQAASTTFEAQTHCYGACKQYSYPWQTAKCFFEKDYQYETGWGCNPGDCPGVGTGCTAC

SEQ ID NO: 91 - protein, 126 aa, synthetic construct
QVQLVESGGGVVQPGRSLRLSCAASGFTFSSGAMHWVRQAPGKGLEWVAVIWYDGSNKYYADSVKGRFTISRDNSKNTLYLQMNSLRAEDTAVYYCARNLIRYSGSYFPVHGMDVWGQGTTVTVSS

SEQ ID NO: 92 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALHPFTFGGGTKVEIK

SEQ ID NO: 93 - protein, 28 aa, synthetic construct
PUUVWTWEVKSDLAENTQASSTSFQTKS

SEQ ID NO: 94 - protein, 25 aa, synthetic construct
HTNVVSWRGKADQSQSSQNSFETVS

SEQ ID NO: 95 - protein, 25 aa, synthetic construct
SEOVVIWRKKANQESANQNSFEVVE

SEQ ID NO: 96 - protein, 25 aa, synthetic construct
DOBVVSWRKKADKAQAAKDSFETTS

SEQ ID NO: 97 - protein, 28 aa, synthetic construct
ANDVVEWRKKSDTTDTTNAASTTFEAQT

SEQ ID NO: 98 - protein, 27 aa, synthetic construct
SNVVDWTKKSSTTESTNAGATTFEAKT

SEQ ID NO: 99 - protein, 28 aa, synthetic construct
CHOVVNWAKKSTTTETTNAGATTFEATS

SEQ ID NO: 100 - protein, 28 aa, synthetic construct
LGNVLEWRKKAETTDTTQAASTTFEAQT

SEQ ID NO: 101 - protein, 24 aa, synthetic construct
VEWRKKSDTTDTTNAASTTFEAQT

SEQ ID NO: 102 - protein, 24 aa, synthetic construct
VEWRKKSDTTDTTNATSTTFEAQT

SEQ ID NO: 103 - protein, 24 aa, synthetic construct
VDWTKKSSTTESTNAGATTFEAKT

SEQ ID NO: 104 - protein, 24 aa, synthetic construct
VDWTKKSNTTESTNAGATTFEAKT

SEQ ID NO: 105 - protein, 24 aa, synthetic construct
VDWTKKSSTNESTNAGATTFEAKT

SEQ ID NO: 106 - protein, 8 aa, synthetic construct
GFTFSSGA

SEQ ID NO: 107 - protein, 15 aa, synthetic construct
IWYDGSNKYYADSVK

SEQ ID NO: 108 - protein, 17 aa, synthetic construct
RNLIRYSGSYFPVHGMD

SEQ ID NO: 109 - protein, 15 aa, synthetic construct
RSSQSLLHSNGYNYL

SEQ ID NO: 110 - protein, 6 aa, synthetic construct
LGSNRA

SEQ ID NO: 111 - protein, 8 aa, synthetic construct
MQALHPFT